(19) United States
(12) Patent Application Publication
(10) Pub. No.: US 2026/0315352 A1
(43) Pub. Date: Oct. 15, 2026

(54) Odor and Colorant Stabilized Compositions

(71) Applicant: Symrise AG, Holzminden (DE)

(72) Inventors: Jürgen CLAUS, Bevern (DE); Ev SUESS, Bevern (DE); Imke MEYER, Bodenwerder (DE); Sabine LANGE, Holzminden (DE); Nikolas BUGDAHN, Holzminden (DE); Ricarda KRÄLING, Holzminden (DE); Daniel BERNDT, Halle (DE); Lukas SCHAAF, Hameln (DE)

(21) Appl. No.: 19/100,755

(22) PCT Filed: Aug. 7, 2023
(86) PCT No.: PCT/EP2023/071787

§ 371 Date: Feb. 3, 2025

(30) Foreign Application Priority Data
Aug. 5, 2022 (WO) PCT/EP2022/072101
Aug. 4, 2023 (WO) PCT/EP2023/071719

(51) Int. Cl. A61K 8/44; A61K 8/02; A61K 8/04; A61K 8/06; A61K 8/49; A61Q 5/02; A61Q 5/12; A61Q 11/00; A61Q 13/00; A61Q 15/00; A61Q 17/04; A61Q 19/08; A61Q 19/10

(52) U.S. Cl. CPC A61K 8/445; A61K 8/0208; A61K 8/042; A61K 8/06; A61K 8/447; A61K 8/49; A61Q 5/02; A61Q 5/12; A61Q 11/00; A61Q 13/00; A61Q 15/00; A61Q 17/04; A61Q 19/08; A61Q 19/10; A61K 2800/522

(57) ABSTRACT

The present invention refers to food, cosmetic or pharmaceutical preparations or homecare products comprising or consisting of a fragrance or aroma/flavour compound and an effective amount of a certain mycosporine-like amino acid compound. Additionally, the present invention relates to the use of such mycosporine-like amino acid compounds for stabilizing a fragrance or aroma/flavour compound in food, cosmetic or pharmaceutical preparations or homecare products. Furthermore, the present invention relates to the use of such mycosporine-like amino acid compounds for boosting the antioxidative effect of an antioxidant in food, cosmetic or pharmaceutical preparations or homecare products. Finally, the present invention relates to a method of stabilizing a fragrance or aroma/flavour compound in food, cosmetic or pharmaceutical preparations or homecare products comprising the admixing of at least one of said mycosporine-like amino acid compound with a fragrance or aroma/flavour compound in said food, cosmetic or pharmaceutical preparations or homecare products or to a method of boosting the antioxidant efficacy in food, cosmetic or pharmaceutical preparations or homecare products comprising the admixing of at least one of said mycosporine-like amino acid compound with an antioxidant in said food, cosmetic or pharmaceutical preparations or homecare products.

### TECHNICAL FIELD

The present invention refers to food, cosmetic or pharmaceutical preparations or homecare products comprising or consisting of a fragrance or aroma/flavour compound and an effective amount of a certain mycosporine-like amino acid compound. Additionally, the present invention relates to the use of such mycosporine-like amino acid compounds for stabilizing a fragrance or aroma/flavour compound in food, cosmetic or pharmaceutical preparations or homecare products. Furthermore, the present invention relates to the use of such mycosporine-like amino acid compounds for boosting the antioxidative effect of an antioxidant in food, cosmetic or pharmaceutical preparations or homecare products. Finally, the present invention relates to a method of stabilizing a fragrance or aroma/flavour compound in food, cosmetic or pharmaceutical preparations or homecare products comprising the admixing of at least one of said mycosporine-like amino acid compound with a fragrance or aroma/flavour compound in said food, cosmetic or pharmaceutical preparations or homecare products or to a method of boosting the antioxidant efficacy in food, cosmetic or pharmaceutical preparations or homecare products comprising the admixing of at least one of said mycosporine-like amino acid compound with an antioxidant in said food, cosmetic or pharmaceutical preparations or homecare products.

### BACKGROUND ART

Food, cosmetic compositions, such as fragrance compositions, pharmaceutical compositions or homecare products, such as cleansing compositions, are extremely sensitive to oxidation from light and heat. For example, in cleansing compositions, the oxidation induces rancidity, discoloration and odour deviations. Normally fragrances and colorants are used to cover the thus obtained unpleasant and typical smell and/or colour of oxidized compounds.

However, those frequently used additives like fragrances and colorants also suffer from instability issues which result in strong odour and colour deviations over time especially when exposed to oxidants like air, UV, light and heat.

For this reason, antioxidants are often used in food, cosmetic or pharmaceutical preparations or homecare products to protect fatty acids, fragrances, aromas/flavours and colorants from oxidation. Based on the same principle, fragrance compositions are also very susceptible to oxidation and show strong deviations in colour and smell. The most widely used antioxidant on the market for odour or colorant protection in food, cosmetic, such as fragrance compositions, or pharmaceutical preparations or homecare products, such as cleansing compositions, is butylated hydroxytoluene, also known as dibutylhydroxytoluene (BHT).

BHT is a lipophilic organic compound, chemically a derivative of phenol. Industrially, BHT is prepared by the reaction of p-cresol (4-methylphenol) with isobutylene (2-methylpropene) catalysed by sulfuric acid. Alternatively, BHT is prepared from 2,6-di-tert-butylphenol by hydroxymethylation or aminomethylation followed by hydrogenolysis.

However, BHT is also known for its toxicity. Currently there is discussion between the link of BHT to cancer risk and asthma. Furthermore, there is an in vitro indication for endocrine disruption. Furthermore, BHT is suspected of triggering allergy-like symptoms. Especially in the use of BHT in cosmetics allergic skin reactions have been reported.

Therefore, there is a need for food, cosmetic or pharmaceutical preparations or homecare products, especially for fragrance or aroma/flavour compositions, which are stabilized against oxidation.

Furthermore, there is a need for new substances, that protect fragrance or aroma/flavours in food, cosmetic or pharmaceutical preparations or homecare products from degradation, in particular oxidation.

Additionally, there is continued interest in the market to improve both the potential and the range of efficiency of antioxidants, either by achieving a higher performance of an antioxidant with the same amounts, or, vice versa, by achieving the same performance with lower amounts of an antioxidant, so that the overall content of antioxidants can be reduced in the ready-for-use formulation.

The object of the present invention was therefore to provide food, cosmetic or pharmaceutical preparations or homecare products that overcome the disadvantages of the prior art.

Specifically, it was the object of the present invention to provide efficient and cost-effective alternatives to BHT which are able to protect fragrance or aroma/flavour compositions from degradation, odour and/or colour deviations.

Finally, it is an object of the present invention, to provide a method of stabilizing a fragrance or aroma component in food, a cosmetic or a pharmaceutical preparation or homecare product.

This object is solved by the subject matter of the present invention which is the use of mycosporine-like amino acid compounds as defined herein for stabilizing food, cosmetic, specifically fragrance compositions, or pharmaceutical preparations or homecare products, specifically cleansing compositions.

Surprisingly it has been found that mycosporine-like amino acid compounds as defined herein stabilizes fragrances or aroma/flavours in food, cosmetic or pharmaceutical preparations or homecare products, preferably in fragrance or aroma/flavour compositions. A stabilization in the meaning of the present invention is to be understood as a stabilization by which fragrances or aroma/flavours in food, cosmetic or pharmaceutical preparations or homecare products, in particular fragrance or aroma/flavour compositions, are protected from degradation by oxygen, temperature and/or light. Therefore, the use of mycosporine-like amino acid compounds protects said formulations from degradation, odour change, i.e. malodour formation, and/or discoloration.

Additionally, the concomitantly combined use of at least one mycosporine-like amino acid compound as defined herein with well-known antioxidants results in a synergistic stabilizing effect against degradation by oxygen, temperature and/or light. The use of such a combination in food, a cosmetic or pharmaceutic preparation or homecare product in turn results in stable formulations with no or merely minor odor and/or colour deviations.

### SUMMARY OF THE INVENTION

In order to accomplish the above problem, the present invention provides in a first aspect a food, cosmetic or pharmaceutical preparation or homecare product, comprising or consisting of
(a) at least one fragrance or aroma compound; and
(a) at least one mycosporine-like amino acid compound,
   represented either by the general formula (V)

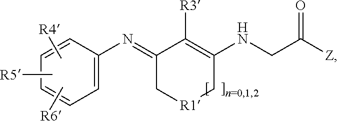

as defined herein, or a tautomer or a salt thereof;
or
represented by the general formula (VI)

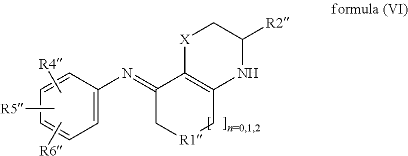

as defined herein, or a tautomer or a salt thereof;
or any mixture of the afore-mentioned compounds.

In a second aspect, the present invention provides for the use of at least one mycosporine-like amino acid compound as defined herein or a mixture thereof for stabilizing a fragrance or aroma compound in food, a cosmetic or pharmaceutical preparation or homecare products.

Finally, the present invention relates in a further aspect to a method of stabilizing a fragrance or aroma compound with an effective amount of at least one mycosporine-like amino acid compound or a mixture thereof, and, thus, reducing or inhibiting food, a cosmetic or a pharmaceutical preparation or a homecare product from fragrance or aroma degradation.

The present invention is specified in the appended claims. The invention itself, and its preferred variants, other objects and advantages, are however also apparent from the following detailed description in conjunction with the accompanying examples and figures.

### DETAILED DESCRIPTION OF THE INVENTION

In the context of the present invention, the following general meanings apply:

As used herein, the singular form “a”, “a” and “the” include plural references unless the context clearly dictates otherwise. Thus, for example reference to “cosmetic preparation”, “the cosmetic preparation” or “a cosmetic preparation” also includes a plurality of cosmetic preparations.

The terms “comprising”, “including”, and “containing” are to be understood as open-ended terms and mean that the named components following said term are essential but not “limited to”, and other components may be added and are still embraced by the present invention.

The term “consisting of” as used according to the present invention means that the total amount of components (a) and (b) adds up to 100% by weight, based on the total weight of the food, cosmetic or pharmaceutical preparation or homecare product, and signifies that the subject matter is closed-ended and can only include the limitations that are expressly recited.

Whenever reference is made to “comprising” it is intended to cover both meanings as alternatives, that is the meaning can be either “comprising” or “consisting of” unless the context dictates otherwise.

The term “or” or “and/or” is used as a function word to indicate that two words or phrases are to be taken together or separately.

The term “optionally” means that the subsequently described compound may but need not to be present in the composition, and that the description includes variants, where the compound is included or variants, where the compound is absent.

The endpoints of all ranges directed to the same component or property are inclusive and independently combinable.

The term “compound(s)” or “compound(s) of the present invention” refers to all compounds encompassed by the structural formulae (V) and/or formula (VI) and/or formula (VII) and/or formula (XIII) and/or formula (IX) and/or formula (X) and/or formula (A) and their sub-formulae disclosed herein and includes each subgenus and all specific compounds within the formula whose structure is disclosed herein.

The compounds may be identified by either their chemical structure and/or chemical name. When the chemical structure and chemical name are in conflict, the chemical structure determines the identity of the compound.

The term “at least one . . . compound” means that the food, cosmetic or pharmaceutical preparation or homecare product according to the present invention, in the following also referred to as “composition”, can comprise either one of said subsequently described individual compound or a mixture of two, three, four, five, six or even more different of said subsequently compounds.

The term “effective amount of a compound” means the amount of compound, that is sufficient to achieve the desired effect or improvement.

With respect to the aroma/flavour or fragrance compound, an effective amount is the amount to impart a perceptible odour or flavour in the fragrance or aroma/flavour composition or in the ready-to-use product. When applied to a combination of aroma/flavour or fragrance substances, the term refers to the total amount of the combination of aroma/flavour or fragrance substances resulting in the flavour or odour.

With respect to the mycosporine-like amino-acid compounds, an effective amount is the amount which is necessary for stabilizing the fragrance or aroma/flavour compounds and protect the composition against degradation.

The term “synergistic effect” is an effect achieved when two different chemical substances or biological structures interact resulting in accumulative overall effect that is greater than the sum of individual effects of any of them. In the present invention, the synergistic effect relates to a synergistic stabilizing or antioxidative effect.

The term “food” in the context of the present invention is any substance or formulation consumed to provide nutritional support for an organism. Food is usually of plant, animal, or fungal origin, and contains essential nutrients, such as carbohydrates, fats, proteins, vitamins or minerals. The substance or formulation is ingested by an organism and assimilated by the organism's cells to provide energy, maintain life, or stimulate growth. The term “food” also encompass food for animals.

The term “cosmetic or pharmaceutical preparations” in the context of the present invention are compositions for cosmetic or pharmaceutical purposes. The term “cosmetic or pharmaceutical preparations” also encompasses sunscreen products, i.e. a photoprotective topical product for the skin including UV-filter compounds that absorbs or reflects some of the sun's ultraviolet (UV) radiation and thus helps protect against sunburn and most importantly prevent skin cancer.

The term “sunscreen composition” or “sunscreen” or “skin-care product”, also known as “sunblock”, “sun cream” or “suntan lotion”, refers to any topical product, which reflects and/or absorbs certain parts of UV radiation and thus helps protect against sunburn and most importantly prevent skin cancer. Thus, the term “sunscreen composition” is to be understood as not only including sunscreen compositions, but also any cosmetic compositions that provide UV protection. According to the present invention the sunscreen composition may comprise one or more active agents, e.g., organic UV-filters, as well as other ingredients or additives, e.g., emulsifiers, emollients, viscosity regulators, stabilizers, preservatives, or fragrances. Sunscreens come as lotions, sprays, gels, foams (such as an expanded foam lotion or whipped lotion), sticks, powders and other topical products. UV-absorbing compounds are used not only in sunscreen, but also in other personal care products, such as lipstick, shampoo, hair spray, body wash, toilet soap, and insect repellent.

The term “homecare products” in the context of the present invention are the essentials for daily care and cleaning purpose in households. They are used for maintaining hygiene and a good aura of the homes. The home care products generally include laundry detergents (powder, liquid and tablet), fabric conditioners, dishwashing detergents (liquid and tablet), hard floor and surface cleaners, glass cleaners, carpet cleaners, oven cleaners, air fresheners, disinfectants, stain removers, car wash products. These products are usually manufactured in the form of a liquid, powder, spray, granules and others.

According to the present invention, the primary object of the present invention is solved by a food, cosmetic, especially dermatological, or pharmaceutical preparation or homecare product (also designated as formulation or composition), comprising or consisting of
(a) at least one fragrance or aroma/flavour compound;
(b) at least one mycosporine-like amino acid compound,
   represented either by the general formula (V)

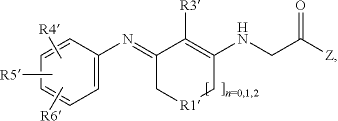

wherein
   Z is selected from the group consisting of O—R2′ or amide′;
   R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
   R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, unsubstituted or substituted heteroaryl;
   amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), and unsubstituted or substituted NR9′R10′, unsubstituted or substituted-N(O-alkyl)(alkl) (Weinreb amide), —N(OH)(H) (hydroxamate), and unsubstituted or substituted-N(OH)(alkyl) (alkylated hydroxamate);
   R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
   the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9′R10′, and unsubstituted or substituted C-spirocycles;
   R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), r substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
   R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or represented by the general formula (VI)

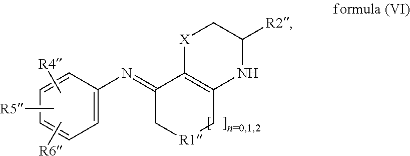

wherein
   R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
   X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
   the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9″R10″, and unsubstituted or substituted C-spirocycles;
   R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
   amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted-N(O-alkyl)(alkyl) (Weinreb amide), —N(OH)(H) (hydroxamate), and unsubstituted or substituted-N(OH)(alkyl) (alkylated hydroxamate);
   R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
   Y is selected from the group consisting of H and unsubstituted or substituted alkyl; and
   R is unsubstituted or substituted alkyl;
or a tautomer or a stereoisomer or a salt of the afore-mentioned compounds;
or any mixture of the afore-mentioned compounds; and
(c) optionally at least one antioxidant.

The component (a) of the food, cosmetic or pharmaceutical preparation or homecare product according to the first aspect of the present invention relates to a fragrance or an aroma/flavour compound or substance, which is commonly used in food, cosmetic preparations, in particular skincare, haircare or body care, or in pharmaceutical preparations or homecare products.

Within the context of the present invention, a fragrance in general is a chemical compound or a class of two or more different chemical compounds to impart an odour or smell.

Within the context of the present invention, an aroma/flavour in general is a chemical compound or class of two or more different chemical compounds to impart a flavour and/or a taste.

Aroma/flavours affect both the sense of taste and smell, whereas fragrances affect only smell.

Additionally, in the context of the present invention, the terms “aroma” or “flavour” are used interchangeable and in an equivalent sense.

The fragrance or aroma/flavouring compound (a) can be chosen from synthetic flavouring liquid and/or oils derived from plants leaves, flowers, fruits and so forth, and combinations thereof. Representative fragrance or aroma/flavouring liquids include artificial, natural or synthetic fruit flavours such as eucalyptus, lemon, orange, banana, grape, lime, apricot and grapefruit oils and fruit essences including apple, strawberry, cherry, orange, pineapple and so forth; bean and nut derived flavours such as coffee, cocoa, cola, peanut, almond and so forth; and root derived flavours such as licorice or ginger.

The food, cosmetic or pharmaceutical preparation or homecare product according to the present invention includes one or more fragrances and/or perfume oils. Suitable perfume oils are mixtures of natural and/or synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert. Butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, betaisomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams.

However, it is preferred to use mixtures of different perfume compounds which, together, produce an agreeable perfume. Other suitable perfume oils are essential oils of relatively low volatility which are mostly used as aroma components. Examples are sage oil, clove oil, melissa oil, mint oil, cinnamon leaf oil, lime-blossom oil, juniper berry oil, vetiver oil, olibanum oil, galbanum oil, and ladanum oil.

Fragrances (a) which are advantageously suitable are listed for example in S. Arctander, Perfume and Flavor Materials, volumes I and II, Montclair, N.J. 1969, private publication, and/or in H. Surburg, J. Panten, Common Fragrance and Flavor Materials, 6th edition, Wiley-VCH, Weinheim 2016. The following list comprises examples of known odorant substances which are advantageously suitable in the present invention:
extracts of natural raw materials such as essential oils, concretes, absolutes, resins, resinoids, balsams, tinctures such as for example: ambergris tincture; amyris oil; angelica seed oil; angelica root oil; anise oil; valerian oil; basil oil; tree moss absolute; bay oil; artemisia oil; benzoin resin; bergamot oil; dihydromyrcenol; lilial; lyral; citronellol; phenylethyl alcohol; hexylcinnamaldehyde; geraniol; benzyl acetone; cyclamen aldehyde; linalool; Boisambrene Forte; Ambroxan; indole; hedione; sandelice; citrus oil; mandarin oil; orange oil; allylamyl glycolate; cyclovertal; lavendin oil; clary oil; damascene; geranium oil bourbon; cyclohexyl salicylate; Vertofix Coeur; Iso-E-Super; Fixolide NP; evernyl; iraldein gamma; phenylacetic acid; geranyl acetate; benzyl acetate; rose oxide; romillat; irotyl, floramat; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; Eucalyptus citriodora oil; eucalyptus oil; fennel oil; pine-needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; blue camomile oil; Roman chamomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; Litsea cubeba oil; bay leaf oil; mace oil; marjoram oil; mandarin oil; massoia bark oil; mimosa absolute; ambrette oil; musk tincture; muscatel sage oil; nutmeg oil; myrrh absolute; myrrh oil; myrtle oil; clove leaf oil; clove bud oil; neroli oil; olibanum absolute; olibanum oil; opopanax oil; orange blossom absolute; orange oil; origanum oil; palmarosa oil; patchouli oil; peri lla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea tree oil; turpentine oil; thyme oil; Tolu balsam; tonka absolute; tuberose absolute; vanilla extract; violet leaf absolute; verbena oil; vetiver oil; juniper berry oil; cognac oil; wormwood oil; Wintergreen oil; ylang ylang oil; hyssop oil; civet absolute; cinnamon leaf oil; cinnamon bark oil, and fractions thereof or constituents isolated therefrom; individual fragrance substances from the group comprising hydrocarbons, such as for example: 3-carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
aliphatic alcohols, such as for example: hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixtures of 3,4,5,6,6-pentamethyl-3,4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and the acetals thereof, such as for example: hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; 1-(1-methoxypropoxy)-(E/Z)-3-hexene;
aliphatic ketones and the oximes thereof, such as for example: 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds, such as for example: 3-methylthio-hexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
aliphatic nitriles, such as for example: 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;
esters of aliphatic carboxylic acids, such as for example: (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl-isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; methyl-3,7-dimethyl-2,6-octadienoate; 4-methyl-2-pentyl-crotonate;
acyclic terpene alcohols, such as for example: citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates acetates, propionates, isobutyrates, butyrates, isovalerates, pentanoates, hexanoates, crotonates, tiglinates and 3-methyl-2-butenoates thereof;
acyclic terpene aldehydes and ketones, such as for example: geranial; neral; citronellal; 7-hydroxy-3,7-dimethyloctanal; 7-methoxy-3,7-dimethyloctanal; 2,6,10-trimethyl-9-undecenal; geranyl acetone; and the dimethyl and diethyl acetals of geranial; neral; 7-hydroxy-3,7-dimethyloctanal;
cyclic terpene alcohols, such as for example: menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;
cyclic terpene aldehydes and ketones, such as for example: menthone; isomenthone; 8-mercaptomenthan-3-one; carvone; camphor; fenchone; alpha-ionone; beta-ionone; alpha-n-methyl ionone; beta-n-methyl ionone; alpha-isomethyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8 (5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedryl ketone);
cyclic alcohols, such as for example: 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol; cycloaliphatic alcohols, such as for example: alpha-3, 3-trimethylcyclohexylmethanol; 1-(4-isopropylcyclohexyl) ethanol; 2-methyl-4-(2,2,3-trimethyl-3-cyclopent-1-yl)butan-ol; 2-methyl-4-(2,2,3-trimethyl-3-cyclopent-1-yl)-2-buten-1-ol; 2-ethyl-4-(2,2,3-tri-methyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl) pen-tan-3-ol; 1-(2,2,6-trimethylcyclohexyl) hexan-3-ol;
cyclic and cycloaliphatic ethers, such as for example: cineole; cedryl methyl ether; cyclododecyl methyl ether; 1,1-dimethoxycyclododecane; (ethoxymethoxy)cyclo-dodecane; alpha-cedrene epoxide; 3a,6,6,9a-tetramethyldodecahydronaphtho[2,1-b]furan; 3a-ethyl-6,6,9a-trimethyldodeca-hydronaphtho[2,1-b]furan; 1,5,9-trimethyl-13-oxabicyclo[10.1,0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones, such as for example: 4-tert-butylcyclohexanone; 2,2,5-trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4 (5H)-indanone; 8-cyclohexadecen-1-one; 9-cycloheptadecen-1-one; cyclopentadecanone; cyclohexadecanone;
cycloaliphatic aldehydes such as for example: 2,4-dimethyl-3-cyclohexene carbaldehyde; 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclo-hexene carbaldehyde;
cycloaliphatic ketones, such as for example: 1-(3,3-dimethyl-cyclohexyl)-4-penten-1-one; 2,2-dimethyl-1-(2,4-dimethyl-3-cyclohexene-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl-(2,4-dimethyl-3-cyclohexen-1-yl) ketone;
esters of cyclic alcohols, such as for example: 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;
esters of cycloaliphatic alcohols, such as for example: 1-cyclohexylethyl crotonate;
esters of cycloaliphatic carboxylic acids, such as for example: allyl-3-cyclohexyl propionate; allylcyclohexyl oxyacetate; cis- and trans-methyl dihydrojasmonate; cis and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolane-2-acetate;
araliphatic alcohols, such as for example: benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl) propanol; 1,1-di methyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl) ethanol;
esters of araliphatic alcohols and aliphatic carboxylic acids, such as for example: benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenyl-ethyl butyrate; cinnamyl acetate; 2-phenoxyethyl isobutyrate; 4-methoxybenzyl acetate;
araliphatic ethers, such as for example: 2-phenyl ethyl methyl ether; 2-phenyl ethyl isoamyl ether; 2-phenyl ethyl 1-ethoxyethyl ether; phenylacetaldehyde dimethylacetal; phenylacetaldehyde diethylacetal; hydratropaldehyde dimethylacetal; phenylacetaldehyde glycerol acetal; 2,4,6-trimethyl-4-phenyl-1,3-dioxane; 4, 4a, 5,9b-tetrahydroindeno[1,2-d]-m-dioxin; 4,4a,5,9b-tetrahydro-2,4-dimethylindeno[1,2-d]-m-dioxin;
aromatic and araliphatic aldehydes, such as for example: benzaldehyde; phenylacetaldehyde; 3-phenylpropanal; hydratropaldehyde; 4-methylbenz-aldehyde; 4-methylphenylacetaldehyde; 3-(4-ethylphenyl)-2,2-dimethylpropanal; 2-methyl-3-(4-isopropylphenyl) propanal; 2-methyl-3-(4-tert-butylphenyl) propanal; 2-methyl-3-(4-isobutylphenyl) propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenz-aldehyde; 4-hydroxy-3-ethoxybenzaldehyde; 3,4-methylenedioxybenzaldehyde; 3,4-dimethoxybenzaldehyde; 2-methyl-3-(4-methoxyphenyl) propanal; 2-methyl-3-(4-methylenedioxyphenyl) propanal;
aromatic and araliphatic ketones, such as for example: acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl) ethanone; 2-benzofuranylethanone; (3-methyl-2-benzofuranyl) ethanone; benzophenone; 1, 1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-5′,6′,7′,8′-tetrahydro-3′,5′,5′,6′,8′,8′-hexamethyl-2-indenyllethanone; acetonaphthone;
aromatic and araliphatic carboxylic acids and the esters thereof, such as for example: benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethylbenzoate; ethyl-3-phenyl glycidate; ethyl-3-methyl-3-phenyl glycidate;
nitrogenous aromatic compounds, such as for example: 2,4,6-trinitro-1,3-dimethyl-5-tert-butylbenzene; 3,5-dinitro-2,6-dimethyl-4-tert-butyl aceto-phenone; cinnamonitrile; 3-methyl-5-phenyl-2-pentenoic acid nitrile; 3-methyl-5-phenylpentanoic acid nitrile; methyl anthranilate; methyl-N-methyl anthranilate; Schiff bases of methyl anthranilate with 7-hydroxy-3,7-dimethyloctanal, 2-methyl-3-(4-tert-butylphenyl) propanal or 2,4-dimethyl-3-cyclohexene carbaldehyde 6-isopropyl quinoline; 6-isobutyl quinoline; 6-sec-butyl quinoline; 2-(3-phenylpropyl)pyridine; indole; skatole; 2-methoxy-3-isopropylpyrazine; 2-isobutyl-3-methoxypyrazine;
phenols, phenyl ethers and phenyl esters, such as for example: estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl) phenol; p-cresyl phenyl acetate;
heterocyclic compounds, such as for example: 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones, such as for example: 1,4-octanolide; 3-methyl-1,4-octanolide; 1,4-nonanolide; 1,4-decanolide; 8-decen-1,4-olide; 1,4-undecanolide; 1,4-dodecan-olide; 1,5-decanolide; 1,5-dodecanolide; 4-methyl-1,4-decanolide; 1,15-penta-decanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin; and any mixture of the above fragrance substances.

Alternatively, the food, cosmetic or pharmaceutical preparation or homecare product according to the first aspect of the present invention encompasses at least one aroma/flavour compound (a) selected from the group consisting of acetophenone, allyl caproate, alpha-ionone, beta-ionone, anisaldehyde, anisyl acetate, anisyl formate, anethole, benzaldehyde, Benzothiazole, benzyl acetate, benzyl alcohol, benzyl benzoate, beta-ionone, butyl butyrate, butyl caproate, butylidenphthalide, carvone, camphene, caryophyllene, cineole, cinnamyl acetate, Citral, Citronellol, Citronellal, citronellyl acetate, cyclohexyl acetate, cymene, damascone, decalactone, dihydrocoumarin, Dimethyl anthranilate, dimethyl anthranilate, dodecalactone, ethoxyethyl acetate, ethyl butyric acid, ethyl butyrate, ethyl caprate, ethyl caproate, ethyl crotonate, ethyl furaneol, ethyl guaiacol, ethyl isobutyrate, ethyl isovalerate, ethyl lactate, ethyl methyl butyrate, ethyl propionate, Eucalyptol, Eugenol, Ethyl heptylate, 4-(p-Hydroxyphenyl)-2-butanone, gamma-decalactone, Geraniol, geranyl acetate, geranyl acetate, grapefruit aldehyde, methyl dihydrojasmonate (e.g. hedione (R)), Heliotropin, 2-heptanone, 3-heptanone, 4-heptanone, trans-2-heptenal, cis-4-heptenal, trans-2-Hexenal, cis-3-Hexenol, trans-2-hexenoic acid, Trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, isobutyl butyrate, isobutyraldehyde, Isoeugenol methyl ether, isopropylmethylthiazole, lauric acid, leavulic acid, Linalool, linalool oxide, linalyl acetate, Menthol, Menthofuran, methyl anthranilate, methylbutanol, methylbutyric acid, 2-methylbutyl acetate, methyl caproate, methyl cinnamate, 5-methylfurfural, 3,2,2-methylcyclopentenolone, 6,5,2-methylheptenone, methyl dihydrojasmonate, methyl jasmonate, 2-methylmethylbutyrate, 2-Methyl-2-pentenoleic acid, methyl thiobutyrate, 3,1-methylthiohexanol, 3-methylthiohexyl acetate, Nerol, neryl acetate, trans, trans-2,4-Nonadienal, 2,4-nonadienol, 2,6-nonadienol, 2,4-nonadienol, nootkatone, Delta-octactone, gamma-octactone, 2-Octanol, 3-Octanol, 1,3-Octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, Piperonal, propionaldehyde, propyl butyrate, pulegone, Pulegol, Sinensal, Sulfurol, terpinene, Terpineol, terpinolene, 8,3-thiomenthanone, 4,4,2-thiomethylpentanone, Thymol, delta-undecalactone, gamma-undecalactone, valencene, valeric acid, Vanillin, Acetoin, ethylvanillin, ethylvanillin isobutyrate (=3-Ethoxy-4-isobutyryloxybenzaldehyde), 2,5-Dimethyl-4-hydroxy-3(2H)-furanone and the derivatives thereof (preferably Homofuraneol (=2-Ethyl-4-hydroxy-5-methyl-3(2H)-furanone), Homofuronol (=2-ethyl-5-methyl-4-hydroxy-3(2H)-furanone and 5-Ethyl-2-methyl-4-hydroxy-3(2H)-furanone), Maltol and maltol derivatives (preferably ethylmaltol), Coumarin and coumarin derivatives, gamma-lactones (preferably gamma-undecalactone, gamma-nonalactone, gamma-decalactone), delta-lactones (preferably 4-methyl-delta-decalactone, massoilactone, delta-decalactone, tuberolactone), methyl sorbate, Divanillin, 4-Hydroxy-2 (or 5)-ethyl-5 (or 2)-methyl-3(2H)-furanone, 2-Hydroxy-3-methyl-2-cyclopentenone, 3-Hydroxy-4,5-dimethyl-2(5H)-furanone, isoamyl acetate, ethyl butyrate, n-butyl butyrate, isoamyl butyrate, ethyl 3-methylbutyrate, ethyl n-hexanoate, allyl n-hexanoate, N-butyl n-hexanoate, ethyl n-octanoate, Ethyl 3-methyl-3-phenylglycidate, Ethyl 2-trans-4-cis-decadienoate, 4-(p-Hydroxyphenyl)-2-butanone, 1,1-Dimethoxy-2,2,5-trimethyl-4-hexane, 2,6-Dimethyl-5-hepten-1-al and phenylacetaldehyde, 2-Methyl-3-(methylthio) furan, 2-Methyl-3-furanthiol, Bis(2-methyl-3-furyl)disulfide, furfuryl mercaptan, Methional, 2-Acetyl-2-thiazoline, 3-Mercapto-2-pentanone, 2,5-Dimethyl-3-furanthiol, 2,4,5-trimethylthiazole, 2-acetylthiazole, 2,4-Dimethyl-5-ethylthiazole, 2-Acetyl-1-pyrroline, 2-Methyl-3-ethylpyrazine, 2-Ethyl-3,5-dimethylpyrazine, 2-Ethyl-3,6-dimethylpyrazine, 2,3-Diethyl-5-methylpyrazine, 3-Isopropyl-2-methoxypyrazine, 3-Isobutyl-2-methoxypyrazine, 2-acetylpyrazine, 2-pentylpyridine, (E,E)-2,4-Decadienal, (E,E)-2,4-Nonadienal, (E)-2-Octenal, (E)-2-Nonenal, 2-undecenal, 12-methyltridecanal, 1-Penten-3-one, 4-Hydroxy-2,5-dimethyl-3(2H)-furanone, Guaiacol, 3-Hydroxy-4,5-dimethyl-2(5H)-furanone, 3-Hydroxy-4-methyl-5-ethyl-2(5H)-furanone, cinnamaldehyde, cinnamyl alcohol, methyl salicylate, Isopulegol, sugar alcohols (e.g. Erythritol, Threitol, Arabitol, ribitol, Xylitol, Sorbitol, Mannitol, Dulcitol, Lactitol); proteins (e.g. Miraculin, Monellin, Thaumatin, Curculin, Brazzein); sugar substitutes/bulk sweetener (e.g. Magap, sodium cyclamate, acesulfame K, neohesperidine dihydrochalcone, Saccharin sodium salt, aspartame, superaspartame, neotame, alitame, Sucralose, steviosides, rebaudiosides, Lugdarme, Carrelame, Sucrononate, Sucrooctate, Monatin, phenylodulcine); SUESS-TASTING amino acids (e.g. glycine, D-leucine, D-threonine, D-asparagine, D-phenylalanine, D-tryptophn, L-proline); SUESS-TASTING low molecular weight substances (e.g. Hernandulcin, dihydrochalcone glycosides, Glycyrrhizin, glycerrhetinic acid, derivatives and salts thereof, extracts of lacritz (Glycyrrhizza glabra ssp.), Lipia dulcis extracts, Momordica ssp. Extracts), plant extracts (e.g. Momordica grosvenori [Luo Han Guo] and the mogrosides obtained therefrom, Hydrangea dulcis or Stevia ssp. (for example Stevia rebaudiana) extracts or steviosides obtained therefrom, and any mixture of the above aroma/flavour substances.

The above specified fragrances (a) are used in the food, cosmetic or pharmaceutical preparation or homecare product either individually or in the form of mixtures of one, two, three, four and even more fragrances.

According to the present invention, the food, cosmetic or pharmaceutical preparations or homecare products comprise at least one fragrance or aroma/flavour compound, preferably two, three, four, five, six, seven, eight or more fragrance or aroma/flavour compounds. Preferably, the fragrance or aroma/flavour is a fragrance composition comprising a mixture of multiple fragrance compounds or an aroma/flavour composition comprising a mixture of multiple aroma/flavour compounds.

In the context of the present invention, therefore the term “fragrance” encompasses similarly “fragrance composition” and the term “aroma/flavour” encompasses similarly “aroma/flavour composition”.

The above specified fragrances or aroma/flavour substances can also be used in encapsulated form. Suitable coating materials here are, for example, starches, including the degradation products thereof and chemically or physically produced derivatives (especially dextrins and maltodextrins), gelatin, gum 27rabic, Agar agar, Ghatti Gum, Gellan Gum, modified and unmodified celluloses, Pullulan, Curdlan, carrageenans, alginic acids, alginates, pectin, Inulin, Xanthan Gum and mixtures of two or more of these substances.

Due to their chemical structure and functionalities, the above specified fragrance or aroma/flavour substances are susceptible against oxidation, temperature, and/or light and degrade easily which results into odour change, i.e. malodour formation, and/or discoloration in the formulations according to the present invention.

The component (b) in the food, cosmetic or pharmaceutical preparation or homecare product according to the first aspect of the present invention is at least one mycosporine-like amino acid compound represented either by the general formula (V)

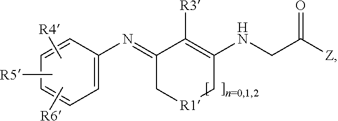

as defined herein;
or
represented by the general formula (VI)

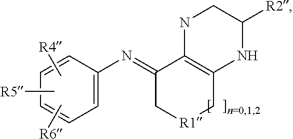

as defined herein.

Mycosporine-like amino are small secondary acids (MAAs) metabolites produced by organisms that live in environments with high volumes of sunlight, usually marine environments. The MAAs are imine derivatives of mycosporines and contain an amino-cyclohexene-imine ring linked to an amino acid, amino alcohol or amino group. Said compounds are known to demonstrate solar radiation-absorbing characteristics, or UV-protection properties. The compounds are capable of electron delocalization. In addition, said compounds demonstrate antioxidant qualities.

Unless stated otherwise, in the context of the present invention, especially for the definition of the mycosporine-like amino acid compounds represented by any of the general formulae the following general meanings apply:

The term “halogen” residue/moiety or group alone or as part of another substituent according to the present invention refers to F, Cl, Br or I.

The term “alkyl” alone or as part of another substituent according to the present invention refers to a saturated or mono- or polyunsaturated linear or branched monovalent hydrocarbon radical obtained by removing a hydrogen atom from a single carbon atom of a corresponding parent alkane.

In a preferred variation, the term “alkyl” also includes any alkyl moieties in radicals derived therefrom, such as alkoxy, alkylthio, alkylsulphonyl saturated linear or branched hydrocarbon radicals having 1 to 10, 1 to 8, 1 to 6, or 1 to 4 carbon atoms.

If the alkyl radical is further bonded to another atom, it becomes an alkylene radical or alkylene group. In other words, the term “alkylene” also refers to a divalent linear or branched alkyl. For example, —CH₂CH₃ is an ethyl, while-CH₂CH₂— is an ethylene.

The term “alkylene” alone or as part of another substituent refers to a saturated linear or branched divalent hydrocarbon radical obtained by removing two hydrogen atoms from a single carbon atom or two different carbon atoms of a starting alkane.

In preferred variants according to the present invention, the linear or branched alkyl group or alkylene group comprises 1 to 10 carbon atoms. In other still more preferred variants, the linear or branched alkyl group or alkylene group comprises 1 to 6 carbon atoms.

More preferred according to the invention are saturated linear or branched C1 to C6 alkyl groups or saturated linear or branched C1 to C6 alkylene groups.

Most preferred the linear or branched alkyl groups or alkylene groups with 1 to 4 carbon atoms.

Preferred alkyl radicals/moieties or alkyl groups include, but are not limited to: C1 to C6 alkyl comprising methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

In a preferred variant, the alkyl radical/moiety is selected from the group consisting of methyl, ethyl, propyl, isopropyl, butyl, isobutyl and tert-butyl, more preferred from the group consisting of methyl and ethyl.

The alkyl group or alkylene group as defined above may further be substituted.

The term “alkyl” or “alkylene” further includes radicals or groups having any degree of saturation, i.e., groups having only single carbon-carbon bonds (“alkyl” or “alkylene”), groups having one or more double carbon-carbon bonds (“alkenyl”), radicals having one or more triple carbon-carbon bonds (“alkynyl”), and groups having a mixture of single, double and/or triple carbon-carbon bonds.

The term “alkenyl” alone or as part of another substituent according to the present invention refers to an unsaturated linear or branched monovalent hydrocarbon radical having at least one carbon-carbon double bond (C═C double bond). The radical may be in either the cis or trans conformation around the double bond(s). So that the term “alkenyl” also includes the corresponding cis/trans isomers.

In preferred variants according to the present invention, the linear or branched alkenyl group comprises 2 to 10 carbon atoms. In other preferred variants, the linear or branched alkenyl group comprises 2 to 6 carbon atoms.

In still further preferred variants, the linear or branched alkenyl group comprises 2 to 4 carbon atoms.

Preferred according to the invention are mono- or di-unsaturated linear or branched C1 to C6 alkenyl groups.

Typical alkenyl radicals or alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl and the like.

The alkenyl group as defined above may further be substituted.

The term “alkynyl” alone or as part of another substituent according to the present invention refers to an unsaturated linear or branched monovalent hydrocarbon radical having at least one carbon-carbon triple bond (C≡C triple bond).

In preferred variants according to the present invention, the linear or branched alkynyl group comprises 2 to 10 carbon atoms. In other preferred variants, the alkynyl group comprises 2 to 6 carbon atoms. In still further preferred variants, the alkynyl group comprises 2 to 4 carbon atoms.

Most preferred according to the invention are mono- or di-unsaturated linear or branched C1 to C6 alkynyl groups.

Typical alkynyl radicals/moieties or alkynyl groups include, but are not limited to, ethynyl; propynyls such as prop-1-yn-1-yl, prop-2-in-1-yl, etc.; butynyls such as but-1-in-1-yl, but-1-in-3-yl, but-3-in-1-yl, and the like.

The alkynyl group as defined above may further be substituted. The alkyl group or alkylene group as defined above may further be substituted.

The term “alkoxy” alone or as part of another substituent according to the present invention refers to a linear or branched radical of the formula —O—R, where R is alkyl or substituted alkyl, as defined herein.

In preferred variants according to the present invention, the linear or branched alkoxy group comprises 2 to 10 carbon atoms. In other preferred variants, the linear or branched alkoxy group comprises 2 to 6 carbon atoms. In still further preferred variants, the linear or branched alkoxy group comprises 2 to 4 carbon atoms.

Most preferred according to the invention are linear or branched C1 to C6 alkoxy groups.

Typical alkoxy radicals/moieties or alkoxy groups include C1 to C6 alkoxy comprising C1 to C4 alkoxy such as. methoxy, ethoxy, n-propoxy, 1-methylethoxy, butoxy, 1-methylpropoxy, 2-methylpropoxy or 1,1-dimethylethoxy; as well as pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

In a preferred variant, the alkoxy radical or alkoxy group is selected from the group consisting of methoxy (—O-methyl), ethoxy (—O-ethyl), propoxy (—O-propyl) and butoxy (—O-butyl), more preferred from the group consisting of methoxy (—O-methyl) and ethoxy (—O-ethyl).

The alkoxy group or alkylene group as defined above may further be substituted.

The term “alkylthio” or “thioalkoxy” alone or as part of another substituent according to the present invention refers to a radical of the formula —S—R, wherein R is alkyl or substituted alkyl, as defined herein.

According to the invention, the term “alkyl” or “alkylene” also includes heteroalkyl radicals or heteroalkyl groups. The term “heteroalkyl” by itself or as part of other substituents refers to alkyl groups in which one or more of the carbon atom(s) is/are independently replaced by the same or another heteroatom or by the same or another heteroatomic group(s). Typical heteroatoms or heteroatomic groups that may replace the carbon atoms include, but are not limited to, —O—, —S—, —N—, —Si—, —NH—, —S(O)—, —S(O)₂—, —S(O)NH—, —S(O)₂NH—, and the like, and combinations thereof. The heteroatoms or heteroatomic groups may be located at any internal position of the alkyl group.

Typical heteroatomic groups that may be included in these groups include, but are not limited to, —O—, —S—, —O—O—, —S—S—, —O—S—, —NRR—, ═NN═, —N═N—, —N═N—NRR, —PR—, —P(O)₂—, —POR—, —O—P(O)₂—, —SO—, —SO₂—, —SR₂OR—, and the like, wherein R is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl as defined herein.

The alkyl group or alkylene group as defined above may further be substituted.

The term “acyl” or “alkanoyl” alone or as part of another substituent according to the present invention refers to a radical R—C(═O)—, wherein R is hydrogen, unsubstituted or substituted alkyl, unsubstituted substituted cycloalkyl, unsubstituted or substituted aryl, arylalkyl, unsubstituted or substituted arylalkyl, unsubstituted or substituted heteroalkyl, unsubstituted or substituted heteroarylalkyl, as defined herein.

Representative examples include, but are not limited to, formyl, acetyl, propionyl, butyryl, valeryl, benzoyl, cyclohexylcarbonyl, cyclohexylmethylcarbonyl, benzylcarbonyl, and the like.

The term “cycloalkyl” alone or as part of another substituent according to the present invention refers to a saturated or mono- or polyunsaturated, non-aromatic, cyclic monovalent hydrocarbon radical in which the carbon atoms are ring-linked and which has no heteroatom.

The carbon ring can occur as a monocyclic compound, which has only a single ring, or as a polycyclic compound, which has two or more rings.

In one preferred variation, the term “cycloalkyl” includes a three- to ten-membered monocyclic cycloalkyl radical or cycloalkyl group or a nine- to twelve-membered polycyclic cycloalkyl radical or cycloalkyl group. In other still more preferred variants, the cycloalkyl moiety comprises a five-, six- or seven-membered monocyclic cycloalkyl moiety or a nine- to twelve-membered bicyclic cycloalkyl moiety.

In a preferred embodiment according to the present invention, a cycloalkyl radical or group comprises 3 to 20 carbon atoms. In an even more preferred embodiment, a cycloalkyl radical comprises 6 to 15 carbon atoms. In a most preferred embodiment, a cycloalkyl radical comprises 6 to 10 carbon atoms. Most preferred are monocyclic C3 to C7 cycloalkyl groups.

Typical cycloalkyl radicals or cycloalkyl groups include, but are not limited to, saturated carbocyclic radicals having 3 to 20 carbon atoms, such as C3 to C12 carbocyclyl, comprising cyclopropyl, cyclobutyl, cyclopentyl, cyclohexyl, cycloheptyl, cyclooctyl, cyclononyl, cyclodecyl, cycloundecyl, and cyclododecyl; cyclopentyl, cyclohexyl, cycloheptyl, as well as cyclopropyl-methyl, cyclopropyl-ethyl, cyclobutyl-methyl, cyclobutyl-ethyl, cyclopentyl-methyl, cyclopentyl-ethyl, cyclohexyl-methyl, or _{C3}- to C7-carbocyclyl comprising cyclopropyl, cyclobutyl, cyclopentyl, cyclohexyl, cycloheptyl, cyclopropyl-methyl, cyclopropyl-ethyl, cyclobutyl-methyl, cyclopentyl-ethyl, cyclohexyl-methyl, cyclobut-1-en-1-yl, cyclobut-1-en-3-yl, 3-dien-1-yl and the like.

Polycyclic cycloalkyl radicals or cycloalkyl groups preferred according to the invention include but are not limited to naphthyl, indenyl, groups and the like.

According to the invention, the term “cycloalkyl” also includes cycloalkenyls, i.e. unsaturated cyclic hydrocarbon radicals containing C═C double bonds between two carbon atoms of the ring molecule. In a broader sense, cycloalkenyls are compounds with one, two or more double bond(s), where the number of possible, mostly conjugated double bonds in the molecule depends on the ring size.

Typical cycloalkenyls include, but are not limited to, cyclopropenyl, cyclopentenyl, cyclohexenyl, cyclopentadienyl, and the like.

According to the invention, the term “cycloalkyl” further includes cycloalkynyls, i.e. unsaturated, —C≡C-triple bonds, containing cyclic hydrocarbon radicals between two carbon atoms of the ring molecule, the triple bond depending on the ring size for reasons of ring tension.

Typical cycloalkynyles include cyclooctin.

The cycloalkyl, cycloalkenyl or cycloalkynyl moieties or groups, as defined above, may further be substituted.

The term “aryl” alone or as part of another substituent according to the present invention refers to a monovalent aromatic hydrocarbon radical derived by removing a hydrogen atom from a single carbon atom of an aromatic ring SY-stem.

In one preferred variation, the term “aryl” includes a three- to ten-membered monocyclic aryl radical or aryl group or a nine- to twelve-membered polycyclic aryl radical or aryl group. In other still more preferred variants, the carboaryl radical comprises a five-, six- or seven-membered monocyclic carboaryl radical or a nine- to twelve-membered bicyclic carboaryl radical.

In a preferred embodiment according to the present invention, the aryl radical comprises 3 to 20 carbon atoms. In a preferred variation, the aryl moiety comprises 6 to 15 ring atoms. In an even more preferred embodiment, an aryl radical comprises 6 to 10 carbon atoms.

Most preferred according to the invention are monocyclic C3 to C10 aryl groups. Most preferred are monocyclic C3 to C7 aryl groups.

Typical aryl radicals include, without being limited thereto, benzene, phenyl, biphenyl, naphthyl such as 1- or 2-naphthyl, tetrahydronaphthyl, fluorenyl, indenyl, and phenanthrenyl. Typical carboaryl moieties further include, but are not limited to, groups derived from aceanthrylene, acenaphthylene, acephenanthrylene, anthracene, azulene, benzene, chrysene, corone, fluoranthene, fluorene, hexacene, hexaphene, hexalene, as-indacene, S-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

Aromatic polycyclic aryl radicals or aryl groups preferred according to the invention include, but are not limited to, naphthalene, biphenyl and the like.

The aryl moiety or group, as defined above, may further be substituted.

The term “arylalkyl” alone or as part of another substituent according to the present invention refers to an acyclic alkyl group in which one of the hydrogen atoms attached to a carbon atom, typically a terminal or sp carbon atom, is replaced by an aryl group as defined herein. In other words, arylalkyl may also be considered as alkyl substituted by aryl. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like.

The term “heteroarylalkyl” alone or as part of another substituent refers to a cyclic alkyl group in which one of the hydrogen atoms attached to a carbon atom is replaced by a heteroaryl group. In a preferred embodiment according to the present invention, the heteroarylalkyl group is a 6- to 20-membered heteroarylalkyl, e.g., the alkyl, alkenyl, or alkynyl group of the heteroarylalkyl is a C1- to C6-alkyl and the heteroaryl group is a 5- to 15-membered heteroaryl group. In other embodiments, the heteroarylalkyl is a 6- to 13-membered heteroarylalkyl, e.g., the alkyl, alkenyl, or alkynyl group is a C1- to C3-alkyl and the heteroaryl group is a 5 to 10-membered heteroaryl.

The term “heterocycloalkyl” alone or as part of another substituent according to the present invention refers to a saturated or mono- or polyunsaturated, non-aromatic, cyclic monovalent hydrocarbon radical in which one or more carbon atom(s) is/are independently replaced by the same or a different heteroatom. Typical heteroatoms to replace the carbon atom(s) include, but are not limited to, N, P, O, S, Si, etc. Typical heterocycloalkyl groups include, without being limited thereto, groups derived from epoxides, azirines, thiiranes, imidazolidine, morpholine, piperazine, piperidine, pyrazolidine, pyrrolidone, quinuclidine and the like.

In a preferred embodiment according to the present invention, the heterocycloalkyl moiety or group comprises 3 to 20 ring atoms. In a preferred variation, the heterocycloalkyl moiety comprises 6 to 15 ring atoms. In an even more preferred embodiment, the heterocycloalkyl moiety comprises 6 to 10 carbon atoms.

The heterocycloalkyl moiety can occur as a monocyclic compound, which has only a single ring, or as a polycyclic compound, which has two or more rings, such as bicyclic, tricyclic or spirocyclic.

Preferably, the term “heterocycloalkyl” includes three- to seven-membered, saturated or mono- or polyunsaturated heterocycloalkyl moieties comprising one, two, three or four heteroatoms selected from the group consisting of O, N and S. The heteroatom or heteroatoms may occupy any position in the heterocycloalkyl ring.

In one preferred variation, the term “heterocycloalkyl” includes a three- to ten-membered monocyclic heterocycloalkyl radical or a nine- to twelve-membered polycyclic heterocycloalkyl radical. In other still more preferred variants, the heterocycloalkyl moiety comprises a five-, six- or seven-membered monocyclic heterocycloalkyl moiety or a nine- to twelve-membered bicyclic heterocycloalkyl moiety.

Most preferred according to the invention are monocyclic heterocycloalkyl radicals comprising 3 to 12 carbon atoms. Most preferred are monocyclic heterocycloalkyl radicals having 5 to 7 ring atoms.

Typical heterocycloalkyl moieties include, but are not limited to: Five- or six-membered, saturated or monounsaturated heterocycloalkyl containing one or two nitrogen atoms and/or one oxygen or sulphur atom or one or two oxygen and/or sulphur atoms as ring members comprising 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-Isoxazolidinyl, 4-Isoxazolidinyl, 5-Isoxazolidinyl, 3-Isothiazolidinyl, 4-Isothiazolidinyl, 5-Isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-Imidazolidinyl, 4-Imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl and the like.

The heterocycloalkyl moiety or group, as defined above, may further be substituted.

The term “heteroaryl” by itself or as part of another substituent according to the present invention refers to a monovalent heteroaromatic radical obtained by removing a hydrogen atom from a single atom of a heteroaromatic ring SY-stem. Typical heteroaryl radicals, or. Heteroaryl groups include, but are not limited to, those derived from acridine, β-carboline, chroman, chromium, cinnoline, furan, imidazole, indazole, indole, indoline, indolizine, isobenzofuran, isochromium, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, thiazole, thiophene, triazole, xanthene and the like are derived.

The heteroaryl moiety can occur as a monocyclic compound having only a single ring or as a polycyclic compound having two or more rings.

In one preferred variation, the term “heteroaryl” includes a three- to ten-membered monocyclic heteroaryl radical or a nine- to twelve-membered polycyclic heteroaryl radical. In other still more preferred variants, the heteroaryl moiety comprises a five-, six- or seven-membered monocyclic heteroaryl moiety or a nine- to twelve-membered bicyclic heteroaryl moiety.

Preferably, the term “heteroaryl” includes three- to seven-membered monocyclic heteroaryl radicals comprising one, two, three or four heteroatoms selected from the group consisting of O, N and S. The heteroatom or heteroatoms may occupy any position in the heteroaryl ring.

In a preferred embodiment according to the present invention, the heteroaryl moiety or group comprises 3 to 20 ring atoms. In an even more preferred variant, the heteroaryl moiety comprises 6 to 15 ring atoms. In a most preferred embodiment, the heteroaryl group comprises 6 to 10 ring atoms. Most preferred according to the invention are monocyclic _{C3} to C7 heteroaryl groups.

Particularly preferred heteroaryl moieties or heteroaryl groups include, but are not limited to, those derived from furan, thiophene, pyrrole, benzothiophene, benzofuran, benzimidazole, indole, pyridine, pyrazole, quinoline, imidazole, oxazole, isoxazole, and pyrazine.

Five-membered aromatic heteroaryl radicals containing, in addition to carbon atoms, one, two or three nitrogen atoms or one or two nitrogen atoms and one sulfur or oxygen atom as ring atoms include 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-Imidazolyl, 4-Imidazolyl, and 1,3,4-triazol-2-yl.

Five-membered aromatic heteroaryl radicals containing one, two, three or four nitrogen atoms as ring atoms include 1-, 2- or 3-pyrrolyl, 1-, 3- or 4-pyrazolyl, 1-, 2- or 4-Imidazolyl, 1,2,3-[1H]-triazol-1-yl, 1,2,3-[2H]-triazol-2-yl, 1,2,3-[1H]-triazol-4-yl, 1,2,3-[1H]-triazol-5-yl, 1,2,3-[2H]-triazol-4-yl, 1,2,4-[1H]-triazol-1-yl, 1,2,4-[1H]-triazol-3-yl, 1,2,4-[1H]-triazol-5-yl, 1,2,4-[4H]-triazol-4-yl, 1,2,4-[4H]-triazol-3-yl, [1H]-tetrazol-1-yl, [1H]-tetrazol-5-yl, [2H]-tetrazol-2-yl, [2H]-tetrazol-5-yl and the like.

Five-membered aromatic heteroaryl radicals containing a heteroatom selected from oxygen or sulphur and optionally one, two or three nitrogen atoms as ring atoms include 2-furyl, 3-furyl, 2-thienyl, 3-thienyl, 3- or 4-Isoxazolyl, 3- or 4-isothiazolyl, 2-, 4- or 5-oxazolyl, 2-, 4- or 5-thiazolyl, 1,2,4-thiadiazol-3-yl, 1,2,4-thiadiazol-5-yl, 1,3,4-thiadiazol-2-yl, 1,2,4-oxadiazol-3-yl, 1,2,4-oxadiazol-5-yl and 1,3,4-oxadiazol-2-yl.

Six-membered heteroaryl radicals containing, in addition to carbon atoms, one or two or one, two or three nitrogen atoms as ring atoms, and comprising, for example. 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

The heteroaryl moiety or group, as defined above, may further be substituted.

The term “C-spirocycles” in the context of the present application means compounds that have at least two molecular rings with only one common atom. The simplest spiro compounds are bicyclic (having just two rings) or have a bicyclic portion as part of the larger ring system, in either case with the two rings connected through the defining single common atom. The one common atom connecting the participating rings distinguishes spiro compounds from other bicyclic structures: from isolated ring compounds like biphenyl that have no connecting atoms, from fused ring compounds like decalin having two rings linked by two adjacent atoms, and from bridged ring compounds like norbornane with two rings linked by two non-adjacent atoms. Spiro compounds may be fully carbocyclic (all carbon) or heterocyclic (having one or more non-carbon atom, such as N, O and S). The common atom that connects the two (or sometimes three) rings is called the spiro atom. Preferably, the spiro atom is a carbon atom. More preferably, the C-spirocycles compound means compounds that are fully carbocyclic (all carbon).

The term “substituted” in the context of the present invention means that one or more hydrogen atoms of the indicated radical or group is/are independently replaced by the same or a different substituent(s). Additionally, the term “substituted” specifically provides for one or more, i.e., two, three, four, five, six or more, substitutions commonly used in the art. However, it is generally known that the substituents should be selected so that they do not adversely affect the useful properties of the compound or its function.

Suitable substituents in the context of the present invention preferably include halogen, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy groups or heteroaryloxy groups, arylalkyl or heteroarylalkyl groups, arylalkoxy or heteroarylalkoxy groups, amino groups, alkyl and dialkylamino groups, carbamoyl groups, alkylcarbonyl groups, carboxyl groups, alkoxycarbonyl groups, alkylaminocarbonyl groups, dialkylaminocarbonyl groups, arylcarbonyl groups, aryloxycarbonyl groups, alkylsulfonyl groups, arylsulfonyl groups, cycloalkyl groups, cyano groups, C1 to C6 alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof.

Substituents or substituent groups useful for substituting saturated carbon atoms in the indicated group or radical more preferably include, but are not limited to, halogen, hydroxyl, alkyl, alkenyl, alkynyl, alkoxyl, —NH₂, amino (primary, secondary, or tertiary), nitro, thiol, thioether, imine, cyano, amido, phosphonato, phosphine, carboxyl, thiocarbonyl, sulfonyl, sulfonamide, ketone, aldehyde, ester, acetyl, acetoxy, carbamoyl, oxygen (O); haloalkyl (e.g., trifluoromethyl); aminoacyl and aminoalkyl, carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g. pyrrolidinyl, piperidinyl, piperazinyl, morpholinyl, or thiazinyl), carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl), —CO₂CH₃, —CONH₂, —OCH₂CONH₂; —SO₂NH₂, —OCHF₂, —CF₃, —OCF₃.

According to the invention, the substituents used to replace a particular radical or radical may in turn be further substituted, typically with one or more of the same or different radicals selected from the various groups indicated above and as defined in detail above. In the case of substituted combinations such as substituted arylalkyl, either the aryl or the alkyl group may be substituted, or both the aryl and the alkyl group may be substituted with one or more substituents.

In a preferred variant, Z in the general formula (V) is either —O—R2′ or amide′.

In a further preferred variant, R1′ in the general formula (V) is CH₂.

In a further preferred variant, R1′ in the general formula (V) is C(alkyl)₂.

In a further preferred variant, R1′ in the general formula (V) is O.

In a further preferred variant, R1′ in the general formula (V) is S.

In a further preferred variant, R1′ in the general formula (V) is SO.

In a further preferred variant, R1′ in the general formula (V) is SO₂.

In a further preferred variant, R1′ in the general formula (V) is NH.

In a still further preferred variant, R1′ in the general formula (V) is N(alkyl).

Most preferred the R1′ in the general formula (V) is either CH₂ or C(alkyl)₂.

In a preferred variant, R2′ in the general formula (V) is H.

In a further preferred variant, R2′ in the general formula (V) is methyl.

In a further preferred variant, R2′ in the general formula (V) is ethyl.

In a further preferred variant, R2′ in the general formula (V) is propyl.

In a further preferred variant, R2′ in the general formula (V) is isopropyl.

In a further preferred variant, R2′ in the general formula (V) is butyl.

In a further preferred variant, R2′ in the general formula (V) is isobutyl.

In a further preferred variant, R2′ in the general formula (V) is tert-butyl.

In a further preferred variant, R2′ in the general formula (V) is

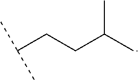

In a further preferred variant, R2′ in the general formula (V) is

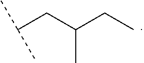

In a preferred variant, R2′ in the general formula (V) is

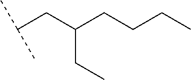

(2-ethyl-hexyl).

In a still further preferred variant, R2′ in the general formula (V) is phenyl.

Most preferred the R2′ in the general formula (V) is either H or ethyl or isopropyl or 2-ethylhexyl or phenyl.

In a preferred variant, amide′ in the general formula (V) is NH₂.

In a further preferred variant, amide′ in the general formula (V) is NH(alkyl), wherein alkyl is selected from the group consisting of methyl, ethyl, propyl, isopropyl, butyl, isobutyl, and tert-butyl.

In a further preferred variant, amide′ in the general formula (V) is N(alkyl)₂, wherein alkyl is selected from the group consisting of methyl, ethyl, propyl, isopropyl, butyl, isobutyl, and tert-butyl.

In a further preferred variant, amide′ in the general formula (V) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide′ in the general formula (V) is —N(OH)(H) (hydroxamate).

Most preferred the amide′ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a preferred variant, R3′ in the general formula (V) is H.

In a further preferred variant, R3′ in the general formula (V) is methyl.

In a further preferred variant, R3′ in the general formula (V) is ethyl.

In a further preferred variant, R3′ in the general formula (V) is —O-methyl.

In a further preferred variant, R3′ in the general formula (V) is —C(═O)—OH.

In a further preferred variant, R3′ in the general formula (V) is —C(═O)—CH₃.

In a further preferred variant, R3′ in the general formula (V) is —C(═O)—C₃H₇.

In a still further preferred variant, R3′ in the general formula (V) is —C(═O)—O—methyl.

Most preferred the R3′ in the general formula (V) is either H or methyl or —O-methyl.

In a preferred variant according to the first aspect of the present invention, the mycosporine-like amino acid compound (b) of the general formula (V) is either an acid compound or an ester compound represented by the general formula (VII)

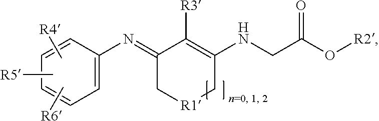

wherein R1′, R2′, R3′, R4′, R5′ and R6′ have the same meaning as defined herein for the general formula (V).

In a preferred variant according to the first aspect of the present invention, the mycosporine-like amino acid compound (b) of the general formula (V) is an acid compound represented by the general formula (VII-acid)

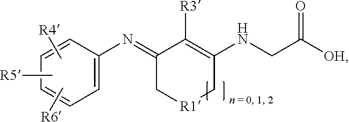

wherein R1′, R3′, R4′, R5′ and R6′ have the same meaning as defined herein for the general formula (V).

Such mycosporine-like amino acid compounds are particularly photo-stable and temperature-stable compounds and show stability in pH solution or in different emulsion types and do not degrade.

Especially, said mycosporine-like amino acid compounds have a better pH stability in a broader pH-range compared to their respective ester compounds which hydrolize, and, thus, are not stable in alkaline solutions having a pH higher than 9.

In addition, said mycosporine-like amino acid compounds have also a better solubility in water compared to their corresponding ester compounds. Due to their better solubility in water, said mycosporine-like amino acid compounds can be incorporated into the water-phase of emulsions in higher concentrations, resulting in an improved boosting effect of an antioxidant in food, cosmetic or pharmaceutical preparations or homecare products.

In a preferred variant according to the first aspect of the present invention, the mycosporine-like amino acid compound (b) of the general formula (V) is an ester compound represented by the general formula (VII-ester)

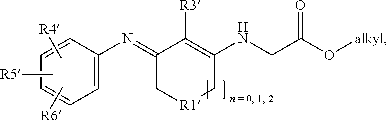

wherein R1′, R3′, R4′, R5′ and R6′ have the same meaning as defined herein for the general formula (V).

Preferably, the alkyl residue in the general formula (VII-ester) is methyl or ethyl or propyl or isopropyl or butyl or isobutyl or tert-butyl. Most preferred the alkyl residue in the general formula (VII-ester) is methyl. Such ester compounds are particularly stable.

In an alternative preferred variant according to the first aspect of the present invention, the mycosporine-like amino acid compound (b) of the general formula (V) is an amide compound represented by the general formula (VIII)

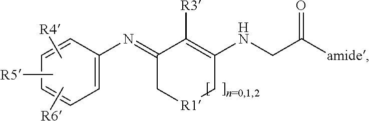

wherein R1′, amide′, R3′, R4′, R5′ and R6′ have the same meaning as defined herein for the general formula (V).

In a more preferred variant according to the first aspect of the present invention, the mycosporine-like amino acid compound (b) of the general formula (V) is an amide compound represented by the general formula (VIII-amide)

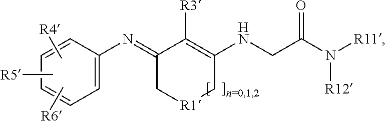

wherein R1′, R3′, R4′, R5′ and R6′ have the same meaning as defined herein for the general formula (V) and R11′ and R12′ are independently from each other selected from the group consisting of H, OH and alkyl.

In a preferred variant according to the first aspect of the present invention, in the general formula (VIII-amide), R11′ and/or R12′ are H.

In a further preferred variant, the R11′ alkyl and R12′ alkyl in the general formula (VIII-amide) are independently from each other selected from the group consisting of methyl, ethyl, propyl, isopropyl, butyl, isobutyl, and tert-butyl.

Preferably, R11′ and/or R12′ in the general formula (VIII-amide) are each H or each methyl.

In a still more preferred variant, the mycosporine-like amino acid compound (b) of the general formula (V) is a Weinreb amide compound represented by the general formula (VIII-Weinreb amide)

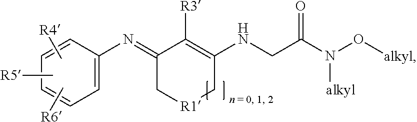

wherein R1′, R3′, R4′, R5′ and R6′ have the same meaning as defined herein for the general formula (V).

In a preferred variant, the alkyl residue in the general formula (VIII-Weinreb amide) is selected from the group consisting of methyl, ethyl, propyl, isopropyl, butyl, isobutyl, and tert-butyl, more preferred the alkyl residue is methyl.

Alternatively, in a still more preferred variant, the mycosporine-like amino acid compound (b) of the general formula (V) is a hydroxamate compound represented by the general formula (VIII-hydroxamate derivative)

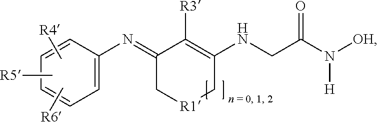

wherein R1′, R3′, R4′, R5′ and R6′ have the same meaning as defined herein for general formula (V).

Alternatively, in a still further preferred variant, the mycosporine-like amino acid compound (b) of the general formula (V) is an alkylated hydroxamate compound represented by the general formula (VIII-alkylated hydroxamate derivative)

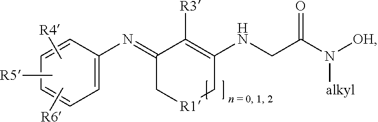

wherein R1′, R3′, R4′, R5′ and R6′ have the same meaning as defined herein for the general formula (V).

In a preferred variant, the alkyl residue in the general formula (VIII-alkylated hydroxamate derivative) is selected from the group consisting of methyl, ethyl, propyl, isopropyl, butyl, isobutyl, and tert-butyl, more preferred the alkyl is methyl.

In a preferred alternatively variant the mycosporine-like amino acid compound (b) is represented by the general formula (A)

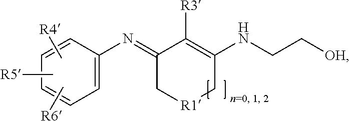

wherein R1′, R3′, R4′, R5′ and R6′ have the same meaning as defined herein for the general formula (V).

Within the present invention, the same preferred variants as defined herein for the mycosporine-like amino acid compounds according to the general formula (V) apply analogously for the mycosporine-like amino acid compounds according to the general formulae (VII) or (VIII) and their variants or general formula (A).

In a further preferred variant, R1″ in the general formula (VI) is CH₂.

In a further preferred variant, R1″ in the general formula (VI) is C(alkyl)₂.

In a further preferred variant, R1″ in the general formula (VI) is O.

In a further preferred variant, R1″ in the general formula (VI) is S.

In a further preferred variant, R1″ in the general formula (VI) is SO.

In a further preferred variant, R1″ in the general formula (VI) is SO₂.

In a further preferred variant, R1″ in the general formula (VI) is NH.

In a still further preferred variant, R1″ in the general formula (VI) is N(alkyl).

Most preferred the R1″ in the general formula (VI) is either CH₂ or C(alkyl)₂.

In a preferred variant, R2″ in the general formula (VI) is —CH₂—OH.

In a further preferred variant, R2″ in the general formula (VI) is —C(═O)—O—Y.

In a further preferred variant, R2″ in the general formula (VI) is —C(═O)-amide″.

Most preferred the R2″ in the general formula (VI) is —C(═O)—O—Y or —C(═O)-amide″.

In a further preferred variant, X in the general formula (VI) is CH₂.

In a further preferred variant, X in the general formula (VI) is C═O.

In a further preferred variant, X in the general formula (VI) is O.

In a further preferred variant, X in the general formula (VI) is S.

In a further preferred variant, X in the general formula (VI) is SO.

In a further preferred variant, X in the general formula (VI) is SO₂.

In a further preferred variant, X in the general formula (VI) is NH.

In a still further preferred variant, X in the general formula (VI) is N(alkyl), wherein alkyl is selected from the group consisting of methyl, ethyl, propyl, isopropyl, butyl, isobutyl, and tert-butyl.

Most preferred the X in the general formula (VI) is either S, SO or SO₂.

In a preferred variant, Y in the general formula (VI) is H.

In a further preferred variant, Y in the general formula (VI) is methyl.

In a further preferred variant, Y in the general formula (VI) is ethyl.

In a further preferred variant, Y in the general formula (VI) is propyl.

In a further preferred variant, Y in the general formula (VI) is isopropyl.

In a further preferred variant, Y in the general formula (VI) is butyl.

In a further preferred variant, Y in the general formula (VI) is isobutyl.

In a further preferred variant, Y in the general formula (VI) is tert-butyl.

In a further preferred variant, Y in the general formula (VI) is

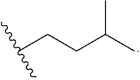

In a further preferred variant, Y in the general formula (VI) is

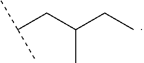

In a preferred variant, Y in the general formula (VI) is

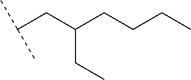

(2-ethyl-hexyl).

In a still further preferred variant, Y in the general formula (VI) is phenyl.

Most preferred the Y in the general formula (VI) is H or ethyl or isopropyl or 2-ethylhexyl or phenyl.

In a preferred variant, amide″ in the general formula (VI) is NH₂.

In a further preferred variant, amide″ in the general formula (VI) is NH(alkyl), wherein alkyl is selected from the group consisting of methyl, ethyl, propyl, isopropyl, butyl, isobutyl, and tert-butyl.

In a further preferred variant, amide″ in the general formula (VI) is N(alkyl)₂, wherein alkyl is selected from the group consisting of methyl, ethyl, propyl, isopropyl, butyl, isobutyl, and tert-butyl.

In a further preferred variant, amide″ in the general formula (VI) is —N(O-alkyl)(alkyl) (Weinreb amide), wherein alkyl is selected from the group consisting of methyl, ethyl, propyl, isopropyl, butyl, isobutyl, and tert-butyl.

In a still further preferred variant, amide″ in the general formula (V) is —N(OH)(H) (hydroxamate).

Most preferred the amide″ in the general formula (V) is NH₂, N(methyl)₂ or N(ethyl)₂ or —N(O-alkyl)(alkyl) (Weinreb amide), wherein alkyl is selected from the group consisting of methyl, ethyl, propyl, isopropyl, butyl, isobutyl, and tert-butyl, or —N(OH)(H) (hydroxamate).

In a more preferred variant according to the first aspect of the present invention, the mycosporine-like amino acid compound (b) is an alcohol compound represented by the general formula (IX-alcohol)

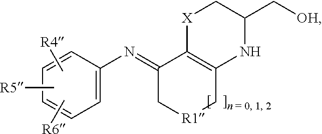

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an acid compound represented by the general formula (IX-acid)

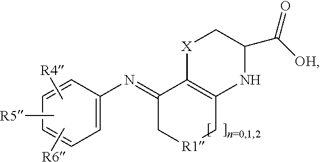

wherein R1″, X, R4″, R5″ and R6″ have the same meaning as defined herein for the general formula (VI).

Alternatively, in a more preferred variant according to the first aspect of the present invention, the mycosporine-like amino acid compound (b) of the general formula (VI) is an ester compound represented by the general formula (VII-ester)

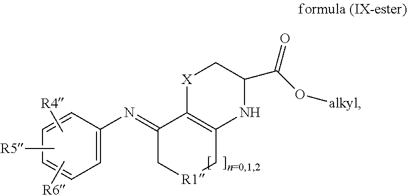

wherein R1″, X, R4″, R5″ and R6″ have the same meaning as defined herein for the general formula (VI).

Preferably, the alkyl residue in the general formula (VII-ester) is methyl or ethyl or propyl or isopropyl or butyl or isobutyl or tert-butyl, more preferred the alkyl residue is methyl.

In an alternative more preferred variant according to the first aspect of the present invention, the mycosporine-like amino acid compound (b) of the general formula (VI) is an amide compound represented by the general formula (X)

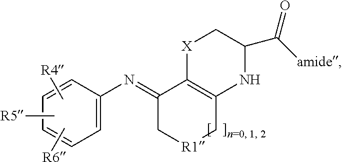

wherein R1″, amide″, X, R4″, R5″ and R6″ have the same meaning as defined herein for the general formula (VI).

In a more preferred alternatively variant according to the first aspect of the present invention, the mycosporine-like amino acid compound (b) of the general formula (VI) is an amide compound represented by the general formula (X-amide),

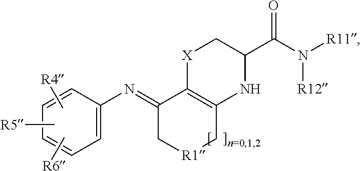

wherein R1″, X, R4″, R5″ and R6″ have the same meaning as defined above for the general formula (VI) and R11″ and R12″ are independently from each other selected from the group consisting of H, OH and alkyl. In a preferred variant according to the first aspect of the present invention, in the general formula (X-amide), R11″ and R12″ are H.

Preferably, the R11″ alkyl and/or R12″ alkyl in the general formula (X-amide) is methyl or ethyl or propyl or isopropyl or butyl or isobutyl or butyl.

Preferably, R11″ and/or R12″ in the general formula (X-amide) are each H or each methyl.

In a still more preferred variant, the mycosporine-like amino acid compound (b) of the general formula (VI) is a Weinreb amide compound represented by the general formula (X-Weinreb amide)

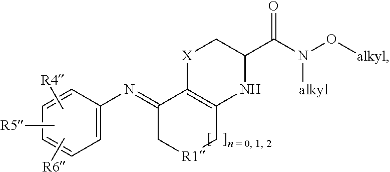

wherein R1″, X, R4″, R5″ and R6″ have the same meaning as defined herein for the general formula (VI).

In a preferred variant, the alkyl residue in the general formula (X-Weinreb amide) is selected from the group consisting of methyl, ethyl, propyl, isopropyl, butyl, isobutyl, and tert-butyl, more preferred the alkyl residue is methyl.

Alternatively, in a still more preferred variant, the mycosporine-like amino acid compound (b) of the general formula (VI) is a hydroxamate compound represented by the general formula (X-hydroxamate acid derivative)

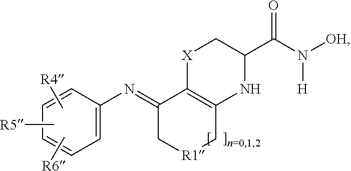

wherein R1″, X, R4″, R5″ and R6″ have the same meaning as defined herein for the general formula (VI).

Alternatively, in a still further preferred variant, the mycosporine-like amino acid compound (b) of the general formula (VI) is an alkylated hydroxamate compound represented by the general formula (VIII-alkylated hydroxamate derivative)

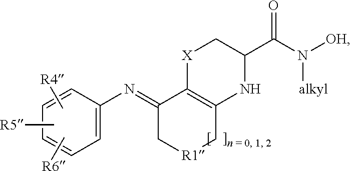

wherein R1″, X, R4″, R5″ and R6″ have the same meaning as defined herein for the general formula (VI).

In a preferred variant, the alkyl residue in formula (X-alkylated hydroxamate derivative) is selected from the group consisting of methyl, ethyl, propyl, isopropyl, butyl, isobutyl, and butyl, more preferred the alkyl residue is methyl.

Within the present invention, the same preferred variants as defined herein for the mycosporine-like amino acid compounds according to the general formula (VI) apply analogously for the mycosporine-like amino acid compounds according to the general formulae (IX) or (X) and their variants.

In the following and throughout the description, the term “according to the general formulae YZ and their variants” includes any sub-formula (e) of said general formulae as disclosed herein. Accordingly, variants of the general formulae (V) and (VI) as defined herein, include formula (VII), formula (VIII), formula (IX) and formula (X) as defined herein. Variants of the general formulae (VII) and (VIII) as defined herein, include sub-formula (VII-acid), sub-formula (VII-ester), sub-formula (VIII-amide), sub-formula (VIII-Weinreb amide), sub-formula (VIII-hydroxamate derivative) and sub-formula (VIII-alkylated hydroxamate derivative) as defined herein. Variants of the general formulae (IX) and (X) as defined herein, include formula (IX-acid), formula (IX-ester), formula (X-amide), formula (X-Weinreb amide), formula (X-hydroxamate derivative) and formula (X-alkylated hydroxamate derivative) as defined herein.

In a further preferred variant, in the general formulae (VI), (IX), (X) and their variants, X is selected from the group consisting of selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or r substituted N(heterocycloalkyl), unsubstituted or substituted N(heteroaryl), and unsubstituted or substituted C-spirocycles.

In a further preferred variant according to the first aspect of the present invention, the mycosporine-like amino acid compound is a compound according to the general formulae (V), wherein
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

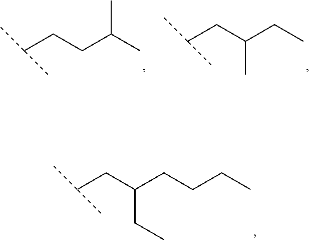

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to the general formulae (VII) or (VIII), wherein
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl,

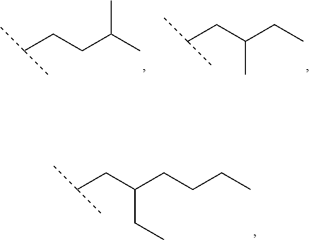

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to the general formula (VI), wherein
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

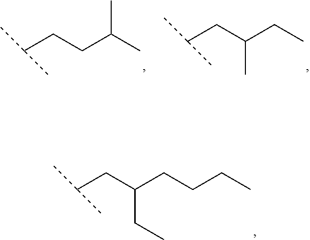

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a further preferred variant according to the first aspect of the present invention, the mycosporine-like amino acid compound is a compound according to the general formula (V), wherein
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl,

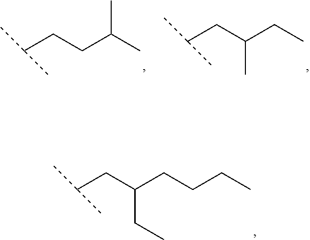

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to the general formulae (VII) or (VIII), wherein
R1′ is selected from the group consisting of CH₂ and C(methyl)₂; and/or
R2′ is selected from the group consisting of H, methyl, ethyl, propyl, isopropyl, butyl, isobutyl, tert-butyl,

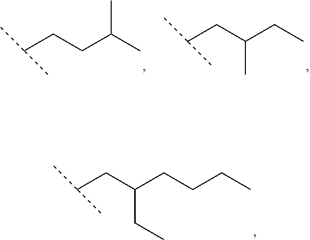

(2-ethyl-hexyl) and phenyl; or
amide′ is selected from the group consisting of NH₂, NH(alkyl), N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In another preferred variant according to the first aspect of the present invention, the mycosporine-like amino acid compound is a compound according to the general formula (VI), wherein
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl,

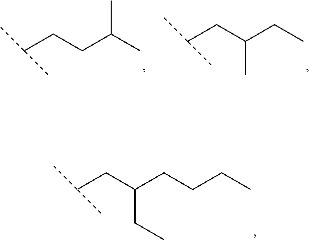

(2-ethyl-hexyl) and phenyl; and/or
amide″ is selected from the group consisting of NH₂, NH(alkyl), N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

In a still more preferred variant according to the present invention, the food, cosmetic or pharmaceutical preparation or homecare product comprises at least one mycosporine-like amino acid compound selected from the group consisting of the following compounds I-1 to 1-128 according to the general formula (V) and II-1 to II-192 according to the general formula (VI):

TABLE 1

| 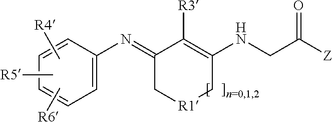 | R1′ | R3′ | Z |
|---|---|---|---|
| I-1 | CH2 | H | —O-R2′ |
| I-2 | CH2 | methyl | —O-R2′ |
| I-3 | CH2 | ethyl | —O-R2′ |
| I-4 | CH2 | —O-methyl | —O-R2′ |
| I-5 | CH2 | —C(═O)—OH | —O-R2′ |
| I-6 | CH2 | —C(═O)—CH3 | —O-R2′ |
| I-7 | CH2 | —C(═O)—C3H7 | —O-R2′ |
| I-8 | CH2 | —C(═O)—O-methyl | —O-R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O-R2′ |
| I-18 | C(methyl)2 | methyl | —O-R2′ |
| I-19 | C(methyl)2 | ethyl | —O-R2′ |
| I-20 | C(methyl)2 | —O-methyl | —O-R2′ |
| I-21 | C(methyl)2 | —C(═O)—OH | —O-R2′ |
| I-22 | C(methyl)2 | —C(═O)—CH3 | —O-R2′ |
| I-23 | C(methyl)2 | —C(═O)—C3H7 | —O-R2′ |
| I-24 | C(methyl)2 | —C(═O)—O-methyl | —O-R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| I-33 | O | H | —O-R2′ |
| I-34 | O | methyl | —O-R2′ |
| I-35 | O | ethyl | —O-R2′ |
| I-36 | O | —O-methyl | —O-R2′ |
| I-37 | O | —C(═O)—OH | —O-R2′ |
| I-38 | O | —C(═O)—CH3 | —O-R2′ |
| I-39 | O | —C(═O)—C3H7 | —O-R2′ |
| I-40 | O | —C(═O)—O-methyl | —O-R2′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)—OH | amide′ |
| I-46 | O | —C(═O)—CH3 | amide′ |
| I-47 | O | —C(═O)—C3H7 | amide′ |
| I-48 | O | —C(═O)—O-methyl | amide′ |
| I-49 | S | H | —O-R2′ |
| I-50 | S | methyl | —O-R2′ |
| I-51 | S | ethyl | —O-R2′ |
| I-52 | S | —O-methyl | —O-R2′ |
| I-53 | S | —C(═O)—OH | —O-R2′ |
| I-54 | S | —C(═O)—CH3 | —O-R2′ |
| I-55 | S | —C(═O)—C3H7 | —O-R2′ |
| I-56 | S | —C(═O)—O-methyl | —O-R2′ |
| I-57 | S | H | amide′ |
| I-58 | S | methyl | amide′ |
| I-59 | S | ethyl | amide′ |
| I-60 | S | —O-methyl | amide′ |
| I-61 | S | —C(═O)—OH | amide′ |
| I-62 | S | —C(═O)—CH3 | amide′ |
| I-63 | S | —C(═O)—C3H7 | amide′ |
| I-64 | S | —C(═O)—O-methyl | amide′ |
| I-65 | SO | H | —O-R2′ |
| I-66 | SO | methyl | —O-R2′ |
| I-67 | SO | ethyl | —O-R2′ |
| I-68 | SO | —O-methyl | —O-R2′ |
| I-69 | SO | —C(═O)—OH | —O-R2′ |
| I-70 | SO | —C(═O)—CH3 | —O-R2′ |
| I-71 | SO | —C(═O)—C3H7 | —O-R2′ |
| I-72 | SO | —C(═O)—O-methyl | —O-R2′ |
| I-73 | SO | H | amide′ |
| I-74 | SO | methyl | amide′ |
| I-75 | SO | ethyl | amide′ |
| I-76 | SO | —O-methyl | amide′ |
| I-77 | SO | —C(═O)—OH | amide′ |
| I-78 | SO | —C(═O)—CH3 | amide′ |
| I-79 | SO | —C(═O)—C3H7 | amide′ |
| I-80 | SO | —C(═O)—O-methyl | amide′ |
| I-81 | SO2 | H | —O-R2′ |
| I-82 | SO2 | methyl | —O-R2′ |
| I-83 | SO2 | ethyl | —O-R2′ |
| I-84 | SO2 | —O-methyl | —O-R2′ |
| I-85 | SO2 | —C(═O)—OH | —O-R2′ |
| I-86 | SO2 | —C(═O)—CH3 | —O-R2′ |
| I-87 | SO2 | —C(═O)—C3H7 | —O-R2′ |
| I-88 | SO2 | —C(═O)—O-methyl | —O-R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)—OH | amide′ |
| I-94 | SO2 | —C(═O)—CH3 | amide′ |
| I-95 | SO2 | —C(═O)—C3H7 | amide′ |
| I-96 | SO2 | —C(═O)—O-methyl | amide′ |
| I-97 | NH | H | —O-R2′ |
| I-98 | NH | methyl | —O-R2′ |
| I-99 | NH | ethyl | —O-R2′ |
| I-100 | NH | —O-methyl | —O-R2′ |
| I-101 | NH | —C(═O)—OH | —O-R2′ |
| I-102 | NH | —C(═O)—CH3 | —O-R2′ |
| I-103 | NH | —C(═O)—C3H7 | —O-R2′ |
| I-104 | NH | —C(═O)—O-methyl | —O-R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)—OH | amide′ |
| I-110 | NH | —C(═O)—CH3 | amide′ |
| I-111 | NH | —C(═O)—C3H7 | amide′ |
| I-112 | NH | —C(═O)—O-methyl | amide′ |
| I-113 | N(alkyl) | H | —O-R2′ |
| I-114 | N(alkyl) | methyl | —O-R2′ |
| I-115 | N(alkyl) | ethyl | —O-R2′ |
| I-116 | N(alkyl) | —O-methyl | —O-R2′ |
| I-117 | N(alkyl) | —C(═O)—OH | —O-R2′ |
| I-118 | N(alkyl) | —C(═O)—CH3 | —O-R2′ |
| I-119 | N(alkyl) | —C(═O)—C3H7 | —O-R2′ |
| I-120 | N(alkyl) | —C(═O)—O-methyl | —O-R2′ |
| I-121 | N(alkyl) | H | amide′ |
| I-122 | N(alkyl) | methyl | amide′ |
| I-123 | N(alkyl) | ethyl | amide′ |
| I-124 | N(alkyl) | —O-methyl | amide′ |
| I-125 | N(alkyl) | —C(═O)—OH | amide′ |
| I-126 | N(alkyl) | —C(═O)—CH3 | amide′ |
| I-127 | N(alkyl) | —C(═O)—C3H7 | amide′ |
| I-128 | N(alkyl) | —C(═O)—O-methyl | amide′ |
| 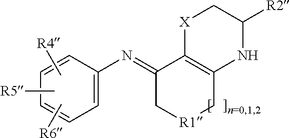 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |
| II-49 | O | CH2 | —CH2—OH |
| II-50 | O | C═O | —CH2—OH |
| II-51 | O | O | —CH2—OH |
| II-52 | O | S | —CH2—OH |
| II-53 | O | SO | —CH2—OH |
| II-54 | O | SO2 | —CH2—OH |
| II-55 | O | NH | —CH2—OH |
| II-56 | O | N(alkyl) | —CH2—OH |
| II-57 | O | CH2 | —C(═O)—O—Y |
| II-58 | O | C═O | —C(═O)—O—Y |
| II-59 | O | O | —C(═O)—O—Y |
| II-60 | O | S | —C(═O)—O—Y |
| II-61 | O | SO | —C(═O)—O—Y |
| II-62 | O | SO2 | —C(═O)—O—Y |
| II-63 | O | NH | —C(═O)—O—Y |
| II-64 | O | N(alkyl) | —C(═O)—O—Y |
| II-65 | O | CH2 | —C(═O)-amide″ |
| II-66 | O | C═O | —C(═O)-amide″ |
| II-67 | O | O | —C(═O)-amide″ |
| II-68 | O | S | —C(═O)-amide″ |
| II-69 | O | SO | —C(═O)-amide″ |
| II-70 | O | SO2 | —C(═O)-amide″ |
| II-71 | O | NH | —C(═O)-amide″ |
| II-72 | O | N(alkyl) | —C(═O)-amide″ |
| II-73 | S | CH2 | —CH2—OH |
| II-74 | S | C═O | —CH2—OH |
| II-75 | S | O | —CH2—OH |
| II-76 | S | S | —CH2—OH |
| II-77 | S | SO | —CH2—OH |
| II-78 | S | SO2 | —CH2—OH |
| II-79 | S | NH | —CH2—OH |
| II-80 | S | N(alkyl) | —CH2—OH |
| II-81 | S | CH2 | —C(═O)—O—Y |
| II-82 | S | C═O | —C(═O)—O—Y |
| II-83 | S | O | —C(═O)—O—Y |
| II-84 | S | S | —C(═O)—O—Y |
| II-85 | S | SO | —C(═O)—O—Y |
| II-86 | S | SO2 | —C(═O)—O—Y |
| II-87 | S | NH | —C(═O)—O—Y |
| II-88 | S | N(alkyl) | —C(═O)—O—Y |
| II-89 | S | CH2 | —C(═O)-amide″ |
| II-90 | S | C═O | —C(═O)-amide″ |
| II-91 | S | O | —C(═O)-amide″ |
| II-92 | S | S | —C(═O)-amide″ |
| II-93 | S | SO | —C(═O)-amide″ |
| II-94 | S | SO2 | —C(═O)-amide″ |
| II-95 | S | NH | —C(═O)-amide″ |
| II-96 | S | N(alkyl) | —C(═O)-amide″ |
| II-97 | SO | CH2 | —CH2—OH |
| II-98 | SO | C═O | —CH2—OH |
| II-99 | SO | O | —CH2—OH |
| II-100 | SO | S | —CH2—OH |
| II-101 | SO | SO | —CH2—OH |
| II-102 | SO | SO2 | —CH2—OH |
| II-103 | SO | NH | —CH2—OH |
| II-104 | SO | N(alkyl) | —CH2—OH |
| II-105 | SO | CH2 | —C(═O)—O—Y |
| II-106 | SO | C═O | —C(═O)—O—Y |
| II-107 | SO | O | —C(═O)—O—Y |
| II-108 | SO | S | —C(═O)—O—Y |
| II-109 | SO | SO | —C(═O)—O—Y |
| II-110 | SO | SO2 | —C(═O)—O—Y |
| II-111 | SO | NH | —C(═O)—O—Y |
| II-112 | SO | N(alkyl) | —C(═O)—O—Y |
| II-113 | SO | CH2 | —C(═O)-amide″ |
| II-114 | SO | C═O | —C(═O)-amide″ |
| II-115 | SO | O | —C(═O)-amide″ |
| II-116 | SO | S | —C(═O)-amide″ |
| II-117 | SO | SO | —C(═O)-amide″ |
| II-118 | SO | SO2 | —C(═O)-amide″ |
| II-119 | SO | NH | —C(═O)-amide″ |
| II-120 | SO | N(alkyl) | —C(═O)-amide″ |
| II-121 | SO2 | CH2 | —CH2—OH |
| II-122 | SO2 | C═O | —CH2—OH |
| II-123 | SO2 | O | —CH2—OH |
| II-124 | SO2 | S | —CH2—OH |
| II-125 | SO2 | SO | —CH2—OH |
| II-126 | SO2 | SO2 | —CH2—OH |
| II-127 | SO2 | NH | —CH2—OH |
| II-128 | SO2 | N(alkyl) | —CH2—OH |
| II-129 | SO2 | CH2 | —C(═O)—O—Y |
| II-130 | SO2 | C═O | —C(═O)—O—Y |
| II-131 | SO2 | O | —C(═O)—O—Y |
| II-132 | SO2 | S | —C(═O)—O—Y |
| II-133 | SO2 | SO | —C(═O)—O—Y |
| II-134 | SO2 | SO2 | —C(═O)—O—Y |
| II-135 | SO2 | NH | —C(═O)—O—Y |
| II-136 | SO2 | N(alkyl) | —C(═O)—O—Y |
| II-137 | SO2 | CH2 | —C(═O)-amide″ |
| II-138 | SO2 | C═O | —C(═O)-amide″ |
| II-139 | SO2 | O | —C(═O)-amide″ |
| II-140 | SO2 | S | —C(═O)-amide″ |
| II-141 | SO2 | SO | —C(═O)-amide″ |
| II-142 | SO2 | SO2 | —C(═O)-amide″ |
| II-143 | SO2 | NH | —C(═O)-amide″ |
| II-144 | SO2 | N(alkyl) | —C(═O)-amide″ |
| II-145 | NH | CH2 | —CH2—OH |
| II-146 | NH | C═O | —CH2—OH |
| II-147 | NH | O | —CH2—OH |
| II-148 | NH | S | —CH2—OH |
| II-149 | NH | SO | —CH2—OH |
| II-150 | NH | SO2 | —CH2—OH |
| II-151 | NH | NH | —CH2—OH |
| II-152 | NH | N(alkyl) | —CH2—OH |
| II-153 | NH | CH2 | —C(═O)—O—Y |
| II-154 | NH | C═O | —C(═O)—O—Y |
| II-155 | NH | O | —C(═O)—O—Y |
| II-156 | NH | S | —C(═O)—O—Y |
| II-157 | NH | SO | —C(═O)—O—Y |
| II-158 | NH | SO2 | —C(═O)—O—Y |
| II-159 | NH | NH | —C(═O)—O—Y |
| II-160 | NH | N(alkyl) | —C(═O)—O—Y |
| II-161 | NH | CH2 | —C(═O)-amide″ |
| II-162 | NH | C═O | —C(═O)-amide″ |
| II-163 | NH | O | —C(═O)-amide″ |
| II-164 | NH | S | —C(═O)-amide″ |
| II-165 | NH | SO | —C(═O)-amide″ |
| II-166 | NH | SO2 | —C(═O)-amide″ |
| II-167 | NH | NH | —C(═O)-amide″ |
| II-168 | NH | N(alkyl) | —C(═O)-amide″ |
| II-169 | N(alkyl) | CH2 | —CH2—OH |
| II-170 | N(alkyl) | C═O | —CH2—OH |
| II-171 | N(alkyl) | O | —CH2—OH |
| II-172 | N(alkyl) | S | —CH2—OH |
| II-173 | N(alkyl) | SO | —CH2—OH |
| II-174 | N(alkyl) | SO2 | —CH2—OH |
| II-175 | N(alkyl) | NH | —CH2—OH |
| II-176 | N(alkyl) | N(alkyl) | —CH2—OH |
| II-177 | N(alkyl) | CH2 | —C(═O)—O—Y |
| II-178 | N(alkyl) | C═O | —C(═O)—O—Y |
| II-179 | N(alkyl) | O | —C(═O)—O—Y |
| II-180 | N(alkyl) | S | —C(═O)—O—Y |
| II-181 | N(alkyl) | SO | —C(═O)—O—Y |
| II-182 | N(alkyl) | SO2 | —C(═O)—O—Y |
| II-183 | N(alkyl) | NH | —C(═O)—O—Y |
| II-184 | N(alkyl) | N(alkyl) | —C(═O)—O—Y |
| II-185 | N(alkyl) | CH2 | —C(═O)-amide″ |
| II-186 | N(alkyl) | C═O | —C(═O)-amide″ |
| II-187 | N(alkyl) | O | —C(═O)-amide″ |
| II-188 | N(alkyl) | S | —C(═O)-amide″ |
| II-189 | N(alkyl) | SO | —C(═O)-amide″ |
| II-190 | N(alkyl) | SO2 | —C(═O)-amide″ |
| II-191 | N(alkyl) | NH | —C(═O)-amide″ |
| II-192 | N(alkyl) | N(alkyl) | —C(═O)-amide″ |

or a tautomer or a stereoisomer or a salt of the afore-mentioned compounds, or any mixture of the afore-mentioned compounds.

In the mycosporine-like amino acid compounds of Table 1, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to the general formulae (V) or (VI) before.

Particular preferred are those mycosporine-like amino acid compounds, wherein in the general formula (V) R1′ is CH₂ or C(methyl)₂ or wherein in the general formula (VI) R1″ is CH₂ or C(methyl)₂:

TABLE 2

| 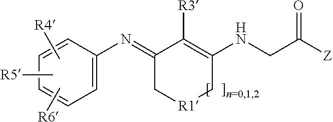 | R1′ | R3′ | Z |
|---|---|---|---|
| I-1 | CH2 | H | —O-R2′ |
| I-2 | CH2 | methyl | —O-R2′ |
| I-3 | CH2 | ethyl | —O-R2′ |
| I-4 | CH2 | —O-methyl | —O-R2′ |
| I-5 | CH2 | —C(═O)—OH | —O-R2′ |
| I-6 | CH2 | —C(═O)—CH3 | —O-R2′ |
| I-7 | CH2 | —C(═O)—C3H7 | —O-R2′ |
| I-8 | CH2 | —C(═O)—O-methyl | —O-R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)—OH | amide′ |
| I-14 | CH2 | —C(═O)—CH3 | amide′ |
| I-15 | CH2 | —C(═O)—C3H7 | amide′ |
| I-16 | CH2 | —C(═O)—O-methyl | amide′ |
| I-17 | C(methyl)2 | H | —O-R2′ |
| I-18 | C(methyl)2 | methyl | —O-R2′ |
| I-19 | C(methyl)2 | ethyl | —O-R2′ |
| I-20 | C(methyl)2 | —O-methyl | —O-R2′ |
| I-21 | C(methyl)2 | —C(═O)—OH | —O-R2′ |
| I-22 | C(methyl)2 | —C(═O)—CH3 | —O-R2′ |
| I-23 | C(methyl)2 | —C(═O)—C3H7 | —O-R2′ |
| I-24 | C(methyl)2 | —C(═O)—O-methyl | —O-R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methyl)2 | —C(═O)—OH | amide′ |
| I-30 | C(methyl)2 | —C(═O)—CH3 | amide′ |
| I-31 | C(methyl)2 | —C(═O)—C3H7 | amide′ |
| I-32 | C(methyl)2 | —C(═O)—O-methyl | amide′ |
| 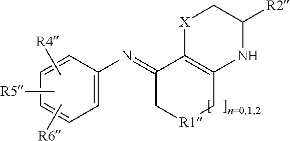 | R1″ | X | R2″ |
| II-1 | CH2 | CH2 | —CH2—OH |
| II-2 | CH2 | C═O | —CH2—OH |
| II-3 | CH2 | O | —CH2—OH |
| II-4 | CH2 | S | —CH2—OH |
| II-5 | CH2 | SO | —CH2—OH |
| II-6 | CH2 | SO2 | —CH2—OH |
| II-7 | CH2 | NH | —CH2—OH |
| II-8 | CH2 | N(alkyl) | —CH2—OH |
| II-9 | CH2 | CH2 | —C(═O)—O—Y |
| II-10 | CH2 | C═O | —C(═O)—O—Y |
| II-11 | CH2 | O | —C(═O)—O—Y |
| II-12 | CH2 | S | —C(═O)—O—Y |
| II-13 | CH2 | SO | —C(═O)—O—Y |
| II-14 | CH2 | SO2 | —C(═O)—O—Y |
| II-15 | CH2 | NH | —C(═O)—O—Y |
| II-16 | CH2 | N(alkyl) | —C(═O)—O—Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2—OH |
| II-26 | C(methyl)2 | C═O | —CH2—OH |
| II-27 | C(methyl)2 | O | —CH2—OH |
| II-28 | C(methyl)2 | S | —CH2—OH |
| II-29 | C(methyl)2 | SO | —CH2—OH |
| II-30 | C(methyl)2 | SO2 | —CH2—OH |
| II-31 | C(methyl)2 | NH | —CH2—OH |
| II-32 | C(methyl)2 | N(alkyl) | —CH2—OH |
| II-33 | C(methyl)2 | CH2 | —C(═O)—O—Y |
| II-34 | C(methyl)2 | C═O | —C(═O)—O—Y |
| II-35 | C(methyl)2 | O | —C(═O)—O—Y |
| II-36 | C(methyl)2 | S | —C(═O)—O—Y |
| II-37 | C(methyl)2 | SO | —C(═O)—O—Y |
| II-38 | C(methyl)2 | SO2 | —C(═O)—O—Y |
| II-39 | C(methyl)2 | NH | —C(═O)—O—Y |
| II-40 | C(methyl)2 | N(alkyl) | —C(═O)—O—Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methyl)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |

or a tautomer or a stereoisomer or a salt of the afore-mentioned compounds, or any mixture of the afore-mentioned compounds.

In the mycosporine-like amino acid compounds of Table 2, the phenyl ring may be unsubstituted or substituted. The substituents R4′, R5′, R6′, R4″, R5″, R6″, amide′, Y, amide″ and alkyl have the same meaning as defined for the mycosporine-like amino acid compounds according to the general formulae (V) or (VI) before.

Preferably, in the mycosporine-like amino acid compounds according to the general formula (V) or the general formula (VI) specified in Table 1 and Table 2
R2′ is selected from the group consisting of H, methyl, ethyl, propyl, isopropyl, butyl, isobutyl, tert-butyl,

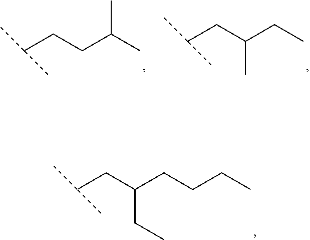

(2-ethylhexyl) and phenyl;
amide′ is selected from the group consisting of NH₂, NH(alkyl), N(alkyl)₂, —N(O-alkyl)(alkyl) (Weinreb amide) and —N(OH)(H) (hydroxamate);
Y is selected from the group consisting of H, methyl, ethyl, propyl, isopropyl, butyl, isobutyl, tert-butyl,

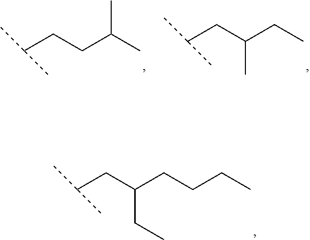

(2-ethylhexyl) and phenyl;
amide″ is selected from the group consisting of NH₂, NH(alkyl), N(alkyl)₂, —N(O-alkyl)(alkyl) (Weinreb amide) and —N(OH)(H) (hydroxamate); and
alkyl is selected from the group consisting of methyl, ethyl, propyl, isopropyl, butyl, isobutyl, and tert-butyl.

In a most preferred variant according to the first aspect of the present invention, the mycosporine-like amino acid compound is a compound according to the general formula (V) and its variants or general formula (VI) and its variants, wherein R1′ or R1″ are CH₂. Such mycosporine-like amino acid compounds have a particular good water solubility.

In a still further preferred variant according to the first aspect of the present invention, the mycosporine-like amino acid compound is a compound according to the general formulae (VI), (IX), (X) and their variants, wherein X is selected from the group consisting of S, SO, and SO₂.

In a still more preferred variant according to the first aspect of the present invention, the mycosporine-like amino acid compound is a compound according to the general formulae (VI), (IX), (X) and their variants, wherein X is S. However, the S atom in said compounds is susceptible to oxidation.

In a most preferred variant according to the first aspect of the present invention, the mycosporine-like amino acid compound is a compound according to the general formulae (VI), (IX), (X) and their variants, wherein X is either SO or SO₂. Such mycosporine-like amino acid compounds are particular stable, due to their oxidation state.

In a still further preferred variant according to the present invention, in the general formulae (V) to (X) and their variants, n is 1.

In a more preferred variant according to the first aspect of the present invention, the mycosporine-like amino acid compound is a compound according to the general formula (V), wherein
Z is —O—R2′ or amide′; and/or
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to the general formulae (VII) or (VIII), wherein
R1′ is either CH₂ or C(methyl)₂; and/or
R2′ is either H or ethyl or isopropyl or 2-ethylhexyl or phenyl; or
amide′ is either NH₂ or N(methyl)₂ or N(ethyl)₂ or —N(O-alkyl)(alkyl) (Weinreb amide) or —N(OH)(H) (hydroxamate); and/or
R3′ is either H or —O-methyl or —C(═O)—OH or —C(═O)—CH₃ or —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl.

In a more preferred variant according to the first aspect of the present invention, the mycosporine-like amino acid compound is a compound according to the general formula (VI), wherein
R1″ is either CH₂ or C(methyl)₂; and/or
R2″ is either —C(═O)—O—Y, or —C(═O)-amide″; and/or
X is S, SO or SO₂; and/or
Y is either H or ethyl or isopropyl or 2-ethylhexyl or phenyl; or
amide″ is either NH₂ or N(methyl)₂ or N(ethyl)₂ or —N(O-alkyl)(alkyl) (Weinreb amide) or —N(OH)(H) (hydroxamate); and/or the substituents R4″, R5″ and R6″ have the same meaning as defined for formula (VI);
wherein alkyl is selected from the group consisting of methyl, ethyl, propyl, isopropyl, butyl, isobutyl, and butyl.

The phenyl ring in the mycosporine-like amino acid compounds according to the general formulae (V) to (X) and their variants can be unsubstituted or substituted.

In a preferred variant, the phenyl ring in the mycosporine-like amino acid compounds according to the general formulae (V) to (X) and their variants is unsubstituted, i.e. the substituents R4′, R5′ and R6′ on the phenyl ring in the general formula (V) is H; or the substituents R4″, R5″ and R6″ on the phenyl ring in the general formula (VI) is H.

In a further preferred variant, the phenyl ring in the mycosporine-like amino acid compounds according to the general formulae (V) to (X) and their variants is substituted.

Most preferred, the phenyl ring in the mycosporine-like amino acid compounds according to the general formula (V), (VII), (VIII) and their variants is unsubstituted, i.e. the substituents R4′, R5′ and R6′ are each H. Alternatively, most preferred, the phenyl ring in the mycosporine-like amino acid compounds according to the general formula (VI), (IX), (X) and their variants is unsubstituted, i.e. the substituents R4″, R5″ and R6″ are each H. Said compounds have an improved water solubility.

Preferably, the phenyl ring in the mycosporine-like amino acid compounds according to the general formulae (V) to (X) and their variants is monosubstituted, disubstituted or trisubstituted.

In a still more preferred variant according to the first aspect of the present invention, the mycosporine-like amino acid is a compound according to the general formulae (V), (VII), (VIII) and their variants, wherein the substituents R4′, R5′ and R6′ which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl, or O-butyl, —S(═O)₂OH, and sulfonyl.

Still more preferred, the substituents R4′, R5′ and R6′ in the general formulae (V), (VII), (VIII) and their variants, which may be identical or different, are independently from each other selected from the group consisting of H, OH, methyl, O-methyl, —S(═O)₂OH, and sulfonyl.

Alternatively, the mycosporine-like amino acid compound is a compound according to the general formulae (VI), (IX), (X) and their variants, wherein the substituents R4″, R5″ and R6″, which may be identical or different, are independently from each other selected from the group consisting of H, OH, alkyl, preferably methyl, ethyl, propyl, iso-propyl, butyl, isobutyl, tert-butyl, alkoxy, preferably O-methyl, O-ethyl, or O-butyl, —S(═O)₂OH, and sulfonyl.

Still more preferred, the substituents R4″, R5″ and R6″ in the general formula (VI), (IX), (X) and their variants, which may be identical or different, are independently from each other selected from the group consisting of H, OH, methyl, O-methyl, —S(═O)₂OH, and sulfonyl.

In the general formulae (V) to (X) and their variants, the following substitution patterns are particularly preferred:
R4′=H and R5′=H and R6′=H; or
R4′=H and R5′=H and R6′=OH; or
R4′=H and R5′=H and R6′=methyl; or
R4′=H and R5′=H and R6′=O-methyl; or
R4′=H and R5′=H and R6′=—S(═O)₂OH; or
R4′=H and R5′=H and R6′=sulfonyl; or
R4′=H and R5′=OH and R6′=H; or
R4′=H and R5′=OH and R6′=OH; or
R4′=H and R5′=OH and R6′=methyl; or
R4′=H and R5′=OH and R6′=O-methyl; or
R4′=H and R5′=OH and R6′=—S(═O)₂OH; or
R4′=H and R5′=OH and R6′=sulfonyl; or
R4′=H and R5′=methyl and R6′=H; or
R4′=H and R5′=methyl and R6′=OH; or
R4′=H and R5′=methyl and R6′=methyl; or
R4′=H and R5′=methyl and R6′=O-methyl; or
R4′=H and R5′=methyl and R6′=—S(═O)₂OH; or
R4′=H and R5′=methyl and R6′=sulfonyl; or
R4′=H and R5′=O-methyl and R6′=H; or
R4′=H and R5′=O-methyl and R6′=OH; or
R4′=H and R5′=O-methyl and R6′=methyl; or
R4′=H and R5′=O-methyl and R6′=O-methyl; or
R4′=H and R5′=O-methyl and R6′=—S(═O)₂OH; or
R4′=H and R5′=O-methyl and R6′=sulfonyl; or
R4′=H and R5′=—S(═O)₂OH and R6′=H; or
R4′=H and R5′=—S(═O)₂OH and R6′=OH; or
R4′=H and R5′=—S(═O)₂OH and R6′=methyl; or
R4′=H and R5′=—S(═O)₂OH and R6′=O-methyl; or
R4′=H and R5′=—S(═O)₂OH and R6′=—S(═O)₂OH; or
R4′=H and R5′=—S(═O)₂OH and R6′=sulfonyl; or
R4′=H and R5′=sulfonyl and R6′=H; or
R4′=H and R5′=sulfonyl and R6′=OH; or
R4′=H and R5′=sulfonyl and R6′=methyl; or
R4′=H and R5′=sulfonyl and R6′=O-methyl; or
R4′=H and R5′=sulfonyl and R6′=—S(═O)₂OH; or
R4′=H and R5′=sulfonyl and R6′=sulfonyl; or
R4′=OH and R5′=H and R6′=H; or
R4′=OH and R5′=H and R6′=OH; or
R4′=OH and R5′=H and R6′=methyl; or
R4′=OH and R5′=H and R6′=O-methyl; or
R4′=OH and R5′=H and R6′=—S(═O)₂OH; or
R4′=OH and R5′=H and R6′=sulfonyl; or
R4′=OH and R5′=OH and R6′=H; or
R4′=OH and R5′=OH and R6′=OH; or
R4′=OH and R5′=OH and R6′=methyl; or
R4′=OH and R5′=OH and R6′=O-methyl; or
R4′=OH and R5′=OH and R6′=—S(═O)₂OH; or
R4′=OH and R5′=OH and R6′=sulfonyl; or
R4′=OH and R5′=methyl and R6′=H; or
R4′=OH and R5′=methyl and R6′=OH; or
R4′=OH and R5′=methyl and R6′=methyl; or
R4′=OH and R5′=methyl and R6′=O-methyl; or
R4′=OH and R5′=methyl and R6′=—S(═O)₂OH; or
R4′=OH and R5′=methyl and R6′=sulfonyl; or
R4′=OH and R5′=O-methyl and R6′=H; or
R4′=OH and R5′=O-methyl and R6′=OH; or
R4′=OH and R5′=O-methyl and R6′=methyl; or
R4′=OH and R5′=O-methyl and R6′=O-methyl; or
R4′=OH and R5′=O-methyl and R6′=—S(═O)₂OH; or
R4′=OH and R5′=O-methyl and R6′=sulfonyl; or
R4′=OH and R5′=—S(═O)₂OH and R6′=H; or
R4′=OH and R5′=—S(═O)₂OH and R6′=OH; or
R4′=OH and R5′=—S(═O)₂OH and R6′=methyl; or
R4′=OH and R5′=—S(═O)₂OH and R6′=O-methyl; or
R4′=OH and R5′=—S(═O)₂OH and R6′=—S(═O)₂OH; or
R4′=OH and R5′=—S(═O)₂OH and R6′=sulfonyl; or
R4′=OH and R5′=sulfonyl and R6′=H; or
R4′=OH and R5′=sulfonyl and R6′=OH; or
R4′=OH and R5′=sulfonyl and R6′=methyl; or
R4′=OH and R5′=sulfonyl and R6′=O-methyl; or
R4′=OH and R5′=sulfonyl and R6′=—S(═O)₂OH; or
R4′=OH and R5′=sulfonyl and R6′=sulfonyl; or
R4′=methyl and R5′=H and R6′=H; or
R4′=methyl and R5′=H and R6′=OH; or
R4′=methyl and R5′=H and R6′=methyl; or
R4′=methyl and R5′=H and R6′=O-methyl; or
R4′=methyl and R5′=H and R6′=—S(═O)₂OH; or
R4′=methyl and R5′=H and R6′=sulfonyl; or
R4′=methyl and R5′=OH and R6′=H; or
R4′=methyl and R5′=OH and R6′=OH; or
R4′=methyl and R5′=OH and R6′=methyl; or
R4′=methyl and R5′=OH and R6′=O-methyl; or
R4′=methyl and R5′=OH and R6′=—S(═O)₂OH; or
R4′=methyl and R5′=OH and R6′=sulfonyl; or
R4′=methyl and R5′=methyl and R6′=H; or
R4′=methyl and R5′=methyl and R6′=OH; or
R4′=methyl and R5′=methyl and R6′=methyl; or
R4′=methyl and R5′=methyl and R6′=O-methyl; or
R4′=methyl and R5′=methyl and R6′=—S(═O)₂OH; or
R4′=methyl and R5′=methyl and R6′=sulfonyl; or
R4′=methyl and R5′=O-methyl and R6′=H; or
R4′=methyl and R5′=O-methyl and R6′=OH; or
R4′=methyl and R5′=O-methyl and R6′=methyl; or
R4′=methyl and R5′=O-methyl and R6′=O-methyl; or
R4′=methyl and R5′=O-methyl and R6′=—S(═O)₂OH; or
R4′=methyl and R5′=O-methyl and R6′=sulfonyl; or
R4′=methyl and R5′=—S(═O)₂OH and R6′=H; or
R4′=methyl and R5′=—S(═O)₂OH and R6′=OH; or
R4′=methyl and R5′=—S(═O)₂OH and R6′=methyl; or
R4′=methyl and R5′=—S(═O)₂OH and R6′=O-methyl; or
R4′=methyl and R5′=—S(═O)₂OH and R6′=—S(═O)₂OH; or
R4′=methyl and R5′=—S(═O)₂OH and R6′=sulfonyl; or
R4′=methyl and R5′=sulfonyl and R6′=H; or
R4′=methyl and R5′=sulfonyl and R6′=OH; or
R4′=methyl and R5′=sulfonyl and R6′=methyl; or
R4′=methyl and R5′=sulfonyl and R6′=O-methyl; or
R4′=methyl and R5′=sulfonyl and R6′=—S(═O)₂OH; or
R4′=methyl and R5′=sulfonyl and R6′=sulfonyl; or
R4′=O-methyl and R5′=H and R6′=H; or
R4′=O-methyl and R5′=H and R6′=OH; or
R4′=O-methyl and R5′=H and R6′=methyl; or
R4′=O-methyl and R5′=H and R6′=O-methyl; or
R4′=O-methyl and R5′=H and R6′=—S(═O)₂OH; or
R4′=O-methyl and R5′=H and R6′=sulfonyl; or
R4′=O-methyl and R5′=OH and R6′=H; or
R4′=O-methyl and R5′=OH and R6′=OH; or
R4′=O-methyl and R5′=OH and R6′=methyl; or
R4′=O-methyl and R5′=OH and R6′=O-methyl; or
R4′=O-methyl and R5′=OH and R6′=—S(═O)₂OH; or
R4′=O-methyl and R5′=OH and R6′=sulfonyl; or
R4′=O-methyl and R5′=methyl and R6′=H; or
R4′=O-methyl and R5′=methyl and R6′=OH; or
R4′=O-methyl and R5′=methyl and R6′=methyl; or
R4′=O-methyl and R5′=methyl and R6′=O-methyl; or
R4′=O-methyl and R5′=methyl and R6′=—S(═O)₂OH; or
R4′=O-methyl and R5′=methyl and R6′=sulfonyl; or
R4′=O-methyl and R5′=O-methyl and R6′=H; or
R4′=O-methyl and R5′=O-methyl and R6′=OH; or
R4′=O-methyl and R5′=O-methyl and R6′=methyl; or
R4′=O-methyl and R5′=O-methyl and R6′=O-methyl; or
R4′=O-methyl and R5′=O-methyl and R6′=—S(═O)₂OH; or
R4′=O-methyl and R5′=O-methyl and R6′=sulfonyl; or
R4′=O-methyl and R5′=—S(═O)₂OH and R6′=H; or
R4′=O-methyl and R5′=—S(═O)₂OH and R6′=OH; or
R4′=O-methyl and R5′=—S(═O)₂OH and R6′=methyl; or
R4′=O-methyl and R5′=—S(═O)₂OH and R6′=O-methyl; or
R4′=O-methyl and R5′=—S(═O)₂OH and R6′=—S(═O)₂OH; or
R4′=O-methyl and R5′=—S(═O)₂OH and R6′=sulfonyl; or
R4′=O-methyl and R5′=sulfonyl and R6′=H; or
R4′=O-methyl and R5′=sulfonyl and R6′=OH; or
R4′=O-methyl and R5′=sulfonyl and R6′=methyl; or
R4′=O-methyl and R5′=sulfonyl and R6′=O-methyl; or
R4′=O-methyl and R5′=sulfonyl and R6′=—S(═O)₂OH; or
R4′=O-methyl and R5′=sulfonyl and R6′=sulfonyl; or
R4′=—S(═O)₂OH and R5′=H and R6′=H; or
R4′=—S(═O)₂OH and R5′=H and R6′=OH; or
R4′=—S(═O)₂OH and R5′=H and R6′=methyl; or
R4′=—S(═O)₂OH and R5′=H and R6′=O-methyl; or
R4′=—S(═O)₂OH and R5′=H and R6′=—S(═O)₂OH; or
R4′=—S(═O)₂OH and R5′=H and R6′=sulfonyl; or
R4′=—S(═O)₂OH and R5′=OH and R6′=H; or
R4′=—S(═O)₂OH and R5′=OH and R6′=OH; or
R4′=—S(═O)₂OH and R5′=OH and R6′=methyl; or
R4′=—S(═O)₂OH and R5′=OH and R6′=O-methyl; or
R4′=—S(═O)₂OH and R5′=OH and R6′=—S(═O)₂OH; or
R4′=—S(═O)₂OH and R5′=OH and R6′=sulfonyl; or
R4′=—S(═O)₂OH and R5′=methyl and R6′=H; or
R4′=—S(═O)₂OH and R5′=methyl and R6′=OH; or
R4′=—S(═O)₂OH and R5′=methyl and R6′=methyl; or
R4′=—S(═O)₂OH and R5′=methyl and R6′=O-methyl; or
R4′=—S(═O)₂OH and R5′=methyl and R6′=—S(═O)₂OH; or
R4′=—S(═O)₂OH and R5′=methyl and R6′=sulfonyl; or
R4′=—S(═O)₂OH and R5′=O-methyl and R6′=H; or
R4′=—S(═O)₂OH and R5′=O-methyl and R6′=OH; or
R4′=—S(═O)₂OH and R5′=O-methyl and R6′=methyl; or
R4′=—S(═O)₂OH and R5′=O-methyl and R6′=O-methyl; or
R4′=—S(═O)₂OH and R5′=O-methyl and R6′=—S(═O)₂OH; or
R4′=—S(═O)₂OH and R5′=O-methyl and R6′=sulfonyl; or
R4′=—S(═O)₂OH and R5′=—S(═O)₂OH═ and R6′=H; or
R4′=—S(═O)₂OH and R5′=—S(═O)₂OH and R6′=OH; or
R4′=—S(═O)₂OH and R5′=—S(═O)₂OH and R6′=methyl; or
R4′=—S(═O)₂OH and R5′=—S(═O)₂OH and R6′=O-methyl; or
R4′=—S(═O)₂OH and R5′=sulfonyl═ and R6′=H; or
R4′=—S(═O)₂OH and R5′=sulfonyl and R6′=OH; or
R4′=—S(═O)₂OH and R5′=sulfonyl and R6′=methyl; or
R4′=—S(═O)₂OH and R5′=sulfonyl and R6′=O-methyl; or
R4′=sulfonyl and R5′=H and R6′=H; or
R4′=sulfonyl and R5′=H and R6′=OH; or
R4′=sulfonyl and R5′=H and R6′=methyl; or
R4′=sulfonyl and R5′=H and R6′=O-methyl; or
R4′=sulfonyl and R5′=H and R6′=—S(═O)₂OH; or
R4′=sulfonyl and R5′=H and R6′=sulfonyl; or
R4′=sulfonyl and R5′=OH and R6′=H; or
R4′=sulfonyl and R5′=OH and R6′=OH; or
R4′=sulfonyl and R5′=OH and R6′=methyl; or
R4′=sulfonyl and R5′=OH and R6′=O-methyl; or
R4′=sulfonyl and R5′=OH and R6′=—S(═O)₂OH; or
R4′=sulfonyl and R5′=OH and R6′=sulfonyl; or
R4′=sulfonyl and R5′=methyl and R6′=H; or
R4′=sulfonyl and R5′=methyl and R6′=OH; or
R4′=sulfonyl and R5′=methyl and R6′=methyl; or
R4′=sulfonyl and R5′=methyl and R6′=O-methyl; or
R4′=sulfonyl and R5′=methyl and R6′=—S(═O)₂OH; or
R4′=sulfonyl and R5′=methyl and R6′=sulfonyl; or
R4′=sulfonyl and R5′=O-methyl and R6′=H; or
R4′=sulfonyl and R5′=O-methyl and R6′=OH; or
R4′=sulfonyl and R5′=O-methyl and R6′=methyl; or
R4′=sulfonyl and R5′=O-methyl and R6′=O-methyl; or
R4′=sulfonyl and R5′=O-methyl and R6′=—S(═O)₂OH; or
R4′=sulfonyl and R5′=O-methyl and R6′=sulfonyl; or
R4′=sulfonyl and R5′=—S(═O)₂OH and R6′=H; or
R4′=sulfonyl and R5′=—S(═O)₂OH and R6′=OH; or
R4′=sulfonyl and R5′=—S(═O)₂OH and R6′=methyl; or
R4′=sulfonyl and R5′=—S(═O)₂OH and R6′=O-methyl; or
R4′=sulfonyl and R5′=sulfonyl and R6′=H; or
R4′=sulfonyl and R5′=sulfonyl and R6′=OH; or
R4′=sulfonyl and R5′=sulfonyl and R6′=methyl; or
R4′=sulfonyl and R5′=sulfonyl and R6′=O-methyl; or
R4″=H and R5″=H and R6″=H; or
R4″=H and R5″=H and R6″=OH; or
R4″=H and R5″=H and R6″=methyl; or
R4″=H and R5″=H and R6″=O-methyl; or
R4″=H and R5″=H and R6″=—S(═O)₂OH; or
R4″=H and R5″=H and R6″=sulfonyl; or
R4″=H and R5″=OH and R6″=H; or
R4″=H and R5″=OH and R6″=OH; or
R4″=H and R5″=OH and R6″=methyl; or
R4″=H and R5″=OH and R6″=O-methyl; or
R4″=H and R5″=OH and R6″=—S(═O)₂OH; or
R4″=H and R5″=OH and R6″=sulfonyl; or
R4″=H and R5″=methyl and R6″=H; or
R4″=H and R5″=methyl and R6″=OH; or
R4″=H and R5″=methyl and R6″=methyl; or
R4″=H and R5″=methyl and R6″=O-methyl; or
R4″=H and R5″=methyl and R6″=—S(═O)₂OH; or
R4″=H and R5″=methyl and R6″=sulfonyl; or
R4″=H and R5″=O-methyl and R6″=H; or
R4″=H and R5″=O-methyl and R6″=OH; or
R4″=H and R5″=O-methyl and R6″=methyl; or
R4″=H and R5″=O-methyl and R6″=O-methyl; or
R4″=H and R5″=O-methyl and R6″=—S(═O)₂OH; or
R4″=H and R5″=O-methyl and R6″=sulfonyl; or
R4″=H and R5″=—S(═O)₂OH and R6″=H; or
R4″=H and R5″=—S(═O)₂OH and R6″=OH; or
R4″=H and R5″=—S(═O)₂OH and R6″=methyl; or
R4″=H and R5″=—S(═O)₂OH and R6″=O-methyl; or
R4″=H and R5″=—S(═O)₂OH and R6″=—S(═O)₂OH; or
R4″=H and R5″=—S(═O)₂OH and R6″=sulfonyl; or
R4″=H and R5″=sulfonyl and R6″=H; or
R4″=H and R5″=sulfonyl and R6″=OH; or
R4″=H and R5″=sulfonyl and R6″=methyl; or
R4″=H and R5″=sulfonyl and R6″=O-methyl; or
R4″=H and R5″=sulfonyl and R6″=—S(═O)₂OH; or
R4″=H and R5″=sulfonyl and R6″=sulfonyl; or
R4″=OH and R5″=H and R6″=H; or
R4″=OH and R5″=H and R6″=OH; or
R4″=OH and R5″=H and R6″=methyl; or
R4″=OH and R5″=H and R6″=O-methyl; or
R4″=OH and R5″=H and R6″=—S(═O)₂OH; or
R4″=OH and R5″=H and R6″=sulfonyl; or
R4″=OH and R5″=OH and R6″=H; or
R4″=OH and R5″=OH and R6″=OH; or
R4″=OH and R5″=OH and R6″=methyl; or
R4″=OH and R5″=OH and R6″=O-methyl; or
R4″=OH and R5″=OH and R6″=—S(═O)₂OH; or
R4″=OH and R5″=OH and R6″=sulfonyl; or
R4″=OH and R5″=methyl and R6″=H; or
R4″=OH and R5″=methyl and R6″=OH; or
R4″=OH and R5″=methyl and R6″=methyl; or
R4″=OH and R5″=methyl and R6″=O-methyl; or
R4″=OH and R5″=methyl and R6″=—S(═O)₂OH; or
R4″=OH and R5″=methyl and R6″=sulfonyl; or
R4″=OH and R5″=O-methyl and R6″=H; or
R4″=OH and R5″=O-methyl and R6″=OH; or
R4″=OH and R5″=O-methyl and R6″=methyl; or
R4″=OH and R5″=O-methyl and R6″=O-methyl; or
R4″=OH and R5″=O-methyl and R6″=—S(═O)₂OH; or
R4″=OH and R5″=O-methyl and R6″=sulfonyl; or
R4″=OH and R5″=—S(═O)₂OH and R6″=H; or
R4″=OH and R5″=—S(═O)₂OH and R6″=OH; or
R4″=OH and R5″=—S(═O)₂OH and R6″=methyl; or
R4″=OH and R5″=—S(═O)₂OH and R6″=O-methyl; or
R4″=OH and R5″=—S(═O)₂OH and R6″=—S(═O)₂OH; or
R4″=OH and R5″=—S(═O)₂OH and R6″=sulfonyl; or
R4″=OH and R5″=sulfonyl and R6″=H; or
R4″=OH and R5″=sulfonyl and R6″=OH; or
R4″=OH and R5″=sulfonyl and R6″=methyl; or
R4″=OH and R5″=sulfonyl and R6″=O-methyl; or
R4″=OH and R5″=sulfonyl and R6″=—S(═O)₂OH; or
R4″=OH and R5″=sulfonyl and R6″=sulfonyl; or
R4″=methyl and R5″=H and R6″=H; or
R4″=methyl and R5″=H and R6″=OH; or
R4″=methyl and R5″=H and R6″=methyl; or
R4″=methyl and R5″=H and R6″=O-methyl; or
R4″=methyl and R5″=H and R6″=—S(═O)₂OH; or
R4″=methyl and R5″=H and R6″=sulfonyl; or
R4″=methyl and R5″=OH and R6″=H; or
R4″=methyl and R5″=OH and R6″=OH; or
R4″=methyl and R5″=OH and R6″=methyl; or
R4″=methyl and R5″=OH and R6″=O-methyl; or
R4″=methyl and R5″=OH and R6″=—S(═O)₂OH; or
R4″=methyl and R5″=OH and R6″=sulfonyl; or
R4″=methyl and R5″=methyl and R6″=H; or
R4″=methyl and R5″=methyl and R6″=OH; or
R4″=methyl and R5″=methyl and R6″=methyl; or
R4″=methyl and R5″=methyl and R6″=O-methyl; or
R4″=methyl and R5″=methyl and R6″=—S(═O)₂OH; or
R4″=methyl and R5″=methyl and R6″=sulfonyl; or
R4″=methyl and R5″=O-methyl and R6″=H; or
R4″=methyl and R5″=O-methyl and R6″=OH; or
R4″=methyl and R5″=O-methyl and R6″=methyl; or
R4″=methyl and R5″=O-methyl and R6″=O-methyl; or
R4″=methyl and R5″=O-methyl and R6″=—S(═O)₂OH; or
R4″=methyl and R5″=O-methyl and R6″=sulfonyl; or
R4″=methyl and R5″=—S(═O)₂OH and R6″=H; or
R4″=methyl and R5″=—S(═O)₂OH and R6″=OH; or
R4″=methyl and R5″=—S(═O)₂OH and R6″=methyl; or
R4″=methyl and R5″=—S(═O)₂OH and R6″=O-methyl; or
R4″=methyl and R5″=—S(═O)₂OH and R6″=—S(═O)₂OH; or
R4″=methyl and R5″=—S(═O)₂OH and R6″=sulfonyl; or
R4″=methyl and R5″=sulfonyl and R6″=H; or
R4″=methyl and R5″=sulfonyl and R6″=OH; or
R4″=methyl and R5″=sulfonyl and R6″=methyl; or
R4″=methyl and R5″=sulfonyl and R6″=O-methyl; or
R4″=methyl and R5″=sulfonyl and R6″=—S(═O)₂OH; or
R4″=methyl and R5″=sulfonyl and R6″=sulfonyl; or
R4″=O-methyl and R5″=H and R6″=H; or
R4″=O-methyl and R5″=H and R6″=OH; or
R4″=O-methyl and R5″=H and R6″=methyl; or
R4″=O-methyl and R5″=H and R6″=O-methyl; or
R4″=O-methyl and R5″=H and R6″=—S(═O)₂OH; or
R4″=O-methyl and R5″=H and R6″=sulfonyl; or
R4″=O-methyl and R5″=OH and R6″=H; or
R4″=O-methyl and R5″=OH and R6″=OH; or
R4″=O-methyl and R5″=OH and R6″=methyl; or
R4″=O-methyl and R5″=OH and R6″=O-methyl; or
R4″=O-methyl and R5″=OH and R6″=—S(═O)₂OH; or
R4″=O-methyl and R5″=OH and R6″=sulfonyl; or
R4″=O-methyl and R5″=methyl and R6″=H; or
R4″=O-methyl and R5″=methyl and R6″=OH; or
R4″=O-methyl and R5″=methyl and R6″=methyl; or
R4″=O-methyl and R5″=methyl and R6″=O-methyl; or
R4″=O-methyl and R5″=methyl and R6″=—S(═O)₂OH; or
R4″=O-methyl and R5″=methyl and R6″=sulfonyl; or
R4″=O-methyl and R5″=O-methyl and R6″=H; or
R4″=O-methyl and R5″=O-methyl and R6″=OH; or
R4″=O-methyl and R5″=O-methyl and R6″=methyl; or
R4″=O-methyl and R5″=O-methyl and R6″=O-methyl; or
R4″=O-methyl and R5″=O-methyl and R6″=—S(═O)₂OH; or
R4″=O-methyl and R5″=O-methyl and R6″=sulfonyl; or
R4″=O-methyl and R5″=—S(═O)₂OH and R6″=H; or
R4″=O-methyl and R5″=—S(═O)₂OH and R6″=OH; or
R4″=O-methyl and R5″=—S(═O)₂OH and R6″=methyl; or
R4″=O-methyl and R5″=—S(═O)₂OH and R6″=O-methyl; or
R4″=O-methyl and R5″=—S(═O)₂OH and R6″=—S(═O)₂OH; or
R4″=O-methyl and R5″=—S(═O)₂OH and R6″=sulfonyl; or
R4″=O-methyl and R5″=sulfonyl and R6″=H; or
R4″=O-methyl and R5″=sulfonyl and R6″=OH; or
R4″=O-methyl and R5″=sulfonyl and R6″=methyl; or
R4″=O-methyl and R5″=sulfonyl and R6″=O-methyl; or
R4″=O-methyl and R5″=sulfonyl and R6″=—S(═O)₂OH; or
R4″=O-methyl and R5″=sulfonyl and R6″=sulfonyl; or
R4″=—S(═O)₂OH and R5″=H and R6″=H; or
R4″=—S(═O)₂OH and R5″=H and R6″=OH; or
R4″=—S(═O)₂OH and R5″=H and R6″=methyl; or
R4″=—S(═O)₂OH and R5″=H and R6″=O-methyl; or
R4″=—S(═O)₂OH and R5″=H and R6″=—S(═O)₂OH; or
R4″=—S(═O)₂OH and R5″=H and R6″=sulfonyl; or
R4″=—S(═O)₂OH and R5″=OH and R6″=H; or
R4″=—S(═O)₂OH and R5″=OH and R6″=OH; or
R4″=—S(═O)₂OH and R5″=OH and R6″=methyl; or
R4″=—S(═O)₂OH and R5″=OH and R6″=O-methyl; or
R4″=—S(═O)₂OH and R5″=OH and R6″=—S(═O)₂OH; or
R4″=—S(═O)₂OH and R5″=OH and R6″=sulfonyl; or
R4″=—S(═O)₂OH and R5″=methyl and R6″=H; or
R4″=—S(═O)₂OH and R5″=methyl and R6″=OH; or
R4″=—S(═O)₂OH and R5″=methyl and R6″=methyl; or
R4″=—S(═O)₂OH and R5″=methyl and R6″=O-methyl; or
R4″=—S(═O)₂OH and R5″=methyl and R6″=—S(═O)₂OH; or
R4″=—S(═O)₂OH and R5″=methyl and R6″=sulfonyl; or
R4″=—S(═O)₂OH and R5″=O-methyl and R6″=H; or
R4″=—S(═O)₂OH and R5″=O-methyl and R6″=OH; or
R4″=—S(═O)₂OH and R5″=O-methyl and R6″=methyl; or
R4″=—S(═O)₂OH and R5″=O-methyl and R6″=O-methyl; or
R4″=—S(═O)₂OH and R5″=O-methyl and R6″=—S(═O)₂OH; or
R4″=—S(═O)₂OH and R5″=O-methyl and R6″=sulfonyl; or
R4″=—S(═O)₂OH and R5″=—S(═O)₂OH═ and R6″=H; or
R4″=—S(═O)₂OH and R5″=—S(═O)₂OH and R6″=OH; or
R4″=—S(═O)₂OH and R5″=—S(═O)₂OH and R6″=methyl; or
R4″=—S(═O)₂OH and R5″=—S(═O)₂OH and R6″=O-methyl; or
R4″=—S(═O)₂OH and R5″=sulfonyl═ and R6″=H; or
R4″=—S(═O)₂OH and R5″=sulfonyl and R6″=OH; or
R4″=—S(═O)₂OH and R5″=sulfonyl and R6″=methyl; or
R4″=—S(═O)₂OH and R5″=sulfonyl and R6″=O-methyl; or
R4″=sulfonyl and R5″=H and R6″=H; or
R4″=sulfonyl and R5″=H and R6″=OH; or
R4″=sulfonyl and R5″=H and R6″=methyl; or
R4″=sulfonyl and R5″=H and R6″=O-methyl; or
R4″=sulfonyl and R5″=H and R6″=—S(═O)₂OH; or
R4″=sulfonyl and R5″=H and R6″=sulfonyl; or
R4″=sulfonyl and R5″=OH and R6″=H; or
R4″=sulfonyl and R5″=OH and R6″=OH; or
R4″=sulfonyl and R5″=OH and R6″=methyl; or
R4″=sulfonyl and R5″=OH and R6″=O-methyl; or
R4″=sulfonyl and R5″=OH and R6″=—S(═O)₂OH; or
R4″=sulfonyl and R5″=OH and R6″=sulfonyl; or
R4″=sulfonyl and R5″=methyl and R6″=H; or
R4″=sulfonyl and R5″=methyl and R6″=OH; or
R4″=sulfonyl and R5″=methyl and R6″=methyl; or
R4″=sulfonyl and R5″=methyl and R6″=O-methyl; or
R4″=sulfonyl and R5″=methyl and R6″=—S(═O)₂OH; or
R4″=sulfonyl and R5″=methyl and R6″=sulfonyl; or
R4″=sulfonyl and R5″=O-methyl and R6″=H; or
R4″=sulfonyl and R5″=O-methyl and R6″=OH; or
R4″=sulfonyl and R5″=O-methyl and R6″=methyl; or
R4″=sulfonyl and R5″=O-methyl and R6″=O-methyl; or
R4″=sulfonyl and R5″=O-methyl and R6″=—S(═O)₂OH; or
R4″=sulfonyl and R5″=O-methyl and R6″=sulfonyl; or
R4″=sulfonyl and R5″=—S(═O)₂OH and R6″=H; or
R4″=sulfonyl and R5″=—S(═O)₂OH and R6″=OH; or
R4″=sulfonyl and R5″=—S(═O)₂OH and R6″=methyl; or
R4″=sulfonyl and R5″=—S(═O)₂OH and R6″=O-methyl; or
R4″=sulfonyl and R5″=sulfonyl and R6″=H; or
R4″=sulfonyl and R5″=sulfonyl and R6″=OH; or
R4″=sulfonyl and R5″=sulfonyl and R6″=methyl; or
R4″=sulfonyl and R5″=sulfonyl and R6″=O-methyl.

In the general formulae (V), (VI), (VII), (VIII), (IX), (X) and their variants, the substituted phenyl ring bonded to the imino functionality is preferably selected from the group consisting of

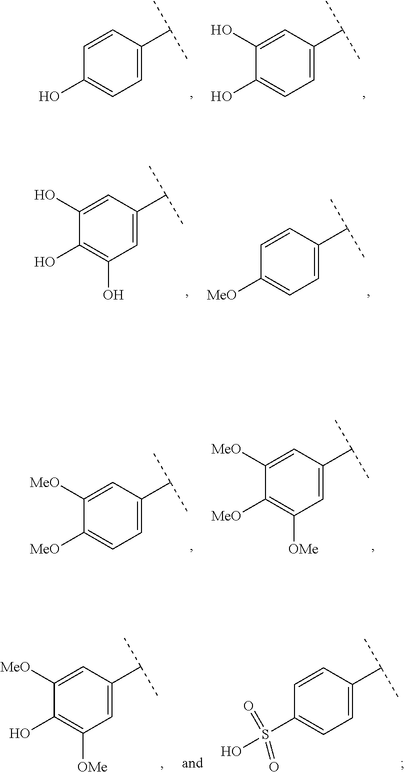

wherein the dotted line designates the binding site to the cyclohexene imine ring.

In a still more preferred variant according to the first aspect of the present invention, in the general formulae (V), (VII), (VIII) and their variants, the substituents R4′, R5′ and R6′, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (V), (VII) and (VIII), but not in the para position to the imino-functionality.

Alternatively, in a still more preferred variant according to the first aspect of the present invention, in the general formulae (VI), (IX), (X) and their variants, the substituents R4″, R5″ and R6″, which may be identical or different, are independently from each other selected from the group consisting of H, OH, alkyl, preferably methyl, ethyl, propyl, iso-propyl, butyl, isobutyl or tert.-butyl, alkoxy, preferably, O-methyl, O-ethyl or O-butyl, —S(═O)₂OH, and sulfonyl; with the proviso, that if the substituted phenyl ring is monosubstituted, the substituent is either in the ortho or meta-position to the imino-functionality of the general formulae (VI), (IX) and (X), but not in the para position to the imino-functionality.

Still more preferred according to the first aspect of the present invention, the food, cosmetic or pharmaceutical preparation or homecare product comprises at least one mycosporine-like amino acid compound selected from the group consisting of:

TABLE 3

| Com- | | |
|---|---|---|
| pound | Structure | INCI/chemical name |
| MAA-1 | 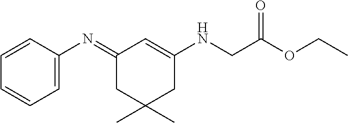 [SMILES: CCOC(=O)CNC1=C/C(=N/c2ccccc2)CC(C)(C)C1] | ethyl 2-[[5,5-dimethyl-3- phenylimino-cyclohexen-1- yl]amino]acetate |
| MAA-2 | 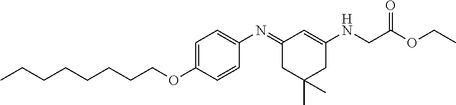 [SMILES: CCCCCCCCOc1ccc(/N=C2/C=C(NCC(=O)OCC)CC(C)(C)C2)cc1] | ethyl 2-((5,5-dimethyl-3-((4- octyoxy)phenyl)imino)cyclohex-1- en-1-yl)amino)acetate |
| MAA-3 | 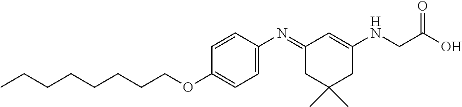 [SMILES: CCCCCCCCOc1ccc(/N=C2/C=C(NCC(=O)O)CC(C)(C)C2)cc1] | 5,5-dimethyl-3-((4- (octyloxy)phenyl)imino)cyclohex- 1-en-1-yl-glycine |
| MAA-4 | 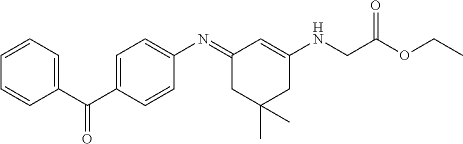 [SMILES: CCOC(=O)CNC1=C/C(=N/c2ccc(C(=O)c3ccccc3)cc2)CC(C)(C)C1] | ethyl 2-((3-((4- benzoylphenyl)imino)-5,5- dimethylcyclohex-1-en-1- yl)amino)acetate |
| MAA-5 | 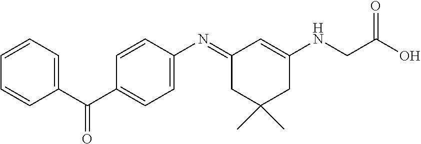 [SMILES: CC1(C)CC(NCC(=O)O)=C/C(=N/c2ccc(C(=O)c3ccccc3)cc2)C1] | 3-((4-benzoylphenyl)imino)-5,5- dimethylcyclohex-1-en-1-yl- glycine |
| MAA-6 | 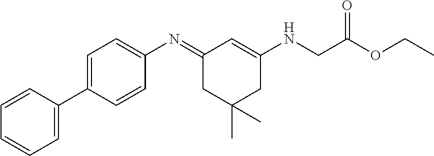 [SMILES: CCOC(=O)CNC1=C/C(=N/c2ccc(-c3ccccc3)cc2)CC(C)(C)C1] | ethyl 2-((3-([1,1′-biphenyl]-4- ylimino)-5,5-dimethylcyclohex-1- en-1.yl)amino)acetate |
| MAA-7 | 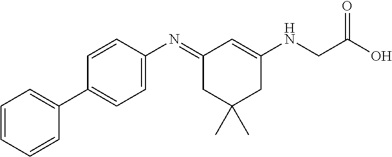 [SMILES: CC1(C)CC(NCC(=O)O)=C/C(=N/c2ccc(-c3ccccc3)cc2)C1] | 3-([1,1′-biphenyl]-4-ylimino)-5,5- dimethylcyclohex-1-en-1-yl- glycine |
| MAA-8 | 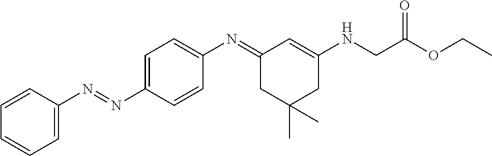 [SMILES: CCOC(=O)CNC1=C/C(=N/c2ccc(/N=N/c3ccccc3)cc2)CC(C)(C)C1] | ethyl 2-((5,5-dimethyl-3-((4- (phenyldiazenyl)phenyl)inino (cyclohex-1-en-1-yl)amino)acetate |
| MAA-9 | 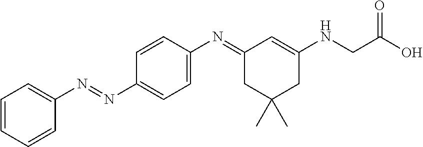 [SMILES: CC1(C)CC(NCC(=O)O)=C/C(=N/c2ccc(/N=N/c3ccccc3)cc2)C1] | 5,5-dimethyl-3-((4-((E)- phenyldiazenyl)phenyl)imino) cyclohex-1-en-1-yl-glycine |
| MAA-11 | 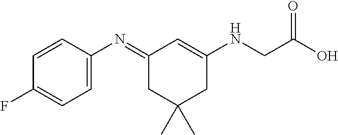 [SMILES: CC1(C)CC(NCC(=O)O)=C/C(=N/c2ccc(F)cc2)C1] | 3-((4-fluorophenyl)imino)-5,5- dimethylcyclohex-1-en-1-yl- glycine |
| MAA-12 | 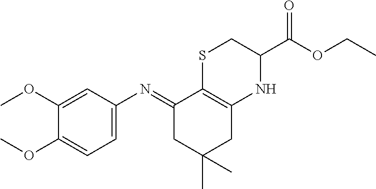 [SMILES: CCOC(=O)C1CSC2=C(CC(C)(C)C/C2=N\c2ccc(OC)c(OC)c2)N1] | ethyl 8-((3,4- dimethoyxphenyl)imino)-6,6- dimethyl-3,4,5,6,7,8-hexahydro-2- H-benzol[b][1,4]thiazine-3- carboxylate |
| MAA-13 | 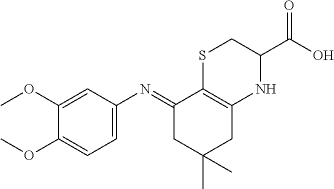 [SMILES: COc1ccc(/N=C2\CC(C)(C)CC3=C2SCC(C(=O)O)N3)cc1OC] | 8-((3,4-dimethoyxphenyl)imino)- 6,6-dimethyl-3,4,5,6,7,8- hexahydro-2-H- benzol[b][1,4]thiazine-3- carboxylic acid |
| MAA-14 | 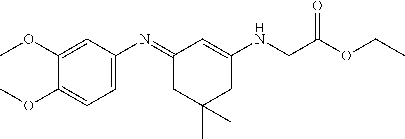 [SMILES: CCOC(=O)CNC1=C/C(=N/c2ccc(OC)c(OC)c2)CC(C)(C)C1] | ethyl 2-((3-((3,4- dimethoxyphenyl)imino)-5,5- dimethylcyclohex-1-en-1- yl)amino)acetate |
| MAA-16 | 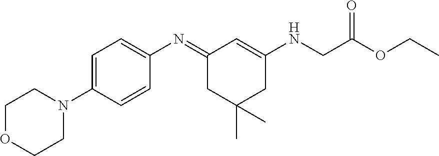 [SMILES: CCOC(=O)CNC1=C/C(=N/c2ccc(N3CCOCC3)cc2)CC(C)(C)C1] | ethyl 2-((5,5-dimethyl-3-((4- morpholinophenyl)imino)cyclohex- 1-en-1-yl)amino)acetate |
| MAA-17 | 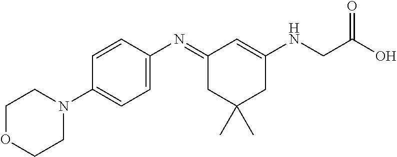 [SMILES: CC1(C)CC(NCC(=O)O)=C/C(=N/c2ccc(N3CCOCC3)cc2)C1] | 5,5-dimethyl-3-((4- morpholinophenyl)imino)cyclohex- 1-en-1-yl-glycine |
| MAA-20 | 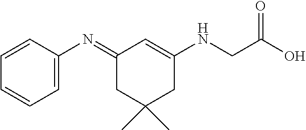 [SMILES: CC1(C)CC(NCC(=O)O)=C/C(=N/c2ccccc2)C1] | 2-[(5,5-dimethyl-3-phenylimino- cyclohexen-1-yl)amino]acetic acid |
| MAA-21 | 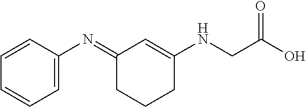 [SMILES: O=C(O)CNC1=C/C(=N/c2ccccc2)CCC1] | 2-[(3-phenyliminocyclohexen-1- yl)amino]acetic acid |
| MAA-22 | 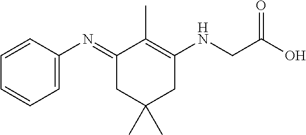 [SMILES: CC1=C(NCC(=O)O)CC(C)(C)C/C1=N\c1ccccc1] | 2,5,5-trimethyl-3- (phenylimino)cyclohex-1-en-1-yl- glycine |
| MAA-23 | 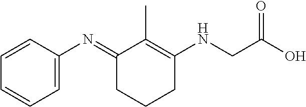 [SMILES: CC1=C(NCC(=O)O)CCC/C1=N\c1ccccc1] | 2-[(2-methyl-3-phenylimino- cyclohexen-1-yl)amino]acetic acid |
| MAA-24 | 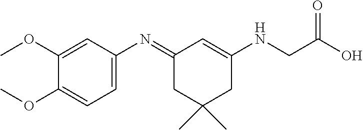 [SMILES: COc1ccc(/N=C2/C=C(NCC(=O)O)CC(C)(C)C2)cc1OC] | 3-((3,4-dimethoxyphenyl)imino)- 5,5-dimethylcyclohex-1-en-1-yl- glycine |
| MAA-25 | 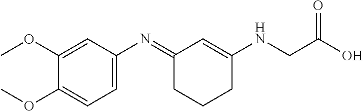 [SMILES: COc1ccc(/N=C2/C=C(NCC(=O)O)CCC2)cc1OC] | 3-((3,4- dimethoxyphenyl)imino)cyclohex- 1-en-1-yl-glycine |
| MAA-26 | 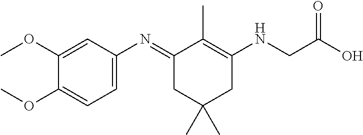 [SMILES: COc1ccc(/N=C2\CC(C)(C)CC(NCC(=O)O)=C2C)cc1OC] | 3-((3,4-dimethoxyphenyl)imino)- 2,5,5-trimethylcyclohex-1-en-1-yl- glycine |
| MAA-27 | 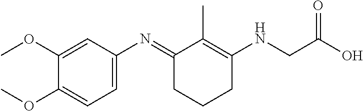 [SMILES: COc1ccc(/N=C2\CCCC(NCC(=O)O)=C2C)cc1OC] | 2-[(3-(3,4-dimethoxyphenyl)imino- 2-methyl-cyclohexen-1- yl)amino]acetic acid |
| MAA-28 | 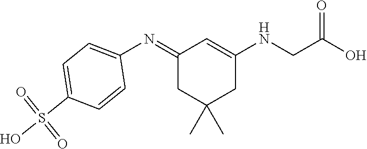 [SMILES: CC1(C)CC(NCC(=O)O)=C/C(=N/c2ccc(S(=O)(=O)O)cc2)C1] | 2-[(5,5-dimethyl-3-(4- sulfophenyl)imino-cyclohexen-1- yl)amino]acetic acid |
| MAA-29 | 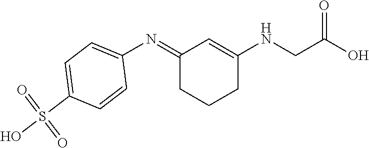 [SMILES: O=C(O)CNC1=C/C(=N/c2ccc(S(=O)(=O)O)cc2)CCC1] | 2-[(3-(4- sulfophenyl)iminocyclohexen-1- yl)amino]acetic acid |
| MAA-30 | 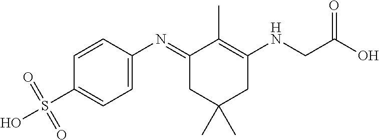 [SMILES: CC1=C(NCC(=O)O)CC(C)(C)C/C1=N\c1ccc(S(=O)(=O)O)cc1] | 2,5,5-trimethyl-3-((4- sulfophenyl)imino)cyclohex-1-en- 1-yl-glycine |
| MAA-31 | 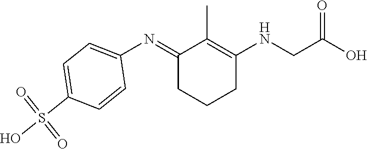 [SMILES: CC1=C(NCC(=O)O)CCC/C1=N\c1ccc(S(=O)(=O)O)cc1] | 2-methyl-3-((4- sulfophenyl)imino)cyclohex-1-en- 1-yl-glycine |
| MAA-32 | 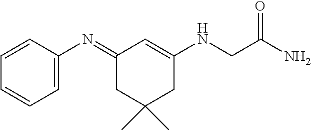 [SMILES: CC1(C)CC(NCC(N)=O)=C/C(=N/c2ccccc2)C1] | 2-[(5,5-dimethyl-3-phenylimino- cyclohexen-1-yl)amino]acetamide |
| MAA-33 | 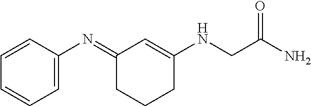 [SMILES: NC(=O)CNC1=C/C(=N/c2ccccc2)CCC1] | 2-[(3-phenyliminocyclohexen-1- yl)amino]acetamide |
| MAA-34 | 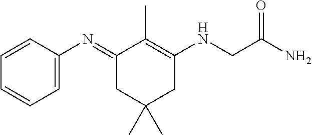 [SMILES: CC1=C(NCC(N)=O)CC(C)(C)C/C1=N\c1ccccc1] | 2-((2,5,5-trimethyl-3- (phenylimino)cyclohex-1-en-1- yl)amino)acetamide |
| MAA-35 | 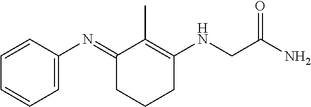 [SMILES: CC1=C(NCC(N)=O)CCC/C1=N\c1ccccc1] | 2-((2-methyl-3- (phenylimino)cyclohex-1-en-1- yl)amino)acetamide |
| MAA-36 | 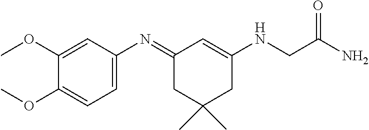 [SMILES: COc1ccc(/N=C2/C=C(NCC(N)=O)CC(C)(C)C2)cc1OC] | 2-((3-((3,4- dimethoxyphenyl)imino)-5,5- dimethylcyclohex-1-en-1- yl)amino)acetamide |
| MAA-37 | 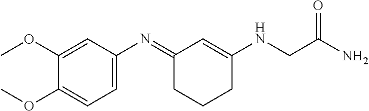 [SMILES: COc1ccc(/N=C2/C=C(NCC(N)=O)CCC2)cc1OC] | 2-[(3-(3,4- dimethoxyphenyl)iminocyclo- hexen-1-yl)amino]acetamide |
| MAA-38 | 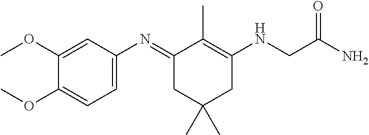 [SMILES: COc1ccc(/N=C2\CC(C)(C)CC(NCC(N)=O)=C2C)cc1OC] | 2-((3-((3,4- dimethoxyphenyl)imino)-2,5,5- trimethylcyclohex-1-en-1- yl)amino)acetamide |
| MAA-39 | 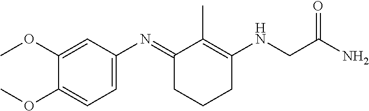 [SMILES: COc1ccc(/N=C2\CCCC(NCC(N)=O)=C2C)cc1OC] | 2-((3-((3,4- dimethoxyphenyl)imino)-2- methylcyclohex-1-en-1- yl)amino)acetamide |

or a tautomer or a stereoisomer or a salt of the afore-mentioned compounds, or any mixture of the afore-mentioned compounds.

Among the above specified mycosporine-like amino acid compounds the compounds MAA-1 and MAA-13 are particularly preferred, since they have a pronounced stabilizing effect. Most preferred are the acid compounds MAA-20, MAA-21, MAA-22, MAA-23, MAA-24, MAA-24, MAA-26, MAA-27, MAA-28, MAA-29, MAA-30, MAA-31, and the amide compounds MAA-32, MAA-33, MAA-34, MAA-35, MAA-36, MAA-37, MAA-38, and MAA-39. Said compounds having an acid functionality or an amide functionality are photo-stable and temperature-stable and show stability in pH solution or in oil-in-water emulsions and does not degrade compared to the respective ester analogues. Particularly preferred are the acid compounds MAA-20, MAA-21, MAA-22, MAA-23, MAA-24, MAA-24, MAA-26, MAA-27, MAA-28, MAA-29, MAA-30 and MAA-31 due to their stability and their improved solubility in water.

Especially, the mycosporine-like amino acid compounds MAA-20, MAA-21, MAA-22, MAA-23, MAA-24, MAA-24, MAA-26, MAA-27, MAA-28, MAA-29, MAA-30 and MAA-31 have a better stability in a broader pH-range compared to their respective ester compounds which hydrolize, and, thus, are not stable in alkaline solutions/formulations having a pH higher than 9.

Due to their better solubility in water, said mycosporine-like amino acid compounds can be incorporated into the water-phase of emulsions in higher concentrations, resulting in an improved boosting effect of an antioxidant in food, cosmetic or pharmaceutical preparations or homecare products, compared to their respective ester compounds.

Still more preferred according to the first aspect of the present invention, the food, cosmetic or pharmaceutical preparation or homecare product comprises at least one mycosporine-like amino acid compound selected from the group consisting of:

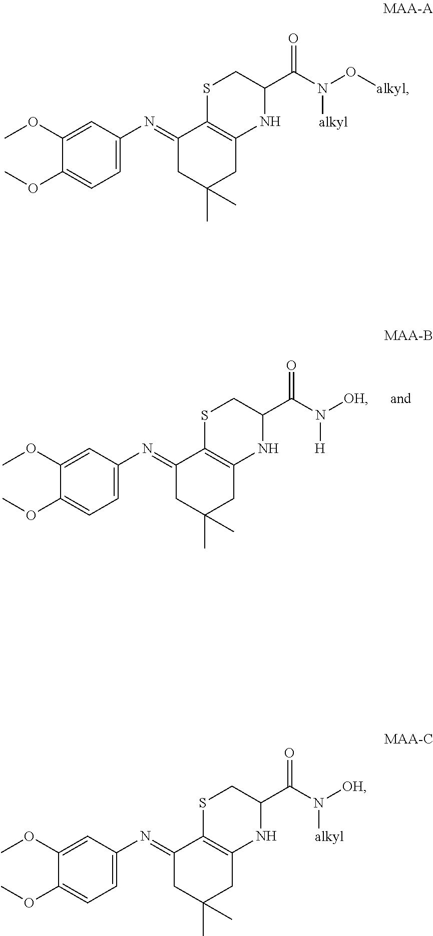

or a tautomer or a stereoisomer or a salt of the afore-mentioned compounds, or any mixture of the afore-mentioned compounds, wherein alkyl is preferably either methyl or ethyl or propyl or isopropyl, or butyl or isobutyl or tert-butyl, most preferred methyl.

The mycosporine-like amino acid compounds according to the present invention and defined herein are used either as single substance or in a mixture with one, two or more different mycosporine-like amino acid compounds as defined herein.

Particular preferred are mixtures of two mycosporine-like amino acid compounds according to the present invention and defined herein. Such mixtures show a particular enhanced stabilizing and antioxidant boosting effect, as it is demonstrated by the following examples.

The food, cosmetic or pharmaceutical composition or homecare product according to the first aspect of the present invention is further characterized in that it does not contain one or more of the following mycosporine-like amino acid compounds:

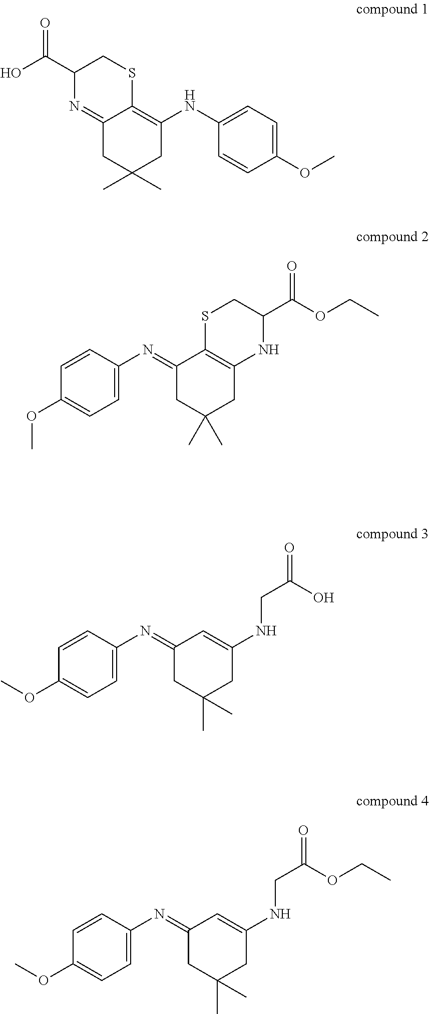

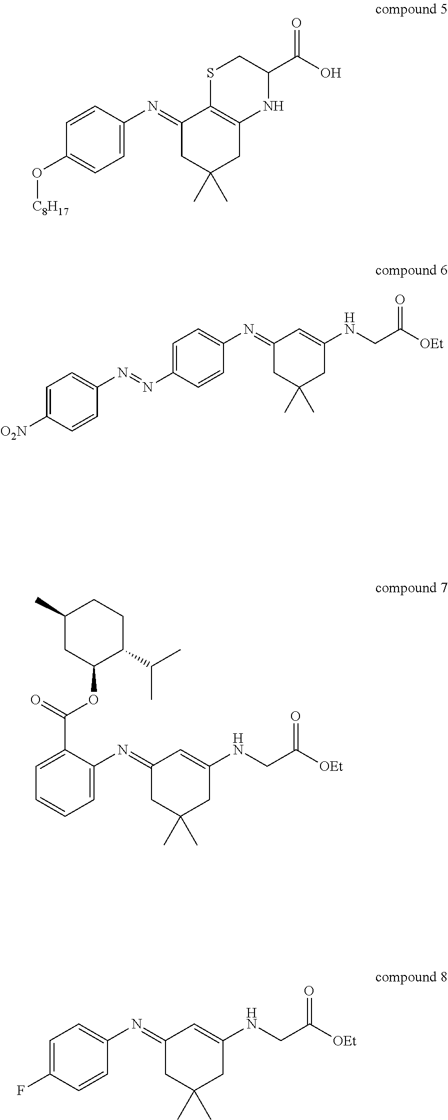

Various mycosporine-like amino acid compounds (b) as described herein contain one or more chiral centers, and, thus, can exist as racemic mixtures of enantiomers, mixtures of diastereomers or enantiomerically or optically pure compounds. It should also be noted the compounds of the invention include E and Z isomers, or a mixture thereof, and cis and trans isomers or a mixture thereof.

Accordingly, the chemical structures of the mycosporine-like amino acid compounds according to the general formulae (V) to (X) and their variants and compounds explicitly disclosed herein include all possible enantiomers and diastereomers or stereoisomers thereof.

In certain embodiments, the mycosporine-like amino acid compounds (b) of the invention are isolated as either the E or Z isomer. In other embodiments, the compounds of the invention are a mixture of the E and Z isomers.

Alternatively, stereomerically pure compounds are used in the food, cosmetic or pharmaceutical preparation or homecare product according to the present invention. As used herein and unless otherwise indicated, the term “stereomerically pure” means that one stereoisomer of a compound is substantially free of other stereoisomers of that compound or one geometric isomer (e.g., about a double bond) is substantially free of the other geometric isomer. For example, a stereomerically pure compound of the invention having one chiral center, will be substantially free of the opposite enantiomer of the compound. A stereomerically pure compound of the invention having two chiral centers, or a composition thereof, will be substantially free of other diastereomers of the compound. A stereomerically pure compound of the invention having a double bond capable of E/Z isomerism, or a composition thereof, will be substantially free of one of the E/Z isomers.

A typical stereomerically pure compound comprises greater than about 80% by weight of one stereoisomer or E/Z isomer of the compound and less than about 20% by weight of other stereoisomers or E/Z isomer of the compound, more preferably greater than about 90% by weight of one stereoisomer or E/Z isomer of the compound and less than about 10% by weight of the other stereoisomers or E/Z isomer of the compound, even more preferably greater than about 95% by weight of one stereoisomer or E/Z isomer of the compound and less than about 5% by weight of the other stereoisomers or E/Z isomer of the compound, and most preferably greater than about 97% by weight of one stereoisomer or E/Z isomer of the compound and less than about 3% by weight of the other stereoisomers or E/Z isomer of the compound.

As used herein and unless otherwise indicated, the term “stereomerically enriched” means a compound of the invention, or a composition thereof, that comprises greater than about 60% by weight of one stereoisomer or E/Z isomer of a compound of the invention, preferably greater than about 70% by weight, more preferably greater than about 80% by weight of one stereoisomer or E/Z isomer of a compound of the invention.

Thus, it should be noted that unless stated otherwise, the term mycosporine-like amino acid compound according to the general formulae (V) to (X) and their variants as well as individual mycosporine-like amino acid compounds specified herein, are to be interpreted as encompassing racemic mixtures of enantiomers, mixtures of diastereomers or enantiomerically or optically pure compounds.

Due to their amino cyclohexene imine structure and their capability of electron delocalization, the mycosporine-like amino acid compounds according to the general formulae (V) to (X) and their variants are in equilibrium with their tautomer structures, in which the hydrogen of the amino group changes its places with the double bond of the cyclohexene ring.

Tautomerism is defined as each of two or more isomers of a compound which exist together in equilibrium and are readily interchanged by migration of an atom or group within the molecule. Tautomeric pure or enriched systems will readily interchange into the equilibrium state over time.

The tautomers of general formula (V) are represented by general formulae (V-tau):

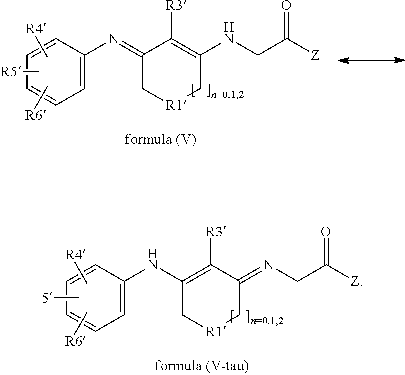

The tautomers of general formula (VI) are represented by general formula (VI-tau):

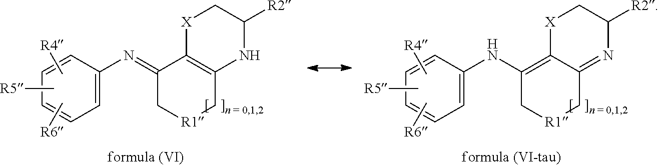

The above principles of tautomerism and redeposition explained on the basis of the mycosporine-like amino acid compounds according to the general formulae (V) and (VI) are likewise applicable with respect to any of the mycosporine-like amino acid compounds defined by the general formulae (VII), (VIII), (IX), (X) and their variants or with respect to any individual mycosporine-like amino acid compounds specified herein.

Thus, in the following and throughout the description, the reference to a mycosporine-like amino acid compound represented by any of the general formulae (V) to (X) and their variants or to an individual mycosporine-like amino acid compound specified herein encompasses likewise its tautomer isomer(s).

The mycosporine-like amino acid compounds according to the general formulae (V) to (X) and their variants as well as individual mycosporine-like amino acid compounds specified herein are used according to the first aspect of the present invention either in neutral, i.e. uncharged form, or in the form of their salts, such as an acid addition salt, with inorganic or organic acids.

The term “salt” in the context of the present invention refers to a salt of a compound that possesses the desired effect or pharmacological activity of the parent compound. Such salts include:
(1) acid addition salts formed with inorganic acids, or formed with organic acids, preferably monovalent or polyvalent carboxylic acids; or
(2) salts formed when an acidic proton present in the starting compound is replaced by a metal ion, e.g., an alkali metal ion, an alkaline earth ion, or an aluminium ion; or coordinated with an organic base.

Among the salts, acid addition salts are again particularly preferred, since the mycosporine-like amino acid compounds according to the general formulae (V) to (X) and their variants or the mycosporine-like amino acid compounds specified herein comprise a protonable N atom.

The inorganic acids that form acid addition salts with the mycosporine-like amino acid compounds used according to the present invention are preferably selected from the group consisting of hydrochloric acid, hydrobromic acid, sulfuric acid, nitric acid, phosphoric acid and the like. Particularly preferred is the hydrochloride salt or the sulfate salt or the phosphate salt.

Even more preferred are acid addition salts with organic mono- or polycarboxylic acids. Further preferred are acid addition salts with organic mono- or polycarboxylic acids, wherein the carboxylic acid is selected from saturated or mono- or polyunsaturated C1 to C30 monocarboxylic acids, saturated or mono- or polyunsaturated C3 to 10 di- or tricarboxylic acids. The carboxylic acid may be mono- or poly-substituted with hydroxy groups, preferably α-hydroxycarboxylic acids in which the hydroxy group is located on the carbon atom adjacent to the carboxy group. Many representatives occur naturally as so-called fruit acids. Preferred α-hydroxycarboxylic acids are malic acid, citric acid, 2-hydroxy-4-methylmercaptobutyric acid, glycolic acid, isocitric acid, mandelic acid, lactic acid, tartronic acid, or tartaric acid.

The organic acids that form acid addition salts with the mycosporine-like amino acid compounds used according to the present invention are preferably selected from the group consisting of amino acids, acetic acid, trifluoroacetic acid, propionic acid, hexanoic acid, cyclopentane propionic acid, glycolic acid, pyruvic acid, lactic acid, malonic acid, oxalic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tert. butylacetic acid, laurylsulfuric acid, gluconic acid, glutamic acid, hydroxynaphthoic acid, salicylic acid, stearic acid, muconic acid, 4-hydroxybutanoic acid, and the like.

Suitable anionic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C1- to C4-alkanoic acids, preferably formate, acetate, trifluoroacetate, propionate and butyrate, furthermore lactate, gluconate, and the anions of poly acids such as succinate, oxalate, maleate, fumarate, malate, tartrate and citrate, furthermore sulfonate anions such as besylate (benzenesulfonate), tosylate (p-toluenesulfonate), napsylate (naphthalene-2-sulfonate), mesylate (methanesulfonate), esylate (ethanesulfonate), and ethanedisulfonate. They can be formed by reacting compounds according to the invention that have a basic functionality with an acid of the corresponding anion. Preferred salts of the compounds of formulae (I) to (III) as well as of the individual mycosporine-like amino acid compounds specified herein are chloride salts.

The metal ions for salt formation that replace an acidic proton present in the starting compound are selected from the group consisting of alkali metal ions, preferably Na+, K+, or Lit, alkaline earth metal ions, preferably Ca++ or Mg++, aluminium+++, or NH4+.

The coordinating organic base for salt formation is selected from the group consisting of ethanolamine, diethanolamine, triethanolamine, N-methylglucamine, triethylamine, and the like.

In the following description and claims, and unless stated otherwise, the term mycosporine-like amino acid compound includes both the neutral, uncharged form of the compound/molecule and equally the salt form of the compound.

The salt form of the mycosporine-like amino acid compounds leads to a lower logPow and, thus, making the compounds more hydrophilic, which results in a better water solubility. In particular, the cationic salts of the mycosporine-like amino acid compounds support emulsification processes in emulsions, due to their surface reducing activity, i.e. co-emulsifying, properties. In addition, the cationic salt form of the mycosporine-like compounds shows excellent substantivity behaviour on skin and hair and other non-biological surfaces: most conditioning actives are cationic; the conditioning effect leads to a softer skin feel, which makes the end products more accepted by the consumer. In addition, the mycosporine-like amino acid compounds in the cationic salt form exhibit antimicrobial activity.

Surprisingly, the addition of a mycosporine-like amino acid compound to a fragrance or aroma/flavour compound in food, a cosmetic or pharmaceutical preparation or homecare product leads to an improvement in fragrance or aroma/flavour stability. In the presence of a mycosporine-like amino acid compound the fragrance or aroma/flavour compound are protected from degradation by oxygen, temperature and/or light. Therefore, the use of mycosporine-like amino acid compounds prevents said formulations from degradation, odour change, i.e. malodour formation, and/or discoloration, as it is demonstrated in the following examples.

Due to the outstanding stabilizing effects as described above, the combined use of at least one fragrance or aroma/flavour compound (a) and at least one mycosporine-like amino acid compound (b) is suitable to improve storage stability and to prolong shelf life of the food, cosmetic or pharmaceutical preparations or homecare products.

The food, cosmetic or pharmaceutical preparation or homecare product according to the present invention optionally contains at least one antioxidant (c). Optionally means that the antioxidant (c) is not mandatory in the food, cosmetic or pharmaceutical preparation or homecare product according to the present invention but can be advantageously incorporated.

The term “antioxidant” as used in the present application refers to a substance or composition which significantly delays, prevents or even inhibits oxidation. Antioxidants react with free radicals, reducing them to stable, unreactive products. Oxidation is a chemical reaction that produces free radicals chain reactions which generate highly reactive peroxides and hydroperoxides. In turn, these peroxides and hydroperoxides react with substrates present in a cosmetic composition or in food which result in decomposition products such as carboxylic acids, aldehydes and ketones, among other, which alter the sensory properties of the productions and lead to degradation of the composition.

Antioxidants play an important physiological role as radical scavenger. In the organism antioxidants inactivate biologically important reactive free radicals or other reactive oxygen species (ROS) which excessively occurrence leads to oxidative stress. In a biological context, reactive oxygen species (ROS) are formed as a natural by-product of the normal metabolism of oxygen and have important roles in cell signalling and homeostasis. However, at times of environmental stress (for example UV or heat exposure), ROS levels can increase dramatically.

With the use of an antioxidant, the above-described oxidation processes in the formulations according to the present invention or the above described biochemically reactions caused by oxidative stress and excess reactive oxygen species (ROS) can be down regulated.

The food, cosmetic or pharmaceutical composition or homecare product according to the first aspect of the present invention encompasses at least one antioxidant selected from the group consisting of dimethylmethoxy chromanol, arbutin, amino acids (for example arginine, lysine, glycine, histidine, tyrosine, tryptophan) and their derivatives (for example acetylcysteine), tert-butylhydroquinone, caffeic acid, chlorogenic acid, imidazoles (for example urocanic acid) and their derivatives, cannabinoids, cannabidiol and its extracts (Cannabis sativa seed oil, Cannabis sativa extract), cannabinol, kojic acid, peptides such as D,L-carnosine, D-carnosine, L-carnosine and their derivatives (for example anserine), hydroxyphenyl propamidobenzoic acid (dihydroavenanthramide D), diethylhexyl syringylidene malonate, phenylethyl resorcinol, gallic acid and their derivatives, quercetin, hydroxyacetophenone, rosmarinic acid, carotenoids, carotenes (for example α-carotene, β-carotene, lycopene), phytoene, phytofluene and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, pentaerythrityl tetra-di-t-butyl hydroxyhydrocinnamate, distearyl thiodipropionate, thiodipropionic acid and their derivatives (esters, ethers, peptides, lipids, nucleotides, nucleosides and salts) as well as sulphoximine compounds (for example buthionine sulphoximines, lactobacillus (ferment, filtrate, lysate), homocysteine sulphoximines, buthionine sulphones, penta-, hexa-, hepta-thionine sulphoximine) in very low tolerated doses, and also (metal) chelating agents (for example α-hydroxy fatty acids, palmitic acid, phytic acid, lactoferrin, α-hydroxy acids (for example citric acid, lactic acid, malic acid), humic acid, bile acid, bile extracts, bilirubin, biliverdin, hinokitiol, EDTA, EGTA, Trisodium Dicarboxymethyl Alaninate, MGDA (methylglycinic diacetic acid), GLDA (Glutamic acid diacetic acid)) and their derivatives, unsaturated fatty acids and their derivatives (for example γ-linolenic acid, linoleic acid, oleic acid), folic acid and its derivatives, ubiquinone and ubiquinol and their derivatives, triethyl citrate, Vitamin C and its derivatives (for example ascorbyl palmitate, magnesium ascorbyl phosphate, ascorbyl acetate), tocopherols and their derivatives (for example α-tocopherol, ß-tocopherol, γ-tocopherol, δ-tocopherol, tocopheryl acetate), dexpanthenol, Vitamin A and its derivatives (for example Vitamin A palmitate, Hydroxypinacolone Retinoate) and also coniferyl benzoate of benzoin resin, rutinic acid and its derivatives, ferrulic acid and its derivatives, butylhydroxytoluene, butylhydroxyanisole, hydroxymethoxyphenyl decanone, nordihydroguaiacic acid, nordihydroguaiaretic acid, allantoin, tropolone, trihydroxybutyrophenone, uric acid and its derivatives, urea, mannose and its derivatives, hydroxy acetophenone (ortho/para), zinc and its derivatives (for example ZnO, ZnSO₄), beta-aspartyl arginine, selenium and its derivatives (such as selenium methionine), Vitis Vinifera (Grape) seed extract, oat extract, Cichorium intubybus (chicory) leaf extract, Leon-topodium alpinum extract, green tea extract, Curcumin, Zingiber officinalis (Ginger) Root) Extract, Silymarin, stilbenes and their derivatives (such as stilbene oxide, trans-stilbene oxide) as well as the derivatives of said antioxidants, such as salts, esters, ethers, sugars, nucleotides, nucleosides, peptides and lipids, and mixtures of two or more of said antioxidants.

In a preferred variant, the antioxidant of the cosmetic or pharmaceutical composition or homecare product of the present invention is selected from the group consisting of dimethylmethoxy chromanol, hydroxyacetophenone, acorbic acid and its salts, hydroxymethoxyphenyl decanone, beta-aspartyl arginine, uric acid, urea, hydroxypinacolone retinoate, vitamin A and vitamin A derivatives, butylhydroxytoluol (BHT), butylhydroxyanisol (BHA), hydroxyphenyl propamidobenzoic acid, ascorbyl palmitate, ascorbyl phosphate and salts thereof, carnosine, rutin, tocopherol, tocopheryl acetate, ubiquinone-10, dilauryl thiodipropionate, pentaerythrityl tetra-di-t-butyl hydroxyhydrocinnamate, triethyl citrate, diethylhexyl syringylidene malonate, lactobacillus (ferment, filtrate, lysate), cannabinoids, cannabidiol and its extracts (Cannabis sativa seed oil, Cannabis sativa extract), cannabinol, hydroxyphenyl propamidobenzoic acid (dihydroavenanthramide D), green tea extract, Zingiber officinalis (Ginger) root extract, tropolone, allantoin, and mixtures of two or more of said antioxidants.

In a particularly preferred variant, the antioxidant of the cosmetic or pharmaceutical composition of the present invention is tocopherol or tocopheryl acetate. Also preferred is dimethylmethoxy chromanol.

Still more preferred from the above antioxidants are antioxidants selected from the group consisting of cannabinoids, cannabidiol and its extracts (Cannabis sativa seed oil, Cannabis sativa extract), hydroxyacetophenone, cannabinol, hydroxymethoxyphenyl decanone hydroxyphenyl propamidobenzoic acid (dihydroavenanthramide D).

The aforesaid antioxidants can be used either as a single component or in admixture with one or more further antioxidants as specified above.

Surprisingly it turned out that with the addition of a mycosporine-like amino acid compound (b) as defined herein to a food, cosmetic or pharmaceutical preparation or homecare product according to the present invention comprising an antioxidant, the antioxidative efficacy of the antioxidant (c) can significantly be improved. Preferably, the combination of a mycosporine-like amino acid compound (b) and an antioxidant (c) result in a synergistic stabilizing effect against oxidative degradation. The use of such a combination in food, a cosmetic or pharmaceutic preparation or homecare product in turn results in stable formulations with no or merely minor malodour and/or colour deviations. The improved antioxidative effect in turn results in formulations with improved storage stability and prolonged shelf live.

Thus, in a preferred advantageously variant, the food, cosmetic or pharmaceutical compositions or homecare products according to the first aspect of the present invention comprises one of the following combinations of components (b) and (c):
one or more of the following antioxidants: dimethylmethoxy chromanol, and/or hydroxyacetophenone, and/or acorbic acid and its salts, and/or hydroxymethoxyphenyl decanone, and/or beta-aspartyl arginine, and/or uric acid, and/or urea, and/or hydroxypinacolone retinoate, and/or vitamin A and vitamin A derivatives, and/or butylhydroxytoluol (BHT), and/or butylhydroxyanisol (BHA), and/or hydroxyphenyl propamidobenzoic acid, and/or ascorbyl palmitate, and/or ascorbyl phosphate and salts thereof, and/or carnosine, and/or rutin, and/or tocopherol, and/or tocopheryl acetate, and/or ubiquinone-10, and/or dilauryl thiodipropionate, and/or pentaerythrityl tetra-di-t-butyl hydroxyhydrocinnamate, and/or triethyl citrate, and/or diethylhexyl syringylidene malonate, and/or lactobacillus (ferment, filtrate, lysate), and/or cannabinoids, cannabidiol and its extracts (Cannabis sativa seed oil, Cannabis sativa extract), and/or cannabinol, and/or hydroxyphenyl propamidobenzoic acid (dihydroavenanthramide D), and/or green tea extract, and/or Zingiber officinalis (ginger) root extract, and/or tropolone, and/or allantoin, combined with a mycosporine-like amino acid compound of formula (V); or
one or more of the following antioxidants: dimethylmethoxy chromanol, and/or hydroxyacetophenone, and/or acorbic acid and its salts, and/or hydroxymethoxyphenyl decanone, and/or beta-aspartyl arginine, and/or uric acid, and/or urea, and/or hydroxypinacolone retinoate, and/or vitamin A and vitamin A derivatives, and/or butylhydroxytoluol (BHT), and/or butylhydroxyanisol (BHA), and/or hydroxyphenyl propamidobenzoic acid, and/or ascorbyl palmitate, and/or ascorbyl phosphate and salts thereof, and/or carnosine, and/or rutin, and/or tocopherol, and/or tocopheryl acetate, and/or ubiquinone-10, and/or dilauryl thiodipropionate, and/or pentaerythrityl tetra-di-t-butyl hydroxyhydrocinnamate, and/or triethyl citrate, and/or diethylhexyl syringylidene malonate, and/or lactobacillus (ferment, filtrate, lysate), and/or cannabinoids, cannabidiol and its extracts (Cannabis sativa seed oil, Cannabis sativa extract), and/or cannabinol, and/or hydroxyphenyl propamidobenzoic acid (dihydroavenanthramide D), and/or green tea extract, and/or Zingiber officinalis (ginger) root extract, and/or tropolone, and/or allantoin, combined with a mycosporine-like amino acid compound of formula (VI).

The above antioxidant/mycosporine-like amino acid compound combinations display a remarkably synergistic antioxidative efficacy against oxidative degradation and are clearly superior to the individually corresponding compounds and having the same concentration.

Most preferred antioxidant/mycosporine-like amino acid compound combinations in the food, cosmetic or pharmaceutical preparation or homecare product according to the first aspect of the present invention are:
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with a mycosporine-like amino acid compound of formula (V); or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with a mycosporine-like amino acid compound of formula (VI); or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-1; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-2; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-3; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-4; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-5; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-6; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-7; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-8; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-9; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-11; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-12; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-13; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-14; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-16; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-17; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-20; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-21; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-22; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-23; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-24; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-25; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-26; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-27; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-28; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-29; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-30; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-31; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-32; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-33; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-34; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-35; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-36; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-37; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or hydroxyacetophenone, and/or cannabinol, and/or hydroxymethoxyphenyl decanone, and/or hydroxyphenyl propamidobenzoic acid (dihydroavenanthramide D), combined with MAA-38; or
one or more of the following antioxidants: tocopherol and/or tocopheryl acetate, and/or dimethylmethoxy chromanol, and/or cannabinoids, and/or cannabidiol and its extracts (Cannabis sativa seed oil, Cannabis sativa extract), and/or decanone, and/or hydroxyphenyl propamidobenzoic acid (dihydroavenanthramide D), combined with MAA-39.

For the above combinations a synergistic stabilizing effect against oxidative degradation and an antioxidant boosting effect can be observed.

A synergistic antioxidant boosting effect is also observed for the mycosporine-like amino acid compounds MAA-A, MAA-B and MAA-C, when combined with any of the antioxidants as specified above.

The at least one fragrance or aroma/flavour compound (a) is present in the food, cosmetic or pharmaceutical preparation or homecare product in an amount of 0.01 to 40.0% by weight, based on the total weight of the food, cosmetic or pharmaceutical preparation or homecare product. In a preferred variant, the food, cosmetic or pharmaceutical preparation or homecare product comprises the fragrance or aroma/flavour compound in an amount of 0.02 to 35.0% by weight, based on the total weight of the food, cosmetic or pharmaceutical preparation or homecare product. In a particular preferred variant, the fragrance or aroma/flavour compound is advantageously used in the food, cosmetic or pharmaceutical preparation or homecare product in an amount of at 0.03 to 30.0% by weight, based on the total weight of the composition or final food, cosmetic or pharmaceutical preparation or homecare product. In a most preferred variant, the fragrance or aroma/flavour compound is advantageously used in the food, cosmetic or pharmaceutical preparation or homecare product in an amount of at 0.04 to 25.0% by weight, based on the total weight of the composition or final food, cosmetic or pharmaceutical preparation or homecare product.

For a mixture of fragrances, the above amounts relate to the total content of the fragrances in the mixture, i.e. the amount is the sum of the content of all fragrances in the mixture. Alternatively, for a mixture of aroma/flavours, the above amounts relate to the total content of the aroma in the mixture, i.e. the amount is the sum of the content of all aroma/flavours in the mixture.

The at least one mycosporine-like amino acid compound (b) is present in the food, cosmetic or pharmaceutical preparation or homecare product in an amount of 0.001 to 15.0% by weight, based on the total weight of the final formulation. In a preferred variant, the food, cosmetic or pharmaceutical preparation or homecare product comprises the at least one mycosporine-like amino acid compound (b) in an amount of 0.01 to 10.0% by weight, based on the total weight of the final formulation. In a more preferred variant, the at least one mycosporine-like amino acid compound (b) is advantageously used in the food, cosmetic or pharmaceutical preparation or homecare product in an amount of at 0.05 to 7.5% by weight, based on the total weight of the final formulation. In a most preferred variant, the at least one mycosporine-like amino acid compound is advantageously used in the food, cosmetic or pharmaceutical preparation or homecare product in an amount of at 0.1 to 5.0% by weight, based on the total weight of the final formulation.

For a mixture of mycosporine-like amino acid compounds (b), the above amounts relate to the total content of the mycosporine-like amino acid compounds in the mixture, i.e. the amount is the sum of the content of all mycosporine-like amino acid compounds (b) in the mixture.

The optionally at least one antioxidant (c) according to the first aspect of the present invention is present in the food, cosmetic or pharmaceutical preparation or homecare product in an amount of 0.0001 to 10.0% by weight, based on the total weight of the final formulation. In a preferred variant, the food, cosmetic or pharmaceutical preparation or homecare product comprises the antioxidant (c) in an amount of 0.0005 to 8.0% by weight, based on the total weight of the final formulation. In a most preferred variant, the antioxidant (c) is advantageously used in the food, cosmetic or pharmaceutical preparation or homecare product in an amount of 0.001 to 6.0% by weight, based on the total weight of the final formulation.

For a mixture of antioxidants (c), the above amounts relate to the total content of the antioxidants in the mixture, i.e. the amount is the sum of the content of all antioxidants (c) in the mixture.

Advantageously, the food, cosmetic or pharmaceutical preparation or homecare product according to the first aspect of the present invention comprises
(ii) 0.01 to 40.0% by weight of at least one fragrance or aroma/flavour compound;
(iii) 0.001 to 15.0% by weight of at least one mycosporine-like amino acid compound as defined herein; and
(iv) optionally 0.0001 to 10.0% by weight of at least one antioxidant;
based on the based on the total weight of the final formulation.

In a preferred variant, the cosmetic or pharmaceutical preparation according to the first aspect of the present invention comprises
(v) 0.04 to 25.0% by weight of at least one fragrance or aroma/flavour;
(vi) 0.1 to 5.0% by weight of at least one mycosporine-like amino acid compound as defined herein; and
(vii) optionally 0.001 to 6.0% by weight of at least one antioxidant;
based on the based on the total weight of the final formulation.

Within the context of the present invention, it is also possible and in some cases advantageous to combine the food, cosmetic or pharmaceutical preparation or homecare product according to the first aspect of the present invention with other customary active substances, adjuvants or additives, customarily used for sunscreen prouducts, cosmetic or pharmaceutical compositions or homecare products, as further described below, in order to obtain a ready-for-use preparation or formulation.

The other active agents and/or adjuvants and/or additives or auxiliaries are for example abrasives, anti-acne agents, agents against ageing of the skin, anti-cellulitis agents, anti-dandruff agents, anti-inflammatory agents, irritation-preventing agents, irritation-inhibiting agents, antioxidants, alkanediols, astringents, odour absorbers, perspiration-inhibiting agents, antiseptic agents, anti-statics, antimicrobial agents, binders, buffers, carrier materials, oil components, chelating agents, cell stimulants, cleansing agents, depilatory agents, surface-active substances, deodorizing agents, antiperspirants, softeners, emulsifiers, enzymes, enzyme inhibitors, essential oils, fibres, film-forming agents, water resistance improving agents fixatives, foam-forming agents, foam stabilizers, substances for preventing foaming, foam boosters, gelling agents, gel-forming agents, hair care agents, hair-setting agents, hair-straightening agents, moisture-donating agents, moisturizing substances, moisture-retaining substances, bleaching agents, strengthening agents, stain-removing agents, optically brightening agents, impregnating agents, dirt-repellent agents, dyes, friction-reducing agents, lubricants, moisturizing creams, ointments, opacifying agents, plasticizing agents, covering agents, polish, preservatives, gloss agents, green and synthetic polymers, powders, proteins, re-oiling agents, abrading agents, silicones, skin-soothing agents, skin-cleansing agents, skin care agents, skin-healing agents, skin-lightening agents, skin-protecting agents, skin-softening agents, hair promotion agents, cooling agents, skin-cooling agents, warming agents, skin-warming agents, stabilizers, surfactants, antimicrobial agents, detergents/surfactants, fabric conditioning agents, suspending agents, skin-tanning agents, actives modulating skin or hair pigmentation, matrix-metalloproteinase inhibitors, skin moisturizing agents, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxy fatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the aforementioned substances, as further described below.

Of the above cosmetically or pharmaceutically excipients and/or active substances and/or additives and/or auxiliaries carriers, oil components, rheology additives, hydrotropes, solubilizing agents, powders, film formers, water resistance improving agents, skin moisturizing and/or moisture-retaining substances, lenitive substances, physiological cooling agents, alkanediols, antioxidants, agents against ageing of the skin, matrix-metalloproteinase inhibitors (MMPI), anti-inflammatory agents, TRPV1 antagonists, emulsifiers, antimicrobial agents, preservatives, antibacterial or antimycotic active substances, chelating agents, surfactants, fragrances/aroma or perfume oils, and any mixture of two or more of the afore-mentioned substances are particularly preferred in the preparation of food, cosmetic and pharmaceutical preparations, such as sunscreen products, or homecare products.

Carriers: The compositions according to the present invention may comprise a carrier or a mixture of different carriers. Most common acceptable carrier is water. Acceptable carries other than water include glycerin, C1-4 alcohols, organic solvents, fatty alcohols, fatty ethers, fatty esters, polyols, glycols, vegetable oils, mineral oils, liposomes, laminar lipid materials, water, or a mixture thereof. Non-limiting examples of organic solvents include mono alcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds. The total amount of carrier in the compositions can vary but is typically 40 to 90% by weight, based on the total weight of the composition.

The products or preparations of the present invention may include at least one water-soluble or organic solvent. Non-limiting examples of organic solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, 1,2-pentanediol, 1,2-hexanediol, 1,2-heptanediol, 1,2-octanediol, 1,2-decanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and any mixture thereof. The total amount water-soluble or organic solvent(s) in the composition according to the present invention may vary but is typically 0.1 to 50% by weight, based on the total weight of the composition.

Solubilizing agents: The products or preparations of the present invention may include at least one solubilizing agent. Solubilizing agents are compounds that help solubilize the components in the compositions. A particularly useful but non limiting example of a solubilizing agent is a hydrotrope. Hydrotropes (or hydrotropic agents) are a diverse class of typically water-soluble compounds that may be characterized by an amphiphilic molecular structure and an ability to dramatically increase the solubility of poorly soluble organic molecules in water. Non-limiting examples of hydrotopes include sodium 1,3-benzenedisulfonate, sodium benzoate, sodium 4-pyridinecarboxylate, sodium salicylate, sodium benzene sulfonate, caffeine, sodium p-toluene sulfonate, sodium butyl monoglycolsulfate, 4-aminobenzoic acid HCl, sodium cumene sulfonate, N, N-diethylnicotinamide, N-picolylnicotinamide, N-allylnicotinamide, 2-methacryloyloxyethyl phosphorylcholine, resorcinol, butylurea, pyrogallol, N-picolylacetamide 3.5, procaine HCl, proline HCl, nicotinamide, pyridine, 3-picolylamine, sodium ibuprofen, sodium xylenesulfonate, ethyl carbamate, pyridoxal hydrochloride, sodium benzoate, 2-pyrrolidone, ethylurea, N,N-dimethylacetamide, N-methylacetamide, and isoniazid and any mixture thereof. In some cases, particularly useful hydrotropes include nicotinamide (niacinamide), caffeine, sodium PCA, sodium salicylate, urea, and dhydroxyethyl urea, in particular, nicotinamide (niacinamide) and/or caffeine. A combination of two or more, three or more, or four or more hydrotopes may also be used in the compositions according to the present invention. The total amount of solubilizing agent(s) in the compositions of the present invention may vary but are typically in an amount of 0.01 to 20% by weight, based on the total weight of the composition.

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine; and
dialcoholamines, such as diethanolamine or 2-aminopropane-1,3-diol.

Oil components: The products or preparations according to the present invention may include at least one oil phase or at least one oil component, waxes, starch, tragacanth, cellulose derivatives, polyethylene glycols, silicones, bentonites, silica, talc and zinc oxide or any mixture thereof. Powders include carriers such as lactose, talc, silica, aluminium hydroxide, calcium silicate and polyamide powder or any mixture thereof. Solutions and emulsions include carriers such as solvents such as water, ethanol, isopropanol, ethyl carbonate, ethyl acetate, benzyl alcohol, propylene glycol, etc. or any mixture thereof. However, preparations solely based on water are also possible. Oils, oil-in-water and water-in-oil emulsions, etc. are preferred.

The oil phase or the oil component in the products or preparations according to the present invention which may be suitable are for example plant oils, hydrocarbons, fatty alcohols, fatty acid esters, or mixtures of two or more of the aforesaid oil components.

The oil phase or oil component in the products or preparations according to the present invention is preferably a plant oil and even more preferably a liquid plant oil. It can also advantageously be a mixture of two or more plant oils components, especially liquid plant oil mixtures.

Plant oils or vegetable oils are oils extracted from seeds, or less often, from other parts of fruits. Like animal fats, plant oils are mixtures of triglycerides. Soybean oil, rapeseed oil and cocoa butter are examples of plant oils from seeds. Olive oil, palm oil and rice bran oil are examples of oils from other parts of fruits. In common usage, plant oil or vegetable oil may refer exclusively to vegetable fats which are liquid at room temperature or at 35 to 37° C. skin temperature. Vegetable oils are usually edible.

The term “plant oils” also includes unsaturated plant oils. Unsaturated oils or vegetable oils can be transformed through partial or complete “hydrogenation” into oils of higher melting point. The hydrogenation process involves “sparging” the oil at high temperature and pressure with hydrogen in the presence of a catalyst, typically a powdered nickel compound. As each carbon-carbon double-bond is chemically reduced to a single bond, two hydrogen atoms each form single bonds with the two carbon atoms. The elimination of double bonds by adding hydrogen atoms is called saturation; as the degree of saturation increases, the oil progresses toward being fully hydrogenated. An oil may be hydrogenated to increase resistance to rancidity (oxidation) or to change its physical characteristics. As the degree of saturation increases, the oil's viscosity and melting point increase.

In a preferred variant, the plant oil is selected from the group consisting of Persea gratissima (Avocado Oil), Abies alba Seed Oil, Acacia victoriae Seed Oil, Actinidia chinensis (Kiwi) Seed Oil, Amaranthus hypochondriacus Seed Oil, Arachis hypogaea (Peanut) Oil, Astrocaryum murumuru Seed Butter, Astrocaryum tucuma Seed Butter, Astrocaryum tucuma Seed Oil, Astrocaryum vulgare Fruit Oil, Astrocaryum vulgare Kernel Oil, Avena sativa (Oat) Kernel Oil, Brassica alba Seed Oil, Brassica campestris (Rapeseed) Seed Oil, Butyrospermum parkii (Shea) Butter, butyrospermum parkii (Shea) Oil, Calendula officinalis Seed Oil, Calophyllum inophyllum Seed Oil, Calophyllum tacamahaca Seed Oil, Camellia oleifera Seed Oil, Camellia reticulata Seed Oil, Camellia sinensis Seed Oil, Cannabis sativa Seed Oil, Cannabis sativa Seed/Stem Oil, Canola Oil, Carthamus tinctorius (Safflower) Seed Oil, Chlorella vulgaris Oil, Citrullus lanatus (Watermelon) Seed Oil, Citrus aurantifolia (Lime) Seed Oil, Citrus aurantium dulcis (Orange) Seed Oil, Citrus grandis (Grapefruit) Seed Oil, Cocos nucifera (Coconut) Oil, Cocos nucifera (Coconut) Seed Butter, Coffea arabica (Coffee) Seed Oil, Chlorella Oil (biotech), Corylus americana (Hazelnut) Seed Oil, Corylus avellana (Hazelnut) Seed Oil, Cucumis melo (Melon) Seed Oil, Cucumis sativus (Cucumber) Seed Oil, Cucurbita pepo (Pumpkin) Seed Oil, Elaeis guineensis (Palm) Oil, Elaeis (Palm) Fruit Oil, Glycine soja (Soybean) Oil, GosSY-pium herbaceum (Cotton) Seed Oil, GosSY-pium hirsutum (Cotton) Seed Oil, Helianthus annuus (Sunflower) Seed Oil, Macadamia integrifolia Seed Oil, Macadamia ternifolia Seed Oil, Mangifera indica (Mango) Seed Butter, Mangifera indica (Mango) Seed Oil, Melissa officinalis Seed Oil, Microalgae Oil, Moringa oleifera Seed Oil, Moringa peregrina Seed Oil, Oenothera biennis (Evening Primrose) Oil, Olea europaea (Olive) Fruit Oil, Olus Oil, Orbignya oleifera Seed Oil, Orbignya speciosa Kernel Oil, Oryza sativa (Rice) Bran/Germ Oil, Oryza sativa (Rice) Bran Oil, Oryza sativa (Rice) Germ Oil, Oryza sativa (Rice) Lipids, Oryza sativa (Rice) Seed Oil, Papaver somniferum Seed Oil, Passiflora edulis Seed Oil, Persea gratissima (Avocado) Butter, Persea gratissima (Avocado) Oil, Prunus amygdalus dulcis (Sweet Almond) Oil, Prunus armeniaca (Apricot) Kernel Oil, Prunus persica (Peach) Kernel Oil, Punica granatum Seed Oil, Pyrus malus (Apple) Seed Oil, Ricinoleic/Caproic/Caprylic/Capric Triglyceride(s), Ricinus communis (Castor) Seed Oil, Rosa canina Fruit Oil, Rosa moschata Seed Oil, Rubus idaeus (Raspberry) Seed Oil, Sesamum indicum (Sesame) Seed Butter, Soybean Glycerides, Theobroma cacao (Cocoa) Seed Butter, Theobroma grandiflorum Seed Butter, Triticum vulgare (Wheat) Bran Lipids, Triticum vulgare (Wheat) Germ Oil, Vitis vinifera (Grape) Seed Oil, Zea mays (Corn) Germ Oil, and Zea mays (Corn) Oil.

The above specified plant oils are the most common natural lipid components used as basic substances for the manufacture of cosmetics or pharmaceutical formulations.

Hydrocarbons (mineral oils) are in general organic compounds consisting entirely of hydrogen and carbon. As defined by IUPAC nomenclature or organic chemistry, the classifications for hydrocarbons are:
1. Saturated hydrocarbons are the simplest of the hydrocarbon species. They are composed entirely of single bonds and are saturated with hydrogen. The formula for acyclic saturated hydrocarbons (i.e., alkanes) is CₙH₂ₙ₊₂. The most general form of saturated hydrocarbons is CₙH₂ₙ₊₂₍₁₋ᵣ₎, where r is the number of rings. Those with exactly one ring are the cycloalkanes. Saturated hydrocarbons are the basis of petroleum fuels and are found as either linear or branched species.
2. Unsaturated hydrocarbons have one or more double or triple bonds between carbon atoms. Those with double bond are called alkenes. Those with one double bond have the formula CₙH₂ₙ (assuming non-cyclic structures). Those containing triple bonds are called alkynes. Those with one triple bond have the formula CₙH₂ₙ₋₂.
3. Aromatic hydrocarbons, also known as arenes, are hydrocarbons that have at least one aromatic ring.

Hydrocarbons can be inter alia liquids (e.g. hexane and benzene), waxes or low melting solids (e.g. paraffin wax and naphthalene). The term ‘aliphatic’ refers to non-aromatic hydrocarbons. Saturated aliphatic hydrocarbons are sometimes referred to as “paraffins”. Mineral oils and waxes are mixtures of predominantly saturated hydrocarbons consisting of straight-chain, branched and ring structures with carbon chain lengths greater than C14. Mineral oils and waxes are chemical substances prepared from naturally occurring crude petroleum oil. They mainly consist of mineral oil saturated hydrocarbons (MOSH) and mineral oil aromatic hydrocarbons (MOAH). Hydrocarbons have been used for many decades in skin and lip care cosmetic products due to their excellent skin tolerance as well as their high protecting and cleansing performance and broad viscosity options. In contrast to vegetable oils, mineral oils are non-allergenic since they are highly stable and not susceptible to oxidation or rancidity.

A fatty alcohol (or long-chain alcohol) is usually a high-molecular-weight, straight-chain primary alcohol, but can also range from as few as 4 to 6 carbons to as many as 22 to 26, derived from natural fats and oils. The precise chain length varies with the source. Some commercially important fatty alcohols are lauryl, stearyl and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. Most fatty alcohols in nature are found as waxes which are esters with fatty acids and fatty alcohols. The traditional sources of fatty alcohols have largely been various vegetable oils and these remain a large-scale feedstock. The alcohols are obtained from the triglycerides (fatty acid triesters), which form the bulk of the oil. The process involves the transesterification of the triglycerides to give methyl esters which are then hydrogenated to give the fatty alcohols. Fatty alcohols are also prepared from petrochemical sources. In the Ziegler process, ethylene is oligomerized using triethylaluminium followed by air oxidation. Alternatively, ethylene can be oligomerized to give mixtures of alkenes, which are subjected to hydroformylation, this process affording odd-numbered aldehyde, which is subsequently hydrogenated. Fatty alcohols are mainly used in the production of detergents and surfactants. They are components also of cosmetic solvents. They find use as co-emulsifiers, emollients and thickeners in cosmetics.

In a preferred variant, the fatty alcohol is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, and isotridecyl alcohol. In a more preferred variant, the fatty alcohol is selected from the group consisting of hexyldecanol, octyldodecanol, phenylpropanol, isoamylalcohol, and mixtures of two or more of the aforesaid fatty alcohols. The fatty alcohol can be used either as a single component or in a mixture with one or more further different fatty alcohol(s) as specified above.

A fatty acid ester is a type of ester that results from the combination of a fatty acid with an alcohol. When the alcohol component is glycerol, the fatty acid esters produced can be monoglycerides, diglycerides or triglycerides. Fatty acid esters have a conditioning effect of softening the skin to create a smoothing sensation. They are also added to cosmetics to dissolve high-polarity active ingredients and UV absorbers. Esters of straight-chain fatty acids and lower alcohols are effective for dissolving slightly soluble ingredients for oils with a light touch during application. Isostearic acids and other liquid oils with branched fatty acids and unsaturated fatty acids are commonly used as emollients. Higher fatty acid esters and esters of higher alcohols with relatively high melting points are added to skin creams to adjust the application touch.

Oil bodies and emollients: In addition to the oil phase or oil component as defined above, the products or preparations according to the present invention preferably includes one or more oil bodies or emollients. An emollient is an oleaginous or oily substance which helps to smooth and soften the skin, and may also reduce its roughness, cracking or irritation. Especially, in sunscreen products emollients counteract the known stickiness of some UV-filters and keep the crystalline and oil-soluble organic and inorganic UV-filters solubilized and prevent them from recrystallisation. In addition, emollients serve as wetting agents for inorganic UV-filters for a homogeneous dispersion in the formulations. Suitable oil bodies, which form constituents of the O/W emulsions, are, for example, Guerbet alcohols based on fatty alcohols having 6 to 18, preferably 8 to 10, carbon atoms, esters of linear C5-C22 fatty acids with linear or branched C5-C22 fatty alcohols or esters of branched C1-C13 carboxylic acids with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22 fatty acids with branched alcohols, in particular 2-ethylhexanol, esters of C18-C38 alkylhydroxy carboxylic acids with linear or branched C6-C22 fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6-C10 fatty acids, liquid mono-/di-/triglyceride mixtures based on C6-C18 fatty acids, esters of C6-C22 fatty alcohols and/or Guerbet alcohols with aromatic carboxylic acids, in particular benzoic acid, esters of C2-C12 dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched CC—C22 fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C6-C22-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes, and any mixture thereof.

Powders: The compositions as defined herein may optionally include powders. The optional powders provide formulas that are smoother and softer on the skin. Representative powders include talc, mica, magnesium carbonate, calcium carbonate, magnesium silicate, aluminium magnesium silicate, silica, titanium dioxide, zinc oxide, red iron oxide, yellow iron oxide, black iron oxide, polyethylene powder, methacrylate powder, polystyrene powder, silk powder, Preferred solid powder materials, which may be a component of the composition according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin, inulin, and mixtures of two or more of these solids, in particular maltodextrins (preferably with a dextrose equivalent value of 15-20), lactose, silicon dioxide and/or glucose.

Rheology modifiers: The food, cosmetic or pharmaceutical preparation or homecare products according to the present invention advantageously includes one or more thickening agents and/or rheology modifier. The rheology modifier is present in an amount that prevents significant dripping or pooling of the composition after application to the skin or to surfaces. Preferably, the rheology modifier is a carbomer. In some embodiments, the rheology modifier is selected from stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional example of rheology modifiers include thickener or gelling agents, including substances which can increase the viscosity of a composition. Thickening agents include those that can increase the viscosity of a composition without substantially modifying the efficacy of the active ingredient within the formulation. Thickening agents can also increase the stability of the formulations of the present application. In certain aspects of the present application, thickening agents include hydrogenated polyisobutene or trihydroxy stearin, or a mixture of both. Additional non-limiting examples of additional thickening agents that can be used in the context of the present application include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see CTFA International Cosmetic Ingredient Dictionary, Fourth Edition, 1991, pp. 12 and 80). Examples of commercially available carboxylic acid polymers include carbomers, which are homopolymers of acrylic acid crosslinked with allyl ethers of sucrose or pentaerythritol (e.g., Carbopol™ 900 series from B. F. Goodrich). Non-limiting examples of crosslinked polyacrylate polymers include cationic and non-ionic polymers.

Non-limiting examples of polyacrylamide polymers (including non-ionic polyacrylamide polymers including substituted branched or unbranched polymers) include polyacrylamide, isoparaffin and Laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer are hydroxyalkylated (preferably hydroxy ethylated or hydroxypropylated) to form a hydroxyalkylated cellulose which is then further modified with a C10-C30 straight chain or branched chain alkyl group through an ether linkage. Typically, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present compositions include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof. In one embodiment, the thickening agent is Chondrus crispus (carrageenan) extract.

Silicones: In order to impart a silky, spreadable, and luxurious texture and to make skin look and feel smoother, and additionally to improve processability (antifoaming) the food, cosmetic or pharmaceutical preparation or homecare product according to the present invention preferably includes one or more silicones or silicone derivatives. The total amount of silicone compound(s) in the compositions according to the present invention can vary but is typically 0.1 to 35.0% by weight, based on the total weight of the composition.

Dry-feel modifiers: The food, cosmetic or pharmaceutical preparation or homecare product according to the present invention is preferably combined with dry-feel modifiers. A dry-feel modifier is an agent which, when incorporated in an emulsion, imparts a “dry feel” to the skin when the emulsion dries. Dry-feel modifiers may also reduce sunscreen migration on the skin. Dry feel modifiers can include starches, talc, kaolin, chalk, zinc oxide, Hydroxyapatite (such as Tinomax CC), silicone fluids, inorganic salts such as barium sulfate and sodium chloride, C6 to C12 alcohols such as octanol; sulfonated oils; surface treated silica, precipitated silica, fumed silica such as Aerosil® available from the Degussa Inc. of New York, N.Y. U.S.A. or mixtures thereof; dimethicone, a mixture of mixture of methylated linear siloxane polymers, available as DC200 fluid, tradename of Dow Corning, Midland, Mich. U.S.A. One or more dry-feel modifiers can optionally be included in the sunscreen in amounts ranging from 0.01 to 20% by weight, more preferably from 0.1 to 10.0% by weight.

Lenitive substances: The sunscreen product or cosmetic or pharmaceutical preparation according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica montana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Physiological cooling agents: The food, cosmetic or pharmaceutical preparation or homecare product according to the present invention can be particularly advantageously combined with one or more physiological cooling agent(s). The use of cooling agents can alleviate itching. Preferred individual cooling agents for use within the framework of the present invention are listed below. The person skilled in the art can add many other cooling agents to this list; the cooling agents listed can also be used in combination with one another: which are preferably selected here from the following list: menthol and menthol derivatives (for example L-menthol, D-menthol, racemic menthol, isomenthol, neoisomenthol, neomenthol) menthylethers (for example (I-menthoxy)-1,2-propanediol, (I-menthoxy)-2-methyl-1,2-propanediol, I-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyroglutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or Na-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (I-(−)-isopulegol, I-(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2 (1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarboxamide or related compounds), (1R,2S,5R)—N-(4-Methoxyphenyl)-5-methyl-2-(1-isopropyl)cyclohexane-carboxamide [WS12], oxamates and [(1R,2S,5R)-2-isopropyl-5-methyl-cyclohexyl] 2-(ethylamino)-2-oxo-acetate (X Cool). Cooling agents which are preferred due to their particular synergistic effect are I-menthol, d-menthol, racemic menthol, menthone glycerol acetal (trade name: Frescolat® MGA), menthyl lactate (preferably I-menthyl lactate, in particular I-menthyl I-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are I-menthol, racemic menthol, menthone glycerol acetal (trade name: Frescolat® MGA), menthyl lactate (preferably I-menthyl lactate, in particular I-menthyl I-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are I-menthol, menthone glycerol acetal (trade name: Frescolat® MGA) and menthyl lactate (preferably I-menthyl lactate, in particular I-menthyl I-lactate (trade name: Frescolat® ML).

Alkanediols: In a further preferred variant, the food, cosmetic or pharmaceutical preparation or homecare product according to the invention comprises one or more linear alkanediol(s) having a carbon chain of 5 to 12 carbon atoms. Preferably, said one or more linear alkanediol(s) is/are selected from the group consisting of 1,2-alkanediols, 2,3-alkanediols, 3,4-alkanediols and 1,3-alkanediols, more preferably of 2,3-alkanediols and 1,3-alkanediols. Most preferably, said linear alkanediol(s) is/are selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol and 2,3-dodecanediol.

Initial observations of the inventors surprisingly indicated that compositions according to the invention comprising one or more linear alkanediol(s) having a carbon chain of 5 to 12 carbon atoms, preferably as defined herein, display particularly advantageous emulsifying properties. In particular, it was surprisingly found that the one or more of said linear alkanediol(s) (if not emulsions themselves) support(s) the formation of water or oil droplets with a particularly small average size in emulsions or (if emulsions themselves) contain water or oil droplets with a particularly small average size.

Additionally, it was found that the presence of an alkanediol, in particular a 1,2-alkanediol or 2,3-alkanediol, improves the solubility of oil components, such as one or more oil body/bodies or wax(es) in the food, cosmetic or pharmaceutical preparation or homecare product according to the present invention. Preferably the oil body/bodies or wax(es) are selected from the group consisting of natural fats and oils, fatty alcohols and alcohols, glyceryl esters and derivatives thereof, esters, ethers, siloxanes and silanes, waxes, and naphthenic hydrocarbons, preferably selected from the group consisting of squalene, dialkylcyclohexanes, tetradecane, isohexadecane, dodecane, docosane, paraffin, and mineral oils, and further described below.

A combination of one or more of said alkanediol(s) with 4-hydroxyacetophenon, typically in amounts from 0.1 to 2% by weight, more preferably from 0.1 to 1% by weight, improves the solubility of the oil components even in a synergistic manner.

Preferably, the composition comprises from 0.01 to 10% by weight, more preferably from 0.1 to 5% by weight, still more preferably from 0.3 to 3% by weight, most preferably from 0.5 to 1% by weight, of said linear alkanediol(s) having a carbon chain of 5 to 12 carbon atoms and defined herein, based on the total weight of the composition.

Emulsifiers: In addition, the food, cosmetic or pharmaceutical preparation or homecare product according to the present invention can also advantageously contain one or more emulsifiers. Emulsifiers include amphoteric, anionic, cationic or nonionic emulsifiers, used alone or as a mixture. The emulsifiers are chosen in an appropriate manner according to the emulsion to be obtained. Preferred examples include:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C 22 fatty acids, such as palmitic acid, stearic acid or behenic acid for example, and C12 to C22 dicarboxylic acids, such as azelaic acid or sebacic acid for example, potassium cetyl phosphate, and hydrogenated palm glycerides (Emulsiphos/Emulsiphos F).

Amphoteric emulsifiers: Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N, N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C12/18 acyl sarcosine.

Anti-ageing actives: A composition according to the present invention is preferably combined with one or more anti-ageing actives. In the context of the invention, anti-ageing or biogenic agents are, for example antioxidants, matrix-metalloproteinase inhibitors (MMPI), skin moisturizing agents, glycosaminglycan stimulators, anti-inflammatory agents, TRPV1 antagonists and plant extracts.

Matrix-Metalloproteinase inhibitors (MMPI): The compositions according to the present invention are preferably combined with one or more matrix-metalloproteinase inhibitors, especially those inhibiting matrix-metalloproteinases enzymatically cleaving collagen, selected from the group consisting of ursolic acid, retinyl palmitate, propyl gallate, precocenes, 6-hydroxy-7-methoxy-2,2-dimethyl-1 (2H)-benzopyran, 3,4-dihydro-6-hydroxy-7-methoxy-2,2-dimethyl-1 (2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, MGDA (Trisodiumdicarboxymethyl alaninante), galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and Lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus Fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Skin moisturizing and/or moisture-retaining substances: The compositions according to the present invention advantageously comprise one or more skin-moisturizing and/or moisture-retaining substances. Preferred skin moisturizing and/or moisture-retaining substances are selected from the group consisting of alkane diols or alkane triols comprising 3 to 12 carbon atoms, preferably C3-C10-alkane diols and C3-C10-alkane triols. More preferably the skin moisturizing agents are selected from the group consisting of glycerol, 1,2-propylene glycol, 1,2-butylene glycol, 1,3-butylene glycol, 1,2-pentanediol, 1,2-hexanediol, 1,2-heptanediol, 1,2-octanediol and 1,2-decanediol. Further skin moisturizing and/or moisture-retaining substances include sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-) ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Glycosaminoglycan stimulators: Preferred compositions according to the present invention comprise one or more substances stimulating the synthesis of glycosaminoglycans which are selected from the group consisting of hyaluronic acid and derivatives or salts, Subliskin (Sederma, INCI: Sinorhizobium meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (−)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (−)-alpha-bisabolol or synthetic alpha-bisabolol such as e.g. Dragosantol and Dragosantol 100 from Symrise, oat glucan, Echinacea purpurea extract, Sinorhizobium meliloti Ferment Filtrate, Calcium ketogluconate, Alpinia galanga leaf extract and tetradecyl aminobutyroylvalylaminobutyric urea trifluoroacetate.

Anti-inflammatory agents: The compositions according to the present invention are preferably combined with anti-inflammatory and/or redness and/or itch ameliorating ingredients, in particular steroidal substances of the corticosteroid type selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatoiren-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone,
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone,
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof,
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occurring avenanthramides, preferably tranilast, avenanthramide A, avenanthramide B, avenanthramide C, non-natural or non-naturally occurring avenanthramides, preferably dihydroavenanthramide D, dihydroavenanthramide E, avenanthramide D, avenanthramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-) ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and ß-glucans, in particular 1,3->1,4-ß-glucan from oats.

TRPV1 antagonists: Preferred compositions according to the present invention comprise one or more TRPV1 antagonists. Suitable compounds which reduce the hypersensitivity of skin nerves based on their action as TRPV1 antagonists, encompass e.g., trans-4-tert-butyl cyclohexanol, or indirect modulators of TRPV1 by an activation of the μ-receptor, e.g., acetyl tetrapeptide-15, are preferred.

Antimicrobial agents: The food, cosmetic or pharmaceutical preparation or homecare product according to the present invention can advantageously combined with an antimicrobial agent which act primarily against microorganisms, in particular bacteria, yeast and/or fungi and includes one or more antimicrobial agents such as Caprylhydroxamic Acid, o-Cymen-5-ol, Isopropylparaben, Capryloyl Glycine, Phenylpropanol, Tropolone, PCA Ethyl Cocoyl Arginate, 2-Methyl 5-Cyclohexylpentanol, Phenoxyethanol, Disodium EDTA, Methylparaben and its salts, Sodium Benzoate, Benzyl Alcohol, Potassium Sorbate, Benzyl Salicylate, Propylparaben, Sodium Methylparaben, Methylchloroisothiazolinone, Methylisothiazolinone, Ethylhexylglycerin, Butylparaben, Ethylparaben, Sodium Propylparaben, DMDM Hydantoin, Dehydroacetic Acid, Salicylic Acid, Chlorphenesin, Isobutylparaben, Sodium Ethylparaben, Diazolidinyl Urea, Diazolidinyl Urea, Farnesol, Bisabolol, Glyceryl Caprylate, Sodium Phytate or Phytic Acid, Sodium Levulinate or Levulinic Acid and esters and/or ketals thereof, Chlorhexidine, Glyceryl Laurate, Anisic Acid and its salts, Chlorhexidine Digluconate, TEA-salicylate, Phenethyl Alcohol, Glyceryl Caprate, Sorbitan Caprylate, Tetrasodium Glutamate Diacetate, Trisodium Ethylenediamine Disuccinate, Hydroxyethoxyphenyl Butanone, Hydroxyethoxyphenyl Butanol, Itaconic Acid, Octopirox, Propanediol Caprylate, Climbazole, Undecylenoyl Glycine, Thymol, 4-Hydroxyacetophenone, Lactic Acid, Sodium Lactate, Trisodium Dicarboxymethyl Alaninate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, and any mixture thereof.

Preferably, the food, cosmetic or pharmaceutical preparation or homecare product according to the present invention comprises one or more antimicrobial agent selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 2,3-pentanediol, 2,3-hexanediol, 2,3-heptanediol, 2,3-octanediol, 2,3-nonanediol, 2,3-decanediol, 2,3-undecanediol, 2,3-dodecanediol, 2-benzylheptanol, 2-hydroxyacetophenone, 2-methyl 5-cyclohexylpentanol, 3-hydroxypropyl 2-hydroxybenzoate, 4-hydroxyacetophenone, and any mixture thereof. More preferably, the food, cosmetic or pharmaceutical preparation or homecare product according to the present invention comprises one or more antimicrobial agent selected from the group consisting of 1,2-pentanediol, 1,2-hexanediol, 1,2-heptanediol, 1,2-octanediol, 1,2-nonanediol, 1,2-decanediol, 1,2-undecanediol, 1,2-dodecanediol, 1,2-tridecanediol, 4-hydroxyacetophenone, and any mixture thereof.

In order to further enhance antimicrobial efficacy, the food, cosmetic or pharmaceutical preparation or homecare product as defined herein, is advantageously combined with at least one further antimicrobial agent which is different from the anti-microbial agents specified above. The combination with a further antimicrobial agent provides reliable protection against microbial degradation and deterioration of the preparation, in particular during storage. Additionally, the further different antimicrobial agent provides reliable protection against other microorganisms as described above, for example Corynebacterium, Anaerococcus, Finegoldia, Moraxella, Porphyromonas, Fusobacterium, Malassezia, Peptoniphilus, Streptococcus, Lactobacillus, Gardnerella, Fannyhessea, Epidermophyton, Trichophyton, Cutibacterium. Hence, the combination with a further different antimicrobial agent allows antimicrobial protection against different groups of microorganisms, and, thus, a broader spectrum of microorganism. The further different antimicrobial agent in the context of the present invention is preferably selected from the group consisting of 2-benzylheptanol, alkyl (C 12-22) trimethyl ammonium bromide and chloride, ascorbic acid and salts thereof, benzalkonium bromide, benzalkonium chloride, benzalkonium saccharinate, benzethonium chloride, Benzoic Acid, camphor, cetylpyridinium chloride, chlorhexidine diacetate, chlorhexidine dihydrochloride, chlorocresol, chloroxylenol, Cyclohexylglycerin, Dimethyl phenylbutanol, Dimethyl phenylpropanol, ethanol, ethyl lauroyl arginate HCl, Ethyl Lauroyl Arginate Laurate, eucalyptol, gluconic acid and salts thereof, glycerin, hexamidine, hexamidine diisethionate, Hexylglycerin, lodopropynyl Butylcarbamate, jasmol, lauryl alcohol, Levulinic Acid and esters and/or ketals thereof, mannitol, menthol, methyl salicylate, Octenidine HCl, polyarginine, Polyglyceryl-10 Laurate, polyglyceryl-2 caprate, Polyglyceryl-3 caprylate, polylysine, Salvia officinalis (Sage) Oil, silver chloride, silver citrate, sodium caproyl lactylate, Sodium Caproyl/Lauroyl Lactylate, sodium lauroyl lactylate, Sorbic Acid, sorbitol, Tetraselmis extract, triclocarban, triclosan, Triethyl citrate, Xylityl Sesquicaprylate, zinc citrate, zinc pyrithione, Zinc ricinoleate, and any mixture thereof.

The aforesaid antimicrobial agents are used in the food, cosmetic or pharmaceutical preparation or homecare product either as a single component or preferably in a mixture with two or more further of said antimicrobial agents as specified above.

The antimicrobial agent is present in the food, cosmetic or pharmaceutical preparation or homecare product in an amount of 0.01 to 25.0% by weight, preferably 0.02 to 15.0% by weight, most preferred in an amount of 0.05 to 6.0% by weight, based on the total weight of the final formulation.

Advantageously, the at least one antimicrobial alkanediol is present in the food, cosmetic or pharmaceutical preparation or homecare product in an amount of 0.1 to 5% by weight, based on the total weight of the final formulation and/or the 4-hydroxyacetophenone in an amount of 0.01 to 2% by weight, based on the total weight of the final formulation.

Preservatives: For preservative purposes, the compositions according to the present invention preferably comprise one or more preservatives. Suitable and advantageously preservatives are, for example, benzoic acid, sodium benzoate, ammonium benzoate, butyl benzoate, calcium benzoate, ethyl benzoate, isobutyl benzoate, isopropyl benzoate, magnesium benzoate, mea-benzoate, methyl benzoate, phenyl benzoate, potassium benzoate, propyl benzoate, propionic acid, ammonium propionate, calcium propionate, magnesium propionate, potassium propionate, sodium propionate, salicylic acid, calcium salicylate, magnesium salicylate, measalicylate, sodium salicylate, potassium salicylate, teasalicylate, sorbic acid, calcium sorbate, sodium sorbate, potassium sorbate, o-phenylphenol, sodium sulfite, ammonium bisulfite, ammonium sulfite, potassium sulfite, potassium hydrogen sulfite, sodium bisulfite, sodium metabisulfite, potassium metabisulfite, chlorobutanol, 4-hydroxybenzoic acid, methylparaben, potassium ethylparaben, potassium paraben. sodium methylparaben, sodium ethylparaben, ethylparaben, sodium paraben, potassium methylparaben, calcium paraben, butylparaben, propylparaben, sodium propoylparaben, sodium butylparaben, potassium butylparaben, potassium propylparaben, dehydroacetic acid, sodium dehydroacetate, formic acid, sodium formate, dibromohexamidine isethionate, thimerosal, phenyl mercuric acetate, phenyl mercuric benzoate, undecylenic acid, potassium undecylenate, sodium undecylenate, calcium undecylenate, mea-undecylenate, tea-undecylenate, hexetidine, 5-bromo-5-nitro-1,3-dioxane, 2-bromo-2-nitropropane-1,3-diol, dichlorobenzyl alcohol, triclocarban, p-chloro-m-cresol, triclosan, chloroxylenol, imidazolidinyl urea, polyaminopropyl; biguanide, phenoxyethanol, methenamine, climbazole, DMDM hydantoin, benzyl alcohol, 1-hydroxy-4-methyl-6-(2,4,4-trimethylpentyl)-2 pyridon, piroctone olamine, bromochlorophene, o-cymen-5-ol, methylchloroisothiazolinone and methylisothiazolinone, chlorhexidine, chlorhexidine diacetate, chlorhexidine digluconate, chlorhexidine dihydrochloride, phenoxyisopropanol, behentrimonium chloride (1), cetrimonium bromide, cetrimonium chloride (2), laurtrimoniumbromide, laurtrimonium chloride, steartrimonium bromide, steartrimonium chloride (2), dimethyl oxazolidine, diazolidinyl urea, hexamidine, hexamidine diisethionate, hexamidine hexamidine diparaben, paraben, glutaral, 7-ethylbicyclooxazolidine, chlorphenesin, sodium hydroxymethylglycinate, silver chloride, benzethonium chloride, benzalkonium chloride, benzalkonium bromide, benzalkonium saccharinate, benzylhemiformal, iodopropynyl butylcarbamate, methylisothiazolinone, citric acid, silver citrate, hydroxyethoxy-phenyl butanone (HEPB) and any mixture thereof.

Antibacterial or antimycotic active substances: In addition to the antimicrobials as described therein, antibacterial or antimycotic active substances can also particularly advantageously be used in the compositions according to the present invention, wherein any antibacterial or antimycotic active substances can be used which are suitable or customary in cosmetic or pharmaceutical, in particular dermatological applications. In addition to the large group of conventional antibiotics, other products which are advantageous here include those relevant to cosmetics such as in particular triclosan, climbazole, octoxyglycerin, Octopirox® (1-hydroxy-4-methyl-6-(2,4,4-trimethylpentyl)-2 (1H)-pyridone 2-aminoethanol salt), chitosan, farnesol, glycerol monolaurate or combinations of said substances.

UV-filters: The composition as defined herein, is advantageously combined with at least one primary sun protection factor and/or with at least one secondary sun protection factor, in order to increase the SPF, i.e. to obtain a high SPF and to cover a broad UVA and UVB range.

The compositions according to the invention advantageously contains at least one UVA filter and/or at least one further UVB filter and/or a broadband filter and/or at least one inorganic pigment, preferably at least one UVA filter and at least one UVB filter for their use in stopping UV radiation.

For example, the at least one primary UV-filter may be one or more organic UV-filters and/or one or more inorganic UV-filters. Non-limiting examples of UV-filters include:
(i) sparingly soluble UV-filters (not appreciably soluble in either water or oil) such as Methylene Bis-benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl-]phenyl];
(ii) oil soluble organic UV-filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
(iii) inorganic UV-filters such as titanium oxide and zinc oxide, iron oxide, zirconium oxide and cerium oxide; and
(iv) water soluble UV-filters such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PAB; and any mixture thereof.

Many different organic compounds can serve as UV-filters. They fall into several structural classes:
(3) Benzophenones
   a. Benzophenone-3 (BP3)
   b. Benzophenone-4 (BP4)
(4) Salicylates
   a. Homosalate (HMS)
   b. 2-ethylhexyl salicylate (EHS)
(5) p-Aminobenzoic acid and derivatives
   a. Ethylhexyl dimethyl PABA (OD-PABA)
   b. 4-p-aminobenzoic acid (PABA)
(6) Benzimidazole derivatives
   a. Phenylbenzimidazole sulfonic acid (PMDSA)
   b. Disodium phenyl dibenzimidazole tetrasulfonate (bisdisulizole disodium)
(7) Triazines
   a. Ethylhexyltriazone (OT)
   b. Diethylhexyl butamido triazone (DBT)
   c. Bis-ethylhexyloxyphenol methoxyphenyl triazine (EMT)
(8) Benzotriazoles
   a. Drometrizole trisiloxane (DRT)
   b. Methylene bis-benzotriazolyl tetramethylbutylphenol (MBP, biscotrizole)
(9) Dibenzoylmethane derivatives
   a. 4-tert-Butyl-4′-methoxydibenzoylmethane (BM-DBM, avobenzone)
(10) Cinnamates
   a. Ethylhexyl methoxycinnamate (OMC)
   b. Isoamyl p-methoxycinnamate (IMC, amiloxate)
(11) Camphor derivatives
   a. Terephtalydene dicamphor sulfonic acid (PDSA)
   b. 3-benzylidene camphor (3BC)
   c. Benzylidene camphor sulfonic acid (BCSA)
   d. 4-methylbenzylidene camphor (4-MBC)
   e. Polyacrylamidomethyl benzylidene camphor (PBC)
(12) Camphor benzalkonium methosulfate (CBM)

According to the present invention, the at least one inorganic UV-filter is selected from the group of pigments consisting of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), and mixtures thereof.

Preferably, the at least one inorganic UV-filter is titanium dioxide, zinc oxide, and mixtures thereof, more preferably the at least one inorganic UV-filter is titanium oxide and/or zinc oxide, and most preferably, the at least one inorganic UV-filter is zinc oxide. ZnO has a broad UVA/UVB absorption curve, while TiO₂ provides better UVB protection.

According to another variant, the at least one inorganic UV-filter is in form of particles having a weight medium particle size d₅₀ from 1 nm to 1000 nm, preferably from 3 nm to 800 nm, more preferably from 5 nm to 600 nm, and most preferably from 10 nm to 400 nm.

The inorganic UV-filters also encompass nano pigments (mean size of the primary particles: generally from 1 nm to 100 nm, preferably from 3 nm to 90 nm, more preferred from 5 nm to 80 nm and most preferred from 10 to 70 nm) of untreated or treated metal oxides such as, for example, nano pigments of titanium dioxide (TiO₂) (amorphous or crystallized in rutile and/or anatase form), zinc oxide (ZnO), iron oxide (Fe₂O₃), zirconium oxide (ZrO₂), silicon dioxide (SiO₂), manganese oxide (e.g. MnO), aluminium oxide (Al₂O₃), cerium oxide (Ce₂O₃), barium carbonate (BaCO₃), calcium carbonate (CaCO₃), or mixtures thereof.

The treated nano pigments and non-nano pigments are pigments that have undergone one or more surface treatments of chemical, electronic, mechanochemical and/or mechanical nature with compounds, such as amino acids, beeswax, fatty acids, fatty acid esters, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol, hydrated silica, stearic acid, jojoba esters, or glutamic acid derivates,

The treated nano pigments and non-nano pigments may more particularly be titanium oxides treated with silica and alumina, alumina and aluminium stearate, alumina and aluminium laurate, aluminium hydroxide, iron oxides and iron stearate, silica, alumina and silicone, sodium hexametaphosphate, octyltrimethoxysilane, alumina and stearic acid, alumina and glycerol, or alumina and silicone or polyhydroxy stearic acid, hydrated silica, jojoba esters, steaoryl glutamic acid, glutamic acid and derivates, trimethoxycaprylylsilane, glycerin, dimethicone, hydrogen dimethicone, simethicone or combinations thereof. Other titanium oxide nano pigments treated with a silicone are preferably TiO₂ treated with octyltrimethylsilane, preferably for which the mean size of the elementary particles is from 25 to 40 nm; TiO₂ treated with a polydimethylsiloxane, preferably for which the mean size of the elementary particles is 21 nm; or TiO₂ treated with a polydimethylhydrogenosiloxane, preferably for which the mean size of the elementary particles is 25 nm.

The coated zinc oxide nano pigments and zinc oxide non-nano pigments are for example ZnO coated with polymethylhydrogenosiloxane; ZnO dispersions in cyclopolymethylsiloxane/oxyethylenated polydimethylsiloxane, containing 30% or 80% of nano or non-nano zinc oxides coated with silica and polymethylhydrogenosiloxane; ZnO coated with perfluoroalkyl phosphate and copolymer based on perfluoroalkylethyl as a dispersion in cyclopentasiloxane; ZnO coated with dimethoxydiphenylsilanetriethoxycaprylylsilane cross-polymer; ZnO coated with glutamic acid; ZnO coated with octyltriethoxy silane; ZnO coated with dimethicone; ZnO coated with silicone-grafted acrylic polymer, dispersed in cyclodimethylsiloxane; ZnO coated with triethoxycaprylylsilane; ZnO coated with polyhydroxystearic acid; alumina-treated ZnO dispersed in an ethylhexyl methoxycinnamate/PVP-hexadecene/methicone copolymer mixture; ZnO coated with silica and polymethylsilsesquioxane; ZnO dispersed in hydroxystearic acid polycondensate; or ZnO coated with a mixture of steaoryl glutamic acid, jojoba esters, polyhydroxystearic acid, isopropyl titanium triisostearate, hydrogenated olive oil stearyl esters, cetearyl nonanoate, cera alba, dimethicone, dimethoxydiphenylsilanetriethoxycaprylylsilane cross-polymer, octyltriethoxysilane, glutamic acid and sodium stearoyl lactylate.

Also preferred are particulate UV-filters or inorganic pigments, which can optionally be hydrophobed, such as the oxides of iron (Fe₂O₃), zirconium (ZrO₂), silicon (SiO₂), manganese (e.g. MnO), aluminium (Al₂O₃), cerium (e.g. Ce₂O₃), barium carbonate (BaCO₃), calcium carbonate (CaCO₃), or mixtures thereof.

The at least one primary organic and/or inorganic UV-filter is selected from the group consisting of Camphor Benzalkonium Methosulfate, Homosalate, Benzophenone-3, Phenylbenzimidazole Sulfonic Acid, Terephthalylidene Dicamphor Sulfonic Acid, Butyl Methoxydibenzoylmethane, Benzylidene Camphor Sulfonic Acid, Octocrylene, Polyacrylamidomethyl Benzylidene Camphor, Ethylhexyl Methoxycinnamate, PEG-25 PABA, Isoamyl p-Methoxycinnamate, Ethylhexyl Triazone, Drometrizole Trisiloxane, Diethylhexyl Butamido Triazone, 4-Methylbenzylidene Camphor, Ethylhexyl Salicylate, Ethylhexyl Dimethyl PABA, Benzophenone-4, Benzophenone-5, Methylene Bis-Benzotriazolyl Tetramethylbutylphenol (nano), Disodium Phenyl Dibenzimidazole Tetrasulfonate, Bis-Ethylhexyloxyphenol Methoxyphenyl Triazine, Polysilicone-15, Titanium Dioxide, Titanium Dioxide (nano), Diethylamino Hydroxy benzoyl Hexyl Benzoate, Tris-biphenyl triazine (nano), Zinc Oxide, Zinc Oxide (nano), Phenylene Bis-Diphenyltriazine, Methoxypropylamino Cyclohexenylidene Ethoxyethylcyanoacetate, Bis-(Diethylaminohydroxybenzoyl Benzoyl) Piperazine, TEA-Salicylate, Benzophenone-8, PABA, Ethylhexyl Dimethyl PABA, Menthyl Anthranilate, and any mixture thereof.

The aforesaid primary organic or inorganic UV-filters are used in the compositions according to the present invention either as a single component or preferably in a mixture with two, three, four or more further of said UV-filters as specified above.

In order to optimize the SPF, i.e. to obtain a high SPF in a range of 6 to 100, preferably in a range of 6 to 70, and to cover a broad UVA and UVB range, the compositions comprises at least two, more preferably at least three, most preferably at least four or even more different primary organic and/or inorganic UV-filters, i.e. in particular organic UV-filters and/or inorganic pigments (UV-filtering pigments). The combination of effective sun protection UV-filters of different categories such as UVA filter, UVB, broadband filter, inorganic pigments provides reliable protection against the different UV rays in the wavelength range of 290 to 400 nm.

Advantageous the amount of primary organic and/or inorganic sun protection substances (UV-filters) in the compositions according to the present invention is advantageously from 0.01 to 80.0% by weight, preferably from 0.1 to 75.0% by weight, most preferred from 0.5 to 70.0% by weight, based on the total weight of the ready-to-use formulation.

Besides the group of primary organic and/or inorganic sun protection UV-filters mentioned above, secondary sun protection ingredients of the antioxidant type may also be advantageously used in the compositions according to the present invention in order to further optimize the UV protection.

Secondary sun protection ingredients of the antioxidant type interrupt the photochemical reaction chain which is initiated when UV rays penetrate into the skin.

Typical examples of secondary sun protection ingredients are amino acids (for example arginine, lysine, glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxy fatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, sinapic acid, sinapoyl malate, sinapate ester derivatives, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The group of secondary sun protection ingredients also encompasses plant-based extract(s). Said plant-based extracts have antioxidative and in general photoprotective properties and interrupt the photochemical reaction chain which is initiated when UV rays penetrate into the skin, and, thus, are effective in preventing skin aging. The plant extracts with UV protection properties are selected from the group consisting of Lignin/Cellulose, Lignin, Lignin Hydrolyzed, Propolis and green propolis, Galanga Extract, macro and micro algae (Porphyra, red algae (Porphyra umbilicalis), Palmaria palmata, Saccharina latissimi, Corallina pilulifera, Eckloina cava, Sargassum sagamianum, Porphyra rosengurttii, Sargassum siliquastrum, Thalassiosira weissflogii, Green Microalgae (e.g. Interfilum and Klebsormidium), Seaweed (e.g. Padina australis, Euchema, cottonii), Pongamia glabra Seed Oil, Karanja oil (Millettia pinnata), Rhatania Extrakt (Krameria lappacea), Lichens, Soybean, Caper (Capparis spinosa), Bamboo, Green Coffee oil, Buriti Oil (Mauritia flexuosa), Black Sea Cucumber (Holothuria atra), Cyanobacteria, Fernblock (Polypodium leucotomos), Green Tee, Black Tea (Camellia sinensis), Aloe vera, Walnut, Borage oil, Evening primrose oil, Avocado oil, Tea tree oil, Red Clover (Trifolium pratense L.), Soybean (Glycine max L.), Caper (Capparis spinosa), Almonds, Flame of the forest (Spathodea campanulata), Milk thistle (Silybum marianum L.), Cashew nut shell, Grape, Red Orange, Pomegranate (Punica granatu), Bilberry (Vaccinium myrtillus L.), Bog blueberry (Vaccinium uliginosum L.), Strawberry (Fragaria ananassa), Foti (Polygonum multiflorum Thunb), Turmeric (Curcuma longa L.), Ginseng (Panax ginseng), English ivy (Hedera helix), Broccoli (Brassica oleracea var italica), Tamarind (Tamarindus indica), Opuntia ficus-indica, Coffee, Basil (Ocimum basilicum, Linn.), pomegranate seed oil (Punica granatum), wheat germ oil (Triticum vulgare), blackcurrant seed oil (Ribes nigrum), sesameoil (Sesamum indicum), carrot root (Daucuscarota sativa), raspberry seed oil (Rubus idaeus), traditional medicinal plants from Sri Lanka (Aporosa lindleyana (Euphorbiaceae), Atalantia ceylanica (Rutaceae), Hibiscus furcatus (Malvaceae), Olaxzeylanica (Olacaceae), Ophiorrhiza mungos (Rubiaceae), Argyreia populifolia (Convolvulaceae), Ipomoea mauritiana (Convolvulaceae), Lasia spinosa (Araceae), Leucas zeylanica (Lamiaceae), Plectranthus zeylanicus (Lamiaceae) and Brazil (D. gardneriana seeds, L. microphylla leafs, Amburana cearensis, Aspidosperma cuspa, Aspidosperma pyrifolium, Croton sonderianus, Curatella americana, Dimophandra gardneriana, Lippia microphylla, Luehea paniculata, Sida galheirensis and any mixture thereof.

Preferably, the plant extracts with UV protection properties are selected from the group consisting of Lignin/Cellulose, Lignin, Lignin Hydrolyzed, Galanga Extract, Porphyra, red algae (Porphyra umbilicalis), Green Microalgae (e.g. Interfilum and Klebsormidium), Seaweed (e.g. Padina australis, Euchema, cottonii), Pongamia glabra Seed Oil, Karanja oil (Millettia pinnata), Rhatania Extrakt (Krameria lappacea), Soybean, Bamboo, Buriti Oil (Mauritia flexuosa), Cyanobacteria, Fernblock (Polypodium leucotomos), Green Tee, Black Tea (Camellia sinensis), Evening primrose oil, Cashew nut shell, Pomegranate (Punica granatu), and any mixture thereof.

Advantageously, the amount of secondary sun protection substances in the cosmetic or pharmaceutical preparation or homecare product according to the present invention is advantageously from 0.005 to 5.0% by weight, preferably from 0.01 to 4.0% by weight, most preferred from 0.05 to 3.0% by weight, based on the total weight of the ready-to-use formulation.

Film formers: The food, cosmetic or pharmaceutical preparation, in particular sunscreen product, or homecare product according to the present invention may advantageously include one or more film formers or film forming agent. A film forming agent is a hydrophobic material that imparts water resistance and film forming characteristics to the sunscreen products, cosmetic or pharmaceutical preparations or homecare products. Standard film formers are preferably chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds. Preferred film formers for improving water resistance of the composition are selected from the group consisting of Polyamide-8 (such as Oleocraft LP 20 PA (MV)), Polyamide-3 (such as Oleocraft MP 32 PA (MV)), Trimethylpentanediol/Adipic Acid/Glycerin Crosspolymer (such as WetFilm), Octyldodecyl Citrate Crosspolymer (such as CosmoSurf CE 100), Polyglyceryl-3 Methyl Glucose Distearate (such as TegoCare 450), C28-52 Olefin/Undecylenic Acid Copolymer (such as Performa V6112), Hydrolyzed Jojoba esters, Jojoba Esters, Aqua (such as Floraester Jojoba K100), Cyclopentasiloxane Acrylates/Dimethicone copolymer (such as KP545), Acrylates/Beheneth-25 Methacylate Copolymer (such as Volarest), Styrene/Acrylates Copolymer (such as Dermacryl E Polymer), Polyurethane (such as Baycusan C1001), Polyurethane-2 and Polymethyl Methacrylate (such as Hybridur 875 Polymer), Acrylates/Octylacrylamide Copolymer (such as Dermacryl 2.0 Polymer), Beeswax (Cera Alba) (and) Sodium Stearoyl Lactylate (such as SymEffect Sun), Pullulan, Xanthan Gum, Hydrogenated Palm Oil, Saccharum Officinarum Extract (SymEffect™MUV), Microcrystalline cellulose (and) Sphingomonas ferment extract (and) Cellulose gum (such as PemuPur™ START Polymer), Acacia senegal Gum and Xanthan Gum (such as SOLAGUM™ AX), Microcrystalline Cellulose (and) Cellulose Gum (such as Natpure Cellgum Plus), Sodium Stearoyl Glutamate (such as AMISOFT® HS-11P), Hydrogenated Polycyclopentadiene (and) Caprylic/Capric Triglyceride (such as Koboguard® 5400 CCT), Glyceryl Hydrogenated Rosinate (and) Caprylic/Capric Triglyceride (and) Tocopherol (such as KOBOGUARD® NATURAL 2063-CCT), Galactoarabinan (such as LaraCare® A 200), C26-28 Alkyl Dimethicone (such as Wacker BELSIL® CDM 3526 VP), Caprylyl Methicone (such as Silsoft 034), Stearyl Dimethicone (and) Octadecene (such as Dowsil 2503 Cosmetic Wax), VP/Hexadecene Copolymer (such as Antaron™ V 216), Triacontanyl PVP (such as Antaron™ WP-660), Capryloyl Glycerin/Sebacic Acid Copolymer (such as LexFilm™ Sun Natural MB), Poly C10-30 Alkyl Acrylate (such as Tego SP 13 Sun Up), Microcrystalline Cellulose (such as Sunspheres™ BIO SPF Booster), and any mixture thereof. The aforesaid film forming agents are used in the sunscreen, cosmetic or pharmaceutical preparation or homecare product either as a single component or preferably in a mixture with two or more further of said film forming agents as specified above. The film forming agents are used in amounts effective to allow the final formulation to remain on the skin or surface after exposure to circulating water for at least 40 minutes, preferably 80 minutes. The film forming agent is present in the food, cosmetic or pharmaceutical preparation or homecare product in an amount of 0.01 to 10.0% by weight, preferably 0.05 to 8.0% by weight, based on the total weight of the final formulation.

Water resistance improving agents: The food, cosmetic or pharmaceutical preparation, in particular sunscreen product, or homecare product according to the present invention may advantageously include one or more agents for improving water resistance. An agent for improving water resistance is a hydrophobic material that imparts film forming and water resistance characteristics to an emulsion. Typical suitable water resistance improving agents include copolymers derived from polymerization of octadecene-1 and maleic anhydride. A preferred water resistance improving agent is a polyanhydride resin. Another preferred water resistance improving agent is a copolymer of vinyl pyrrolidone and eicosene monomers. The water resistance improving agent(s) is/are used in amounts effective to allow the final formulation to remain on the skin or surface after exposure to circulating water for at least 40 minutes, preferably 80 minutes. One or more water resistance improving agents can optionally be included in the final formulation in an amount ranging from 0.01 to 10.0% by weight, preferably 0.05 to 8.0% by weight, more preferably 0.1 to 5.0% by weight, based on the total weight of the final formulation.

Desquamating agents: The cosmetic or pharmaceutical compositions according to the present invention preferably contain one or more desquamating agents. The expression “desquamating agent” is understood to mean any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic, citric, lactic, tartaric, malic or mandelic acids; urea; gentisic acid; oligofucoses; cinnamic acid; extract of Sophora japonica; resveratrol and some derivatives of jasmonic acid;
or on the enzymes involved in the desquamation or the degradation of the corneodesmosomes, glycosidases, stratum corneum chymotryptic enzyme (SCCE) or other proteases (trypsin, chymotrypsin-like). There may be mentioned agents chelating inorganic salts: EDTA; N-acyl-N,N′,N′-ethylenediaminetriacetic acid; aminosulphonic compounds and in particular (N-2-hydroxyethylpiperazine-N-2-ethane) sulphonic acid (HEPES); derivatives of 2-oxothiazolidine-4-carboxylic acid (procysteine); derivatives of alpha-amino acids of the glycine type (as described in EP-0 852 949, and sodium methylglycine diacetate marketed by BASF under the trade name TRILON M); honey; sugar derivatives such as O-octanoyl-6-D-maltose and N-acetylglucosamine; chestnut extracts such as those marketed by the company SILAB under the name Recoverine®, prickly pear extracts such as those marketed under the name Exfolactive® by the company SILAB, or Phytosphingosine SLC® (phytosphingosine grafted with a salicylic acid) marketed by the company Degussa.

Desquamating agents suitable for the invention may be chosen in particular from the group comprising sulphonic acids, calcium chelators, α-hydroxy acids such as glycolic, citric, lactic, tartaric, malic or mandelic acids; ascorbic acid and its derivatives such as ascorbyl glucoside and magnesium ascorbyl phosphate; nicotinamide; urea; (N-2-hydroxyethylpiperazine-N-2-ethane) sulphonic acid (HEPES), β-hydroxy acids such as salicylic acid and its derivatives, retinoids such as retinol and its esters, retinal, retinoic acid and its derivatives, chestnut or prickly pear extracts, in particular marketed by SILAB; reducing compounds such as cysteine or cysteine precursors. Desquamating agents which can be used are also nicotinic acid and its esters and nicotinamide, also called vitamin B3 or vitamin PP, and ascorbic acid and its precursors.

Anti-dandruff substances: In addition, the compositions according to the present invention can also advantageously be used in combination with one or more anti-dandruff substances, including triclosan, climbazole, octoxyglycerin, Octopirox® (1-hydroxy-4-methyl-6-(2,4,4-trimethylpentyl)-2 (1H)-pyridone 2-aminoethanol salt), chitosan, farnesol, glycerol monolaurate, Propanediol Monocaprylate or combinations of said substances, which are used inter alia against dandruff.

Further suitable anti-dandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

(Metal) chelating agents: A combination with one or more (metal) chelating agents can also be advantageous used in the compositions according to the present invention, wherein any metal chelating agents can be used which are suitable or customary in cosmetic or pharmaceutical applications. Preferred (metal) chelating agents include α-hydroxy fatty acids, phytic acid, lactoferrin, α-hydroxy acids, such as inter alia gluconic acid, glyceric acid, glycolic acid, isocitric acid, citric acid, lactic acid, malic acid, mandelic acid, tartaric acid, as well as humic acids, bile acids, bile extracts, bilirubin, biliverdin or EDTA, EGTA, MGDA (Trisodiumdicarboxymethyl alaninante), and their derivatives. The use of one or more chelating agent(s) improves the stability of the composition according to the present invention.

Surfactants: The compositions according to the present invention preferably include one or more anionic and/or amphoteric or zwitterionic surfactants. Typical examples encompass: Almondamidopropylamine Oxide, Almondamidopropyl Betaine, Aminopropyl Laurylglutamine, Ammonium C12-15 Alkyl Sulfate, Ammonium C12-16 Alkyl Sulfate, Ammonium Capryleth Sulfate, Ammonium Cocomonoglyceride Sulfate, Ammonium Coco-Sulfate, Ammonium Cocoyl Isethionate, Ammonium Cocoyl Sarcosinate, Ammonium C12-15 Pareth Sulfate, Ammonium C9-10 Perfluoroalkylsulfonate, Ammonium Dinonyl Sulfosuccinate, Ammonium Dodecylbenzenesulfonate, Ammonium Isostearate, Ammonium Laureth-6 Carboxylate, Ammonium Laureth-8 Carboxylate, Ammonium Laureth Sulfate, Ammonium Laureth-5 Sulfate, Ammonium Laureth-7 Sulfate, Ammonium Laureth-9 Sulfate, Ammonium Laureth-12 Sulfate, Ammonium Lauroyl Sarcosinate, Ammonium Lauryl Sulfate, Ammonium Lauryl Sulfosuccinate, Ammonium Myreth Sulfate, Ammonium Myristyl Sulfate, Ammonium Nonoxynol-4 Sulfate, Ammonium Nonoxynol-30 Sulfate, Ammonium Oleate, Ammonium Palm Kernel Sulfate, Ammonium Stearate, Ammonium Tallate, AMPD-Isostearoyl Hydrolyzed Collagen, AMPD-Rosin Hydrolyzed Collagen, AMP-Isostearoyl Hydrolyzed Collagen, AMP-Isostearoyl Hydrolyzed Keratin, AMP-Isostearoyl Hydrolyzed Soy Protein, AMP-Isostearoyl Hydrolyzed Wheat Protein, Apricotamidopropyl Betaine, Arachidic Acid, Arginine Hexyldecyl Phosphate, Avocadamidopropyl Betaine, Avocado Oil Glycereth-8 Esters, Babassu Acid, Babassuamidopropylamine Oxide, Babassuamidopropyl Betaine, Beeswax Acid, Behenamidopropyl Betaine, Behenamine Oxide, Beheneth-25, Beheneth-30, Behenic Acid, Behenyl Betaine, Bis-Butyldimethicone Polyglyceryl-3, Butoxynol-5 Carboxylic Acid, Butoxynol-19 Carboxylic Acid, Butyldimoniumhydroxypropyl Butylglucosides Chloride, Butyldimoniumhydroxypropyl Laurylglucosides Chloride, Butyl Glucoside, Butylglucoside Caprate, Butylglucosides Hydroxypropyltrimonium Chloride, Butyloctanoic Acid, C18-36 Acid, C20-40 Acid, C30-50 Acid, C16-22 Acid Amide MEA, Calcium Dodecylbenzenesulfonate, Calcium Lauroyl Taurate, C9-16 Alkane/Cycloalkane, C10-14 Alkyl Benzenesulfonic Acid, C12-14 Alkyl Diaminoethylglycine HCL, C9-15 Alkyl Phosphate, Candida bombicola/Glucose/Methyl Rapeseedate Ferment, Canolamidopropyl Betaine, Capric Acid, Caproic Acid, Caproyl Ethyl Glucoside, Capryl/Capramidopropyl Betaine, Capryleth-4 Carboxylic Acid, Capryleth-6 Carboxylic Acid, Capryleth-9 Carboxylic Acid, Caprylic Acid, Capryloyl Collagen Amino Acids, Capryloyl Glycine, Capryloyl Hydrolyzed Collagen, Capryloyl Hydrolyzed Keratin, Capryloyl Keratin Amino Acids, Capryloyl Silk Amino Acids, Caprylyl/Capryl Glucoside, Caprylyl/Capryl Wheat Bran/Straw Glycosides, Caprylyl Glucoside, Caprylyl Glyceryl Ether, Caprylyl Pyrrolidone, Carnitine, Ceteareth-20, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Ceteareth-25 Carboxylic Acid, Ceteareth-2 Phosphate, Ceteareth-4 Phosphate, Ceteareth-5 Phosphate, Ceteareth-10 Phosphate, Ceteth-20, Ceteth-23, Ceteth-24, Ceteth-25, Ceteth-30, Ceteth-40, Ceteth-45, Ceteth-150, Ceteth-8 Phosphate, Ceteth-10 Phosphate, Ceteth-20 Phosphate, Cetoleth-22, Cetoleth-24, Cetoleth-25, Cetoleth-30, Cetyl Betaine, Chrysanthemum sinense Flower Extract, C12-14 Hydroxyalkyl Hydroxyethyl Beta-Alanine, C12-14 Hydroxyalkyl Hydroxyethyl Sarcosine, Cocamidoethyl Betaine, Cocamidopropylamine Oxide, Cocamidopropyl Betainamide MEA Chloride, Cocamidopropyl Betaine, Cocamidopropyl Hydroxysultaine, Cocamine Oxide, Cocaminobutyric Acid, Cocaminopropionic Acid, Coceth-7 Carboxylic Acid, Coceth-4 Glucoside, Cocoamphodipropionic Acid, Cocobetainamido Amphopropionate, Coco-Betaine, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Coco-Glucoside, Cocoglucosides Hydroxypropyltrimonium Chloride, Coco-Hydroxysultaine, Coco-Morpholine Oxide, Coconut Acid, Coconut Oil Glycereth-8 Esters, Coco/Oleamidopropyl Betaine, Coco-Sultaine, Coco/Sunfloweramidopropyl Betaine, Cocoylcholine Methosulfate, Cocoyl Glutamic Acid, Cocoyl Hydrolyzed Collagen, Cocoyl Hydrolyzed Keratin, Cocoyl Hydrolyzed Oat Protein, Cocoyl Hydrolyzed Rice Protein, Cocoyl Hydrolyzed Silk, Cocoyl Hydrolyzed Soy Protein, Cocoyl Hydrolyzed Wheat Protein, Cocoyl Sarcosine, Corn Acid, Cottonseed Acid, Cottonseed Oil Glycereth-8 Esters, C10-16 Pareth-1, C10-16 Pareth-2, C11-13 Pareth-6, C11-13 Pareth-9, C11-13 Pareth-10, C11-15 Pareth-30, C11-15 Pareth-40, C12-13 Pareth-1, C12-13 Pareth-23, C12-14 Pareth-5, C12-14 Pareth-9, C13-15 Pareth-21, C14-15 Pareth-8, C20-22 Pareth-30, C20-40 Pareth-40, C20-40 Pareth-95, C22-24 Pareth-33, C30-50 Pareth-40, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, C6-10 Pareth-4 Phosphate, C12-13 Pareth-2 Phosphate, C12-13 Pareth-10 Phosphate, C12-15 Pareth-6 Phosphate, C12-15 Pareth-8 Phosphate, C12-15 Pareth-10 Phosphate, C12-16 Pareth-6 Phosphate, C4-18 Perfluoroalkylethyl Thiohydroxypropyltrimonium Chloride, Cupuassuamidopropyl Betaine, DEA-C12-13 Alkyl Sulfate, DEA-C12-15 Alkyl Sulfate, DEA-Ceteareth-2 Phosphate, DEA-Cetyl Sulfate, DEA-Cocoamphodipropionate, DEA-C12-13 Pareth-3 Sulfate, DEA-Cyclocarboxypropyloleate, DEA-Dodecylbenzenesulfonate, DEA-Isostearate, DEA-Laureth Sulfate, DEA-Lauryl Sulfate, DEA-Linoleate, DEA-Methyl Myristate Sulfonate, DEA-Myreth Sulfate, DEA-Myristate, DEA-Myristyl Sulfate, DEA-Oleth-5 Phosphate, DEA-Oleth-20 Phosphate, DEA PG-Oleate, Deceth-7 Carboxylic Acid, Deceth-7 Glucoside, Deceth-9 Phosphate, Decylamine Oxide, Decyl Betaine, Decyl Glucoside, Decyltetradeceth-30, Decyltetradecylamine Oxide, Diammonium Lauramido-MEA Sulfosuccinate, Diammonium Lauryl Sulfosuccinate, Diammonium Oleamido PEG-2 Sulfosuccinate, Dibutoxymethane, Di-Cl 2-15 Pareth-2 Phosphate, Di-Cl 2-15 Pareth-4 Phosphate, Di-Cl 2-15 Pareth-6 Phosphate, Di-C12-15 Pareth-8 Phosphate, Di-Cl 2-15 Pareth-10 Phosphate, Didodecyl Butanetetracarboxylate, Diethylamine Laureth Sulfate, Diethylhexyl Sodium Sulfosuccinate, Dihydroxyethyl C8-10 Alkoxypropylamine Oxide, Dihydroxyethyl C9-11 Alkoxypropylamine Oxide, Dihydroxyethyl C12-15 Alkoxypropylamine Oxide, Dihydroxyethyl Cocamine Oxide, Dihydroxyethyl Lauramine Oxide, Dihydroxyethyl Stearamine Oxide, Dihydroxyethyl Tallowamine Oxide, Dimethicone PEG-7 Phosphate, Dimethicone PEG-10 Phosphate, Dimethicone PEG/PPG-7/4 Phosphate, Dimethicone PEG/PPG-12/4 Phosphate, Dimethicone/Polyglycerin-3 Crosspolymer, Dimethicone Propyl PG-Betaine, Dimyristyl Phosphate, Dioleoylamidoethyl Hydroxyethylmonium Methosulfate, DIPA-Hydrogenated Cocoate, DIPA-Lanolate, Dipotassium DIPA-Myristate, Capryloyl Glutamate, Dipotassium Lauryl Sulfosuccinate, Dipotassium Undecylenoyl Glutamate, Disodium Babassuamido MEA-Sulfosuccinate, Disodium Caproamphodiacetate, Disodium Caproamphodipropionate, Disodium Capryloamphodiacetate, Disodium Capryloamphodipropionate, Disodium Capryloyl Glutamate, Disodium Cetearyl Sulfosuccinate, Disodium Cetyl Phenyl Ether Disulfonate, Disodium Cetyl Sulfosuccinate, Disodium Cocamido MEA-Sulfosuccinate, Disodium Cocamido MIPA PEG-4 Sulfosuccinate, Disodium Cocamido MIPA-Sulfosuccinate, Disodium Cocamido PEG-3 Sulfosuccinate, Disodium Coceth-3 Sulfosuccinate, Disodium Cocoamphocarboxyethylhydroxypropylsulfonate, Disodium Cocoamphodiacetate, Disodium Cocoamphodipropionate, Disodium Coco-Glucoside Sulfosuccinate, Disodium Coco-Sulfosuccinate, Disodium Cocoyl Butyl Gluceth-10 Sulfosuccinate, Disodium Cocoyl Glutamate, Disodium C12-14 Pareth-1 Sulfosuccinate, Disodium C12-14 Pareth-2 Sulfosuccinate, Disodium C12-15 Pareth Sulfosuccinate, Disodium C12-14 Sec-Pareth-3 Sulfosuccinate, Disodium C12-14 Sec-Pareth-5 Sulfosuccinate, Disodium C12-14 Sec-Pareth-7 Sulfosuccinate, Disodium C12-14 Sec-Pareth-9 Sulfosuccinate, Disodium C12-14 Sec-Pareth-12 Sulfosuccinate, Disodium Deceth-5 Sulfosuccinate, Disodium Deceth-6 Sulfosuccinate, Disodium Decyl Phenyl Ether Disulfonate, Disodium Dihydroxyethyl Sulfosuccinylundecylenate, Disodium Ethylene Dicocamide PEG-15 Disulfate, Disodium Hydrogenated Cottonseed Glyceride Sulfosuccinate, Disodium Hydrogenated Tallow Glutamate, Disodium Hydroxydecyl Sorbitol Citrate, Disodium Isodecyl Sulfosuccinate, Disodium Isostearamido MEA-Sulfosuccinate, Disodium Isostearamido MIPA-Sulfosuccinate, Disodium Isostearoamphodiacetate, Disodium Isostearoamphodipropionate, Disodium Isostearyl Sulfosuccinate, Disodium Laneth-5 Sulfosuccinate, Disodium Lauramido MEA-Sulfosuccinate, Disodium Lauramido MIPA Glycol Sulfosuccinate, Disodium Lauramido PEG-2 Sulfosuccinate, Disodium Lauramido PEG-5 Sulfosuccinate, Disodium Laureth-5 Carboxyamphodiacetate, Disodium Laureth-7 Citrate, Disodium Laureth Sulfosuccinate, Disodium Laureth-6 Sulfosuccinate, Disodium Laureth-9 Sulfosuccinate, Disodium Laureth-12 Sulfosuccinate, Disodium Lauriminobishydroxypropylsulfonate, Disodium Lauriminodiacetate, Disodium Lauriminodipropionate, Disodium Lauriminodipropionate Tocopheryl Phosphates, Disodium Lauroamphodiacetate, Disodium Lauroamphodipropionate, Disodium N-Lauroyl Aspartate, Disodium Lauroyl Glutamate, Disodium Lauryl Phenyl Ether Disulfonate, Disodium Lauryl Sulfosuccinate, Disodium Myristamido MEA-Sulfosuccinate, Disodium Nonoxynol-10 Sulfosuccinate, Disodium Oleamido MEA-Sulfosuccinate, Disodium Oleamido MIPA-Sulfosuccinate, Disodium Oleamido PEG-2 Sulfosuccinate, Disodium Oleoamphodipropionate, Disodium Oleth-3 Sulfosuccinate, Disodium Oleyl Phosphate, Disodium Oleyl Sulfosuccinate, Disodium Palmitamido PEG-2 Sulfosuccinate, Disodium Palmitoleamido PEG-2 Sulfosuccinate, Disodium PEG-4 Cocamido MIPA-Sulfosuccinate, Disodium PEG-12 Dimethicone Sulfosuccinate, Disodium PEG-8 Palm Glycerides Sulfosuccinate, Disodium PPG-2-Isodeceth-7 Carboxyamphodiacetate, Disodium Ricinoleamido MEA-Sulfosuccinate, Disodium Sitostereth-14 Sulfosuccinate, Disodium Soyamphodiacetate, Disodium Stearamido MEA-Sulfosuccinate, Disodium Steariminodipropionate, Disodium Stearoamphodiacetate, Disodium Stearoyl Glutamate, Disodium Stearyl Sulfosuccinamate, Disodium Stearyl Sulfosuccinate, Disodium 2-Sulfolaurate, Disodium 2-Sulfopalmitate, Disodium Tallamido MEA-Sulfosuccinate, Disodium Tallowamido MEA-Sulfosuccinate, Disodium Tallowamphodiacetate, Disodium Tallowiminodipropionate, Disodium Tallow Sulfosuccinamate, Disodium Tridecylsulfosuccinate, Disodium Undecylenamido MEA-Sulfosuccinate, Disodium Undecylenamido PEG-2 Sulfosuccinate, Disodium Undecylenoyl Glutamate, Disodium Wheat Germamido MEA-Sulfosuccinate, Disodium Wheat Germamido PEG-2 Sulfosuccinate, Disodium Wheatgermamphodiacetate, Di-TEA-Cocamide Diacetate, Di-TEA-Oleamido PEG-2 Sulfosuccinate, Di-TEA-Palmitoyl Aspartate, Ditridecyl Sodium Sulfosuccinate, Dodecylbenzene Sulfonic Acid, Erucamidopropyl Hydroxysultaine, Ethylhexeth-3 Carboxylic Acid, Ethyl PEG-15 Cocamine Sulfate, Glyceryl Capryl Ether, Hexyldecanoic Acid, Hydrogenated Coconut Acid, Hydrogenated Laneth-25, Hydrogenated Menhaden Acid, Hydrogenated Palm Acid, Hydrogenated Palm Kernel Amine Oxide, Hydrogenated Tallow Acid, Hydrogenated Tallowamine Oxide, Hydrogenated Tallow Betaine, Hydrogenated Talloweth-25, Hydrogenated Tallowoyl Glutamic Acid, Hydrolyzed Candida bombicola Extract, Hydroxyceteth-60, Hydroxyethyl Acetomonium PG-Dimethicone, Hydroxyethylbutylamine Laureth Sulfate, Hydroxyethyl Carboxymethyl Cocamidopropylamine, Hydroxyethyl Hydroxypropyl C12-15 Alkoxypropylamine Oxide, Hydroxylauryl/Hydroxymyristyl Betaine, Hydroxystearic Acid, Hydroxysuccinimidyl C10-40 Isoalkyl Acidate, Hydroxysuccinimidyl C21-22 Isoalkyl Acidate, Hydroxysultaines, IPDI/PEG-15 Soyamine Oxide Copolymer, IPDI/PEG-15 Soyethonium Ethosulfate Copolymer, IPDI/PEG-15 Soy Glycinate Copolymer, Isoceteth-30, Isolaureth-4 Phosphate, Isopolyglyceryl-3 Dimethicone, Isopolyglyceryl-3 Dimethiconol, Isopropanolamine Lanolate, Isopropylamine Dodecylbenzenesulfonate, Isostearamidopropylamine Oxide, Isostearamidopropyl Betaine, Isostearamidopropyl Morpholine Oxide, Isosteareth-8, Isosteareth-16, Isosteareth-22, Isosteareth-25, Isosteareth-50, Isostearic Acid, Isostearoyl Hydrolyzed Collagen, Jojoba Oil PEG-150 Esters, Jojoba Wax PEG-80 Esters, Jojoba Wax PEG-120 Esters, Laneth-20, Laneth-25, Laneth-40, Laneth-50, Laneth-60, Laneth-75, Lanolin Acid, Lauramidopropylamine Oxide, Lauramidopropyl Betaine, Lauramidopropyl Hydroxysultaine, Lauramine Oxide, Lauraminopropionic Acid, Laurdimoniumhydroxypropyl Decylglucosides Chloride, Laurdimoniumhydroxypropyl Laurylglucosides Chloride, Laureth-16, Laureth-20, Laureth-21, Laureth-23, Laureth-25, Laureth-30, Laureth-38, Laureth-40, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Laureth-6 Citrate, Laureth-7 Citrate, Laureth-1 Phosphate, Laureth-2 Phosphate, Laureth-3 Phosphate, Laureth-4 Phosphate, Laureth-7 Phosphate, Laureth-8 Phosphate, Laureth-7 Tartrate, Laurie Acid, Laurimino Bispropanediol, Lauriminodipropionic Acid, Lauroamphodipropionic Acid, Lauroyl Beta-Alanine, Lauroyl Collagen Amino Acids, Lauroyl Ethyltrimonium Methosulfate, Lauroyl Hydrolyzed Collagen, Lauroyl Hydrolyzed Elastin, Lauroyl Methyl Glucamide, Lauroyl Sarcosine, Lauroyl Silk Amino Acids, Lauryl Betaine, Lauryl Dimethicone/Polyglycerin-3 Crosspolymer, Lauryldimoniumhydroxypropyl Cocoglucosides Chloride, Lauryl Glucoside, Laurylglucosides Hydroxypropyltrimonium Chloride, Lauryl Glycol Hydroxypropyl Ether, Lauryl Hydroxysultaine, Lauryl Malamide, Lauryl Methylglucamide, Lauryl/Myristyl Glycol Hydroxypropyl Ether, Lauryl/Myristyl Wheat Bran/Straw Glycosides, Lauryl Polyglyceryl-3 Polydimethylsiloxyethyl Dimethicone, Lauryl Pyrrolidone, Lauryl Sultaine, Linoleic Acid, Linolenic Acid, Linseed Acid, Lysine Cocoate, Macadamia Seed Oil Glycereth-8 Esters, Magnesium Coceth Sulfate, Magnesium Coco-Sulfate, Magnesium Isododecylbenzenesulfonate, Magnesium Laureth-11 Carboxylate, Magnesium Laureth Sulfate, Magnesium Laureth-5 Sulfate, Magnesium Laureth-8 Sulfate, Magnesium Laureth-16 Sulfate, Magnesium Laureth-3 Sulfosuccinate, Magnesium Lauryl Hydroxypropyl Sulfonate, Magnesium Lauryl Sulfate, Magnesium Methyl Cocoyl Taurate, Magnesium Myreth Sulfate, Magnesium Oleth Sulfate, Magnesium/TEA-Coco-Sulfate, Manicouagan Clay, MEA-Cocoate, MEA-Laureth-6 Carboxylate, MEA-Laureth Sulfate, MEA-Lauryl Sulfate, MEA PPG-6 Laureth-7 Carboxylate, MEA-PPG-8-Steareth-7 Carboxylate, MEA-Undecylenate, Meroxapol 108, Meroxapol 174, Meroxapol 178, Meroxapol 254, Meroxapol 255, Meroxapol 258, Meroxapol 314, Methoxy PEG-450 Amidoglutaroyl Succinimide, Methoxy PEG-450 Amido Hydroxysuccinimidyl Succinamate, Methoxy PEG-450 Maleimide, Methyl Morpholine Oxide, Milkamidopropyl Amine Oxide, Milkamidopropyl Betaine, Minkamidopropylamine Oxide, Minkamidopropyl Betaine, MIPA C12-15 Pareth Sulfate, MIPA-Dodecylbenzenesulfonate, MIPA-Laureth Sulfate, MIPA-Lauryl Sulfate, Mixed Isopropanolamines Lanolate, Mixed Isopropanolamines Lauryl Sulfate, Mixed Isopropanolamines Myristate, Morpholine Oleate, Morpholine Stearate, Myreth-3 Carboxylic Acid, Myreth-5 Carboxylic Acid, Myristalkonium Chloride, Myristamidopropylamine Oxide, Myristamidopropyl Betaine, Myristamidopropyl Dimethylamine Phosphate, Myristamidopropyl Hydroxysultaine, Myristamidopropyl PG-Dimonium Chloride Phosphate, Myristamine Oxide, Myristaminopropionic Acid, Myristic Acid, Myristoyl Ethyltrimonium Methosulfate, Myristoyl Glutamic Acid, Myristoyl Hydrolyzed Collagen, Myristoyl Sarcosine, Myristyl Betaine, Myristyl/Cetyl Amine Oxide, Myristyldimoniumhydroxypropyl Cocoglucosides Chloride, Myristyl Glucoside, Myristyl Phosphate, Nonoxynol-20, Nonoxynol-23, Nonoxynol-25, Nonoxynol-30, Nonoxynol-35, Nonoxynol-40, Nonoxynol-44, Nonoxynol-50, Nonoxynol-100, Nonoxynol-120, Nonoxynol-5 Carboxylic Acid, Nonoxynol-8 Carboxylic Acid, Nonoxynol-10 Carboxylic Acid, Nonoxynol-3 Phosphate, Nonoxynol-4 Phosphate, Nonoxynol-6 Phosphate, Nonoxynol-9 Phosphate, Nonoxynol-10 Phosphate, Nonyl Nonoxynol-30, Nonyl Nonoxynol-49, Nonyl Nonoxynol-100, Nonyl Nonoxynol-150, Nonyl Nonoxynol-7 Phosphate, Nonyl Nonoxynol-8 Phosphate, Nonyl Nonoxynol-9 Phosphate, Nonyl Nonoxynol-10 Phosphate, Nonyl Nonoxynol-11 Phosphate, Nonyl Nonoxynol-15 Phosphate, Nonyl Nonoxynol-24 Phosphate, Oatamidopropyl Betaine, Octoxynol-16, Octoxynol-25, Octoxynol-30, Octoxynol-33, Octoxynol-40, Octoxynol-70, Octoxynol-20 Carboxylic Acid, Octyldodeceth-20, Octyldodeceth-25, Octyldodeceth-30, Oleamidopropylamine Oxide, Oleamidopropyl Betaine, Oleamidopropyl Hydroxysultaine, Oleamine Oxide, Oleic Acid, Oleoyl Hydrolyzed Collagen, Oleoyl Sarcosine, Oleth-20, Oleth-23, Oleth-24, Oleth-25, Oleth-30, Oleth-35, Oleth-40, Oleth-44, Oleth-50, Oleth-3 Carboxylic Acid, Oleth-6 Carboxylic Acid, Oleth-10 Carboxylic Acid, Oleyl Betaine, Olivamidopropylamine Oxide, Olivamidopropyl Betaine, Olive Acid, Olivoyl Hydrolyzed Wheat Protein, Ophiopogon Extract Stearate, Ozonized Oleth-10, Ozonized PEG-10 Oleate, Ozonized PEG-14 Oleate, Ozonized Polysorbate 80, Palm Acid, Palmamidopropyl Betaine, Palmeth-2 Phosphate, Palmitamidopropylamine Oxide, Palmitamidopropyl Betaine, Palmitamine Oxide, Palmitic Acid, Palmitoyl Collagen Amino Acids, Palmitoyl Glycine, Palmitoyl Hydrolyzed Collagen, Palmitoyl Hydrolyzed Milk Protein, Palmitoyl Hydrolyzed Wheat Protein, Palmitoyl Keratin Amino Acids, Palmitoyl Oligopeptide, Palmitoyl Silk Amino Acids, Palm Kernel Acid, Palm Kernelamidopropyl Betaine, Peach Kernel Oil Glycereth-8 Esters, Peanut Acid, PEG-10 Castor Oil, PEG-40 Castor Oil, PEG-44 Castor Oil, PEG-50 Castor Oil, PEG-54 Castor Oil, PEG-55 Castor Oil, PEG-60 Castor Oil, PEG-80 Castor Oil, PEG-100 Castor Oil, PEG-200 Castor Oil, PEG-11 Cocamide, PEG-6 Cocamide Phosphate, PEG-4 Cocamine, PEG-8 Cocamine, PEG-12 Cocamine, PEG-150 Dibehenate, PEG-90 Diisostearate, PEG-75 Dilaurate, PEG-150 Dilaurate, PEG-75 Dioleate, PEG-150 Dioleate, PEG-75 Distearate, PEG-120 Distearate, PEG-150 Distearate, PEG-175 Distearate, PEG-190 Distearate, PEG-250 Distearate, PEG-30 Glyceryl Cocoate, PEG-40 Glyceryl Cocoate, PEG-78 Glyceryl Cocoate, PEG-80 Glyceryl Cocoate, PEG-30 Glyceryl Isostearate, PEG-40 Glyceryl Isostearate, PEG-50 Glyceryl Isostearate, PEG-60 Glyceryl Isostearate, PEG-90 Glyceryl Isostearate, PEG-23 Glyceryl Laurate, PEG-30 Glyceryl Laurate, PEG-25 Glyceryl Oleate, PEG-30 Glyceryl Oleate, PEG-30 Glyceryl Soyate, PEG-25 Glyceryl Stearate, PEG-30 Glyceryl Stearate, PEG-40 Glyceryl Stearate, PEG-120 Glyceryl Stearate, PEG-200 Glyceryl Stearate, PEG-28 Glyceryl Tallowate, PEG-80 Glyceryl Tallowate, PEG-82 Glyceryl Tallowate, PEG-130 Glyceryl Tallowate, PEG-200 Glyceryl Tallowate, PEG-45 Hydrogenated Castor Oil, PEG-50 Hydrogenated Castor Oil, PEG-54 Hydrogenated Castor Oil, PEG-55 Hydrogenated Castor Oil, PEG-60 Hydrogenated Castor Oil, PEG-80 Hydrogenated Castor Oil, PEG-100 Hydrogenated Castor Oil, PEG-200 Hydrogenated Castor Oil, PEG-30 Hydrogenated Lanolin, PEG-70 Hydrogenated Lanolin, PEG-50 Hydrogenated Palmamide, PEG-2 Isostearate, PEG-3 Isostearate, PEG-4 Isostearate, PEG-6 Isostearate, PEG-8 Isostearate, PEG-10 Isostearate, PEG-12 Isostearate, PEG-20 Isostearate, PEG-30 Isostearate, PEG-40 Isostearate, PEG-26 Jojoba Acid, PEG-40 Jojoba Acid, PEG-15 Jojoba Alcohol, PEG-26 Jojoba Alcohol, PEG-40 Jojoba Alcohol, PEG-35 Lanolin, PEG-40 Lanolin, PEG-50 Lanolin, PEG-55 Lanolin, PEG-60 Lanolin, PEG-70 Lanolin, PEG-75 Lanolin, PEG-85 Lanolin, PEG-100 Lanolin, PEG-150 Lanolin, PEG-75 Lanolin Oil, PEG-2 Lauramide, PEG-3 Lauramine Oxide, PEG-20 Laurate, PEG-32 Laurate, PEG-75 Laurate, PEG-150 Laurate, PEG-70 Mango Glycerides, PEG-20 Mannitan Laurate, PEG-8 Methyl Ether Dimethicone, PEG-120 Methyl Glucose Dioleate, PEG-80 Methyl Glucose Laurate, PEG-120 Methyl Glucose Trioleate, PEG-4 Montanate, PEG-30 Oleamine, PEG-20 Oleate, PEG-23 Oleate, PEG-32 Oleate, PEG-36 Oleate, PEG-75 Oleate, PEG-150 Oleate, PEG-20 Palmitate, PEG-150 Polyglyceryl-2 Tristearate, PEG/PPG-28/21 Acetate Dimethicone, PEG/PPG-24/18 Butyl Ether Dimethicone, PEG/PPG-3/17 Copolymer, PEG/PPG-5/35 Copolymer, PEG/PPG-8/55 Copolymer, PEG/PPG-10/30 Copolymer, PEG/PPG-10/65 Copolymer, PEG/PPG-12/35 Copolymer, PEG/PPG-16/17 Copolymer, PEG/PPG-20/9 Copolymer, PEG/PPG-20/20 Copolymer, PEG/PPG-20/60 Copolymer, PEG/PPG-20/65 Copolymer, PEG/PPG-22/25 Copolymer, PEG/PPG-28/30 Copolymer, PEG/PPG-30-35 Copolymer, PEG/PPG-30/55 Copolymer, PEG/PPG-35/40 Copolymer, PEG/PPG-50/40 Copolymer, PEG/PPG-150/35 Copolymer, PEG/PPG-160/30 Copolymer, PEG/PPG-190/60 Copolymer, PEG/PPG-200/40 Copolymer, PEG/PPG-300/55 Copolymer, PEG/PPG-20/22 Methyl Ether Dimethicone, PEG-26-PPG-30 Phosphate, PEG/PPG-4/2 Propylheptyl Ether, PEG/PPG-6/2 Propylheptyl Ether, PEG-7/PPG-2 Propylheptyl Ether, PEG/PPG-8/2 Propylheptyl Ether, PEG/PPG-10/2 Propylheptyl Ether, PEG/PPG-14/2 Propylheptyl Ether, PEG/PPG-40/2 Propylheptyl Ether, PEG/PPG-10/2 Ricinoleate, PEG/PPG-32/3 Ricinoleate, PEG-55 Propylene Glycol Oleate, PEG-25 Propylene Glycol Stearate, PEG-75 Propylene Glycol Stearate, PEG-120 Propylene Glycol Stearate, PEG-5 Rapeseed Sterol, PEG-10 Rapeseed Sterol, PEG-40 Ricinoleamide, PEG-75 Shea Butter Glycerides, PEG-75 Shorea Butter Glycerides, PEG-20 Sorbitan Cocoate, PEG-20 Sorbitan Isostearate, PEG-40 Sorbitan Lanolate, PEG-75 Sorbitan Lanolate, PEG-10 Sorbitan Laurate, PEG-40 Sorbitan Laurate, PEG-44 Sorbitan Laurate, PEG-75 Sorbitan Laurate, PEG-80 Sorbitan Laurate, PEG-20 Sorbitan Oleate, PEG-80 Sorbitan Palmitate, PEG-40 Sorbitan Stearate, PEG-60 Sorbitan Stearate, PEG-160 Sorbitan Triisostearate, PEG-40 Soy Sterol, PEG-2 Stearamide Carboxylic Acid, PEG-9 Stearamide Carboxylic Acid, PEG-20 Stearate, PEG-23 Stearate, PEG-25 Stearate, PEG-30 Stearate, PEG-32 Stearate, PEG-35 Stearate, PEG-36 Stearate, PEG-40 Stearate, PEG-45 Stearate, PEG-50 Stearate, PEG-55 Stearate, PEG-75 Stearate, PEG-90 Stearate, PEG-100 Stearate, PEG-120 Stearate, PEG-150 Stearate, PEG-45 Stearate Phosphate, PEG-20 Tallate, PEG-50 Tallow Amide, PEG-2 Tallowamide DEA, PEG-20 Tallowate, PEG-66 Trihydroxystearin, PEG-200 Trihydroxystearin, PEG-60 Tsubakiate Glycerides, Pelargonic Acid, Pentadoxynol-200, Pheneth-6 Phosphate, Poloxamer 105, Poloxamer 108, Poloxamer 182, Poloxamer 183, Poloxamer 184, Poloxamer 188, Poloxamer 217, Poloxamer 234, Poloxamer 235, Poloxamer 237, Poloxamer 238, Poloxamer 288, Poloxamer 334, Poloxamer 335, Poloxamer 338, Poloxamine 908, Poloxamine 1508, Polydimethylsiloxy PEG/PPG-24/19 Butyl Ether Silsesquioxane, Polydimethylsiloxy PPG-13 Butyl Ether Silsesquioxane, Polyglyceryl-6 Caprate, Polyglyceryl-10 Dilaurate, Polyglyceryl-20 Heptacaprylate, Polyglyceryl-20 Hexacaprylate, Polyglyceryl-2 Lauryl Ether, Polyglyceryl-10 Lauryl Ether, Polyglyceryl-20 Octaisononanoate, Polyglyceryl-6 Pentacaprylate, Polyglyceryl-10 Pentacaprylate, Polyglyceryl-3 Polydimethylsiloxyethyl Dimethicone, Polyglyceryl-6 Tetracaprylate, Polyglyceryl-10 Tetralaurate, Polyglyceryl-6 Tricaprylate, Polyglyceryl-10 Trilaurate, Polyquaternium-77, Polyquaternium-78, Polyquaternium-79, Polyquaternium-80, Polyquaternium-81, Polyquaternium-82, Pomaderris Kumerahou Flower/Leaf Extract, Poria Cocos Extract, Potassium Abietoyl Hydrolyzed Collagen, Potassium Babassuate, Potassium Behenate, Potassium C9-15 Alkyl Phosphate, Potassium C11-15 Alkyl Phosphate, Potassium C12-13 Alkyl Phosphate, Potassium C12-14 Alkyl Phosphate, Potassium Caprate, Potassium Capryloyl Glutamate, Potassium Capryloyl Hydrolyzed Rice Protein, Potassium Castorate, Potassium Cocoate, Potassium Cocoyl Glutamate, Potassium Cocoyl Glycinate, Potassium Cocoyl Hydrolyzed Casein, Potassium Cocoyl Hydrolyzed Collagen, Potassium Cocoyl Hydrolyzed Corn Protein, Potassium Cocoyl Hydrolyzed Keratin, Potassium Cocoyl Hydrolyzed Oat Protein, Potassium Cocoyl Hydrolyzed Potato Protein, Potassium Cocoyl Hydrolyzed Rice Bran Protein, Potassium Cocoyl Hydrolyzed Rice Protein, Potassium Cocoyl Hydrolyzed Silk, Potassium Cocoyl Hydrolyzed Soy Protein, Potassium Cocoyl Hydrolyzed Wheat Protein, Potassium Cocoyl Hydrolyzed Yeast Protein, Potassium Cocoyl PCA, Potassium Cocoyl Sarcosinate, Potassium Cocoyl Taurate, Potassium Cornate, Potassium Cyclocarboxypropyloleate, Potassium Dihydroxyethyl Cocamine Oxide Phosphate, Potassium Dimethicone PEG-7 Phosphate, Potassium Dodecylbenzenesulfonate, Potassium Hempseedate, Potassium Hydrogenated Cocoate, Potassium Hydrogenated Palmate, Potassium Hydrogenated Tallowate, Potassium Hydroxystearate, Potassium Isostearate, Potassium Lanolate, Potassium Laurate, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Laureth Phosphate, Potassium Lauroyl Collagen Amino Acids, Potassium Lauroyl Glutamate, Potassium Lauroyl Hydrolyzed Collagen, Potassium Lauroyl Hydrolyzed Pea Protein, Potassium Lauroyl Hydrolyzed Soy Protein, Potassium Lauroyl PCA, Potassium Lauroyl Pea Amino Acids, Potassium Lauroyl Sarcosinate, Potassium Lauroyl Silk Amino Acids, Potassium Lauroyl Wheat Amino Acids, Potassium Lauryl Phosphate, Potassium Lauryl Sulfate, Potassium Linoleate, Potassium Metaphosphate, Potassium Methyl Cocoyl Taurate, Potassium Myristate, Potassium Myristoyl Glutamate, Potassium Myristoyl Hydrolyzed Collagen, Potassium Octoxynol-12 Phosphate, Potassium Oleate, Potassium Oleoyl Hydrolyzed Collagen, Potassium Olivate, Potassium Olivoyl Hydrolyzed Oat Protein, Potassium Olivoyl Hydrolyzed Wheat Protein, Potassium Olivoyl/Lauroyl Wheat Amino Acids, Potassium Olivoyl PCA, Potassium Palmate, Potassium Palmitate, Potassium Palmitoyl Hydrolyzed Corn Protein, Potassium Palmitoyl Hydrolyzed Oat Protein, Potassium Palmitoyl Hydrolyzed Rice Protein, Potassium Palmitoyl Hydrolyzed Sweet Almond Protein, Potassium Palmitoyl Hydrolyzed Wheat Protein, Potassium Palm Kernelate, Potassium Peanutate, Potassium Rapeseedate, Potassium Ricinoleate, Potassium Safflowerate, Potassium Soyate, Potassium Stearate, Potassium Stearoyl Hydrolyzed Collagen, Potassium Tallate, Potassium Tallowate, Potassium Taurate, Potassium Taurine Laurate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, Potassium Trideceth-6 Phosphate, Potassium Trideceth-7 Phosphate, Potassium Tsubakiate, Potassium Undecylenate, Potassium Undecylenoyl Hydrolyzed Collagen, Potassium Undecylenoyl Hydrolyzed Rice Protein, PPG-30-Buteth-30, PPG-36-Buteth-36, PPG-38-Buteth-37, PPG-30-Capryleth-4 Phosphate, PPG-10 Cetyl Ether Phosphate, PPG-2 C9-11 Pareth-8, PPG-1-Deceth-5, PPG-3-Deceth-2 Carboxylic Acid, PPG-30 Ethylhexeth-4 Phosphate, PPG-20-Glycereth-30, PPG-2 Hydroxyethyl Coco/Isostearamide, PPG-2-Isodeceth-8, PPG-2-Isodeceth-10, PPG-2-Isodeceth-18, PPG-2-Isodeceth-25, PPG-4-Isodeceth-10, Propyltrimonium Hydrolyzed Collagen, Quaternium-24, Quaternium-52, Quaternium-87, Rapeseed Acid, Rice Bran Acid, Rice Oil Glycereth-8 Esters, Ricinoleamidopropyl Betaine, Ricinoleic Acid, Ricinoleth-40, Safflower Acid, Sapindus oahuensis Fruit Extract, Saponaria officinalis Root Powder, Saponins, Sekken-K, Sekken-Na/K, Sekken Soji, Sekken Soji-K, Sesame Oil Glycereth-8 Esters, Sesamidopropylamine Oxide, Sesamidopropyl Betaine, Shea Butteramidopropyl Betaine, Shea Butter Glycereth-8 Esters, Sodium Arachidate, Sodium Arganampohoacetate, Sodium Astrocaryum Murumuruate, Sodium Avocadoate, Sodium Babassuamphoacetate, Sodium Babassuate, Sodium Babassu Sulfate, Sodium Behenate, Sodium Bisglycol Ricinosulfosuccinate, Sodium Bis-Hydroxyethylglycinate Coco-Glucosides Crosspolymer, Sodium Bis-Hydroxyethylglycinate Lauryl-Glucosides Crosspolymer, Sodium Borageamidopropyl PG-Dimonium Chloride Phosphate, Sodium Butoxynol-12 Sulfate, Sodium Butylglucosides Hydroxypropyl Phosphate, Sodium C13-17 Alkane Sulfonate, Sodium C14-18 Alkane Sulfonate, Sodium C12-15 Alkoxypropyl Iminodipropionate, Sodium C10-16 Alkyl Sulfate, Sodium C11-15 Alkyl Sulfate, Sodium C12-13 Alkyl Sulfate, Sodium C12-15 Alkyl Sulfate, Sodium C12-18 Alkyl Sulfate, Sodium C16-20 Alkyl Sulfate, Sodium C9-22 Alkyl Sec Sulfonate, Sodium C14-17 Alkyl Sec Sulfonate, Sodium Caprate, Sodium Caproamphoacetate, Sodium Caproamphohydroxypropylsulfonate, Sodium Caproamphopropionate, Sodium Caproyl Methyltaurate, Sodium Caprylate, Sodium Capryleth-2 Carboxylate, Sodium Capryleth-9 Carboxylate, Sodium Capryloamphoacetate, Sodium Capryloamphohydroxypropylsulfonate, Sodium Capryloamphopropionate, Sodium Capryloyl Glutamate, Sodium Capryloyl Hydrolyzed Wheat Protein, Sodium Caprylyl PG-Sulfonate, Sodium Caprylyl Sulfonate, Sodium Castorate, Sodium Ceteareth-13 Carboxylate, Sodium Cetearyl Sulfate, Sodium Ceteth-13 Carboxylate, Sodium Cetyl Sulfate, Sodium Cocamidopropyl PG-Dimonium Chloride Phosphate, Sodium Cocaminopropionate, Sodium Coceth Sulfate, Sodium Coceth-30 Sulfate, Sodium Cocoabutteramphoacetate, Sodium Cocoa Butterate, Sodium Cocoamphoacetate, Sodium Cocoamphohydroxypropylsulfonate, Sodium Cocoamphopropionate, Sodium Cocoate, Sodium Coco/Babassu/Andiroba Sulfate, Sodium Coco/Babassu Sulfate, Hydroxypropyl Sodium Cocoglucosides Phosphate, Sodium Cocoglucosides Hydroxypropylsulfonate, Sodium Coco-Glucoside Tartrate, Sodium Cocoglyceryl Ether Sulfonate, Sodium Coco/Hydrogenated Tallow Sulfate, Sodium Cocoiminodiacetate, Sodium Cocomonoglyceride Sulfate, Sodium Cocomonoglyceride Sulfonate, Sodium Coco PG-Dimonium Chloride Phosphate, Sodium Coco-Sulfate, Sodium Coco Sulfoacetate, Sodium Cocoyl Alaninate, Sodium Cocoyl Amino Acids, Sodium Cocoyl Collagen Amino Acids, Sodium Cocoyl Glutamate, Sodium Cocoyl Glutaminate, Sodium Cocoyl Glycinate, Sodium Cocoyl/Hydrogenated Tallow Glutamate, Sodium Cocoyl Hydrolyzed Collagen, Sodium Cocoyl Hydrolyzed Keratin, Sodium Cocoyl Hydrolyzed Rice Protein, Sodium Cocoyl Hydrolyzed Silk, Sodium Cocoyl Hydrolyzed Soy Protein, Sodium Cocoyl Hydrolyzed Sweet Almond Protein, Sodium Cocoyl Hydrolyzed Wheat Protein, Sodium Cocoyl Hydrolyzed Wheat Protein Glutamate, Sodium Cocoyl Isethionate, Sodium Cocoyl Methylaminopropionate, Sodium Cocoyl Oat Amino Acids, Sodium Cocoyl/Palmoyl/Sunfloweroyl Glutamate, Sodium Cocoyl Proline, Sodium Cocoyl Sarcosinate, Sodium Cocoyl Taurate, Sodium Cocoyl Threoninate, Sodium Cocoyl Wheat Amino Acids, Sodium C12-14 Olefin Sulfonate, Sodium C14-16 Olefin Sulfonate, Sodium C14-18 Olefin Sulfonate, Sodium C16-18 Olefin Sulfonate, Sodium Cornamphopropionate, Sodium Cottonseedamphoacetate, Sodium C13-15 Pareth-8 Butyl Phosphate, Sodium C9-11 Pareth-6 Carboxylate, Sodium C11-15 Pareth-7 Carboxylate, Sodium C12-13 Pareth-5 Carboxylate, Sodium C12-13 Pareth-8 Carboxylate, Sodium C12-13 Pareth-12 Carboxylate, Sodium C12-15 Pareth-6 Carboxylate, Sodium C12-15 Pareth-7 Carboxylate, Sodium C12-15 Pareth-8 Carboxylate, Sodium C14-15 Pareth-8 Carboxylate, Sodium C12-14 Sec-Pareth-8 Carboxylate, Sodium C14-15 Pareth-PG Sulfonate, Sodium C12-13 Pareth-2 Phosphate, Sodium C13-15 Pareth-8 Phosphate, Sodium C9-15 Pareth-3 Sulfate, Sodium C10-15 Pareth Sulfate, Sodium C10-16 Pareth-2 Sulfate, Sodium C12-13 Pareth Sulfate, Sodium C12-15 Pareth Sulfate, Sodium C12-15 Pareth-3 Sulfate, Sodium C13-15 Pareth-3 Sulfate, Sodium C12-14 Sec-Pareth-3 Sulfate, Sodium C12-15 Pareth-3 Sulfonate, Sodium C12-15 Pareth-7 Sulfonate, Sodium C12-15 Pareth-15 Sulfonate, Sodium Deceth-2 Carboxylate, Sodium Deceth Sulfate, Sodium Decylbenzenesulfonate, Sodium Decylglucosides Hydroxypropyl Phosphate, Sodium Decylglucosides Hydroxypropylsulfonate, Sodium Dilaureth-7 Citrate, Sodium Dilaureth-10 Phosphate, Sodium Dilinoleamidopropyl PG-Dimonium Chloride Phosphate, Sodium Dilinoleate, Sodium Dioleth-8 Phosphate, Sodium Dodecylbenzenesulfonate, Sodium Ethyl 2-Sulfolaurate, Sodium Glyceryl Oleate Phosphate, Sodium Grapeseedamidopropyl PG-Dimonium Chloride Phosphate, Sodium Grapeseedamphoacetate, Sodium Grapeseedate, Sodium Hempseedamphoacetate, Sodium Hexeth-4 Carboxylate, Sodium Hydrogenated Cocoate, Sodium Hydrogenated Cocoyl Methyl Isethionate, Sodium Hydrogenated Palmate, Sodium Hydrogenated Tallowate, Sodium Hydrogenated Tallowoyl Glutamate, Sodium Hydroxylauryldimonium Ethyl Phosphate, Sodium Hydroxypropyl Palm Kernelate Sulfonate, Sodium Hydroxypropylphosphate Decylglucoside Crosspolymer, Sodium Hydroxypropylphosphate Laurylglucoside Crosspolymer, Sodium Hydroxypropylsulfonate Cocoglucoside Crosspolymer, Sodium Hydroxypropylsulfonate Decylglucoside Crosspolymer, Sodium Hydroxypropylsulfonate Laurylglucoside Crosspolymer, Sodium Hydroxystearate, Sodium Isostearate, Sodium Isosteareth-6 Carboxylate, Sodium Isosteareth-11 Carboxylate, Sodium Isostearoamphoacetate, Sodium Isostearoamphopropionate, Sodium N-Isostearoyl Methyltaurate, Sodium Laneth Sulfate, Sodium Lanolate, Sodium Lardate, Sodium Lauramido Diacetate, Sodium Lauraminopropionate, Sodium Laurate, Sodium Laureth-3 Carboxylate, Sodium Laureth-4 Carboxylate, Sodium Laureth-5 Carboxylate, Sodium Laureth-6 Carboxylate, Sodium Laureth-8 Carboxylate, Sodium Laureth-11 Carboxylate, Sodium Laureth-12 Carboxylate, Sodium Laureth-13 Carboxylate, Sodium Laureth-14 Carboxylate, Sodium Laureth-16 Carboxylate, Sodium Laureth-17 Carboxylate, Sodium Laureth Sulfate, Sodium Laureth-5 Sulfate, Sodium Laureth-7 Sulfate, Sodium Laureth-8 Sulfate, Sodium Laureth-12 Sulfate, Sodium Laureth-40 Sulfate, Sodium Laureth-7 Tartrate, Sodium Lauriminodipropionate, Sodium L Lauroamphoacetate, Sodium Lauroamphohydroxypropylsulfonate, Sodium Lauroampho PG-Acetate Phosphate, Sodium Lauroamphopropionate, Sodium Lauroyl Aspartate, Sodium Lauroyl Collagen Amino Acids, Sodium Lauroyl Glycine Propionate, Sodium Lauroyl Hydrolyzed Collagen, Sodium Lauroyl Hydrolyzed Silk, Sodium Lauroyl Hydroxypropyl Sulfonate, Sodium Lauroyl Isethionate, Sodium Lauroyl Methylaminopropionate, Sodium Lauroyl Methyl Isethionate, Sodium Lauroyl Millet Amino Acids, Sodium Lauroyl/Myristoyl Aspartate, Sodium Lauroyl Oat Amino Acids, Sodium Lauroyl Sarcosinate, Sodium Lauroyl Silk Amino Acids, Sodium Lauroyl Taurate, Sodium Lauroyl Wheat Amino Acids, Sodium Lauryl Diethylenediaminoglycinate, Sodium Lauryl Glucose Carboxylate, Sodium Laurylglucosides Hydroxypropyl Phosphate, Sodium Laurylglucosides Hydroxypropylsulfonate, Sodium Lauryl Glycol Carboxylate, Sodium Lauryl Hydroxyacetamide Sulfate, Sodium Lauryl Phosphate, Sodium Lauryl Sulfate, Sodium Lauryl Sulfoacetate, Sodium Linoleate, Sodium Macadamiaseedate, Sodium Mangoamphoacetate, Sodium Mangoseedate, Sodium/MEA Laureth-2 Sulfosuccinate, Sodium Methoxy PPG-2 Acetate, Sodium Methyl Cocoyl Taurate, Sodium Methyl Lauroyl Taurate, Sodium Methyl Myristoyl Taurate, Sodium Methyl Oleoyl Taurate, Sodium Methyl Palmitoyl Taurate, Sodium Methyl Stearoyl Taurate, Sodium Methyl 2-Sulfolaurate, Sodium Methyl 2-Sulfopalmitate, Sodium Methyltaurate Isopalmitamide, Sodium Methyltaurine Cocoyl Methyltaurate, Sodium Myreth Sulfate, Sodium Myristate, Sodium Myristoamphoacetate, Sodium Myristoyl Glutamate, Sodium Myristoyl Hydrolyzed Collagen, Sodium Myristoyl Isethionate, Sodium Myristoyl Sarcosinate, Sodium Myristyl Sulfate, Sodium Nonoxynol-6 Phosphate, Sodium Nonoxynol-9 Phosphate, Sodium Nonoxynol-1 Sulfate, Sodium Nonoxynol-3 Sulfate, Sodium Nonoxynol-4 Sulfate, Sodium Nonoxynol-6 Sulfate, Sodium Nonoxynol-8 Sulfate, Sodium Nonoxynol-10 Sulfate, Sodium Nonoxynol-25 Sulfate, Sodium Octoxynol-2 Ethane Sulfonate, Sodium Octoxynol-2 Sulfate, Sodium Octoxynol-6 Sulfate, Sodium Octoxynol-9 Sulfate, Sodium Oleate, Sodium Oleoamphoacetate, Sodium Oleoamphohydroxypropylsulfonate, Sodium Oleoamphopropionate, Sodium Oleoyl Hydrolyzed Collagen, Sodium Oleoyl Isethionate, Sodium Oleth Sulfate, Sodium Oleyl Methyl Isethionate, Sodium Oleyl Sulfate, Sodium Olivamphoacetate, Sodium Olivate, Sodium Olivoyl Glutamate, Sodium Palmamphoacetate, Sodium Palmate, Sodium Palm Glyceride Sulfonate, Sodium Palmitate, Sodium Palmitoyl Hydrolyzed Collagen, Sodium Palmitoyl Hydrolyzed Wheat Protein, Sodium Palmitoyl Sarcosinate, Sodium Palm Kernelate, Sodium Palm Kerneloyl Isethionate, Sodium Palmoyl Glutamate, Sodium Passiflora edulis Seedate, Sodium Peanutamphoacetate, Sodium Peanutate, Sodium PEG-6 Cocamide Carboxylate, Sodium PEG-8 Cocamide Carboxylate, Sodium PEG-4 Cocamide Sulfate, Sodium PEG-3 Lauramide Carboxylate, Sodium PEG-4 Lauramide Carboxylate, Sodium PEG-8 Palm Glycerides Carboxylate, Sodium Pentaerythrityl Hydroxypropyl Iminodiacetate Dendrimer, Sodium Propoxy PPG-2 Acetate, Sodium Rapeseedate, Sodium Ricebranamphoacetate, Sodium Ricinoleate, Sodium Ricinoleoamphoacetate, Sodium Rose Hipsamphoacetate, Sodium Rosinate, Sodium Safflowerate, Sodium Saffloweroyl Hydrolyzed Soy Protein, Sodium Sesameseedate, Sodium Sesamphoacetate, Sodium Sheabutteramphoacetate, Sodium Soyate, Sodium Soy Hydrolyzed Collagen, Sodium Stearate, Sodium Stearoamphoacetate, Sodium Stearoamphohydroxypropylsulfonate, Sodium Stearoamphopropionate, Sodium Stearoyl Casein, Sodium Stearoyl Glutamate, Sodium Stearoyl Hyaluronate, Sodium Stearoyl Hydrolyzed Collagen, Sodium Stearoyl Hydrolyzed Corn Protein, Sodium Stearoyl Hydrolyzed Silk, Sodium Stearoyl Hydrolyzed Soy Protein, Sodium Stearoyl Hydrolyzed Wheat Protein, Sodium Stearoyl Lactalbumin, Sodium Stearoyl Methyl Isethionate, Sodium Stearoyl Oat Protein, Sodium Stearoyl Pea Protein, Sodium Stearoyl Soy Protein, Sodium Stearyl Dimethyl Glycine, Sodium Stearyl Sulfate, Sodium Sunflowerseedamphoacetate, Sodium Surfactin, Sodium Sweetalmondamphoacetate, Sodium Sweet Almondate, Sodium Tallamphopropionate, Sodium Tallate, Sodium Tallowamphoacetate, Sodium Tallowate, Sodium Tallow Sulfate, Sodium Tamanuseedate, Sodium Taurate, Sodium Taurine Cocoyl Methyltaurate, Sodium Taurine Laurate, Sodium/TEA-Lauroyl Collagen Amino Acids, Sodium/TEA-Lauroyl Hydrolyzed Collagen, Sodium/TEA-Lauroyl Hydrolyzed Keratin, Sodium/TEA-Lauroyl Keratin Amino Acids, Sodium/TEA-Undecylenoyl Collagen Amino Acids, Sodium/TEA-Undecylenoyl Hydrolyzed Collagen, Sodium/TEA-Undecylenoyl Hydrolyzed Corn Protein, Sodium/TEA-Undecylenoyl Hydrolyzed Soy Protein, Sodium/TEA-Undecylenoyl Hydrolyzed Wheat Protein, Sodium Theobroma Grandiflorum Seedate, Sodium Trideceth-3 Carboxylate, Sodium Trideceth-4 Carboxylate, Sodium Trideceth-6 Carboxylate, Sodium Trideceth-7 Carboxylate, Sodium Trideceth-8 Carboxylate, Sodium Trideceth-12 Carboxylate, Sodium Trideceth-15 Carboxylate, Sodium Trideceth-19 Carboxylate, Sodium Trideceth Sulfate, Sodium Tridecylbenzenesulfonate, Sodium Tridecyl Sulfate, Sodium Trimethylolpropane Hydroxypropyl Iminodiacetate Dendrimer, Sodium Undeceth-5 Carboxylate, Sodium Undecylenate, Sodium Undecylenoamphoacetate, Sodium Undecylenoamphopropionate, Sodium Undecylenoyl Glutamate, Sodium Wheat Germamphoacetate, Sorbeth-160 Tristearate, Soy Acid, Soyamidopropylamine Oxide, Soyamidopropyl Betaine, Soybean Oil Glycereth-8 Esters, Stearamidopropylamine Oxide, Stearamidopropyl Betaine, Stearamine Oxide, Steareth-15, Steareth-16, Steareth-20, Steareth-21, Steareth-25, Steareth-27, Steareth-30, Steareth-40, Steareth-50, Steareth-80, Steareth-100, Steareth-2 Phosphate, Steareth-3 Phosphate, Stearic Acid, Stearoxypropyltrimonium Chloride, Stearoyl Glutamic Acid, Stearoyl Sarcosine, Stearyl Betaine, Stearyldimoniumhydroxypropyl Butylglucosides Chloride, Decylglucosides Chloride, Stearyldimoniumhydroxypropyl Stearyldimoniumhydroxypropyl Laurylglucosides Chloride, Sulfated Castor Oil, Sulfated Coconut Oil, Sulfated Glyceryl Oleate, Sulfated Olive Oil, Sulfated Peanut Oil, Sunfloweramide MEA, Sunflower Seed Acid, Sunflowerseedamidopropyl Hydroxyethyldimonium Chloride, Sunflower Seed Oil Glycereth-8 Esters, Tall Oil Acid, Tallow Acid, Tallowamidopropylamine Oxide, Tallowamidopropyl Betaine, Tallowamidopropyl Hydroxysultaine, Tallowamine Oxide, Tallow Betaine, Tallow Dihydroxyethyl Betaine, Tallowoyl Ethyl Glucoside, TEA-Abietoyl Hydrolyzed Collagen, TEA-C12-14 Alkyl Phosphate, TEA-C10-15 Alkyl Sulfate, TEA-C11-15 Alkyl Sulfate, TEA-C12-13 Alkyl Sulfate, TEA-C12-14 Alkyl Sulfate, TEA-C12-15 Alkyl Sulfate, TEA C14-17 Alkyl Sec Sulfonate, TEA-Canolate, TEA-Cocamide Diacetate, TEA-Cocoate, TEA-Coco-Sulfate, TEA-Cocoyl Alaninate, TEA-Cocoyl Glutamate, TEA-Cocoyl Glutaminate, TEA-Cocoyl Glycinate, TEA-Cocoyl Hydrolyzed Collagen, TEA-Cocoyl Hydrolyzed Soy Protein, TEA-Cocoyl Sarcosinate, TEA-Dimethicone PEG-7 Phosphate, TEA-Dodecylbenzenesulfonate, TEA-Hydrogenated Cocoate, TEA-Hydrogenated Tallowoyl Glutamate, TEA-Isostearate, TEA-Isostearoyl Hydrolyzed Collagen, TEA-Lauraminopropionate, TEA-Laurate, TEA-Laurate/Myristate, TEA-Laureth Sulfate, TEA-Lauroyl Collagen Amino Acids, TEA-Lauroyl Glutamate, TEA-Lauroyl Hydrolyzed Collagen, TEA-Lauroyl Keratin Amino Acids, TEA-Lauroyl Methylaminopropionate, TEA-Lauroyl/Myristoyl Aspartate, TEA-Lauroyl Sarcosinate, TEA-Lauryl Phosphate, TEA-Lauryl Sulfate, TEA-Myristaminopropionate, TEA-Myristate, TEA-Myristoyl Hydrolyzed Collagen, TEA-Oleate, TEA-Oleoyl Hydrolyzed Collagen, TEA-Oleoyl Sarcosinate, TEA-Oleyl Sulfate, TEA-Palmitate, TEA-Palm Kernel Sarcosinate, TEA-PEG-3 Cocamide Sulfate, TEA-Rosinate, TEA-Stearate, TEA-Tallate, TEA-T ridecylbenzenesulfonate, TEA-Undecylenate, TEA-Undecylenoyl Hydrolyzed Collagen, Tetramethyl Decynediol, Tetrasodium Dicarboxyethyl Stearyl Sulfosuccinamate, TIPA-Laureth Sulfate, TIPA-Lauryl Sulfate, TIPA-Myristate, TIPA-Stearate, Tocopheryl Phosphate, Trehalose Undecylenoate, TM-C12-15 Pareth-2 Phosphate, TM-C12-15 Pareth-6 Phosphate, TM-C12-15 Pareth-8 Phosphate, TM-C12-15 Pareth-10 Phosphate, Trideceth-20, Trideceth-50, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid, Trideceth-10 Phosphate, Tridecylbenzenesulfonic Acid, Trilaureth-9 Citrate, Trimethylolpropane Hydroxypropyl Bis-Hydroxyethylamine Dendrimer, Trisodium Lauroampho PG-Acetate Chloride Phosphate, Undecanoic Acid, Undeceth-5 Carboxylic Acid, Undecylenamidopropylamine Oxide, Undecylenamidopropyl Betaine, Undecylenic Acid, Undecylenoyl Collagen Amino Acids, Undecylenoyl Glycine, Undecylenoyl Hydrolyzed Collagen, Undecylenoyl Wheat Amino Acids, Undecyl Glucoside, Wheat Germ Acid, Wheat Germamidopropylamine Oxide, Wheat Germamidopropyl Betaine, Yucca schidigera Leaf/Root/Stem Extract, Yucca schidigera Stem Extract, Zinc Coceth Sulfatea and Zinc Coco-Sulfate.

In addition to the above-described substances, further ingredients commonly used in the food industry, cosmetic or pharmaceutical industry or for homecare products, which are suitable or customary in the compositions of the present invention, can be used.

The cosmetic or pharmaceutical, in particular dermatological, preparations according to the present invention are intended for topical application. The term “topical” is understood to mean external applications on a mammal's skin or mucosa, which are in particular for the protection, treatment, care and cleansing of the skin, scalp, eyelashes, eyebrows, nails, mucous membranes and hair. The mammal is preferably a human.

For topical application, the cosmetic or pharmaceutical preparation is either a rinse off or a leave on preparation.

The cosmetic or pharmaceutical or homecare products according to the present invention may be in the form of an aqueous solution, dispersions, a hydro alcoholic vehicle, a stick, an ointment, a gel, an aerosol (foams, sprays, propellant pump) and the like.

The cosmetic or pharmaceutical, in particular dermatological, preparation or homecare product according to the first aspect of the present invention can be present in different forms, e.g. in the form of a dispersion, in the form of a liquid surfactant formulation, in the form of a solid surfactant formulation, in the form of a water free formulation, or in the form of an aqueous, aqueous/alcoholic, in particular aqueous/ethanolic, aqueous/glycolic or alcoholic/glycolic, in particular ethanolic/glycolic, based solution.

In a preferred variant, the cosmetic or pharmaceutical preparation or homecare product according to the first aspect of the invention is a dispersion. The term “dispersion” in the context of the present invention means, that the food, cosmetic or pharmaceutical preparation or homecare product is a disperse two-phase system consisting of colloidal particles (disperse phase) and a medium in which they are suspended (disperse medium). Both phases are not miscible with each other, only with an emulsifier. Such dispersions, for example emulsions, comprise the at least one oil component (without UV-filter(s)) preferably in an amount of ≥1% by weight, more preferably in an amount of ≥3% by weight.

Preferably, the food, cosmetic or pharmaceutical preparations or homecare products according to the present invention take various forms such as an emulsion, in particular a O/W emulsion, a W/O emulsion, a multiple emulsion, a hydrodispersion gel, a balm, a multiple emulsion of the water-in-oil type (W/O/WO) or of the oil-in-water type (O/W/O), PIT emulsion, Pickering emulsion, a micro-emulsion, a liquid, a lotion, a suspension, a milk, an ointment, a paste, a gel, a cream based, an oil based or a liposome-based formulation or a an aerosol such as foams and sprays, including all types of silicon based emulsions.

In a most preferred variant, the food, cosmetic or pharmaceutical preparations or homecare products according to the present invention are in the form of an emulsion as defined herein, advantageously in the form of an oil-in-water (O/W) emulsion comprising an oily phase dispersed in an aqueous phase in the presence of an O/W or polymeric emulsifier.

If the food, cosmetic or pharmaceutical preparation or homecare product according to the present invention is a dispersion, preferably an emulsion, the oil component (without UV-filter(s)) is present in the food, cosmetic or pharmaceutical preparation in an amount of 0.01 to 50.0% by weight, based on the total weight of the composition. In a preferred variant, the food cosmetic or pharmaceutical preparation comprises the oil component in an amount of 0.1 to 45.0% by weight, based on the total weight of the composition. In a particular preferred variant, the oil component is advantageously used in the food, cosmetic or pharmaceutical preparation in an amount of at 1.0 to 40% by weight, based on the total weight of the composition.

In a further variant, the cosmetic or pharmaceutical, preferably dermatological, preparation or homecare product according to the first of the invention is a water free formulation, i.e. an oil formulation.

If the cosmetic or pharmaceutical preparation or homecare product according to the present invention is a water free formulation, i.e. an oil formulation, the oil component is present in the cosmetic or pharmaceutical preparation in an amount of ≥60% by weight, preferably in an amount of ≥75% by weight, more preferably in an amount of ≥90% by weight, based on the total weight of the composition.

Such water free formulations include e.g. oils, skin butters, powders, lip stick, antiperspirant/deo sticks, and decorative cosmetics.

In addition, further water free formulations are likewise formulations on the basis of ethanol/diols/triols/glycols such as sprays or gels.

In a further alternative, the cosmetic or pharmaceutical preparation or homecare product according to the present invention is a liquid surfactant formulation.

Such liquid surfactant formulations include form example shampoo, shower gel, micellar water, liquid soap, cleansing preparations.

In a further alternative, the cosmetic or pharmaceutical preparation according to the present invention is a solid surfactant formulation.

Such solid surfactant formulations include for example solid shampoos, solid body wash, bar soaps.

If the cosmetic or pharmaceutical preparation according to the present invention is a liquid or solid surfactant composition, the surfactant component is present in the cosmetic or pharmaceutical preparation in an amount of 1.0 to 90% by weight, preferably in an amount of 1.5 to 80.0% by weight, more preferably in an amount of 2.0 to 70.0% by weight, based on the total weight of the composition.

Alternatively, the cosmetic or pharmaceutical preparation or homecare product as disclosed herein is an aqueous or an aqueous/alcoholic, preferably aqueous/ethanolic, or an aqueous/glycolic, or an alcoholic/glycolic, preferably ethanolic/glycolic, based solution. Typically, this could be glycerin in water or alcohol compositions. Said solutions are homogeneous one phase system of water/alcohol/glycol and additional components. The aqueous/alcoholic or aqueous/glycolic or alcoholic/glycolic based solution comprises an aliphatic alcohol or a glycol in an amount of 0.1 to 70.0% by weight, preferably in an amount of 0.5 to 60.0% by weight, more preferably in an amount of 1.0 to 50.0% by weight, based on the total weight of the solution. The aliphatic alcohol is preferably selected from the group consisting of ethanol, isopropanol, n-propanol. The glycol is preferably selected from the group consisting of glycerin, propylene glycol, 1,3-Propanediol, 1,2-Propanediol, 1,2-C5 to C10-alkanediols, butylene glycol or dipropylene glycol.

Preferably, the overall water content in the final aqueous based solutions can be ≥30% by weight, more preferably ≥40% by weight, most preferably ≥50% by weight. New applications such as those including water wipes have high water content. In a particular variant, the inventive compositions can be used for such wet wipe applications. They may then most preferably contain ≥90% water, or even ≥95% water.

Such aqueous or aqueous/alcoholic or aqueous/glycolic or alcoholic/glycolic based solutions include for example deo/antiperspirant preparations, after shave, cleansing preparations, or anti-acne preparations, or wet wipe solutions.

In a further preferred variant, the inventive composition is an impregnation solution in the form of an emulsion spray, aqueous/alcoholic, aqueous/glycolic, alcoholic/glycolic or alcoholic/oil spray for wet wipes.

The above formulations or compositions are prepared according to usual and known methods.

The cosmetic preparation according to the present invention is a preparation for cosmetic and/or non-therapeutic use for personal care, skin protection, skin care, scalp protection, scalp care, hair care, nail care, in particular for the prevention and/or treatment of skin conditions, intolerant or sensitive skin, skin irritation, skin reddening, rosacea, wheals, pruritus (itching), skin aging, wrinkle formation, loss of skin volume, loss of skin elasticity, pigment spots, pigment abnormalities, skin dryness, flaking, greasiness, hypopigmentation and/or hyperpigmentation of the skin; or a preparation for animal care.

Examples of personal care are preferably anti-ageing preparations, skin care emulsions, body oils, body lotions, cleansing lotions, face or body balms, after shave balms, after sun balms, deo emulsions, cationic emulsions, body gels, treatment creams, skin protection ointments, moisturizing gels, face and/or body moisturizers, light protective preparations (sunscreens), micellar water, hair spray, colour protection hair care products, skin lightening product, anti-dark spot preparations, etc.

The cosmetic preparations include also make-ups, eye-care preparation, eye shadows, mascara, eyeliner, lip care preparation such as lip stick, lip gloss, nail care preparations, such as nail varnish, nail varnish removers, etc.

Alternatively, the preparation according to the present invention is a preparation for medical use.

The pharmaceutical, in particular dermatological, preparation according to the present invention is preferably a preparation for the prevention and treatment of a condition of the skin, mucosa, hair or nails.

In order to be used, the cosmetic or pharmaceutical, in particular dermatological preparation according to the present invention is applied to the skin, hair, scalp and/or nails in an adequate amount in such manner as is customary with cosmetics and dermatological products.

In addition, the products and preparations according to the present invention are homecare products. The homecare products are predominantly detergent formulations, usually liquids, powders, sprays, granules or tablets, used to remove dirt, including dust, stains, bad smells and clutter on surfaces or other types of housecleaning or laundry detergent that is added for cleaning laundry or liquid soap. Typical homecare products include all-purpose cleaners, dishwashing detergents, hard floor and surface cleaners, glass cleaners, carpet cleaners, oven cleaners, laundry detergents (powder, liquid and table), fabric softeners, laundry scent, scent lotions, air fresheners, disinfectants, stain removers, carwash products, rim block gel, all aforementioned goods also in encapsulated form, air fresheners or furniture polish, etc.

In a preferred variant according to the present invention, the formulation is a fragrance composition or an aroma/flavour composition.

Surprisingly, the addition of a mycosporine-like amino acid compound to a fragrance or aroma/flavour compound in food, a cosmetic or pharmaceutical preparation or homecare product leads to an improvement in fragrance or aroma/flavour stability. In the presence of a mycosporine-like amino acid compound the fragrance or aroma/flavour compound are protected from degradation by oxygen, temperature and/or light. Therefore, the use of mycosporine-like amino acid compounds prevents said formulations from degradation, odour change, i.e. malodour formation, and/or discoloration, as it is demonstrated in the following examples.

Said stabilizing effect is observed for the mixture including MAA-1 in combination with a fragrance or aroma/flavour compound.

The mixture including MAA-2 in combination with a fragrance or aroma/flavour compound is particularly beneficial since it has a particularly pronounced stabilizing effect.

The mixture comprising MAA-3 in combination with an fragrance or aroma/flavour compound or a mixture comprising MAA-4 in combination with a fragrance or aroma/flavour compound show less degradation and discoloration.

Also, the mixture comprising MAA-5 or the mixture comprising MAA-6 significantly reduces degradation and discoloration, when used in combination with a fragrance or aroma/flavour compound.

A fragrance or aroma/flavour stabilizing effect is also true for a mixture including MAA-7 in combination with a fragrance or aroma/flavour compound or for a mixture including MAA-8 in combination with a fragrance or aroma/flavour compound.

A fragrance or aroma/flavour stabilizing effect is particularly distinctive for a combination including MAA-9 and a fragrance or aroma/flavour compound.

The mixtures comprising ethyl MAA-11 when used in combination with a fragrance or aroma/flavour result in a better stabilization effect.

The combinations of MAA-12, MAA-13, MAA-14, MAA-16, and MAA-17 and a fragrance or aroma/flavour are also beneficial since they lead to less degradation, odor change, i.e. malodour formation, and/or discoloration

Particular distinguished stabilizing effects can be obtained with a combination of any of the MAA-20 to MAA-39 and a fragrance or aroma/flavour.

With the use of the above-described mixtures/combinations including a mycosporine-like amino acid and a fragrance or aroma/flavour compound, the degradation of a fragrance or aroma/flavour compound in food, or cosmetic or pharmaceutical compositions or homecare products can be considerably reduced or even inhibited, and, thus, destabilization of such formulations in terms of malodour formation and/or discoloration, can be reduced.

Hence, the storage stability of such formulations can be considerably improved, and, thus, the shelf live can be prolonged.

Due to the above described advantageously effects, the afore specified mixtures including a mycosporine-like amino acid compound in combination with a fragrance or aroma/flavour compound display a remarkably stability against oxidation, temperature and light and are clearly superior to ready-to-use formulations without the addition of mycosporine-like amino acid compound.

For instance, the results in the following example clearly demonstrate that the addition of MAA-2, MAA-9, MAA-11 and MAA-12 to Aldehyde C12 results in reduced degradation and significant lower peroxide values in comparison to the reference sample which does not contain a MAA. This is a clear indication for a better stability of Aldehyde C12 in the presence of a MAA.

A similar effect could be observed for MAA-2, MAA-9, MAA-11 and MAA-12 with respect to Lilybelle. The addition of the MAA results in reduced degradation and significant lower peroxide values in comparison to the reference sample which does not contain a MAA. This is a clear indication for a better stability of Lilybelle in the presence of a MAA.

The MAA-2, MAA-9, MAA-11 or MAA-12 in combination with Globalide have reduced degradation and significant lower peroxide values in comparison to the reference sample which does not contain a MAA. This is a clear indication for a better stability of Globalide in the presence of a MAA.

A similar effect could also be observed for MAA-2, MAA-9, MAA-11 and MAA-12 with respect to Linalylacetate. The addition of the MAA results in reduced degradation and significant lower peroxide values in comparison to the reference sample which does not contain a MAA. This is a clear indication for a better stability of Linalylacetate in the presence of MAA-9 and MAA-12. MMA-2 and MMA-12 containing samples do not lead to improved stability but to lower peroxide values.

Furthermore, Citronellylacetate is more stable in the presence of MAA-9, MAA-11 and MMA-12 in comparison to the reference sample which does not contain a MAA.

Surprisingly it has been found that the addition of an effective amount of a mycosporine-like amino acid as defined herein to a composition comprising an antioxidant has a significant antioxidative capacity.

The above specified mixtures/combinations according to the first aspect of the present invention, comprising at least one antioxidant and at least a mycosporine-like amino acid compound (b) are characterized by a synergistically antioxidative efficacy, compared to their respective single mycosporine-like amino acid substance and antioxidant.

Oxidative degradation of a composition can considerably be reduced or minimized with an antioxidant in the presence of MAA-1.

The same effect is achieved with the addition of MAA-2.

Advantageous antioxidative effects are also achieved with MAA-3 or MAA-4 in combination with an antioxidant.

Also, the mixture MAA-5 or the mixture comprising MAA-6 synergistically reduces oxidative degradation when used in combination with an antioxidant.

A synergistic effect is also true for a mixture including MAA-7 in combination with an antioxidant or for a mixture including MAA-8 in combination with an antioxidant.

A synergistic effect is particularly distinctive for a combination including MAA-9 and an antioxidant.

The mixture comprising MAA-11 when used in combination with an antioxidant act synergistically against oxidative degradation.

The combinations of MAA-12, MAA-13, MAA-14, MAA-16, and MAA-17 and an antioxidant are also beneficial since they have a particular pronounced synergistic antioxidative effect.

A remarkable synergistic antioxidative effect is also obtained with a combination of any of the MAA-20 to MAA-39 and an antioxidant.

This means that the compositions are more stable against oxidative degradation. This comes along with a lower acid value, which is a measure of decomposition of triglycerides, and a less rancid odour.

Hence, the storage stability of such formulations can be considerably improved, and, thus, the shelf live can be prolonged.

The afore specified mixtures including a mycosporine-like amino acid compound in combination with an antioxidant display a remarkably synergistic activity and are clearly superior to the individually corresponding mycosporine-like amino acid compounds alone or the individually corresponding antioxidants and having the same concentration.

The boosted antioxidative effect obtained by use of at least one mycosporine-like amino acid compound has the advantage that the antioxidative efficiency of the antioxidant is broadened, either by achieving a higher performance of an antioxidant with the same amounts, or, vice versa, by achieving the same performance with lower amounts of an antioxidant, so that the overall content of the antioxidant in the final product can be reduced, compared to a final product including an antioxidant without the addition of a mycosporine-like amino acid compound.

Hence, the use of a mycosporine-like amino acid compound as defined herein in general allows for the use of less antioxidant in a food, cosmetic or pharmaceutical composition or homecare product for achieving the same antioxidative effect compared to a food, cosmetic or pharmaceutical composition comprising the same antioxidant but without the use of the mycosporine-like amino-acid compound.

Due to the described advantageously properties, a mycosporine-like amino acid compound (b) as defined herein can be used for the preparation of food, cosmetic or pharmaceutical preparations or homecare products in which a fragrance or aroma/flavour can be stabilized against oxidative degradation or degradation through temperature or light. Said stabilization allows the food, cosmetic or pharmaceutical preparations or homecare product to be stored without degradation, odour deterioration and discoloration, thus improving the shelf life of food, cosmetic or pharmaceutical preparations or homecare products.

Due to the outstanding stabilizing effects as described above, the combined use of at least one fragrance or aroma/flavour compound (a) and at least one mycosporine-like amino acid compound (b) is suitable to improve storage stability and prolong shelf life of the food, cosmetic or pharmaceutical preparations or homecare products.

Thus, in a further aspect the present invention relates to the use of at least one mycosporine-like amino acid compound as defined herein to stabilize a fragrance or aroma/flavour compound in a food, a cosmetic or pharmaceutical preparations or homecare products against degradation through oxidation, temperature or light. By stabilizing the fragrance or aroma/flavour compound the degradation and deterioration of the cosmetic or pharmaceutical preparation, in particular during storage, which goes along with impairment of the sensory quality of the products, can be prevented or at least minimized. Preferably, the at least one mycosporine-like amino acid compound as defined herein is used for stabilizing a fragrance composition or aroma/flavour composition.

Additionally, the present invention relates in a further aspect to the use of at least one mycosporine-like amino acid compound as defined herein for boosting an antioxidant, preferably in food, a cosmetic or pharmaceutical preparation or a homecare product against oxidative degradation.

Finally, the present invention also relates to a method of stabilizing a fragrance or aroma/flavour compound as defined herein in food, a cosmetic or a pharmaceutical preparation or homecare product. In the method according to the invention a food, a cosmetic or a pharmaceutical preparation or homecare product is provided. Next, an effective amount of a fragrance or aroma/flavour compound as defined herein is combined with an effective amount of a mycosporine-like amino acid compound and incorporated into the food, cosmetic or pharmaceutical preparation or homecare product, or an effective amount of a mycosporine-like amino acid compound as defined herein is added to a food, cosmetic or pharmaceutical preparation comprising a fragrance or aroma/flavour compound as defined herein, thereby achieving an enhanced stabilization of said products.

The present invention shall now be described in detail with reference to the following examples, which are merely illustrative of the present invention, such that the content of the present invention is not limited by or to the following examples.

### EXAMPLES

### Example 1: Influence of MAAs (Mycosporine-Like Amino Acid Compounds) on the Stability of Fragrance/Aroma/Flavour Ingredients

### Tested Fragrances/Aroma/Flavours:

The tests were performed with Alcohol C12, Globalide, Lilybelle, Linalylacetate and Citronellylacetate (dissolved 10% in dipropyleneglycol) plus/minus the addition of MAA's.

Treating the corresponding blends in the Oxipres device with heat and oxygen triggers and accelerates altering processes in a defined and reproducible way.

TABLE 4
Fragrance ingredients

| | Product | |
|---|---|---|
| Name | code | Structure |
| Aldehyd C12 (CAS 110-4I-8) | 161405 | 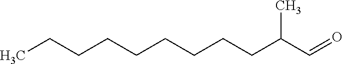 |
| Lilybelle (Cas 1378867-8I-2) | 633687 | 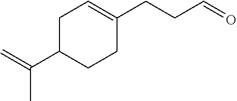 |
| Globalide (CAS 34902-57-3) | 106114 | 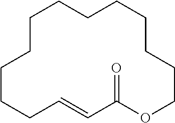 [SMILES: O=C1/C=C/CCCCCCCCCCCCO1] |
| Linalyl Acetate (CAS Linalyl Acetate) | 762262 | 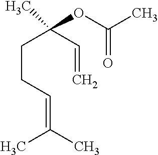 |
| Citronellylacetat (CAS 150-84-5) | 106216 | 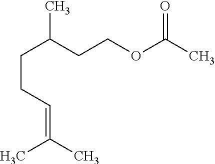 |

### Test Samples:

Sample A1: Aldehyde C12 without further additives
Sample A2: Aldehyde C12 plus 0.3% MAA-1
Sample A3: Aldehyde C12 plus 0.3% MAA-13
Sample A5: Aldehyde C12 plus 0.3% MAA-14
Sample A6: Aldehyde C12 plus 0.3% MAA-16
Sample A7: Aldehyde C12 plus 0.3% MAA-20
Sample B1: Lilybelle without further additives
Sample B2: Lilybelle plus 0.3% MAA-1
Sample B3: Lilybelle plus 0.3% MAA-13
Sample B5: Lilybelle plus 0.3% MAA-14
Sample B6: Lilybelle plus 0.3% MAA-16
Sample B7: Lilybelle plus 0.3% MAA-20
Sample C1: Globalide without further additives
Sample C2: Globalide plus 0.3% MAA-1
Sample C3: Globalide plus 0.3% MAA-13
Sample C5: Globalide plus 0.3% MAA-14
Sample C6: Globalide plus 0.3% MAA-16
Sample D1: Linalylacetate without further additives
Sample D2: Linalylacetate plus 0.3% MAA-1
Sample D3: Linalylacetate plus 0.3% MAA-13
Sample D5: Linalylacetate plus 0.3% MAA-14
Sample D6: Linalylacetate plus 0.3% MAA-16
Sample E1: Citronellylacetate without further additives
Sample E2: Citronellylacetate plus 0.3% MAA-1
Sample E3: Citronellylacetate plus 0.3% MAA-13
Sample E5: Citronellylacetate plus 0.3% MAA-14
Sample E6: Citronellylacetate plus 0.3% MAA-16

### Test Procedure:

For the evaluation of fragrance stabilizing properties, an Oxipres test was conducted. The Oxipres test is an accelerated shelf-life test by analyzing the oxidation stability. The sample is subjected to a high oxidative-stress environment (high temperature and oxygen over-pressure) in order to evaluate in short period of time the resistance to oxidation. The Oxipres method is based on oxygen consumption at high temperatures and pressures and allows the determination of oxidative resistance (shelf life) of e.g. oils. In the Oxipres test the sample is placed inside a hermetically closed iron vessel that is subjected to high oxygen pressures and temperatures of 90 to 120° C. The above-described samples were treated in Oxipres device as specified below. The consumption of oxygen results in a pressure drop in the vessel during the test. Higher decrease of pressure indicates more consumption of oxygen and higher oxidation of the test product.

### Equipment:

The tests were performed with an Oxipres device ex Mikrolab. The instrument is a modification of the bomb method (ASTM D941), which is based on oxidation with oxygen.

### Sample Preparation:

The respective fragrance ingredient Aldehyde C12, Globalide, Lilybelle, Linalylacetate and Citronellylacetate were dissolved 10% in Dipropyleneglycol. These solutions without further additives serves as a comparative sample. Afterwards 0.3% of the respective MAA's were dissolved in the 10% solution of fragrance ingredients/dipropyleneglycol. The above-described samples were treated in an Oxipres device. The sample was placed inside a hermetically closed iron vessel that is subjected to high oxygen pressures and temperatures of 50 to 70° C. The test conditions are specified below.

Before and after Oxipres treatment the samples were analyzed by GC.

Furthermore, the peroxide value was measured before and after treatment. The peroxide value is a parameter specifying the content of oxygen as peroxide, especially hydroperoxides in a substance. This method is used to analyze animal oils and fats, vegetable oils and fats, as well as for fragrance and aroma/flavour substances. Peroxide values are expressed either in milliequivalents of peroxide/kg or in millimoles of peroxide/L.

### Example 1.1: Aldehyde C12 (10% Solution in Dipropylene Glycol+/−MAA's)

TABLE 5
Preparation of Aldehyde C12 samples A1, A2, A3, A5, A6 and A7

| | A1 | A2 | A3 | A5 | A6 | A7 |
|---|---|---|---|---|---|---|
| Aldehyd | 100.0 | 99.7 | 99.7 | 99.7 | 99.7 | 99.7 |
| C12 sol., | | | | | | |
| 10% in | | | | | | |
| Dipropylene | | | | | | |
| Glycol | | | | | | |
| MAA-1 | — | 0.3 | — | — | — | — |
| MAA-13 | — | — | 0.3 | — | — | — |
| MAA-14 | — | — | — | 0.3 | — | — |
| MAA-16 | — | — | — | — | 0.3 | — |
| MAA-20 | — | — | — | — | — | 0.3 |

The ingredients as listed in Table 5 were blended and stirred by heating up to 70° C. (magnetic stirrer) until a homogeneous solution was achieved.

TABLE 6
Results of degradation [GC area %] and peroxide values
before and after Oxipres treatment (48 h; 50° C.; 5 bar)

| | | | | | Peroxide values | | |
|---|---|---|---|---|---|---|---|
| | | GC [area %] | | | [meq O₂/KG] | | |
| | | | | degra- | | | Δ |
| | Sample | | | dation | | | peroxide |
| No. | without | start | end | [%] | start | end | value |
| A1 | MAA | 18.0 | 2.6 | 85.7 | 0 | 297 | 297 |
| | additive | | | | | | |
| | (comparative | | | | | | |
| | sample) | | | | | | |
| A2 | plus 0.3% | 17.7 | 7.4 | 58.7 | 0 | 71 | 71 |
| | MAA-1 | | | | | | |
| A3 | plus 0.3% | 18.1 | 6.7 | 62.8 | 0 | 37 | 37 |
| | MAA-13 | | | | | | |
| A5 | plus 0.3% | 5.7 | 4.9 | 13.6 | 0 | 35 | 35 |
| | MAA-14 | | | | | | |
| A6 | plus 0.3% | 18.0 | 12.5 | 30.7 | 0 | 33 | 33 |
| | MAA-16 | | | | | | |

The results in Table 6 clearly show that the MAA containing samples A2, A3, A5 and A6 have reduced degradation and significant lower peroxide values in comparison to sample A1 which does not contain a MAA. This is a clear indication for a better stability of Aldehyd C12 in the presence of an MAA.

TABLE 7
Results of degradation [GC area %] and peroxide values
before and after Oxipres treatment (48 h; 50° C.; 5 bar)

| | | GC | | | Peroxide values | | |
|---|---|---|---|---|---|---|---|
| | | [area %] | | | [meq O₂/KG] | | |
| | | | | degra- | | | Δ |
| | | | | dation | | | peroxide |
| No. | Sample | start | end | [%] | start | end | value |
| A1 | without MAA | 17.9 | 3.6 | 79.9 | 2.1 | 204 | 202 |
| (other | additive | | | | | | |
| batch) | (comparative | | | | | | |
| | sample) | | | | | | |
| A7 | plus 0.3% | 17.7 | 15.0 | 15.3 | 0 | 11 | 11 |
| | MAA-20 | | | | | | |

The results in Table 7 clearly show that the MAA containing sample A7 has reduced degradation and significant lower peroxide values in comparison to sample A1 which does not contain a MAA. This is a clear indication for a better stability of Aldehyd C12 in the presence of MAA-20.

### Example 1.2: Lilybelle (10% Solution in Dipropylene Glycol+/−MAA's)

TABLE 8
Preparation of Lilybelle samples B1, B2, B3, B5, B6 and B7

| | B1 | B2 | B3 | B5 | B6 | B7 |
|---|---|---|---|---|---|---|
| Lilybelle | 100.0 | 99.7 | 99.7 | 99.7 | 99.7 | 99.7 |
| sol., | | | | | | |
| 10% in | | | | | | |
| Dipro- | | | | | | |
| pylene | | | | | | |
| Glycol | | | | | | |
| MAA-1 | — | 0.3 | — | — | — | — |
| MAA-13 | — | — | 0.3 | — | — | — |
| MAA-14 | — | — | — | 0.3 | — | — |
| MAA-16 | — | — | — | — | 0.3 | — |
| MAA-20 | — | — | — | — | — | 0.3 |

The ingredients as listed in Table 8 were blended and stirred by heating up to 70° C. (magnetic stirrer) until a homogeneous solution was achieved.

TABLE 9
Results of degradation [GC area %] and peroxide values
before and after Oxipres treatment (48 h; 50° C.; 5 bar)

| | | | | | Peroxide values | | |
|---|---|---|---|---|---|---|---|
| | | GC [area %] | | | [meq O₂/KG] | | |
| | | | | degra- | | | Δ |
| | | | | dation | | | peroxide |
| No. | Sample | start | end | [%] | start | end | value |
| B1 | without | 18.3 | 3.2 | 82.8 | 9 | 282 | 273 |
| | MAA | | | | | | |
| | addition | | | | | | |
| | (comparative | | | | | | |
| | sample) | | | | | | |
| B2 | plus 0.3% | 18.4 | 3.4 | 81.5 | 5 | 224 | 219 |
| | MAA-1 | | | | | | |
| B3 | plus 0.3% | 18.4 | 8.7 | 52.8 | 0 | 91 | 91 |
| | MAA-13 | | | | | | |
| B5 | plus 0.3% | 0.8 | 0.4 | 58.5 | 7 | 222 | 215 |
| | MAA-14 | | | | | | |
| B6 | plus 0.3% | 18.2 | 7.8 | 57.4 | 5 | 93 | 88 |
| | MAA-16 | | | | | | |

The results in Table 9 clearly show that the MAA containing samples B2, B3, B5 and B6 have reduced degradation and significant lower peroxide values in comparison to sample B1 which does not contain a MAA. This is a clear indication for a better stability of Lilybelle in the presence of a MAA.

TABLE 10
Results of degradation [GC area %] and peroxide values
before and after Oxipres treatment (48 h; 50° C.; 5 bar)

| | | GC | | | Peroxide values | | |
|---|---|---|---|---|---|---|---|
| | | [area %] | | | [meq O₂/KG] | | |
| | | | | degra- | | | Δ |
| | | | | dation | | | peroxide |
| No. | Sample | start | end | [%] | start | end | value |
| B1 | without | 16.3 | 10.1 | 38.0 | 0 | 126 | 126 |
| (other | MAA | | | | | | |
| batch) | additive | | | | | | |
| | (comparative | | | | | | |
| | sample) | | | | | | |
| B7 | plus 0.3% | 16.4 | 15.6 | 4.9 | 0 | 0.8 | 0.8 |
| | MAA-20 | | | | | | |

The results in Table 10 clearly show that the MAA-20 containing samples B7 has reduced degradation and significant lower peroxide values in comparison to sample B1 which does not contain a MAA. This is a clear indication for a better stability of Lilybelle in the presence of a MAA-20.

### Example 1.3: Globalide (10% Solution in Dipropylene Glycol+/−MAA's)

TABLE 11
Preparation of Globalide samples C1, C2, C3, C5 and C6

| | C1 | C2 | C3 | C5 | C6 |
|---|---|---|---|---|---|
| Globalide sol., | 100.0 | 99.7 | 99.7 | 99.7 | 99.7 |
| 10% in | | | | | |
| Dipropylene | | | | | |
| Glycol | | | | | |
| MAA-1 | — | 0.3 | — | — | — |
| MAA-13 | — | — | 0.3 | — | — |
| MAA-14 | — | — | — | 0.3 | — |
| MAA-16 | — | — | — | — | 0.3 |

The ingredients as listed in Table 11 were blended and stirred by heating up to 70° C. (magnetic stirrer) until a homogeneous solution was achieved.

TABLE 12
Results of degradation [GC area %] and peroxide values
before and after Oxipres treatment (96 h; 70° C.; 5 bar)

| | | GC | | | Peroxide values | | |
|---|---|---|---|---|---|---|---|
| | | [area %] | | | [meq O₂/KG] | | |
| | | | | degra- | | | Δ |
| | | | | dation | | | peroxide |
| No. | Sample | Start | end | [%] | start | end | value |
| C1 | without MAA | 16.2 | 13.1 | 3.1 | 24 | 770 | 746 |
| | addition | | | | | | |
| | (comparative | | | | | | |
| | sample) | | | | | | |
| C2 | plus 0.3% | 16.2 | 11.8 | 4.4 | 16 | 562 | 546 |
| | MAA-1 | | | | | | |
| C3 | plus 0.3% | 15.9 | 15.7 | 0.3 | 11 | 110 | 99 |
| | MAA-13 | | | | | | |
| C5 | plus 0.3% | 16.2 | 13.5 | 2.6 | 11 | 648 | 637 |
| | MAA-14 | | | | | | |
| C6 | plus 0.3% | 16.2 | 15.4 | 0.8 | 16 | 244 | 228 |
| | MAA-16 | | | | | | |

The results in Table 12 clearly show that the MAA containing samples C3, C5 and C6 have reduced degradation. Significant lower peroxide values in comparison to sample C1 could be observed for all used MAAs. This is a clear indication for a better stability of Globalide in the presence of a MAA.

### Example 1.4: Linalylacetate (10% Solution in Dipropylene Glycol+/−MAA's)

TABLE 13
Preparation of Linalylacetate samples D1, D2, D3, D5 and D6

| | D1 | D2 | D3 | D5 | D6 |
|---|---|---|---|---|---|
| Linalylacetate | 100.0 | 99.7 | 99.7 | 99.7 | 99.7 |
| sol., 10% in | | | | | |
| Dipropylene | | | | | |
| Glycol | | | | | |
| MAA-1 | — | 0.3 | — | — | — |
| MAA-13 | — | — | 0.3 | — | — |
| MAA-14 | — | — | — | 0.3 | — |
| MAA-16 | — | — | — | — | 0.3 |

The ingredients as listed in Table 13 were blended and stirred by heating up to 70° C. (magnetic stirrer) until a homogeneous solution was achieved.

TABLE 14
Results of degradation [GC area %] and peroxide values
before and after Oxipres treatment (24 h; 70° C.; 5 bar)

| | | GC | | | Peroxide values | | |
|---|---|---|---|---|---|---|---|
| | | [area %] | | | [meq O₂/KG] | | |
| | | | | degra- | | | Δ |
| | | | | dation | | | peroxide |
| No. | Sample | start | end | [%] | start | end | value |
| D1 | without | 17.8 | 12.7 | 5.1 | 0 | 550 | 550 |
| | MAA | | | | | | |
| | addition | | | | | | |
| | (comparative | | | | | | |
| | sample) | | | | | | |
| D2 | plus 0.3% | 17.8 | 10.7 | 7.1 | 0 | 540 | 540 |
| | MAA-1 | | | | | | |
| D3 | plus 0.3% | 17.8 | 16.6 | 1.2 | 3 | 26 | 23 |
| | MAA-13 | | | | | | |
| D5 | plus 0.3% | 17.3 | 11.0 | 6.3 | 0 | 233 | 233 |
| | MAA-14 | | | | | | |
| D6 | plus 0.3% | 17.7 | 17.4 | 0.3 | 0 | 12 | 12 |
| | MAA-16 | | | | | | |

The results in Table 14 clearly show that MAA-13 and MAA-16 containing samples D3 and D6 have reduced degradation and significant lower peroxide values in comparison to sample D1 which contains no MAA. This is a clear indication for a better stability of Linalylacetate in the presence of MAA-13 and MAA-16. MAA-1 and MAA-14 containing samples do not lead to improved stability but to lower peroxide values.

### Example 1.4: Citronellylacetate (10% Solution in Dipropylene Glycol+/−MAA's)

TABLE 15
Preparation of Linalylacetate samples
E1, E2, E3, E5 and E6

| | E1 | E2 | E3 | E5 | E6 |
|---|---|---|---|---|---|
| Citronellyl- | 100.0 | 99.7 | 99.7 | 99.7 | 99.7 |
| acetate | | | | | |
| sol., 10% in | | | | | |
| Dipropylene | | | | | |
| Glycol | | | | | |
| MAA-1 | — | 0.3 | — | — | — |
| MAA-13 | — | — | 0.3 | — | — |
| MAA-14 | — | — | — | 0.3 | — |
| MAA-16 | — | — | — | — | 0.3 |

The ingredients as listed in Table 15 were blended and stirred by heating up to 70° C. (magnetic stirrer) until a homogeneous solution was achieved.

TABLE 16
Results of degradation [GC area %] and peroxide values
before and after Oxipres treatment (48 h; 70° C.; 5 bar)

| | | | | | Peroxide values | | |
|---|---|---|---|---|---|---|---|
| | | GC [area %] | | | [meq O₂/KG] | | |
| | | | | degra- | | | Δ |
| | | | | dation | | | peroxide |
| No. | Sample | start | end | [%] | start | end | value |
| E1 | without | 16.5 | 12.8 | 3.8 | 36 | 462 | 426 |
| | MAA | | | | | | |
| | addition | | | | | | |
| | (comparative | | | | | | |
| | sample) | | | | | | |
| E2 | plus 0.3% | 16.7 | 11.7 | 5.0 | 31 | 480 | 449 |
| | MAA-1 | | | | | | |
| E3 | plus 0.3% | 16.8 | 16.4 | 0.4 | 18 | 88 | 70 |
| | MAA-13 | | | | | | |
| E5 | plus 0.3% | 16.8 | 16.1 | 0.7 | 17 | 106 | 89 |
| | MAA-14 | | | | | | |
| E6 | plus 0.3% | 16.7 | 16.2 | 0.5 | 23 | 137 | 114 |
| | MAA-16 | | | | | | |

The results in table 16 clearly show that Citronellylacetate is more stable in the presence of MAA-13, MAA-16 and MAA-14. No stabilizing effect could be observed with MAA-1

### Example 2: Formulation Examples

In the following formulation examples the following five perfume oils PFO1, PFO2, PFO3, PFO4 or PFO5 were each used as fragrance.

TABLE F1
Composition of perfume oil 1
(PO1; amounts in % b.w.)

| Ingredients | Amount |
|---|---|
| Aldehyde C12 | 2.0 |
| Allyl amyl glycolate 10% dpg | 5.0 |
| Anisic aldehyde pure | 5.0 |
| Apple oliffac type | 10.0 |
| Benzylacetate | 50.0 |
| Bergamot identoil ® colourless | 15.0 |
| Canthoxal | 5.0 |
| Cetalox 10% ipm | 3.0 |
| Citronellol 950 | 40.0 |
| Damascenone total 1% dpg | 5.0 |
| Damascone alpha 10% dpg | 5.0 |
| Damascone delta 10% dpg | 2.0 |
| Dimethyl benzyl carbinyl butyrate | 2.0 |
| Dipropylene glycol | 178.0 |
| Ebanol | 2.0 |
| Ethyl decadienoate trans cis-2.4 | 2.0 |
| Florosa | 5.0 |
| Frambinon ® 10% dpg | 7.0 |
| Galaxolide 50% in ipm | 100.0 |
| Galbex type base | 1.0 |
| Geranyl acetate pure | 2.0 |
| Hedione | 30.0 |
| Heliotropin | 10.0 |
| Hexenyl acetate cis-3 10% dpg | 1.0 |
| Hexenyl salicylate cis-3 | 5.0 |
| Hexyl cinnamic aldehyde alpha | 70.0 |
| Hexyl salicylate | 50.0 |
| Hydroxy citronellal | 10.0 |
| Iso e super | 15.0 |
| Isoraldeine 70 | 20.0 |
| Leafovert ® | 1.0 |
| Lilial | 60.0 |
| Linalool | 60.0 |
| Linalyl acetate | 20.0 |
| Lyral | 7.0 |
| Manzanate | 2.0 |
| Phenoxanol | 7.0 |
| Phenylethyl alcohol | 120.0 |
| Sandal mysore core | 2.0 |
| Sandranol ® | 7.0 |
| Styralyl acetate | 3.0 |
| Tagetes rco 10% tec | 2.0 |
| Terpineol pure | 20.0 |
| Tetrahydrogeraniol 10% dpg | 5.0 |
| Tonalide | 7.0 |
| Vertocitral 10% dpg | 5.0 |
| Vertofix | 15.0 |
| Total | 1000.0 |

TABLE F2
Composition of perfume oil 2
(PO2; amounts in % b.w.)

| Ingredients | Amount |
|---|---|
| Acetophenone. 10% in DPG | 10 |
| n-Undecanal | 5 |
| Aldehyde C14 (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3. 10% in DPG | 5 |
| Hexenyl salicylate cis-3 | 20 |
| Indole. 10% in DPG | 10 |
| Alpha-ionone | 15 |
| Beta-ionone | 5 |
| Lilybelle ® | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE F3
Composition of perfume oil 3
(PO3; amounts in % b.w.)

| Ingredients | Amount |
|---|---|
| Benzyl acetate | 60 |
| Citronellyl acetate | 60 |
| Cyclamenaldehyde | 20 |
| Dipropylene glycol (DPG) | 60 |
| Ethyllinalool | 40 |
| Florol (2-isobutyl-4-methyltetrahydro-2H- | 30 |
| Globalide ® | 180 |
| Hedione ® (methyldihydrojasmonate) | 140 |
| Hexenyl salicylate. cis-3 | 10 |
| Vertocitral (2.4-dimethyl-3- | 5 |
| Hydratropaldehyde. 10% in DPG | 5 |
| Isodamascone (1-(2.4.4-trimethyl-2- | 5 |
| Isomuscone (cyclohexadecanone) | 40 |
| Jacinthaflor (2-methyl-4-phenyl-1.3- | 10 |
| Cis-jasmone. 10% in DPG | 20 |
| Linalool | 50 |
| Linalyl acetate | 30 |
| Methyl benzoate. 10% in DPG | 25 |
| para-Methyl cresol. 10% in DPG | 10 |
| Nerol | 20 |
| Phenylpropylaldehyde | 5 |
| 2-Phenylethyl alcohol | 82 |
| Tetrahydrogeraniol | 13 |
| 2.2-Dimethyl-3-cyclohexyl-1-propanol | 80 |
| Total: | 1000 |

TABLE F4
Composition of perfume oil 4
(PO4; amounts in % b.w.)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATER 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDER (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL | 1000 |

TABLE F5
Composition of perfume oil 5
(PO5; amounts in %% b.w.)

| Ingredients | Amount |
|---|---|
| AMAROCITE ® | 10 |
| AMBROCENIDE ® 10% in DPG | 5 |
| AMBROXIDE | 15 |
| AURELIONE ® (7/8-Cyclohexadecenone) | 70 |
| (MACRO) | |
| BERGAMOT OIL. bergapten-free | 90 |
| CALONE ® 1951 10% in DPG | 20 |
| CARAWAY OIL | 10 |
| CITRAL | 20 |
| COUMARIN | 10 |
| ALPHA-DAMASCONE 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |
| TOTAL | 1000 |

TABLE F6
Composition of Aroma1
(A1; Peppermint flavour
(amounts in % b.w.))

| Ingredients | Amount |
|---|---|
| Isobutyraldehyde | 0.5 |
| 3-Octanol | 0.5 |
| Dimethyl sulphide | 0.5 |
| Trans-2-Hexanal | 1.0 |
| Cis-3-Hexenol | 1.0 |
| 4-Terpineol, natural | 1.0 |
| Isopulegol | 1.0 |
| Piperitone, natural, from eucalyptus | 2.0 |
| Linalool | 3.0 |
| 8-Ocimenyl acetate 10% in triacetin | 5.0 |
| Isoamyl alcohol | 10.0 |
| Isovaleraldehyde | 10.0 |
| Alpha-Pinene, natural | 25.0 |
| Beta-Pinene, natural | 25.0 |
| Neomenthol. Racemic | 40.0 |
| Eucalyptiol (1.8-cineol). natural | 50.0 |
| L-Menthyl acetate of the formula D | 70.0 |
| L-Menthone | 220.0 |
| D-Isomenthone | 50.0 |
| L-Menthol | 483.5 |
| Nonenolide | 1.0 |
| TOTAL | 1000 |

The above perfume oils PO1, PO2, PO3, PO4 or PO5 or aroma A1 were incorporated into the formulations presented below.

In the following formula the amounts are indicated as % by weight.

### Cosmetic Formulations/Sunscreen Formulations (Compositions)

Deodorant Spray with ACH
Sport Fit Deo
Biotic Acne Control Gel
Cleansing Micellar Gel
Shampoo Sulfate-Free
Antidandruff Shampoo
Fresh Hair Shampoo
Anti-itch hair conditioner, leave on.
Hair Refreshener Mist.
Anti-Wrinkle Night Emulsion.
Antibacterial Body Lotion, sprayable
Refreshing and soothing after sun cream
Emulsion with low emulsifier content
Natural Night Cream Aloe
Clear Anti-Acne Wipe
After-Shave Wipes
Sensi Scalp (Green Solid Shampoo)
Hygiene & Care Hand Cream
Mouthwash without alcohol (ready to use)
Sunscreen lotion (o/w; broadband protection).
Sun protection soft cream (w/o), SPF 40

TABLE F7
Deodorant Spray with ACH

| Ingredients | INCI | Amount |
|---|---|---|
| Triethylcitrate | Triethylcitrate | 3.0 |
| Solubilizer ® | PEG-40 Hydrogenated Castor | 1.0 |
| | Oil, Trideceth-9, Propylene | |
| | Glycol, Aqua | |
| Ethanol 96% | Ethanol | 30.0 |
| Ethylhexylglycerin | Ethylhexylglycerin | 0.3 |
| SymDeo ® B125 | 2-Methyl 5-Cycloheylpentanol | 0.25 |
| SymClariol ® | Decylene Glycol | 0.25 |
| Frescolat ® ML | Menthyl Lactate | 0.6 |
| Tego ® Cosmo P 813MB | Polyglyceryl-3 Caprylate | 0.5 |
| Locron ® L (50%) | Aqua, Aluminium Chlorohydrate | 25.0 |
| Perfume oil PO1, PO2, | Parfum | 0.5 |
| PO3, PO4, or PO5 | | |
| MAA-1 | | 0.8 |
| Water, demin. | Aqua | add 100 |

TABLE F8
Sport Fit Deo

| Ingredients | INCI | Amount |
|---|---|---|
| SymLite ® G8 | Glyceryl caprylate | 0.15 |
| Ethanol | Alcohol denat | add 100 |
| SymClariol ® | Decylene Glycol | 0.25 |
| SymDeo ® B125 | 2-Methyl 5-Cyclohexylpentanol | 0.25 |
| Frescolat ® ML | Menthyl Lactate | 0.5 |
| Extrapone ® Ginseng | Propylene Glycol, Aqua, Panax | 0.1 |
| | ginseng root extract, Lactic acid | |
| Extrapone ® Peppermint | Aqua, Propylene Glycol, Mentha | 0.1 |
| | Piperita Leaf extract | |
| Ethylhexyglycerin | Ethylhexylglycerin | 0.2 |
| MAA-13 | | 0.7 |
| MAA-14 | | 0.3 |
| Perfume oil PO1, PO2, | Parfum | 1.0 |
| PO3, PO4, or PO5 | | |

TABLE F9
Biotic Acne Control Gel

| Ingredients | INCI | Amount |
|---|---|---|
| Water, distilled | Water (Aqua) | add 100 |
| SymSave ® H | Hydroxyacetophenone | 0.5 |
| Carbopol ® Ultrez | Acrylates/C10-30 Alkyl Acrylate | 0.5 |
| 21 Polymer | Crossploymer | |
| MAA-2 | | 0.25 |
| MAA-13 | | 1.0 |
| Solubilizer | PEG-40 Hydrogenated Castor | 2.5 |
| | Oil, Trideceth-9, Propylene | |
| | Glycol, Aqua | |
| Sym Clariol ® | Decylene Glycol | 0.5 |
| SymMollient ® W/S | Trideceth-9, PEG-5 | 1.0 |
| | Isononanoate, Aqua | |
| Hydrolite ®- 5 green | Pentylene Glycol | 5.0 |
| Dragosantol ® | Bisabolol | 0.05 |
| Farnesol | Farnesol | 0.3 |
| Ethanol | Alcohol, Aqua | 5.0 |
| Flowerpone ® Lavender | PEG-40 Hydrogenated Castor | 0.5 |
| | Oil, Trideceth-9, Pentylene | |
| | Glycol, Lavandula Angustifolia | |
| | Flower Extract, Propylene | |
| | Glycol, Bisabolol | |
| SymRebootTM L19 | Maltodextrin, Lactobacillus | 0.5 |
| | Ferment | |
| Sodium Hydroxide | Aqua, Sodium Hydroxide | 1.1 |
| 10% solution | | |
| Colour | Colour | 0.2 |
| Perfume oil PO1; PO2; | Fragrance | 0.3 |
| PO3; PO4 or PO5 | | |

TABLE F10
Cleansing Micellar Gel

| Ingredients | INCI | Amount |
|---|---|---|
| Water, distilled | Water (Aqua) | add 100 |
| MAA-16 | | 0.6 |
| MAA-1 | | 0.4 |
| Carbopol ® ETD | Carbomer | 0.7 |
| 2050 Polymer | | |
| Hydroviton ® PLUS 2290 | Aqua, Pentylene glycol, Glycerin | 2.0 |
| | Fructose, Urea, Citric acid, | |
| | Sodium hydroxide, Maltose | |
| | Sodium pca, Sodium chloride | |
| | Sodium lactate, Trehalose | |
| | Allantoin, Sodium hyaluronate | |
| | Glucose | |
| SymOcide ® PS | Phenoxyethanol, | 1.0 |
| | Decylene glycol, | |
| | 1,2-hexanediol | |
| Sodium Hydroxide | Aqua, Sodium Hydroxide | 3.28 |
| 10% solution | | |
| SymMollient ® W/S | Trideceth-9, PEG-5 | 1.0 |
| | Isononanoate, Aqua | |
| SymSol ® PF-3 | Aqua, Pentylene glycol, Sodium | 3.0 |
| | lauryl sulfoacetate, Sodium | |
| | oleoyl sarcosinate, Sodium | |
| | chloride, Sodium oleate | |
| Perfume oil PO1; PO2; | Parfum | 1.0 |
| PO3; PO4 or PO5 | | |
| SymVital ® AR | Zingiber officinale (ginger) root | 0.2 |
| | extract | |
| Extrapone ® Seaweed | Aqua | 1.0 |
| | Butylene glycol | |
| | Fucus vesiculosus extract | |
| Actipone ® Dandelion Juice | Glycerin | 1.0 |
| (Organic) GW | Taraxacum officinale juice | |
| | Aqua | |
| Colour II | Colour | 0.20 |
| Caprylhydroxamic Acid | Caprylhydroxamic Acid | 0.1 |
| Phenylpropanol | Phenylpropanol | 0.2 |

TABLE F11
Shampoo Sulfate-Free

| Ingredients | INCI | Amount |
|---|---|---|
| Aqua/Water | Aqua | add 100 |
| Akypo ® Foam RL 40 | Sodium Laureth-5 | 15.00 |
| | Carboxylate | |
| MAA-13 | | 1.0 |
| MAA-20 | | 0.15 |
| Plantacare ® 2000 UP | Decyl Glucoside | 5.00 |
| Dissolvine GL-47-S | Tetrasodium Glutamate | |
| | Diacetate | |
| SymOcide ® PH | Phenoxyethanol, | 1.45 |
| | Hydroxyacetophenone, | |
| | Caprylyl Glycol, Aqua | |
| Aqua/Water | Aqua | 10.00 |
| Novethix ™ L-10 Polymer | Acrylates/Beheneth-25 | 7.00 |
| | Methacrylate Copolymer | |
| Dehyton ® PK 45 | Cocamidopropyl Betaine | 10.00 |
| SymMollient ® W/S | Trideceth-9, PEG-5 | 0.50 |
| | Isononanoate, Aqua | |
| SymCalmin ® | Pentylene Glycol, Butylene | 1.00 |
| | Glycol, Hydroxyphenyl | |
| | Propamidobenzoic acid | |
| Antil ® 141 Liquid | Propylene Glycol, PEG-55 | 2.00 |
| | Proplyene Glycol Oleate | |
| Acusol ™ OP 301 | Styrene/Acrylates Copolymer | 1.00 |
| SymHair ® Restore | Glycerin, Triticum Vulgare | 1.00 |
| | Protein, Aqua | |
| Perfume oil PO1, PO2, | Parfum | 1.00 |
| PO3, PO4, or PO5 | | |

TABLE F12
Antidandruff Shampoo

| Ingredients | INCI | Amount |
|---|---|---|
| Water, distilled | Water (Aqua) | add 100 |
| MAA-1 | | 1.5 |
| Polymer JR 400 | Polyquaternium-10 | 0.4 |
| Texapon ® NSO IS | Sodium Laureth Sulfate | 40.0 |
| Dehyton K | Cocamidopropyl Betaine | 8.0 |
| Plantacare 2000 UP | Decyl Glucoside | 2.0 |
| Citric Acid 10% solution | Citric Acid | 0.8 |
| Frescolat ® X-cool | Menthyl Ethylamido Oxalate | 0.5 |
| Sym Triol ® | Caprylyl Glycol, 1,2-Hexanediol, | 0.8 |
| | Methylbenzyl Alcohol | |
| Crinipan ® AD | Climbazole | 0.5 |
| SymMollient ® W/S | Trideceth-9, PEG-5 Isononanoate, | 2.0 |
| | Water (Aqua) | |
| Tinogard ® Q | Tri(tetramethylhydroxypiperodonol) | 0.02 |
| | Citrate | |
| Sodium Chloride | Sodium Chloride | 0.5 |
| Perfume oil PO1; | Fragrance | 0.4 |
| PO2; PO3; PO4 or PO5 | | |

TABLE F13
Fresh Hair shampoo

| Ingredients | INCI | Amount |
|---|---|---|
| Aqua | Aqua | add 100 |
| EDTA B Powder | Disodium EDTA | 0.1 |
| MAA-1 | | 0.8 |
| MAA-13 | | 0.6 |
| Nativacare ™ 5600 | Zea Mays (Corn Starch) | 1.0 |
| Rheocare ® XGN | Xanthan Gum | 0.5 |
| Texapon ® NSO UP | Sodium Laureth sulfate | 35.0 |
| Dehyton ® K | Cocamidopropyl betaine | 8.0 |
| Plantacare ® 2000 UP | Decyl Glucoside | 4.0 |
| Perfume oil PO1, PO2, | Parfum | 1.0 |
| PO3, PO4, or PO5 | | |
| Sym Save ® H | Hydroxyacetophenone | 0.5 |
| Hydrolite ® 6 | 1,2-Hexanediol | 1.0 |
| SymClariol ® | Decylene Glycol | 0.3 |
| Frescolat ® ML cryst | Menthyl Lactate | 1.0 |
| SymCalmin ® | Pentylene Glycol, Butylene | 0.2 |
| | Glycol, Hydroxyphenyl | |
| | propamidobenzoic acid | |
| Crinipan ® AD | Climbazole | 0.2 |
| Propylene Glycol | Propylene Glycol | 2.0 |
| SymHair ® Shield | Pentylene Glycol, Aqua, | 1.0 |
| | Glycerin, Triticum vulgare | |
| | bran extract, 1,2-Hexanediol, | |
| | Capryly glycol | |
| Sodium Hydroxide | Aqua, Sodium hydroxide | 2.2 |
| 10% solution | | |

TABLE F14
Anti-itch hair conditioner, leave on

| Ingredients | INCI | Amount |
|---|---|---|
| Dragosantol ® 100 | Bisabolol | 0.1 |
| Dehyquart ® A CA | Cetrimonium Chloride | 0.5 |
| Dehyquart ® SP | Quaternium-52 | 4.0 |
| Dracorin ® CE | Glyceryl Stearate Citrate | 1.0 |
| Drago-Oat-Active | Water (Aqua), Butylene Glycol, | 2.0 |
| | Avena Sativa (Oat) Kernel | |
| | Extract | |
| Perfume oil PO1, PO2, | Fragrance | 0.1 |
| PO3, PO4, or PO5 | | |
| Nativacare ™ 5600 | Zea (Mays) Corn Starch | 1.5 |
| Lara Care ® A-200 | Galactoarabinan | 1.5 |
| Neutral Oil | Caprylic/Capric Triglyceride | 1.0 |
| PCL Liquid ® 100 | Cetearyl Ethylhexoate | 0.3 |
| Ucare ™ Polymer JR 400 | Polyquaternium-10 | 0.1 |
| Propylene Glycol | Propylene Glycol | 0.8 |
| SymGlucan ® | Aqua, Glycerin, 1,2-Hexandiol, | 5 |
| | Caprylyl Glycol, Beta-Glucan | |
| Crinipan ® PMC green | Propanediol Caprylate | 0.5 |
| SymMollient ® W/S | Trideceth-9, PEG-5 | 2.0 |
| | Isononanoate, Water (Aqua) | |
| SymOcide ® PH | Phenoxyethanol, | 1.2 |
| | Hydroxyacetophenone, | |
| | Caprylyl Glycol, Aqua | |
| SymSol ®PF3 | Water, Pentylene Glycol, | 1.5 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| MAA-1 | | 1.2 |
| MAA-12 | | 0.7 |
| Water, demineralized | Water (Aqua) | add 100 |

TABLE F15
Hair Refresher Mist

| Ingredients | INCI | Amount |
|---|---|---|
| SymDeo ® Plus | Lauryl alcohol, | 0.5 |
| | Phenoxyethanol, 2- | |
| | Benzyleptanol, Decylene | |
| | Glycol | |
| Perfume oil PO1, PO2, | Parfum | 0.1 |
| PO3, PO4, or PO5 | | |
| Frescolat ® ML nat | Mentyl Lactate | 0.3 |
| Crinipan ® PMC green | Propanediol Caprylate | 0.3 |
| Solubilizer | PEG-40 Hydrogenated Castor | 5.0 |
| | oil, Trideceth-9, Propylene | |
| | glycol, Aqua | |
| SymCalmin ® | Pentylene Glycol, Butylene | 0.2 |
| | Glycol, Hydroxyphenyl | |
| | Propamidobenzoic acid | |
| SymControl ® Scalp | Water (Aqua), Glycerin, | 2.0 |
| | Mannitol, Tetraselmis Suecica | |
| | Extract | |
| Genuvisco ® CG-131 | Carrageenan | 0.2 |
| Propylene Glycol | Propylene Glycol | 3.0 |
| Butylene Glycol | Butylene Glycol | 3.0 |
| MAA-13 | | 0.35 |
| Aqua | Aqua | add 100 |
| Sodium Gluconate | Sodium Gluconate | 0.2 |
| Caprylhydroxamic acid | Caprylhydroxamic acid | 0.01 |
| Sodium Benzoate | Sodium benzoate | 0.4 |
| Actipone ® Rosemary GW | Glycerin, Aqua, Rosmarinus | 0.5 |
| | officinalis leaf extract | |

TABLE F16
Anti-Wrinkle Night Emulsion

| Raw material | INCI | Amount |
|---|---|---|
| Emulsiphos ® F | Potassium cetyl phosphate | 2.5 |
| | Hydrogenated palm glycerides | |
| Dragoxat ® 89 | Ethylhexyl isononanoate | 2.0 |
| PCL-Liquid ®100 | Cetearyl Ethylhexanoate | 2.0 |
| SymRepair ® 100 | Hexyldecanol, Bisabolo, | 2.5 |
| | Cetylhydroxyproline Palmitade, | |
| | Stearic Acid, Brassica | |
| | Campestris (Rapeseed) Sterols | |
| Caprylic/Capric | Caprylic/Capric Triglyceride | 3.0 |
| Triglyceride | | |
| SymMollient ® S green | Cetearyl Nonanoate | 2.0 |
| Isoadipate | Diisopropyl Adipate | 2.0 |
| Sym Decanox ™HÁ | Caprylic/Capric Triglyceride, | 2.0 |
| | Hydroxymethdoxyphenyl | |
| | Decanone | |
| MAA-4 | | 0.2 |
| Alpha Tocopherol DL | Tocopherol | 0.5 |
| Argan Oil | Argania Spinosa Kernel Oil | 1.0 |
| Jojoba Oil | Simmondsia Chinensis Seed Oil | 1.0 |
| Avocado Oil CA | Persea Gratissima Oil | 1.0 |
| SymUrban ® | Benzylidene | 0.5 |
| | Dimethoxydimethylindanone | |
| Food Color Iron | CI77491 | 0.005 |
| Oxide RED E172 | | |
| Keltrol ® CG-BT | Xanthan Gum | 0.2 |
| Cosmedia ® SP | Sodium Polyacrylate | 0.5 |
| Aqua/Water | Aqua | add 100 |
| Glycerin | Glycerin | 3.0 |
| MAA-13 | | 1.0 |
| Edeta ® BD | Disodium EDTA | 0.1 |
| SymSave ® H | Hydroxyacetophenone | 0.5 |
| Sym Diol ® 68 | 1,2 Hexanediol, Caprylyl Glycol | \|0.5 |
| Actipone ® | Glycerin, Aqua, Laminaria | 1.0 |
| Laminaria Saccharina | Saccharina Extract | |
| GW CA | | |
| Macadamia Oil | Macadamia Integrifolia Seed Oil | 2.0 |
| SymGlucan ® | Aqua, Glycerin, 1,2-Hexanediol, | 2.0 |
| | Caprylyl Glycol, Beta-Glucan | |
| Hydrovition ® | Aqua, Pentylene Glycol, | 2.0 |
| PLUS 2290 | Glycerin, Fructose, Urea, Citric | |
| | Acid, Sodium hydroxide, Mtose, | |
| | Sodium PCA, Sodium Chloride, | |
| | Sodium Lactate, Trehalose, | |
| | Allantoin Sodium Hyaluronate, | |
| | Glucose | |
| Sym Vital ® AR | Zingiber Officinale (Ginger) Root | 0.2 |
| | Extract | |
| Ethanol 96% | Alcohol Denat. | 3.0 |
| Sodium hydroxide | Aqua, Sodium Hydroxide | 0.7 |
| 10% solution | | |
| Perfume oil PO1; PO2; | Parfum | 0.3 |
| PO3; PO4; or PO5 | | |

TABLE F17
Antibacterial body lotion, sprayable

| Ingredients | INCI | Amount |
|---|---|---|
| Potassium Sorbate | Potassium Sorbate | 0.2 |
| Dow Corning 345 Fluid | Cyclomethicone | 0.5 |
| Dracorin ® GOC | Glyceryl Oleate Citrate, | 2.0 |
| | Caprylic/Capric Triglyceride | |
| Drago-Calm | Water, Glycerin, Avena Sativa | 1.0 |
| | (Oat) Kernel Extract | |
| Dragosantol ® 100 | Bisabolol | 0.1 |
| Perfume oil PO1; | Fragrance | 0.3 |
| PO2; PO3; PO4 or PO5 | | |
| Hydrolite ®-5 | Pentylene Glycol | 5.0 |
| Neutral Oil | Caprylic/Capric Triglyceride | 4.0 |
| MAA-16 | | 0.2 |
| Paraffin Oil | Mineral Oil | 4.0 |
| PCL Liquid 100 | Cetearyl Ethylhexoate | 7.0 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% aqueous sol.) | | |
| SymDeo ® MPP | Dimethyl Phenylbutanol | 0.5 |
| SymRelief ® 100 | Bisabolol, Zingiber Officinale | 0.1 |
| | (Ginger) Root Extract | |
| MAA-13 | | 1.1 |
| SymSave ® H | 4-Hydroxyacetophenone | 0.2 |
| Water (demineralized) | Water (Aqua) | add 100 |

TABLE F18
Refreshing and Soothing After Sun Cream

| Ingredients | INCI | Amount |
|---|---|---|
| Dracorin ® CE | Glyceryl Stearate Citrate | 2.5 |
| Cutina ® GMS V | Glyceryl Stearate | 2.5 |
| Coconut Oil | Cocos Nucifera Oil | 3.0 |
| SymMollient ® PDCC | Propanediol Dicaprylate/ | 8.0 |
| | Caprate | |
| Plantoil Triglyceride | Caprylic/Capric Triglyceride | 4.0 |
| MAA-1 | | 1.5 |
| Tocopherol | Tocopherol | 0.05 |
| Demin. Water | Aqua | Add100 |
| Glycerin | Glycerin | 2.0 |
| SymOcide ® PS | Phenoxyethanol, Decylene | 1.0 |
| | Glycol, 1,2 Hexanediol | |
| Nativacare ® 8600 | Oryza Sativa (Rice) Starch | 1.0 |
| Keltrol ®CG-SFT | Xanthan Gum | 0.4 |
| Sirhamnose | Silanetriol (and) Rhamnose | 3.0 |
| D-Panthenol 75 L | Panthenol | 1.0 |
| Perfume oil PO1, PO2, | Parfum | 0.3 |
| PO3, PO4, or PO5 | | |

TABLE F19
Emulsion with low emulsifier content

| Ingredients | INCI | Amount |
|---|---|---|
| Water, demin. | Aqua | add 100 |
| Hydrolite ® 5 green | Pentylene Glycol | 3.0 |
| Sym Save ® H | Hydroxyacetophenone | 0.5 |
| Glycerin | Glycerin | 1.0 |
| Nativacare ™ 5600 | Zea Mays (Corn) Starch | 1.5 |
| SymMollient ® S green | Cetaryl Nonanoate | 3.0 |
| MAA-20 | | 2.0 |
| Lanette ® O | Cetearyl Alcohol | 2.5 |
| Plant oil Triglyceride | Caprylic/Capric Triglyceride | 4.0 |
| SymMollient ®PDCC | Propanediol Dicaprylate/ | 3.0 |
| | Caprate | |
| Dracorin ® CE | Glyceryl Stearate Citrate | 0.5 |
| Rheocare ® XGN | Xanthan Gum | 0.1 |
| Amigum | Sclerotium Gum | 0.25 |
| Citric Acid 10% solution | Aqua, Citric Acid | 0.1 |
| Perfume oil PO1, | Parfum | 0.3 |
| PO2, PO3, PO4, or PO5 | | |

TABLE F20
Natural Night Cream Aloe

| Ingredients | INCI | Amount |
|---|---|---|
| Deionised Water | Aqua | add 100 |
| MAA-20 | | 0.25 |
| MAA-13 | | 2.0 |
| Glycerin 99.5% | Glycerin | 2.0 |
| Dermofeel PA-3 | Sodium Phytate; Aqua; Alcohol | 0.1 |
| Dermosoft 1388 eco | Glycerin, Aqua, Sodium | 3.0 |
| | Levulinate, Sodium Anisate | |
| Veegum Ultra Granules | Magnesium Aluminium Silicate | 0.5 |
| Keltrol CG-RD | Xanthan Gum | 0.5 |
| Dermofeel SL | Sodium Stearoyl Lactylate | 2.0 |
| Dermofeel PS | Polyglyceryl-3 Stearate | 3.0 |
| Dermosoft decalact MB | Sodium Caproyl/Lauroyl Lactylate | 0.8 |
| Phytosqualan, veg. Grade | Squalane | 4.0 |
| Cegosoft SBE | Butyrosperum Parkii Butter; | 2.0 |
| | Tocopherol | |
| Cutina GMS | Glyceryl Stearate | 2.0 |
| Lanette O | Cetearyl Alcohol | 1.0 |
| Sunflower oil | Helianthus Annuus Seed Oil | 4.0 |
| Dermofeel sensolv | Isoamyl Laurate | 5.0 |
| Aloe Vera Gel | Aloe Barbadensis Leaf Juice | 0.3 |
| Concentrate 10/1 | | |
| Perfume oil PO1; | Parfum | 0.3 |
| PO2; PO3; PO4; or PO5 | | |

TABLE F21
Clear Anti Acne Wipe

| Ingredients | INCI | Amount |
|---|---|---|
| Farnesol | Farnesol | 0.2 |
| Methylisothiazolinone | Methylisothiazolinone | 0.1 |
| Isopropanol | Isopropanol | 45.0 |
| MAA-1 | | 2.5 |
| Water | Water (Aqua) | add 100 |
| Camomile CL | Chamomilla Recutita (Matricaria) | 1.0 |
| | Flower Water, Butylene Glycol, | |
| | Pentylene Glycol | |
| Citric Acid 10% | Citric Acid | 0.03 |
| aqueous solution | | |
| Perfume oil PO1; | Fragrance | 0.1 |
| PO2; PO3; PO4 or PO5 | | |
| Hydroxyethoxyphenyl | Hydroxyethoxyphenyl Butanone | 0.4 |
| Butanone | | |

TABLE F22
After-Shave Wipes

| Ingredients | INCI | Amount |
|---|---|---|
| Acrylates/C10-30 Alkyl | Acrylates/C10-30 Alkyl | 0.1 |
| Acrylate Crosspolymer | Acrylate Crosspolymer | |
| MAA-13 | | 2.8 |
| MAA-1 | | 0.4 |
| Water | Water (Aqua) | add 100 |
| Sodium Hydroxide 10% solution | Aqua, Sodium Hydroxide | 0.13 |
| Glycerin | Glycerin | 4.0 |
| DOWSIL ™ 2501 | Bis-PEG-18 Methyl Ether | 2.0 |
| Cosmetic Wax | Dimethyl Silane | |
| Aloe Vera | Aloe Vera | 0.6 |
| Propylene Glycol | Propylene Glycol | 0.3 |
| Witch Hazel Extract | Hamamelis Virginiana | 0.7 |
| | (Witch Hazel) Extract | |
| C12-15 Alkyl Benzoate | C12-15 Alkyl Benzoate | 2.7 |
| Polysorbate-20 | Polysorbate-20 | 0.5 |
| DOWSIL ™ 9040 Silicone | Cyclopentasiloxane (and) | 3.5 |
| Elastomer Blend | Dimethicone Crosspolymer | |
| XIAMETER ™ PMX-1184 | Dimethicone (and) | 5.8 |
| Silicone Fluid | Trisiloxane | |
| Perfume oil PO1; PO2; | Fragrance | 0.3 |
| PO3; PO4 or PO5 | | |
| DOWSIL ™ 9509 Silicone | Dimethicone/Vinyl | 2.0 |
| Elastomer Suspension | Dimethicone Crosspolymer | |
| | (and) C12-14 Pareth-12 | |
| Phytic Acid | Phytic Acid | 0.1 |
| Salicylic Acid | Salicylic Acid | 0.1 |

TABLE F23
Sensi-Scalp (Green Solid Shampoo)

| Ingredients | INCI | Amount |
|---|---|---|
| AMISOFT ® MS-11 | Sodium Myristoyl Glutamate | 30.0 |
| ELFAN ® AT 84 | Sodium cocoyl Isethionate | 15.0 |
| ImerCare ® 02K-S | Kaolin | 18.8 |
| Hydrolite ® 5 green | Pentylene Glycol | 1.5 |
| Dragosantol ® 100 | Bisabolol | 0.1 |
| MAA-9 | | 0.4 |
| Potassium Sorbate | Potassium Sorbate | 0.3 |
| Aqua/Water | Aqua | add 100 |
| MAA-28 | | 0.7 |
| Sym Decanox ™ HA | Caprylic/Capric Triglyceride, | 1.5 |
| | Hydroxymethoxyphenyl | |
| | decanone | |
| Alpha Tocopherol DL | Tocopherol | 0.5 |
| Cetiol ® SB45 | Butyrospermum parkii butter | 13.0 |
| Extrapone ® Rooibus GW | Aqua, Glycerin, Aspalathus | 1.0 |
| | Linearis leaf extract | |
| SymReboot ™ L19 | Maltodextrin, Lactobacillus | 0.5 |
| | Ferment | |
| SymOleo ® Vita7 | Glycine soja oil, Gossypium | 1.0 |
| | herbaceum seed oil, mangifera | |
| | idica seed butter, olea | |
| | europaea fruitoil, persea | |
| | gratissima oil, prununs | |
| | amygdalus dulcis (sweet | |
| | almond) oil, Theobroma cacao | |
| | seed butter | |
| Perfume oil PO1, PO2, | Parfum | 1.2 |
| PO3, PO4, or PO5 | | |

TABLE F24
Hygiene and Care Hand Cream

| Ingredients | INCI | Amount |
|---|---|---|
| Emulsiphos ® F | Potassium Cetyl Phosphate, | 1.5 |
| | Hydrogenated Palm Glycerides | |
| PCL-Solid | Stearyl Heptanoate, Stearyl | 2.5 |
| | Caprylate | |
| Dragoxat ® 89 | Ethylhexyl Isononanoate | 1.5 |
| Caprylic/Capric | Caprylic/Capric Triglyceride | 2.0 |
| Triglyceride | | |
| Tegosoft ® MM | Myristyl Myristate | 2.5 |
| Sym Decanox ® HÁ | Caprylic/Capric Triglyceride, | 1.0 |
| | Hydroxymethoxyphenyl decanone | |
| Frescolat ® X-Cool | Menthyl ethylamido oxalate | 0.5 |
| SymCare ® O | Hexyldecanol, Pentylene Glycol, | 2.0 |
| | 4-t-Butylcylohexanol, Bisabolol, | |
| | Cetylhydroxyproline Palmitamide, | |
| | Hydroxyphenyl Propamidobenzoic | |
| | acid, Stearic acid, Brassica | |
| | campestris (Rapeseed)Sterols, | |
| | Zingiber officinale (Ginger)Root | |
| | extract | |
| MAA-20 | | 0.5 |
| Beeswax | Beeswax | 1.0 |
| Cosmedia ® SP | Sodium Polyacrylate | 0.8 |
| Aristoflex ® AVC | Ammonium | 0.6 |
| | Acryloydimethyltaurate/VP | |
| | Copolymer | |
| Aqua/Water | Aqua | add 100 |
| MAA-13 | | 1.5 |
| Neutrol ® MGDA | Trisodium Dicarboymethyl | 0.2 |
| | Alaninate | |
| SymSave ® H | Hydroxyacetophenone | 0.5 |
| Hydroviton ® | Aqua, Pentylene Glycol, Glycerin, | 2.0 |
| PLUS 2290 | Fructose, Urea, Citric acid, | |
| | Sodium hydroxide, Maltose, | |
| | Sodium PCA, Sodium chloride, | |
| | Sodium Lactate, Trehalose, | |
| | Allantoin, Sodium Hyaluronate, | |
| | Glucose | |
| Actipone Stinging | Glycerin, Urtica Dioica Juice, | 1.0 |
| Nettle Juice | Aqua | |
| SymEssence ® | Chamomilla Recuitta (Matricaria), | 1.0 |
| Camomile CC | Flower/Leaf/Stem Water | |
| Colour | | 0.9 |
| Alpha Tocopherol DL | Tocopherol | 0.5 |
| Perfume oil P1, P2, | Parfum | 0.4 |
| P3, P4 or P5 | | |
| SymOcide ® C | o-cymen-5-ol | 0.1 |
| SymDeo ® Plus | Lauryl alcohol, Phenoxyethanol, | 0.5 |
| | 2-Benzylheptanol, Decylene | |
| | Glycol | |

TABLE F25
Mouthwash without alcohol (ready to use)

| Ingredients | INCI | Amount |
|---|---|---|
| Cremophor ® CO40 | PEG_40 Hydrogenated castor oil | 2.5 |
| SymGuard ® CD | Phenylpropanol, o-cymen-5-ol, | 0.5 |
| | Decylene Glycol | |
| Propylene Glycol | Propylene Glycol | 5.0 |
| Flavour/Aroma 1 A1 | Aroma | 0.2 |
| Aqua | Aqua | add 100 |
| MAA-13 | | 0.35 |
| Sorbitol Liq. EP 70% | Sorbitol | 10.0 |
| Sodium Saccharin | Sodium Saccharin | 0.07 |
| Sodium Fluorid | Sodium Fluorid | 0.18 |
| SymSave ® H | Hydroxyacetophenone | 0.3 |

TABLE F26
Sunscreen lotion (o/w; broadband protection)

| Ingredients | INCI | Amount |
|---|---|---|
| Hydrolite-7 | 1,2-heptanediol | 0.6 |
| MAA-20 | | 1.5 |
| 1,2-octanediol | 1,2-octanediol | 0.2 |
| 2,3-octanediol | 2,3-octanediol | 0.01 |
| Benzyl Salicylate | Benzyl Salicylate | 0.5 |
| Carbopol Ultrez-10 | Carbomer | 0.2 |
| Dow Corning 246 Fluid | Cyclohexasiloxane and | 2.0 |
| | Cyclopentasiloxane | |
| Dragosantol ® 100* | Bisabolol | 0.3 |
| EDETA BD | Disodium EDTA | 0.1 |
| Emulsiphos ® F | Potassium Cetyl Phosphate, | 1.5 |
| | Hydrogenated Palm Glycerides | |
| Perfume oil PO1, PO2, | Perfume | 0.4 |
| PO3, PO4 or PO5 | | |
| Frescolat ® MGA | Menthone Glycerol Acetal | 0.3 |
| Glycerol 85% | Glycerin | 4.7 |
| Keltrol RD | Xanthan Gum | 0.2 |
| Lanette O | Cetearyl Alcohol | 1.0 |
| Neo Heliopan ® 357 | Butyl Methoxy-dibenzoyl-methane | 1.0 |
| Neo Heliopan ® AP | Disodium Phenyl Dibenzimidazole | 10.0 |
| (10% as sodium salt) | Tetrasulfonate | |
| Neo Heliopan ® AV | Ethylhexyl Methoxy-cinnamate | 3.0 |
| Neo Heliopan ® Hydro | Phenylbenz-imidazole Sulfonic | 6.7 |
| (15% as sodium salt) | Acid | |
| Neo Heliopan ® MBC | 4-Methylbenzyl-idene Camphor | 1.5 |
| Neo Heliopan ® OS | Ethylhexyl Salicylate | 5.0 |
| Neutral Oil | Caprylic/Capric Triglyceride | 2.0 |
| SymMatrix ® | Maltodextrin, Rubus Fruticosus | 0.3 |
| | (Blackberry) Leaf Extract | |
| Methylisothiazolinone | Methylisothiazolinone | 0.1 |
| SymOcide ® BHO | Hydroxyacetophenone, Benzyl | 0.5 |
| | alcohol, Caprylyl glycol, Aqua | |
| Tegosoft TN | C12-15 Alkyl Benzoate | 5.0 |
| Tocopherol Acetate | Tocopheryl Acetate | 0.5 |
| Triethanolamine, 99% | Triethanolamine | 0.5 |
| Water (demineralized) | Water (Aqua) | add 100 |

TABLE F27
Sun protection soft cream (w/o), SPF 40

| Ingredients | INCI | Amount |
|---|---|---|
| Dehymuls PGPH | Polyglyceryl-2 dipolyhydroxystearate | 5.0 |
| Copherol 1250 | Tocopheryl acetate | 0.5 |
| Permulgin 3220 | Ozocerite | 0.5 |
| Zinc stearate | Zinc stearate | 0.5 |
| Tegosoft TN | C12-15 Alkyl benzoate | 10.0 |
| Neo Heliopan ® E1000 | Isoamyl-p-methoxycinnamate | 2.0 |
| Neo Heliopan ® 303 | Octocrylene | 5.0 |
| Neo Heliopan ® MBC | 4-Methylbenzylidene camphor | 3.0 |
| Zinc oxide, neutral | Zinc oxide | 5.0 |
| MAA-28 | | 1.0 |
| Water, distilled | Water (aqua) | add 100 |
| EDETA BD | Disodium EDTA | 0.1 |
| Glycerol | Glycerol | 4.0 |
| Magnesium sulfate | Magnesium sulfate | 0.5 |
| Perfume oil PO1, PO2, | Perfume | 0.3 |
| PO3, PO4 or PO5 | | |
| Symdiol ® 68 | Hexanediol, Caprylylglycol | 0.5 |
| 1,2-heptanediol | 1,2-heptanediol | 0.2 |
| 2,3-heptanediol | 2,3-heptanediol | 0.01 |
| 1,2-octanediol | 1,2-octanediol | 0.2 |
| 2,3-octanediol | 2,3-octanediol | 0.01 |

### Homecare Products (Compositions)

All purpose cleaner
APC alkaline
Detergent liquid light duty
Dishwash liquid manual.
Fabric softener
Fabric softener concentrate, encapsulated
Hand soap, liquid
Scent Lotion, encapsulated.
Cleaner, liquid, citric acid

TABLE F28
All purpose cleaner

| No | Ingredients | Amount |
|---|---|---|
| 1 | Fettalkoholethoxylat | 4 |
| 2 | Sulfonic acid, C 13-17-sec-Alkyl-, | 6.2 |
| | Sodium salt | |
| 3 | 2-octyl-2H-isothiazol-3-one | 0.1 |
| 4 | Fragrance | 3.5 |
| 5 | MAA-13 | 1.0 |
| 6 | 1,2-heptanediol | 0.9 |
| 7 | Aqua | add 100 |

Colorless liquid, pH 7

TABLE F29
APC alkaline

| No | Ingredients | Amount |
|---|---|---|
| 1 | Coco-Glycoside | 1 |
| 2 | Benzalkonium Chloride | 0.2 |
| 3 | Sodiumbicarbonat | 0.1 |
| 4 | Tri-sodiumcitrate-dihydrate | 0.1 |
| 5 | Methylglycinediacetic acid | 0.1 |
| 6 | 1,2-Benzisothiazol-3(2H)-on | 0.1 |
| 7 | Fragrance | 0.1 |
| 8 | 1,2-heptanediol | 0.5 |
| 9 | MAA-2 | 0.5 |
| 10 | Hydroxyacetophenone | 0.3 |
| 11 | Aqua | add 100 |

Colorless liquid, pH 9

TABLE F30
Detergent liquid light duty

| No | Ingredients | Amount |
|---|---|---|
| 1 | Cocos fatty acid | 0.85 |
| 2 | potassium hydroxide | 0.45 |
| 3 | 2-octyl-2H-isothiazol-3-one | 0.2 |
| 4 | Sulfonic acid, C 13-17-sec-Alkyl-, | 20 |
| | Sodium salt | |
| 5 | Sodium Laureth Sulfate | 3 |
| 6 | Trideceth-9 | 5 |
| 7 | Sodium Chloride | 1.3 |
| 8 | MAA-13 | 0.1 |
| 9 | 1,2-heptanediol | 0.9 |
| 10 | Fragrance | 0.5 |
| 11 | Aqua | add 100 |
| 12 | Hydroxyacetophenone | 0.2 |

Liquid, pH 7.3

TABLE F31
Dishwash liquid manual

| No | Ingredients | Amount |
|---|---|---|
| 1 | Sodium Laureth Sulfate | add 100 |
| 2 | Ethanol | 2.9 |
| 3 | lauramine oxide | 7.7 |
| 4 | propylene glycol | 1.9 |
| 5 | Phenoxyethanol | 0.1 |
| 6 | Benzisothiazolinone, Methylisothiazolinone, | 0.1 |
| | Laurylamine Dipropylenediamine | |
| 7 | 1,2-heptanediol | 0.5 |
| 8 | sodium chloride | 3.85 |
| 9 | Fragrance | 0.3 |
| 10 | Benzyl Alcohol | 0.2 |
| 11 | MAA-20 | 1.0 |

Colorless liquid, pH 8.5

TABLE F32
Fabric softener

| No | Ingredients | Amount |
|---|---|---|
| 1 | Di-(Talg Carboxyethyl) Hydroxyethyl | 5.5 |
| | methylammonium-methosulfat | |
| 2 | Simethicone | 0.3 |
| 3 | 2-octyl-2H-isothiazol-3-one | 0.1 |
| 4 | 1,2-heptanediol | 0.5 |
| 5 | Fragrance | 0.2 |
| 6 | Aqua | add 100 |
| 7 | Phenoxyethanol | 0.2 |
| 8 | MAA-28 | 0.5 |

Liquid, pH 3

TABLE F33
Fabric softener concentrate, encapsulated

| No | Ingredients | Amount |
|---|---|---|
| 1 | Di-(Talg Carboxyethyl) Hydroxyethyl | 16.6 |
| | methylammonium-methosulfat | |
| 2 | 2-octyl-2H-isothiazol-3-one | 0.1 |
| 3 | Simethicone | 0.3 |
| 4 | magnesium chloride | 0.8 |
| 5 | encapsulated perfume oil | 0.3 |
| 6 | crosslinked cationic polymer | 0.15 |
| 7 | MAA-13 | 0.8 |
| 8 | 1,2-heptanediol | 0.5 |
| 9 | Fragrance | 0.6 |
| 10 | Aqua | add 100 |
| 11 | Glyceryl Caprylate | 0.1 |

White liquid, pH 2.5

TABLE F34
Hand soap, liquid

| No | Ingredients | Amount |
|---|---|---|
| 1 | Sodium Laureth Sulfate | 18 |
| 2 | Cocoamidopropylbetaine | 6 |
| 3 | Cocamide DEA | 3 |
| 4 | citric acid | 0.5 |
| 5 | sodium chloride | 1.9 |
| 6 | Glycerol | 2 |
| 7 | Fragrance | 0.2 |
| 8 | MAA-13 | 0.25 |
| 9 | Aqua | add 100 |
| 10 | Propanediol Caprylate | 0.3 |

Slightly colored liquid, pH 6

TABLE F35
Scent Lotion with capsules

| No | Ingredients | Amount |
|---|---|---|
| 1 | microcrystalline cellulose/cellulose gum | 1 |
| 2 | PEG-40 hydrogenated Castor oil/Trideceth-9 | 0.5 |
| 3 | encapsulated perfume oil | 1 |
| 4 | 2-octyl-2H-isothiazol-3-one | 0.1 |
| 5 | Fragrance | 5 |
| 6 | 1,2-heptanediol | 1 |
| 7 | Aqua | add 100 |
| 8 | MAA-12 | 0.4 |

White emulsion, PH neutral

TABLE F36
Cleaner, liquid, citric acid

| No | Ingredients | Amount |
|---|---|---|
| 1 | Sulfonic acid, C 13-17-sec-Alkyl-, | 1 |
| | Sodium salt | |
| 2 | Xanthan gum | 0-3 |
| 3 | citric acid | 5 |
| 4 | 1,2heptanediol | 0.25 |
| 5 | Fragrance | 0.3 |
| 6 | Aqua | add 100 |
| 7 | Anisic Acid | 0.1 |
| 8 | MAA-13 | 0.5 |

Liquid, pH 2

1. A food, cosmetic or pharmaceutical preparation or homecare product, comprising
(a) at least one fragrance or aroma/flavour compound;
(b) at least one mycosporine-like amino acid compound,
represented either by the general formula (V) 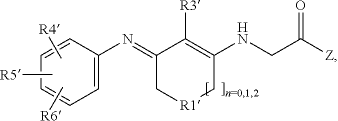
wherein
Z is selected from the group consisting of O—R2′ or amide′;
R1′ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), CR7′R8′, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), and unsubstituted or substituted N(heteroaryl), and unsubstituted or substituted C-spirocycles;
R2′ is selected from the group consisting of H, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted cycloalkyl, unsubstituted or substituted aryl, unsubstituted or substituted heterocycloalkyl, and unsubstituted or substituted heteroaryl;
amide′ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), and unsubstituted or substituted NR9′R10′, unsubstituted or substituted-N(O-alkyl)(alkl) (Weinreb amide), —N(OH)(H) (hydroxamate), and unsubstituted or substituted-N(OH)(alkyl) (alkylated hydroxamate);
R3′ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9′R10′, and unsubstituted or substituted C-spirocycles;
the substituents R4′, R5′ and R6′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9′R10′, and unsubstituted or substituted C-spirocycles;
R7′ and R8′ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9′R10′;
R9′ und R10′ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocycloalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or represented by the general formula (VI) 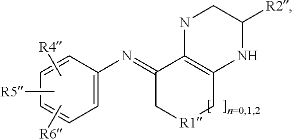
wherein
R1″ is selected from the group consisting of CH₂, unsubstituted or substituted CH(alkyl), unsubstituted or substituted C(alkyl)₂, unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, halogen, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
R2″ is selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —C(═O)-amide″, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, and unsubstituted or substituted C-spirocycles;
X is selected from the group consisting of CH₂, unsubstituted or substituted CH(alkyl), unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles;
the substituents R4″, R5″ and R6″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, —S(═O)₂OH, sulfonyl, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), and unsubstituted or substituted NH(heteroaryl), unsubstituted or substituted NR9″R10″, and unsubstituted or substituted C-spirocycles;
R7″ and R8″ which may be identical or different, are independently from each other selected from the group consisting of H, OH, halogen, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, —CH₂—OH, —R—CH₂—OH, —C(═O)—O—Y, —R—C(═O)—O—Y, unsubstituted or substituted cycloalkyl, unsubstituted or substituted aryl, unsubstituted or substituted heterocycloalkyl, unsubstituted or substituted heteroaryl, SO, SO₂, NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl) and unsubstituted or substituted NR9″R10″;
amide″ is selected from the group consisting of NH₂, unsubstituted or substituted NH(alkyl), unsubstituted or substituted NH(alkenyl), unsubstituted or substituted NH(alkynyl), unsubstituted or substituted NH(alkoxy), unsubstituted or substituted NH(alkylthio), unsubstituted or substituted NH(cycloalkyl), unsubstituted or substituted NH(aryl), unsubstituted or substituted NH(heterocycloalkyl), unsubstituted or substituted NH(heteroaryl), unsubstituted or substituted NR9″R10″, unsubstituted or substituted-N(O-alkyl)(alkyl) (Weinreb amide), —N(OH)(H) (hydroxamate), and unsubstituted or substituted-N(OH)(alkyl) (alkylated hydroxamate);
R9″ und R10″ which may be identical or different, are independently from each other selected from the group consisting of H, OH, unsubstituted or substituted alkyl, unsubstituted or substituted alkenyl, unsubstituted or substituted alkynyl, unsubstituted or substituted alkoxy, unsubstituted or substituted alkylthio, unsubstituted or substituted alkanoyl/acyl R—C(═O)—, unsubstituted or substituted cycloalkyl, unsubstituted or substituted aryl, unsubstituted or substituted heterocylcoalkyl, and unsubstituted or substituted heteroaryl;
Y is selected from the group consisting of H and unsubstituted or substituted alkyl; and
R is unsubstituted or substituted alkyl;
or a tautomer or a stereoisomer or a salt of the afore-mentioned compounds;
or any mixture of the afore-mentioned compounds; and
(c) optionally at least one antioxidant;
with the proviso, that the following compounds are disclaimed/excluded:

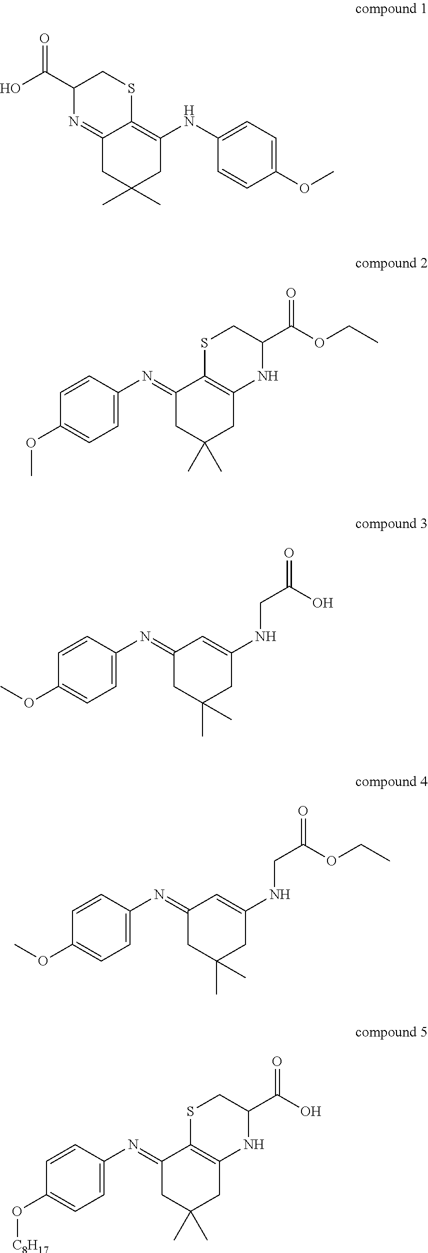

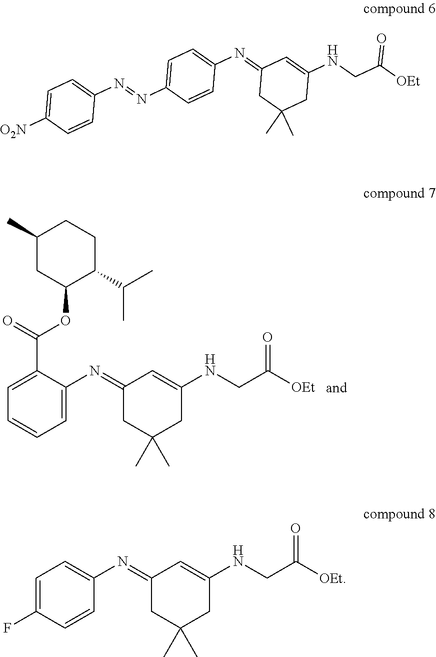 [SMILES: CCOC(=O)CNC1=C/C(=N/c2ccc(/N=N/c3ccc([N+](=O)[O-])cc3)cc2)CC(C)(C)C1 ; CCOC(=O)CNC1=C/C(=N/c2ccccc2C(=O)O[C@H]2C[C@@H](C)CC[C@@H]2C(C)C)CC(C)(C)C1 ; "and" ; CCOC(=O)CNC1=C/C(=N/c2ccc(F)cc2)CC(C)(C)C1]

2. The food, cosmetic or pharmaceutical preparation or homecare product according to claim 1, wherein the mycosporine-like amino acid compound is represented either by the general formula (VII) 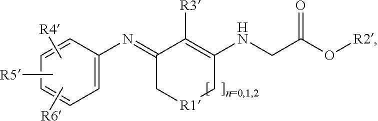
wherein R1′, R2′, R3′, R4′, R5′ and R6′ have the same meaning as defined for formula (V);
or by the general formula (VIII) 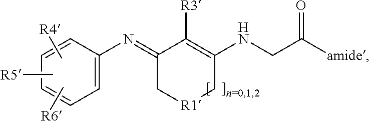
wherein R1′, amide′, R3′, R4′, R5′ and R6′ have the same meaning as defined for formula (V).

3. The food, cosmetic or pharmaceutical preparation or homecare product according to claim 1, wherein in the general formula (VI) X is selected from the group consisting of CH₂, unsubstituted or substituted CH(alkyl), unsubstituted or substituted C(alkyl)₂, unsubstituted or substituted CH(alkenyl), unsubstituted or substituted CH(alkynyl), unsubstituted or substituted CH(alkoxy), unsubstituted or substituted CH(alkylthio), unsubstituted or substituted CH(cycloalkyl), unsubstituted or substituted CH(aryl), unsubstituted or substituted CH(heterocycloalkyl), unsubstituted or substituted CH(heteroaryl), —CR7″R8″, C═O, S, SO, SO₂, NH, unsubstituted or substituted N(alkyl), unsubstituted or substituted N(alkenyl), unsubstituted or substituted N(alkynyl), unsubstituted or substituted N(alkoxy), unsubstituted or substituted N(alkylthio), unsubstituted or substituted N(cycloalkyl), unsubstituted or substituted N(aryl), unsubstituted or substituted N(heterocycloalkyl), unsubstituted or substituted N(heteroaryl), and unsubstituted or substituted C-spirocycles

4. The food, cosmetic or pharmaceutical preparation or homecare product according to claim 1, wherein in the general formula (V)
Z is —O—R2′ or amide′; and/or
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 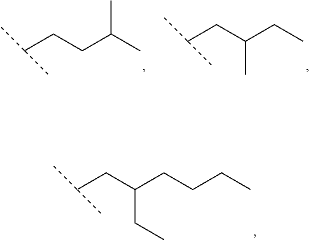
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, C(alkyl)₂, O, S, SO, SO₂, NH and N(alkyl); and/or
R2′ is selected from the group consisting of H, methyl, ethyl, propyl, isopropyl, butyl, isobutyl, tert-butyl, 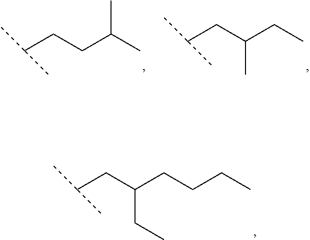
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl;
or wherein in the general formula (VI)
R1″ is selected from the group consisting of CH₂, C(alkyl)₂, O, S, SO, SO₂, NH and N(alkyl); and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl, 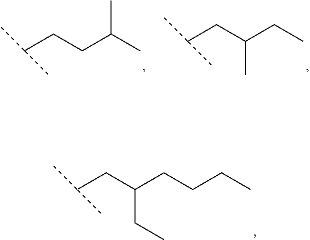
(2-ethylhexyl) and phenyl; and/or
amide″ is selected from the group consisting of NH₂, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

5. The food, cosmetic or pharmaceutical preparation or homecare product according to claim 1, wherein in the general formula (V)
Z is —O—R2′ or amide′; and/or
R1′ is selected from the group consisting of CH₂ and C(methyl)₂; and/or
R2′ is selected from the group consisting of H, methyl, ethyl, propyl, isopropyl, butyl, isobutyl, tert-butyl, 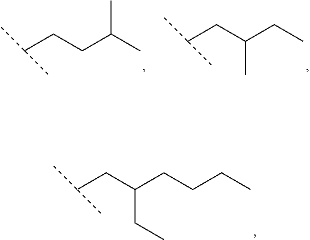
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formula (V);
wherein alkyl is selected from the group consisting of methyl, ethyl, propyl, isopropyl, butyl, isobutyl, and tert-butyl;
or wherein in the general formulae (VII) or (VIII)
R1′ is selected from the group consisting of CH₂, and C(methyl) 2;
R2′ is selected from the group consisting of H, methyl, ethyl, propyl, isopropyl, butyl, isobutyl, tert-butyl, 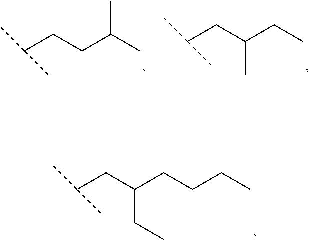
(2-ethylhexyl) and phenyl; or
amide′ is selected from the group consisting of NH₂, NH(alkyl) and N(alkyl)₂, —N(O-alkyl)(alkyl) (Weinreb amide) and —N(OH)(H) (hydroxamate); and/or
R3′ is selected from the group consisting of H, methyl, ethyl, —O-methyl, —C(═O)—OH, —C(═O)—CH₃, —C(═O)—C₃H₇ and —C(═O)—O-methyl; and/or
the substituents R4′, R5′ and R6′ have the same meaning as defined for formulae (VII) and (VIII);
wherein alkyl is selected from the group consisting of methyl, ethyl, propyl, isopropyl, butyl, isobutyl, and tert-butyl; and/or
or wherein in the general formula (VI)
R1″ is selected from the group consisting of CH₂ and C(methyl)₂; and/or
R2″ is selected from the group consisting of —CH₂—OH, —C(═O)—O—Y, and —C(═O)-amide″; and/or
X is selected from the group consisting of CH₂, C═O, O, S, SO, SO₂, NH, and N(alkyl); and/or
Y is selected from the group consisting of H, methyl, ethyl, propyl, isopropyl, butyl, isobutyl, tert-butyl, 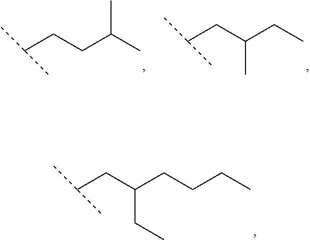
(2-ethylhexyl) and phenyl; and/or
amide″ is selected from the group consisting of NH₂, NH(alkyl) and N(alkyl)₂, —N(O-alkyl)(alkyl) (Weinreb amide) and —N(OH)(H) (hydroxamate); and/or
the substituents R4″, R5″ and R6″ have the same meaning as defined for formula (VI);
wherein alkyl is selected from the group consisting of methyl, ethyl, propyl, isopropyl, butyl, isobutyl, and tert-butyl.

6. The food, cosmetic or pharmaceutical preparation or homecare product according to claim 1, wherein the mycosporine-like amino acid compound is selected from the group consisting of the following compounds I-1 to I-128 according to the general formula (V) and II-1 to II-192 according to the general formula (VI):

| 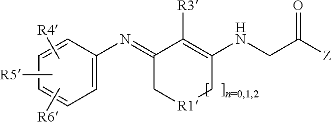 | R1′ | R3′ | Z |
|---|---|---|---|
| I-1 | CH2 | H | —O-R2′ |
| I-2 | CH2 | methyl | —O-R2′ |
| I-3 | CH2 | ethyl | —O-R2′ |
| I-4 | CH2 | —O-methyl | —O-R2′ |
| I-5 | CH2 | —C(═O)-OH | —O-R2′ |
| I-6 | CH2 | —C(═O)-CH3 | —O-R2′ |
| I-7 | CH2 | —C(═O)-C3H7 | —O-R2′ |
| I-8 | CH2 | —C(═O)-O-methyl | —O-R2′ |
| I-9 | CH2 | H | amide′ |
| I-10 | CH2 | methyl | amide′ |
| I-11 | CH2 | ethyl | amide′ |
| I-12 | CH2 | —O-methyl | amide′ |
| I-13 | CH2 | —C(═O)-OH | amide′ |
| I-14 | CH2 | —C(═O)-CH3 | amide′ |
| I-15 | CH2 | —C(═O)-C3H7 | amide′ |
| I-16 | CH2 | —C(═O)-O-methyl | amide′ |
| I-17 | C(methyl)2 | H | —O-R2′ |
| I-18 | C(methyl)2 | methyl | —O-R2′ |
| I-19 | C(methyl)2 | ethyl | —O-R2′ |
| I-20 | C(methyl)2 | —O-methyl | —O-R2′ |
| I-21 | C(methyl)2 | —C(═O)-OH | —O-R2′ |
| I-22 | C(methyl)2 | —C(═O)-CH3 | —O-R2′ |
| I-23 | C(methyl)2 | —C(═O)-C3H7 | —O-R2′ |
| I-24 | C(methyl)2 | —C(═O)-O-methyl | —O-R2′ |
| I-25 | C(methyl)2 | H | amide′ |
| I-26 | C(methyl)2 | methyl | amide′ |
| I-27 | C(methyl)2 | ethyl | amide′ |
| I-28 | C(methyl)2 | —O-methyl | amide′ |
| I-29 | C(methy1)2 | —C(═O)-OH | amide′ |
| I-30 | C(methyl)2 | —C(═O)-CH3 | amide′ |
| I-31 | C(methyl)2 | —C(═O)-C3H7 | amide′ |
| I-32 | C(methyl)2 | —C(═O)-O-methyl | amide′ |
| I-33 | O | H | —O-R2′ |
| I-34 | O | methyl | —O-R2′ |
| I-35 | O | ethyl | —O-R2′ |
| I-36 | O | —O-methyl | —O-R2′ |
| I-37 | O | —C(═O)-OH | —O-R2′ |
| I-38 | O | —C(═O)-CH3 | —O-R2′ |
| I-39 | O | —C(═O)-C3H7 | —O-R2′ |
| I-40 | O | —C(═O)-O-methyl | —O-R2′ |
| I-41 | O | H | amide′ |
| I-42 | O | methyl | amide′ |
| I-43 | O | ethyl | amide′ |
| I-44 | O | —O-methyl | amide′ |
| I-45 | O | —C(═O)-OH | amide′ |
| I-46 | O | —C(═O)-CH3 | amide′ |
| I-47 | O | —C(═O)-C3H7 | amide′ |
| I-48 | O | —C(═O)-O-methyl | amide′ |
| I-49 | S | H | —O-R2′ |
| I-50 | S | methyl | —O-R2′ |
| I-51 | S | ethyl | —O-R2′ |
| I-52 | S | —O-methyl | —O-R2′ |
| I-53 | S | —C(═O)-OH | —O-R2′ |
| I-54 | S | —C(═O)-CH3 | —O-R2′ |
| I-55 | S | —C(═O)-C3H7 | —O-R2′ |
| I-56 | S | —C(═O)-O-methyl | —O-R2′ |
| I-57 | S | H | amide′ |
| I-58 | S | methyl | amide′ |
| I-59 | S | ethyl | amide′ |
| 60 | S | —O-methyl | amide′ |
| I-61 | S | —C(═O)-OH | amide′ |
| I-62 | S | —C(═O)-CH3 | amide′ |
| I-63 | S | —C(═O)-C3H7 | amide′ |
| I-64 | S | —C(═O)-O-methyl | amide′ |
| I-65 | SO | H | —O-R2′ |
| I-66 | SO | methyl | —O-R2′ |
| I-67 | SO | ethyl | —O-R2′ |
| I-68 | SO | —O-methyl | —O-R2′ |
| I-69 | SO | —C(═O)-OH | —O-R2′ |
| I-70 | SO | —C(═O)-CH3 | —O-R2′ |
| I-71 | SO | —C(═O)-C3H7 | —O-R2′ |
| I-72 | SO | —C(═O)-O-methyl | —O-R2′ |
| I-73 | SO | H | amide′ |
| I-74 | SO | methyl | amide′ |
| I-75 | SO | ethyl | amide′ |
| I-76 | SO | —O-methyl | amide′ |
| I-77 | SO | —C(═O)-OH | amide′ |
| I-78 | SO | —C(═O)-CH3 | amide′ |
| I-79 | SO | —C(═O)-C3H7 | amide′ |
| I-80 | SO | —C(═O)-O-methyl | amide′ |
| I-81 | SO2 | H | —O-R2′ |
| I-82 | SO2 | methyl | —O-R2′ |
| I-83 | SO2 | ethyl | —O-R2′ |
| I-84 | SO2 | —O-methyl | —O-R2′ |
| I-85 | SO2 | —C(═O)-OH | —O-R2′ |
| I-86 | SO2 | —C(═O)-CH3 | —O-R2′ |
| I-87 | SO2 | —C(═O)-C3H7 | —O-R2′ |
| I-88 | SO2 | —C(═O)-O-methyl | —O-R2′ |
| I-89 | SO2 | H | amide′ |
| I-90 | SO2 | methyl | amide′ |
| I-91 | SO2 | ethyl | amide′ |
| I-92 | SO2 | —O-methyl | amide′ |
| I-93 | SO2 | —C(═O)-OH | amide′ |
| I-94 | SO2 | —C(═O)-CH3 | amide′ |
| I-95 | SO2 | —C(═O)-C3H7 | amide′ |
| I-96 | SO2 | —C(═O)-O-methyl | amide′ |
| I-97 | NH | H | —O-R2′ |
| I-98 | NH | methyl | —O-R2′ |
| I-99 | NH | ethyl | —O-R2′ |
| I-100 | NH | —O-methyl | —O-R2′ |
| I-101 | NH | —C(═O)-OH | —O-R2′ |
| I-102 | NH | —C(═O)-CH3 | —O-R2′ |
| I-103 | NH | —C(═O)-C3H7 | —O-R2′ |
| I-104 | NH | —C(═O)-O-methyl | —O-R2′ |
| I-105 | NH | H | amide′ |
| I-106 | NH | methyl | amide′ |
| I-107 | NH | ethyl | amide′ |
| I-108 | NH | —O-methyl | amide′ |
| I-109 | NH | —C(═O)-OH | amide′ |
| I-110 | NH | —C(═O)-CH3 | amide′ |
| I-111 | NH | —C(═O)-C3H7 | amide′ |
| I-112 | NH | —C(═O)-O-methyl | amide′ |
| I-113 | N(alkyl) | H | —O-R2′ |
| I-114 | N(alkyl) | methyl | —O-R2′ |
| I-115 | N(alkyl) | ethyl | —O-R2′ |
| I-116 | N(alkyl) | —O-methyl | —O-R2′ |
| I-117 | N(alkyl) | —C(═O)-OH | —O-R2′ |
| I-118 | N(alkyl) | —C(═O)-CH3 | —O-R2′ |
| I-119 | N(alkyl) | —C(═O)-C3H7 | —O-R2′ |
| I-120 | N(alkyl) | —C(═O)-O-methyl | —O-R2′ |
| I-121 | N(alkyl) | H | amide′ |
| I-122 | N(alkyl) | methyl | amide′ |
| I-123 | N(alkyl) | ethyl | amide′ |
| I-124 | N(alkyl) | —O-methyl | amide′ |
| I-125 | N(alkyl) | —C(═O)-OH | amide′ |
| I-126 | N(alkyl) | —C(═O)-CH3 | amide′ |
| I-127 | N(alkyl) | —C(═O)-C3H7 | amide′ |
| I-128 | N(alkyl) | —C(═O)-O-methyl | amide′ |
| 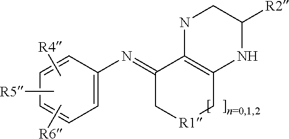 | R1″ | X | R2″ |
| II-1 | CH2 | CH2 | —CH2-OH |
| II-2 | CH2 | C═O | —CH2-OH |
| II-3 | CH2 | O | —CH2-OH |
| II-4 | CH2 | S | —CH2-OH |
| II-5 | CH2 | SO | —CH2-OH |
| II-6 | CH2 | SO2 | —CH2-OH |
| II-7 | CH2 | NH | —CH2-OH |
| II-8 | CH2 | N(alkyl) | —CH2-OH |
| II-9 | CH2 | CH2 | —C(═O)-O-Y |
| II-10 | CH2 | C═O | —C(═O)-O-Y |
| II-11 | CH2 | O | —C(═O)-O-Y |
| II-12 | CH2 | S | —C(═O)-O-Y |
| II-13 | CH2 | SO | —C(═O)-O-Y |
| II-14 | CH2 | SO2 | —C(═O)-O-Y |
| II-15 | CH2 | NH | —C(═O)-O-Y |
| II-16 | CH2 | N(alkyl) | —C(═O)-O-Y |
| II-17 | CH2 | CH2 | —C(═O)-amide″ |
| II-18 | CH2 | C═O | —C(═O)-amide″ |
| II-19 | CH2 | O | —C(═O)-amide″ |
| II-20 | CH2 | S | —C(═O)-amide″ |
| II-21 | CH2 | SO | —C(═O)-amide″ |
| II-22 | CH2 | SO2 | —C(═O)-amide″ |
| II-23 | CH2 | NH | —C(═O)-amide″ |
| II-24 | CH2 | N(alkyl) | —C(═O)-amide″ |
| II-25 | C(methyl)2 | CH2 | —CH2-OH |
| II-26 | C(methyl)2 | C═O | —CH2-OH |
| II-27 | C(methyl)2 | O | —CH2-OH |
| II-28 | C(methyl)2 | S | —CH2-OH |
| II-29 | C(methyl)2 | SO | —CH2-OH |
| II-30 | C(methy1)2 | SO2 | —CH2-OH |
| II-31 | C(methyl)2 | NH | —CH2-OH |
| II-32 | C(methyl)2 | N(alkyl) | —CH2-OH |
| II-33 | C(methyl)2 | CH2 | —C(═O)-O-Y |
| II-34 | C(methyl)2 | C═O | —C(═O)-O-Y |
| II-35 | C(methyl)2 | O | —C(═O)-O-Y |
| II-36 | C(methyl)2 | S | —C(═O)-O-Y |
| II-37 | C(methyl)2 | SO | —C(═O)-O-Y |
| II-38 | C(methyl)2 | SO2 | —C(═O)-O-Y |
| II-39 | C(methyl)2 | NH | —C(═O)-O-Y |
| II-40 | C(methyl)2 | N(alkyl) | —C(═O)-O-Y |
| II-41 | C(methyl)2 | CH2 | —C(═O)-amide″ |
| II-42 | C(methyl)2 | C═O | —C(═O)-amide″ |
| II-43 | C(methyl)2 | O | —C(═O)-amide″ |
| II-44 | C(methyl)2 | S | —C(═O)-amide″ |
| II-45 | C(methy1)2 | SO | —C(═O)-amide″ |
| II-46 | C(methyl)2 | SO2 | —C(═O)-amide″ |
| II-47 | C(methyl)2 | NH | —C(═O)-amide″ |
| II-48 | C(methyl)2 | N(alkyl) | —C(═O)-amide″ |
| II-49 | O | CH2 | —CH2-OH |
| II-50 | O | C═O | —CH2-OH |
| II-51 | O | O | —CH2-OH |
| II-52 | O | S | —CH2-OH |
| II-53 | O | SO | —CH2-OH |
| II-54 | O | SO2 | —CH2-OH |
| II-55 | O | NH | —CH2-OH |
| II-56 | O | N(alkyl) | —CH2-OH |
| II-57 | O | CH2 | —C(═O)-O-Y |
| II-58 | O | C═O | —C(═O)-O-Y |
| II-59 | O | O | —C(═O)-O-Y |
| II-60 | O | S | —C(═O)-O-Y |
| II-61 | O | SO | —C(═O)-O-Y |
| II-62 | O | SO2 | —C(═O)-O-Y |
| II-63 | O | NH | —C(═O)-O-Y |
| II-64 | O | N(alkyl) | —C(═O)-O-Y |
| II-65 | O | CH2 | —C(═O)-amide″ |
| II-66 | O | C═O | —C(═O)-amide″ |
| II-67 | O | O | —C(═O)-amide″ |
| II-68 | O | S | —C(═O)-amide″ |
| II-69 | O | SO | —C(═O)-amide″ |
| II-70 | O | SO2 | —C(═O)-amide″ |
| II-71 | O | NH | —C(═O)-amide″ |
| II-72 | O | N(alkyl) | —C(═O)-amide″ |
| II-73 | S | CH2 | —CH2-OH |
| II-74 | S | C═O | —CH2-OH |
| II-75 | S | O | —CH2-OH |
| II-76 | S | S | —CH2-OH |
| II-77 | S | SO | —CH2-OH |
| II-78 | S | SO2 | —CH2-OH |
| II-79 | S | NH | —CH2-OH |
| II-80 | S | N(alkyl) | —CH2-OH |
| II-81 | S | CH2 | —C(═O)-O-Y |
| II-82 | S | C═O | —C(═O)-O-Y |
| II-83 | S | O | —C(═O)-O-Y |
| II-84 | S | S | —C(═O)-O-Y |
| II-85 | S | SO | —C(═O)-O-Y |
| II-86 | S | SO2 | —C(═O)-O-Y |
| II-87 | S | NH | —C(═O)-O-Y |
| II-88 | S | N(alkyl) | —C(═O)-O-Y |
| II-89 | S | CH2 | —C(═O)-amide″ |
| II-90 | S | C═O | —C(═O)-amide″ |
| II-91 | S | O | —C(═O)-amide″ |
| II-92 | S | S | —C(═O)-amide″ |
| II-93 | S | SO | —C(═O)-amide″ |
| II-94 | S | SO2 | —C(═O)-amide″ |
| II-95 | S | NH | —C(═O)-amide″ |
| II-96 | S | N(alkyl) | —C(═O)-amide″ |
| II-97 | SO | CH2 | —CH2-OH |
| II-98 | SO | C═O | —CH2-OH |
| II-99 | SO | O | —CH2-OH |
| II-100 | SO | S | —CH2-OH |
| II-101 | SO | SO | —CH2-OH |
| II-102 | SO | SO2 | —CH2-OH |
| II-103 | SO | NH | —CH2-OH |
| II-104 | SO | N(alkyl) | —CH2-OH |
| II-105 | SO | CH2 | —C(═O)-O-Y |
| II-106 | SO | C═O | —C(═O)-O-Y |
| II-107 | SO | O | —C(═O)-O-Y |
| II-108 | SO | S | —C(═O)-O-Y |
| II-109 | SO | SO | —C(═O)-O-Y |
| II-110 | SO | SO2 | —C(═O)-O-Y |
| II-111 | SO | NH | —C(═O)-O-Y |
| II-112 | SO | N(alkyl) | —C(═O)-O-Y |
| II-113 | SO | CH2 | —C(═O)-amide″ |
| II-114 | SO | C═O | —C(═O)-amide″ |
| II-115 | SO | O | —C(═O)-amide″ |
| II-116 | SO | S | —C(═O)-amide″ |
| II-117 | SO | SO | —C(═O)-amide″ |
| II-118 | SO | SO2 | —C(═O)-amide″ |
| II-119 | SO | NH | —C(═O)-amide″ |
| II-120 | SO | N(alkyl) | —C(═O)-amide″ |
| II-121 | SO2 | CH2 | —CH2-OH |
| II-122 | SO2 | C═O | —CH2-OH |
| II-123 | SO2 | O | —CH2-OH |
| II-124 | SO2 | S | —CH2-OH |
| II-125 | SO2 | SO | —CH2-OH |
| II-126 | SO2 | SO2 | —CH2-OH |
| II-127 | SO2 | NH | —CH2-OH |
| II-128 | SO2 | N(alkyl) | —CH2-OH |
| II-129 | SO2 | CH2 | —C(═O)-O-Y |
| II-130 | SO2 | C═O | —C(═O)-O-Y |
| II-131 | SO2 | O | —C(═O)-O-Y |
| II-132 | SO2 | S | —C(═O)-O-Y |
| II-133 | SO2 | SO | —C(═O)-O-Y |
| II-134 | SO2 | SO2 | —C(═O)-O-Y |
| II-135 | SO2 | NH | —C(═O)-O-Y |
| II-136 | SO2 | N(alkyl) | —C(═O)-O-Y |
| II-137 | SO2 | CH2 | —C(═O)-amide″ |
| II-138 | SO2 | C═O | —C(═O)-amide″ |
| II-139 | SO2 | O | —C(═O)-amide″ |
| II-140 | SO2 | S | —C(═O)-amide″ |
| II-141 | SO2 | SO | —C(═O)-amide″ |
| II-142 | SO2 | SO2 | —C(═O)-amide″ |
| II-143 | SO2 | NH | —C(═O)-amide″ |
| II-144 | SO2 | N(alkyl) | —C(═O)-amide″ |
| II-145 | NH | CH2 | —CH2-OH |
| II-146 | NH | C═O | —CH2-OH |
| II-147 | NH | O | —CH2-OH |
| II-148 | NH | S | —CH2-OH |
| II-149 | NH | SO | —CH2-OH |
| II-150 | NH | SO2 | —CH2-OH |
| II-151 | NH | NH | —CH2-OH |
| II-152 | NH | N(alkyl) | —CH2-OH |
| II-153 | NH | CH2 | —C(═O)-O-Y |
| II-154 | NH | C═O | —C(═O)-O-Y |
| II-155 | NH | O | —C(═O)-O-Y |
| II-156 | NH | S | —C(═O)-O-Y |
| II-157 | NH | SO | —C(═O)-O-Y |
| II-158 | NH | SO2 | —C(═O)-O-Y |
| II-159 | NH | NH | —C(═O)-O-Y |
| II-160 | NH | N(alkyl) | —C(═O)-O-Y |
| II-161 | NH | CH2 | —C(═O)-amide″ |
| II-162 | NH | C═O | —C(═O)-amide″ |
| II-163 | NH | O | —C(═O)-amide″ |
| II-164 | NH | S | —C(═O)-amide″ |
| II-165 | NH | SO | —C(═O)-amide″ |
| II-166 | NH | SO2 | —C(═O)-amide″ |
| II-167 | NH | NH | —C(═O)-amide″ |
| II-168 | NH | N(alkyl) | —C(═O)-amide″ |
| II-169 | N(alkyl) | CH2 | —CH2-OH |
| II-170 | N(alkyl) | C═O | —CH2-OH |
| II-171 | N(alkyl) | O | —CH2-OH |
| II-172 | N(alkyl) | S | —CH2-OH |
| II-173 | N(alkyl) | SO | —CH2-OH |
| II-174 | N(alkyl) | SO2 | —CH2-OH |
| II-175 | N(alkyl) | NH | —CH2-OH |
| II-176 | N(alkyl) | N(alkyl) | —CH2-OH |
| II-177 | N(alkyl) | CH2 | —C(═O)-O-Y |
| II-178 | N(alkyl) | C═O | —C(═O)-O-Y |
| II-179 | N(alkyl) | O | —C(═O)-O-Y |
| II-180 | N(alkyl) | S | —C(═O)-O-Y |
| II-181 | N(alkyl) | SO | —C(═O)-O-Y |
| II-182 | N(alkyl) | SO2 | —C(═O)-O-Y |
| II-183 | N(alkyl) | NH | —C(═O)-O-Y |
| II-184 | N(alkyl) | N(alkyl) | —C(═O)-O-Y |
| II-185 | N(alkyl) | CH2 | —C(═O)-amide″ |
| II-186 | N(alkyl) | C═O | —C(═O)-amide″ |
| II-187 | N(alkyl) | O | —C(═O)-amide″ |
| II-188 | N(alkyl) | S | —C(═O)-amide″ |
| II-189 | N(alkyl) | SO | —C(═O)-amide″ |
| II-190 | N(alkyl) | SO2 | —C(═O)-amide″ |
| II-191 | N(alkyl) | NH | —C(═O)-amide″ |
| II-192 | N(alkyl) | N(alkyl) | —C(═O)-amide″ |

wherein in the above mycosporine-like amino acid compounds the phenyl ring may be unsubstituted or substituted, and R4′, R5′, R6′, R4″, R5″, R6″, amide′, Y, amide″ and alkyl have the same meaning as defined in any one of the preceding claims, or a tautomer or a stereoisomer or a salt of the afore-mentioned compounds;
or wherein the mycosporine-like amino acid is selected from the group consisting of 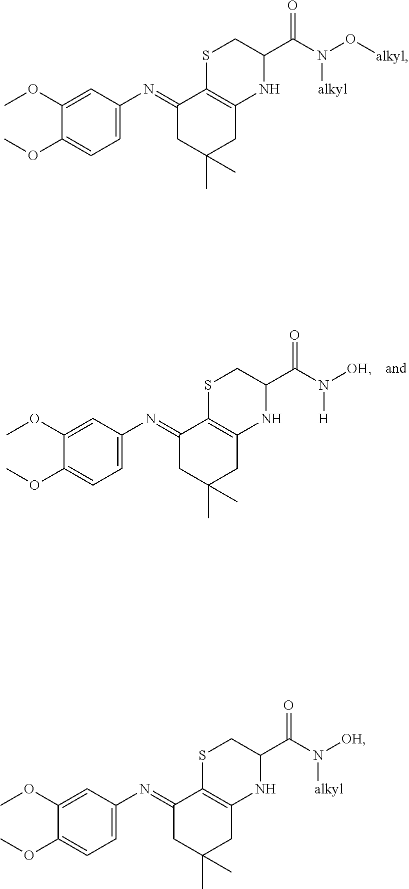
wherein alkyl is either methyl or ethyl or propyl or isopropyl or butyl or isobutyl or tert-butyl, or a tautomer or a stereoisomer or a salt of the afore-mentioned compounds;
or any mixture of the afore-mentioned compounds.

7. The food, cosmetic or pharmaceutical preparation or homecare product according to claim 1, wherein in the general formulae (VI), X is S, SO or SO₂, and/or wherein in the general formulae (V) to (VIII) n=1.

8. The food, cosmetic or pharmaceutical preparation or homecare product according to claim 1, wherein in the general formulae (V), (VII) and (VIII) the substituents R4′, R5′ and R6′ which may be identical or different, are independently from each other selected from the group consisting of H, OH, alkyl, alkoxy, —S(═O)₂OH, and sulfonyl;
or
wherein in the general formulae (VI), the substituents R4″, R5″ and R6″ which may be identical or different, are independently from each other selected from the group consisting of H, OH, alkyl, alkoxy, —S(═O)₂OH, and sulfonyl.

9. The food, cosmetic or pharmaceutical preparation or homecare product according to claim 1, wherein in the general formulae (V) to (VIII) the substituted phenyl bonded to the imino functionality is selected from the group consisting of 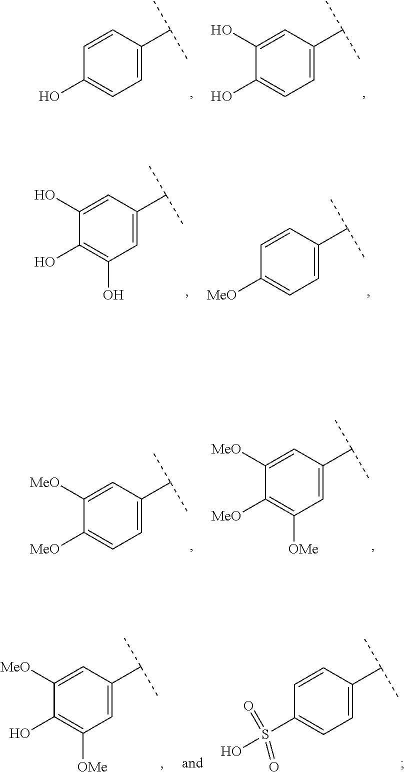
wherein the dotted line designates the binding site.

10. The food, cosmetic or pharmaceutical preparation or homecare product according to claim 1, wherein in the general formulae (V), (VII) and (VIII), the substituents R4′, R5′, R6′, which may be identical or different, are independently from each other selected from the group consisting of H, OH, alkyl, alkoxy, —S(═O)₂OH, and sulfonyl; with the proviso, that if the substituted phenyl ring is monosubstituted, the substituent is either in the ortho or meta-position to the imino-functionality; or wherein in the general formula (VI), the substituents R4″, R5″ and R6″, which may be identical or different, are independently from each other selected from the group consisting of H, OH, alkyl, p alkoxy, —S(═O)₂OH, and sulfonyl; with the proviso, that if the substituted phenyl ring is monosubstituted, the substituent is either in the ortho or meta-position to the imino-functionality of the general formulae (VI), but not in the para position to the imino-functionality.

11. The food, cosmetic or pharmaceutical preparation or homecare product according to claim 1, wherein the mycosporine-like amino acid compound is selected from the group consisting of

| Compound | Structure | INCI/chemical name |
|---|---|---|
| MAA-1 | 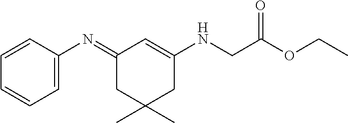 [SMILES: CCOC(=O)CNC1=C/C(=N/c2ccccc2)CC(C)(C)C1] | ethyl 2-[[5,5-dimethyl-3- phenylimino-cyclohexen-1- yl]amino]acetate |
| MAA-2 | 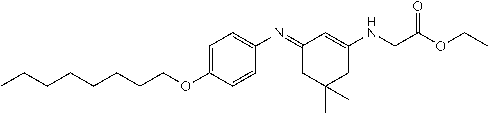 [SMILES: CCCCCCCCOc1ccc(/N=C2/C=C(NCC(=O)OCC)CC(C)(C)C2)cc1] | ethyl 2-((5,5-dimethyl-3-((4- octyoxy)phenyl)imino)cyclohex-1- en-1-yl)amino)acetate |
| MAA-3 | 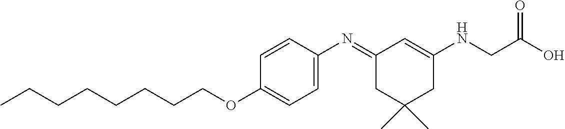 [SMILES: CCCCCCCCOc1ccc(/N=C2/C=C(NCC(=O)O)CC(C)(C)C2)cc1] | 5,5-dimethyl-3-((4- (octyloxy)phenyl)imino) cyclohex-1-en-1-yl-glycine |
| MAA-4 | 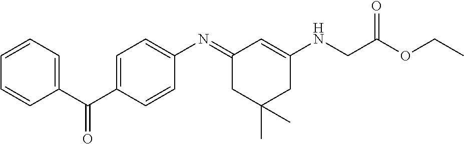 [SMILES: CCOC(=O)CNC1=C/C(=N/c2ccc(C(=O)c3ccccc3)cc2)CC(C)(C)C1] | ethyl 2-((3-((4- benzoylphenyl)imino)-5,5- dimethylcyclohex-1-en-1- yl)amino)acetate |
| MAA-5 | 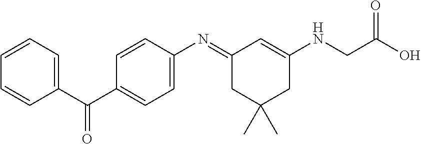 [SMILES: CC1(C)CC(NCC(=O)O)=C/C(=N/c2ccc(C(=O)c3ccccc3)cc2)C1] | 3-((3-benzoylphenyl)imino)-5,5- dimethylcyclohex-1-en-1-yl- glycine |
| MAA-6 | 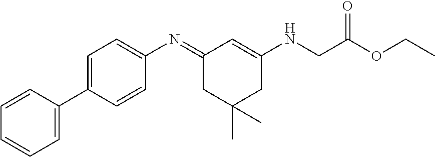 [SMILES: CCOC(=O)CNC1=C/C(=N/c2ccc(-c3ccccc3)cc2)CC(C)(C)C1] | ethyl 2-((3-([1,1′-biphenyl]-4- ylimino)-5,5-dimethylcyclohex-1- en-1.yl)amino)acetate |
| MAA-7 | 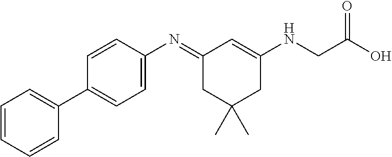 [SMILES: CC1(C)CC(NCC(=O)O)=C/C(=N/c2ccc(-c3ccccc3)cc2)C1] | 3-([1,1′-biphenyl]-4-ylimino)-5,5- dimethylcyclohex-1-en-1-yl- glycine |
| MAA-8 | 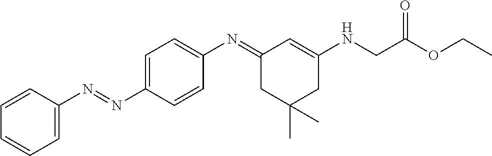 [SMILES: CCOC(=O)CNC1=C/C(=N/c2ccc(/N=N/c3ccccc3)cc2)CC(C)(C)C1] | ethyl 2-((5,5-dimethyl-3-((4- (phenyldiazenyl)phenyl)inino) cyclohex-1-en-1-yl)amino) acetate |
| MAA-9 | 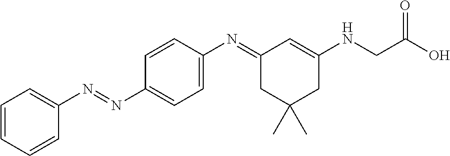 [SMILES: CC1(C)CC(NCC(=O)O)=C/C(=N/c2ccc(/N=N/c3ccccc3)cc2)C1] | 5,5-dimethyl-3-(4-((E)- phenyldiazenyl)phenyl)imino) cyclohex-1-en-1-yl-glycine |
| MAA-11 | 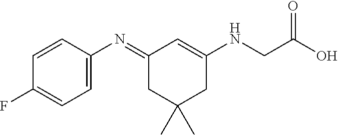 [SMILES: CC1(C)CC(NCC(=O)O)=C/C(=N/c2ccc(F)cc2)C1] | 3-((4-fluorophenyl)imino)-5,5- dimethylcyclohex-1-en-1-yl- glycine |
| MAA-12 | 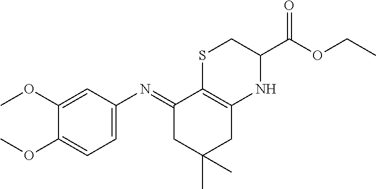 [SMILES: CCOC(=O)C1CSC2=C(CC(C)(C)C/C2=N\c2ccc(OC)c(OC)c2)N1] | ethyl 8-((3,4- dimethoxyphenyl)imino)-6,6- dimethyl-3,4,5,6,7,8-hexahydro- 2-H-benzo[b][1,4]thiazine-3- carboxylate |
| MAA-13 | 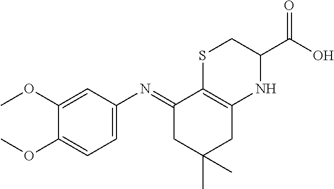 [SMILES: COc1ccc(/N=C2\CC(C)(C)CC3=C2SCC(C(=O)O)N3)cc1OC] | 8-((3,4-dimethoyxphenyl)imino)- 6,6-dimethyl-3,4,5,6,7,8- hexahydro-2-H-benzo[b][1,4] thiazine-3-carboxylic acid |
| MAA-14 | 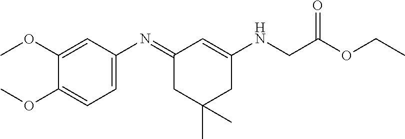 [SMILES: CCOC(=O)CNC1=C/C(=N/c2ccc(OC)c(OC)c2)CC(C)(C)C1] | ethyl 2-((3-((3,4- dimethoxyphenyl)imino)-5,5- dimethylcyclohex-1-en-1- yl)amino)acetate |
| MAA-16 | 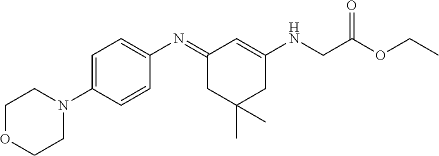 [SMILES: CCOC(=O)CNC1=C/C(=N/c2ccc(N3CCOCC3)cc2)CC(C)(C)C1] | ethyl 2-((5,5-dimethyl-3-((4- morpholinophenyl)imino) cyclohex-1-en-1-yl)amino) acetate |
| MAA-17 | 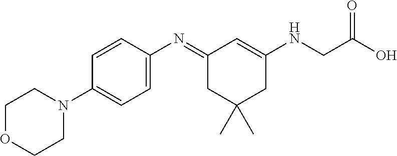 [SMILES: CC1(C)CC(NCC(=O)O)=C/C(=N/c2ccc(N3CCOCC3)cc2)C1] | 5,5-dimethyl-3-((4- morpholinophenyl)imino) cyclohex-1-en-1-yl-glycine |
| MAA-20 | 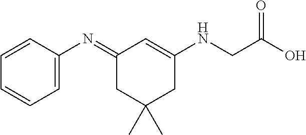 [SMILES: CC1(C)CC(NCC(=O)O)=C/C(=N/c2ccccc2)C1] | 2-[(5,5-dimethyl-3-phenylimino- cyclohexen-1-yl)amino]acetic acid |
| MAA-21 | 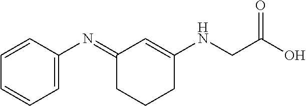 [SMILES: O=C(O)CNC1=C/C(=N/c2ccccc2)CCC1] | 2-[(3-phenyliminocyclohexen-1- yl)amino]acetic acid |
| MAA-22 | 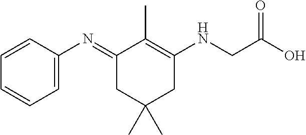 [SMILES: CC1=C(NCC(=O)O)CC(C)(C)C/C1=N\c1ccccc1] | 2,5,5-trimethyl-3- (phenylimino)cyclohex-1-en-1- yl-glycine |
| MAA-23 | 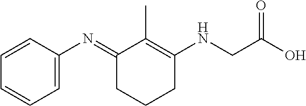 [SMILES: CC1=C(NCC(=O)O)CCC/C1=N\c1ccccc1] | 2-[(2-methyl-3-phenylimino- cyclohexen-1-yl)amino]acetic acid |
| MAA-24 | 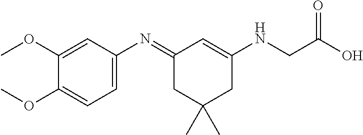 [SMILES: COc1ccc(/N=C2/C=C(NCC(=O)O)CC(C)(C)C2)cc1OC] | 3-((3,4-dimethoxyphenyl)imino)- 5,5-dimethylcyclohex-1-en-1- yl-glycine |
| MAA-25 | 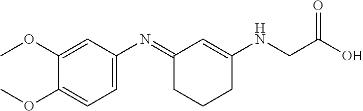 [SMILES: COc1ccc(/N=C2/C=C(NCC(=O)O)CCC2)cc1OC] | 3-((3,4- dimethoxyphenyl)imino) cyclohex-1-en-1-yl-glycine |
| MAA-26 | 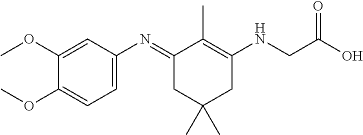 [SMILES: COc1ccc(/N=C2\CC(C)(C)CC(NCC(=O)O)=C2C)cc1OC] | 3-((3,4-dimethoxyphenyl)imino)- 2,5,5-trimethylcyclohex-1-en-1- yl-glycine |
| MAA-27 | 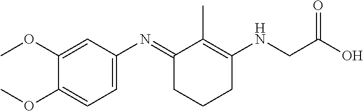 [SMILES: COc1ccc(/N=C2\CCCC(NCC(=O)O)=C2C)cc1OC] | 2-[(3-(3,4-dimethoxyphenyl) imino-2-methyl-cyclohexen-1- yl)amino]acetic acid |
| MAA-28 | 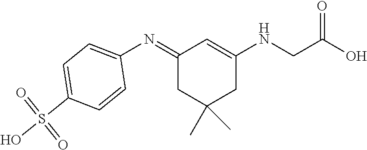 [SMILES: CC1(C)CC(NCC(=O)O)=C/C(=N/c2ccc(S(=O)(=O)O)cc2)C1] | 2-[(5,5-dimethyl-3-(4- sulfophenyl)imino-cyclohexen-1- yl)amino]acetic acid |
| MAA-29 | 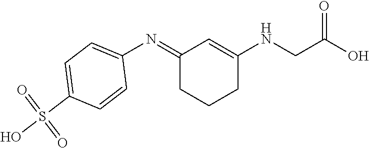 [SMILES: O=C(O)CNC1=C/C(=N/c2ccc(S(=O)(=O)O)cc2)CCC1] | 2-[(3-(4- sulfophenyl)iminocyclohexen-1- yl)amino]acetic acid |
| MAA-30 | 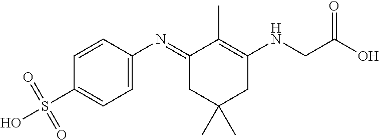 [SMILES: CC1=C(NCC(=O)O)CC(C)(C)C/C1=N\c1ccc(S(=O)(=O)O)cc1] | 2,5,5-trimethyl-3-((4- sulfophenyl)imino)cyclohex-1- en-1-yl-glycine |
| MAA-31 | 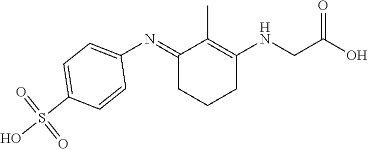 [SMILES: CC1=C(NCC(=O)O)CCC/C1=N\c1ccc(S(=O)(=O)O)cc1] | 2-methyl-3-((4- sulfophenyl)imino)cyclohex-1-en- 1-yl-glycine |
| MAA-32 | 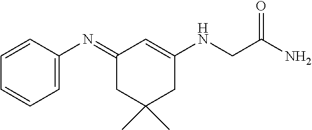 [SMILES: CC1(C)CC(NCC(N)=O)=C/C(=N/c2ccccc2)C1] | 2-[(5,5-dimethyl-3-phenylimino- cyclohexen-1-yl)amino]actamide |
| MAA-33 | 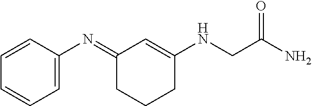 [SMILES: NC(=O)CNC1=C/C(=N/c2ccccc2)CCC1] | 2-[(3-phenyliminocyclohexen-1- yl)amino]acetamide |
| MAA-34 | 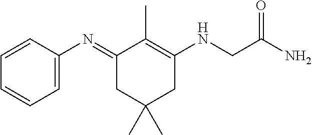 [SMILES: CC1=C(NCC(N)=O)CC(C)(C)C/C1=N\c1ccccc1] | 2-((2,5,5-trimethyl-3- (phenylimino)cyclohex-1-en-1- yl)amino)acetamide |
| MAA-35 | 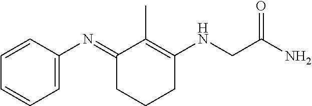 [SMILES: CC1=C(NCC(N)=O)CCC/C1=N\c1ccccc1] | 2-((2-methyl-3- (phenylimino)cyclohex-1-en-1- yl)amino)acetamide |
| MAA-36 | 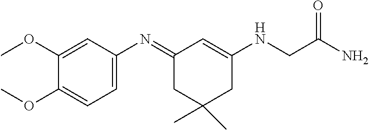 [SMILES: COc1ccc(/N=C2/C=C(NCC(N)=O)CC(C)(C)C2)cc1OC] | 2-((3-((3,4-dimethoxyphenyl) imino)-5,5-dimethylcyclohex-1- en-1-yl)amino)acetamide |
| MAA-37 | 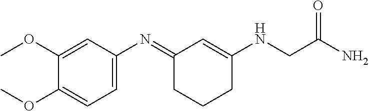 [SMILES: COc1ccc(/N=C2/C=C(NCC(N)=O)CCC2)cc1OC] | 2-[(3-(3,4- dimethoxyphenyl)iminocyclo- hexen-1-yl)amino]acetamide |
| MAA-38 | 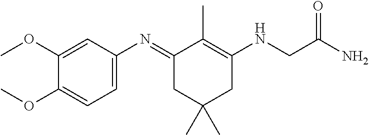 [SMILES: COc1ccc(/N=C2\CC(C)(C)CC(NCC(N)=O)=C2C)cc1OC] | 2-((3-((3,4-dimethoxyphenyl) imino)-2,5,5-trimethylcyclohex- 1-en-1-yl)amino)acetamide |
| MAA-39 | 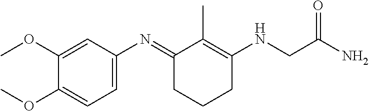 [SMILES: COc1ccc(/N=C2\CCCC(NCC(N)=O)=C2C)cc1OC] | 2-((3-((3,4-dimethoxyphenyl) imino)-2-methylcyclohex-1-en-1- yl)amino)acetamide |

or a tautomer or a stereoisomer or a salt of the afore-mentioned compounds;
or any mixture of the afore-mentioned compounds.

12. The food, cosmetic or pharmaceutical preparation or homecare product according to claim 1, wherein the at least one fragrance compound is selected from the group consisting of
extracts of natural raw;
aliphatic alcohols;
aliphatic aldehydes and the acetals thereof;
aliphatic ketones and the oximes thereof;
aliphatic sulphur-containing compounds;
aliphatic nitriles;
esters of aliphatic carboxylic acids;
acyclic terpene alcohols;
acyclic terpene aldehydes and ketones;
cyclic terpene alcohols;
cyclic terpene aldehydes and ketones;
cyclic alcohols;
cyclic and cycloaliphatic ethers;
cyclic and macrocyclic ketones;
cycloaliphatic aldehydes;
cycloaliphatic ketones;
esters of cyclic alcohols;
esters of cycloaliphatic alcohols;
esters of cycloaliphatic carboxylic acids;
araliphatic alcohols;
esters of araliphatic alcohols and aliphatic carboxylic acids;
araliphatic ethers;
aromatic and araliphatic aldehydes;
aromatic and araliphatic ketones;
aromatic and araliphatic carboxylic acids and the esters thereof;
nitrogenous aromatic compounds;
phenols, phenyl ethers and phenyl esters;
heterocyclic compounds;
lactones; and any mixture of the above fragrance substances;
or wherein the at least one aroma/flavour compound is selected from the group consisting of acetophenone, allyl caproate, alpha-ionone, beta-ionone, anisaldehyde, anisyl acetate, anisyl formate, anethole, benzaldehyde, Benzothiazole, benzyl acetate, benzyl alcohol, benzyl benzoate, beta-ionone, butyl butyrate, butyl caproate, butylidenphthalide, carvone, camphene, caryophyllene, cineole, cinnamyl acetate, Citral, Citronellol, Citronellal, Citronellyl acetate, cyclohexyl acetate, cymene, damascone, decalactone, dihydrocoumarin, Dimethyl anthranilate, dimethyl anthranilate, dodecalactone, ethoxyethyl acetate, ethyl butyric acid, ethyl butyrate, ethyl caprate, ethyl caproate, ethyl crotonate, ethyl furaneol, ethyl guaiacol, ethyl isobutyrate, ethyl isovalerate, ethyl lactate, ethyl methyl butyrate, ethyl propionate, Eucalyptol, Eugenol, Ethyl heptylate, 4-(p-Hydroxyphenyl)-2-butanone, gamma-decalactone, Geraniol, geranyl acetate, geranyl acetate, grapefruit aldehyde, methyl dihydrojasmonate, Heliotropin, 2-heptanone, 3-heptanone, 4-heptanone, trans-2-heptenal, cis-4-heptenal, trans-2-Hexenal, cis-3-Hexenol, trans-2-hexenoic acid, Trans-3-hexenoic acid, cis-2-hexenyl acetate, cis-3-hexenyl acetate, cis-3-hexenyl caproate, trans-2-hexenyl caproate, cis-3-hexenyl formate, cis-2-hexyl acetate, cis-3-hexyl acetate, trans-2-hexyl acetate, cis-3-hexyl formate, para-hydroxybenzylacetone, isoamyl alcohol, isoamyl isovalerate, isobutyl butyrate, isobutyraldehyde, Isoeugenol methyl ether, isopropylmethylthiazole, lauric acid, leavulic acid, Linalool, linalool oxide, linalyl acetate, Menthol, Menthofuran, methyl anthranilate, methylbutanol, methylbutyric acid, 2-methylbutyl acetate, methyl caproate, methyl cinnamate, 5-methylfurfural, 3,2,2-methylcyclopentenolone, 6,5,2-methylheptenone, methyl dihydrojasmonate, methyl jasmonate, 2-methylmethylbutyrate, 2-Methyl-2-pentenoleic acid, methyl thiobutyrate, 3,1-methylthiohexanol, 3-methylthiohexyl acetate, Nerol, neryl acetate, trans, trans-2,4-Nonadienal, 2,4-nonadienol, 2,6-nonadienol, 2,4-nonadienol, nootkatone, Delta-octactone, gamma-octactone, 2-Octanol, 3-Octanol, 1,3-Octenol, 1-octyl acetate, 3-octyl acetate, palmitic acid, paraldehyde, phellandrene, pentanedione, phenylethyl acetate, phenylethyl alcohol, phenylethyl alcohol, phenylethyl isovalerate, Piperonal, propionaldehyde, propyl butyrate, pulegone, Pulegol, Sinensal, Sulfurol, terpinene, Terpineol, terpinolene, 8,3-thiomenthanone, 4,4,2-thiomethylpentanone, Thymol, delta-undecalactone, gamma-undecalactone, valencene, valeric acid, Vanillin, Acetoin, ethylvanillin, ethylvanillin isobutyrate (=3-Ethoxy-4-isobutyryloxybenzaldehyde), 2,5-Dimethyl-4-hydroxy-3(2H)-furanone and the derivatives thereof, Maltol and maltol derivatives, Coumarin and coumarin derivatives, gamma-lactones, delta-lactones, methyl sorbate, Divanillin, 4-Hydroxy-2 (or 5)-ethyl-5 (or 2)-methyl-3(2H)-furanone, 2-Hydroxy-3-methyl-2-cyclopentenone, 3-Hydroxy-4,5-dimethyl-2(5H)-furanone, isoamyl acetate, ethyl butyrate, n-butyl butyrate, isoamyl butyrate, ethyl 3-methylbutyrate, ethyl n-hexanoate, allyl n-hexanoate, N-butyl n-hexanoate, ethyl n-octanoate, Ethyl 3-methyl-3-phenylglycidate, Ethyl 2-trans-4-cis-decadienoate, 4-(p-Hydroxyphenyl)-2-butanone, 1,1-Dimethoxy-2,2,5-trimethyl-4-hexane, 2,6-Dimethyl-5-hepten-1-al and phenylacetaldehyde, 2-Methyl-3-(methylthio) furan, 2-Methyl-3-furanthiol, Bis(2-methyl-3-furyl)disulfide, furfuryl mercaptan, Methional, 2-Acetyl-2-thiazoline, 3-Mercapto-2-pentanone, 2,5-Dimethyl-3-furanthiol, 2,4,5-trimethylthiazole, 2-acetylthiazole, 2,4-Dimethyl-5-ethylthiazole, 2-Acetyl-1-pyrroline, 2-Methyl-3-ethylpyrazine, 2-Ethyl-3,5-dimethylpyrazine, 2-Ethyl-3,6-dimethylpyrazine, 2,3-Diethyl-5-methylpyrazine, 3-Isopropyl-2-methoxypyrazine, 3-Isobutyl-2-methoxypyrazine, 2-acetylpyrazine, 2-pentylpyridine, (E,E)-2,4-Decadienal, (E,E)-2,4-Nonadienal, (E)-2-Octenal, (E)-2-Nonenal, 2-undecenal, 12-methyltridecanal, 1-Penten-3-one, 4-Hydroxy-2,5-dimethyl-3(2H)-furanone, Guaiacol, 3-Hydroxy-4,5-dimethyl-2(5H)-furanone, 3-Hydroxy-4-methyl-5-ethyl-2(5H)-furanone, cinnamaldehyde, cinnamyl alcohol, methyl salicylate, Isopulegol, sugar alcohols; proteins; sugar substitutes/bulk sweeteners; SUESS-TASTING amino acids; SUESS-TASTING low molecular weight substances, plant extracts and any mixture of the above aroma/flavour substances.

13. The food, cosmetic or pharmaceutical preparation or homecare product according to claim 1, wherein the at least one antioxidant is selected from the group consisting of dimethylmethoxy chromanol, arbutin, amino acids and their derivatives, tert-butylhydroquinone, caffeic acid, chlorogenic acid, imidazoles and their derivatives, cannabinoids, cannabidiol and its extracts (Cannabis sativa seed oil, Cannabis sativa extract), cannabinol, kojic acid, peptides, hydroxyphenyl propamidobenzoic acid (dihydroavenanthramide D), diethylhexyl syringylidene malonate, phenylethyl resorcinol, gallic acid and their derivatives, quercetin, hydroxyacetophenone, rosmarinic acid, carotenoids, carotenes, phytoene, phytofluene and their derivatives, lipoic acid and its derivatives, aurothioglucose, propylthiouracil and other thiols and their salts, dilauryl thiodipropionate, pentaerythrityl tetra-di-t-butyl hydroxyhydrocinnamate, distearyl thiodipropionate, thiodipropionic acid and their derivatives, sulphoximine compounds in very low tolerated doses, chelating agents and their derivatives, unsaturated fatty acids and their derivatives, coniferyl benzoate of benzoin resin, rutinic acid and its derivatives, ferrulic acid and its derivatives, butylhydroxytoluene, butylhydroxyanisole, hydroxymethoxyphenyl decanone, nordihydroguaiacic acid, nordihydroguaiaretic acid, allantoin, tropolone, trihydroxybutyrophenone, uric acid and its derivatives, urea, mannose and its derivatives, hydroxy acetophenone (ortho/para), zinc and its derivatives, beta-aspartyl arginine, selenium and its derivatives, Vitis vinifera (Grape) seed extract, oat extract, Cichorium intubybus (chicory) leaf extract, Leon-topodium alpinum extract, green tea extract, Curcumin, Zingiber officinalis (Ginger Root) Extract, Silymarin, stilbenes and their derivatives, derivatives of said antioxidants, and mixtures of two or more of said antioxidants.

14. The food, cosmetic or pharmaceutical preparation or homecare product according to claim 1, comprising the at least one fragrance or aroma/flavour compound or a mixture thereof in an amount of 0.01 to 40.0% by weight, based on the total weight of the composition or product.

15. The food, cosmetic or pharmaceutical preparation or homecare product according to any one of claims 1 to 14, comprising the mycosporine-like amino acid compound or a mixture thereof in an amount of 0.001 to 15.0% by weight, based on the total weight of the composition or product.

16. The food, cosmetic or pharmaceutical preparation or homecare product according to claim 1, comprising the antioxidant or a mixture thereof in an amount of 0.0001 to 10.0% by weight, based on the total weight of the composition or product.

17. The food, cosmetic or pharmaceutical preparation or homecare product according to any one of claims 1 to 16, further comprising at least one 1,2-alkanediol or one 2,3-alkanediol with 5 to 12 carbon atoms, and/or further comprising one or more antimicrobial agents.

18. (canceled)

19. The food, cosmetic or pharmaceutical preparation or homecare product according to claim 1, further comprising at least one excipient and/or active substance selected from the group consisting of agents against ageing of the skin, emulsifiers, preservatives, UV-filters, silicones, chelating agents, surfactants, green and synthetic polymers, skin-cooling agents, rheology additives, oils, carriers and hydrotropes, fragrances or perfume oils, and any mixture of two or more of the afore-mentioned substances.

20. The food, cosmetic or pharmaceutical preparation or homecare product according to claim 1 in the form of
(i) a dispersion;
(ii) a liquid surfactant formulation;
(iii) a solid surfactant formulation;
(iv) a water free formulation; or
(v) an aqueous aqueous/alcoholic, aqueous/glycolic, or alcoholic/glycolic solution.

21-22. (canceled)

23. A method of stabilizing a fragrance or aroma/flavour compound and/or boosting the antioxidative effect of an antioxidant, comprising mixing an effective amount of at least one mycosporine-like amino acid compound or a mixture thereof with a fragrance or aroma/flavour compound, or with an antioxidant.
